(19) United States
(12) Patent Application Publication
(10) Pub. No.: US 2025/0376468 A1
(43) Pub. Date: Dec. 11, 2025

(54) PYRAZOLO[1,5-a]PYRIDINE DERIVATIVES FOR TREATING TRPM3-MEDIATED DISORDERS

(71) Applicants: Katholieke Universiteit Leuven, Leuven (BE); Biohaven Therapeutics Ltd., New Haven, CT (US)

(72) Inventors: Arnaud MARCHAND, Bierbeek (BE); Jean-Christophe VANHERCK, Heverlee (BE); Melanie REICH, Aachen (DE); Sebastian KRÜGER, Aachen (DE); Mohamed KOUKNI, Leuven (BE); Thomas VOETS, Kessel-Lo (BE); Joris VRIENS, Wakkerzeel (BE)

(21) Appl. No.: 18/868,761

(22) PCT Filed: May 25, 2023
(86) PCT No.: PCT/US2023/067446

§ 371 Date: Nov. 24, 2024

Related U.S. Application Data
(60) Provisional application No. 63/345,484, filed on May 25, 2022.

(51) Int. Cl. C07D 471/04; A61K 31/437; A61K 31/444; A61K 31/4545; A61K 31/506

(52) U.S. Cl. CPC C07D 471/04; A61K 31/437; A61K 31/444; A61K 31/4545; A61K 31/506

(57) ABSTRACT

The invention relates to compounds that are useful for the prevention or treatment of TRPM3 mediated disorders, more in particular disorders selected from pain and inflammatory hypersensitivity. The invention also relates to a method for the prevention or treatment of said TRPM3 mediated disorders.

### TECHNICAL FIELD

The invention relates to compounds that are useful for the prevention or treatment of TRPM3 mediated disorders, more in particular disorders selected from pain, inflammatory hypersensitivity and epilepsy. The invention also relates to a method for the prevention or treatment of said TRPM3 mediated disorders.

### BACKGROUND

The TRP superfamily consists of proteins with six transmembrane domains (6TM) that assemble as homo- or heterotetramers to form cation-permeable ion channels. The name TRP originates from the Drosophila trp (transient receptor potential) mutant, which is characterized by a transient receptor potential in the fly photoreceptors in the response to sustained light. In the last 15 years, trp-related channels have been identified in yeast, worms, insects, fish and mammals, including 27 TRPs in humans. Based on sequence homology, TRP channels can be divided into seven subfamilies: TRPC, TRPV, TRPM, TRPA, TRPP, TRPML and TRPN.

Members of the TRP superfamily are expressed in probably all mammalian organs and cell types, and in recent years great progress has been made in the understanding of their physiological role. The tailored selectivity of certain TRP channels enables them to play key roles in the cellular uptake and/or transepithelial transport of Ca²⁺, Mg²⁺ and trace metal ions. Moreover, the sensitivity of TRP channels to a broad array of chemical and physical stimuli, allows them to function as dedicated biological sensors involved in processes ranging from vision to taste, and tactile sensation. In particular, several members of the TRP superfamily exhibit a very high sensitivity to temperature. These so-called thermoTRPs are highly expressed in sensory neurons and/or skin keratinocytes, where they act as primary thermosensors for the detection of innocuous and noxious (painful) temperatures.

It is becoming increasingly clear that TRP channel dysfunction is directly involved in the etiology of various inherited and acquired diseases. Indeed, both loss-of-function and gain-of-function mutations in the TRP channel genes have been identified as the direct cause of inherited diseases, including brachyolmia, hypomagnesemia with secondary hypocalcemia, polycystic kidney disease, mucolipidosis type IV and familial focal segmental glomerulosclerosis. Moreover, TRP channel function/dysfunction has been directly linked to a wide range of pathological conditions, including chronic pain, hypertension, cancer and neurodegenerative disorders.

TRPM3 (Transient receptor potential melastatin 3) represents a promising pharmacological target. TRPM3 is expressed in a large subset of small-diameter sensory neurons from dorsal root and trigeminal ganglia, and is involved in heat sensing. The neurosteroid pregnenolone sulfate is a potent known activator of TRPM3 (Wagner et al., 2008). The neurosteroid pregnenolone sulfate evoked pain in wild type mice but not in knock-out TRPM3 mice. It was also recently shown that CFA induced inflammation and inflammatory pain are eliminated in TRPM3 knock-out mice. Therefore, TRPM3 antagonists could be used as analgesic drugs to counteract pain, such as inflammatory pain (Vriens J. et al. Neuron, May 2011). A relationship between TRPM3 and epilepsy has also been established (see e.g. Eur J Hum Genet. 2019 October; 27(10): 1611-1618; Elife 2020 May 19; 9:e57190. doi: 10.7554/eLife.57190. DOI: 10.7554/eLife.57190; Channels (Austin). 2021; 15(1): 386-397.

TRPM3 is therefore also a potential target for the treatment of epilepsy.

A few TRPM3 antagonists are known, but none of them points towards the compounds of the current invention (Straub I et al. Mol Pharmacol, November 2013). For instance, Liquiritigenin, a postulated TRPM3 blocker has been described to decrease mechanical and cold hyperalgesia in a rat pain model (Chen L et al. Scientific reports, July 2014). There is still a great medical need for novel, alternative and/or better therapeutics for the prevention or treatment of TRPM3 mediated disorders, more in particular for pain such as inflammatory pain and epilepsy. Therapeutics with good potency on a certain type of pain, low level or no side-effects (such as no possibilities for addiction as with opiates, no toxicity) and/or good or better pharmacokinetic or -dynamic properties are highly needed.

The invention provides a class of novel compounds which are antagonists of TRPM3 and can be used as modulators of TRPM3 mediated disorders.

### SUMMARY

The invention provides pyrazolo[1,5-a]pyridine derivatives and pharmaceutical compositions comprising such pyrazolo[1,5-a]pyridine derivatives. The invention also provides pyrazolo[1,5-a]pyridine derivatives for use as a medicament, more in particular for use in the prevention and/or treatment of TRPM3 mediated disorders, especially for use in the prevention and/or treatment of pain and/or inflammatory hypersensitivity and/or epilepsy; and/or for counteracting pain and/or inflammatory hypersensitivity and/or for counteracting epilepsy.

The invention also provides the use of pyrazolo[1,5-a]pyridine derivatives for the manufacture of pharmaceutical compositions or medicaments for the prevention and/or treatment of TRPM3 mediated disorders, especially for the prevention and/or treatment of pain and/or inflammatory hypersensitivity and/or epilepsy; and/or for counteracting pain and/or inflammatory hypersensitivity and/or epilepsy.

The invention also provides a method for the prevention or treatment of a TRPM3 mediated disorder by administering the pyrazolo[1,5-a]pyridine derivatives according to the invention to a subject in need thereof. More in particular, the invention relates to such method for the prevention and/or treatment of pain and/or inflammatory hypersensitivity and/or epilepsy; and/or for counteracting pain and/or inflammatory hypersensitivity and/or epilepsy.

The invention further provides a method for the preparation of the pyrazolo[1,5-a]pyridine derivatives of the invention.

### DETAILED DESCRIPTION OF THE INVENTION

The invention will be further described and in some instances with respect to particular embodiments, but the invention is not limited thereto.

The first aspect of the invention is the provision of a compound of formula (I), a stereo-isomeric form, a physiologically acceptable salt, solvate and/or polymorph thereof

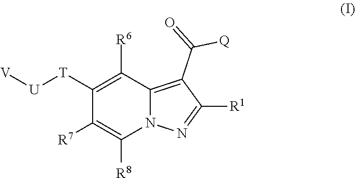

optionally for use in the treatment of pain or epilepsy or methods of treating pain or epilepsy; wherein
R¹ represents —F, —Cl, —Br, —I, —CN, —R^{W}, —OR^{W}, —OC(═O)R^{W}, —NR^{W}R^{X}, —NR^{W}C(═O)R^{X}, —SR^{W}, —S(═O)R^{W}, —S(═O)₂R^{W}, —C(═O)R^{W}, —C(═O)OR^{W}, or —C(═O)NR^{W}R^{X};
Q represents —OR² or —NR³R⁴;
R² represents —R^{Y};
R³ represents —OH or —R^{Y};
R⁴ represents —R^{Y} or —S(═O)₂R^{Y};
or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —CR⁵R^{5′}—; or T represents —CR⁵R^{5′}— and U represents —O—;
R⁵ and R^{5′} independently of one another represent —R^{Y};
R⁶, R⁷ and R⁸ independently of one another represent —F, —Cl, —Br, —I, —CN, —NO₂, —SF₅, —R^{W}, —OR^{W}, —OC(═O)R^{W}, —NR^{W}R^{X}, —NR^{W}C(═O)R^{X}, —SR^{W}, —S(═O)R^{W}, —S(═O)₂R^{W}, —C(═O)R^{W}, —C(═O)OR^{W}, or —C(═O)NR^{W}R^{X};
V represents 3-14-membered heterocycloalkyl, saturated or unsaturated; 3-14-membered cycloalkyl, saturated or unsaturated; 5-14-membered aryl; —C₁-C₆ alkyl, —C₁-C₆ heteroalkyl; or 5-14-membered heteroaryl; in each case unsubstituted, mono- or polysubstituted with substituents independently of one another selected from —F, —Cl, —Br, —I, —CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z};
wherein
R^{W} and R^{X} independently of one another and in each case independently represent —H;
—C₁-C₆-alkyl, saturated or unsaturated, unsubstituted, mono- or polysubstituted; —C₁-C₆-heteroalkyl, saturated or unsaturated, unsubstituted, mono- or polysubstituted;
3-14-membered cycloalkyl, saturated or unsaturated, unsubstituted, mono- or polysubstituted;
wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R^{Y} and R^{Z} independently of one another and in each case independently represent —H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted;
wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
6-14-membered aryl, unsubstituted, mono- or poly-substituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
or R^{Y} and R^{Z} together form a 4, 5, 6, 7 or 8 membered heterocycle containing 1 to 3 heteroatoms selected from N, O and S, saturated or unsaturated, unsubstituted or mono- or polysubstituted;
and wherein “mono- or polysubstituted” in each case independently means substituted with one or more, e.g. 1, 2, 3, 4, or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-O—CF₃, —C₁₋₆-alkylene-O—CF₂H, —C₁₋₆-alkylene-O—CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —N(3-14-membered cycloalkyl)(C₁₋₆-alkyl), —N(C₁₋₆-alkyl)-C₁₋₆-alkylene-OH, —N(H)—C₁₋₆-alkylene-OH, —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁. 6-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered heterocycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, —S(═O)₂-(5 to 14-membered heteroaryl).

In some embodiments of the pyrazolo[1,5-a]pyridine derivatives according to the invention
(a-1) Q represents —NR³R⁴; R¹ represents R^{W}; and R^{W} represents —C₁-C₆-alkyl - and/or
(a-2) Q represents —NR³R⁴; and at least one of R⁵ and R^{5′} represents —H; and/or
(a-3) Q represents —NR³R⁴; and R⁶ represents —H; and/or
(a-4) Q represents —NR³R⁴; and R⁸ represents —H;
or
(b-1)
(b-1) Q represents —NR³R⁴; and R¹ represents —CH₂F, —CHF₂, —CF₃, —CN, -methyl, -ethyl, -propyl or -cyclopropyl; and/or
(b-2) Q represents —NR³R⁴; and at least one of R⁵ and R^{5′} does not represent —H; and/or
(b-3) Q represents —NR³R⁴; and R³ represents —H.

In an embodiment of the pyrazolo[1,5-a]pyridine derivatives according to the invention T represents —O— and U represents —CR⁵R⁵′—. According to this embodiment, the pyrazolo[1,5-a]pyridine derivative according to the invention is a compound of formula (II), a stereo-isomeric form, a physiologically acceptable salt, solvate and/or polymorph thereof

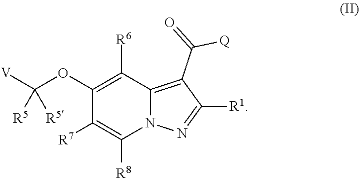

In another embodiment of the pyrazolo[1,5-a]pyridine derivatives according to Formula I, T represents —CR⁵R^{5′}— and U represents —O—.

In an embodiment of the pyrazolo[1,5-a]pyridine derivatives according to Formulas I or II, R¹ is methyl, ethyl, or other C₁-C₆ alkyl. In another preferred embodiment, R¹ is methyl.

In an embodiment of the pyrazolo[1,5-a]pyridine derivatives according to the invention Q represents —NR³R⁴.

In an embodiment of the pyrazolo[1,5-a]pyridine derivatives according to the invention Q represents —OR².

In some embodiments of the pyrazolo[1,5-a]pyridine derivatives according to the invention V represents 3-14-membered cycloalkyl, saturated or unsaturated; 3-14-membered heterocycloalkyl, saturated or unsaturated 5-14-membered aryl; C₁-C₆ alkyl; or 5-14-membered heteroaryl; in each case unsubstituted, mono- or polysubstituted with substituents independently of one another selected from —F, —Cl, —Br, —I, —CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

In some embodiments, the 5-14-membered heteroaryl within the definition of V is selected from benzimidazole, benzisoxazole, benzoxazole, benzodioxole, benzofuran, benzothiadiazole, benzothiazole, benzothiophene, carbazole, cinnoline, dibenzofuran, furane, furazane, imidazole, imidazopyridine, pyrazolo[1,5-a]pyridine, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine; in each case unsubstituted, mono- or polysubstituted with substituents independently of one another selected from —F, —C, —Br, —I, —CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

Preferably, the 5-14-membered heteroaryl within the definition of V is selected from the group consisting of furane, thiophene, imidazole, pyrazole, oxazole, isoxazole, thiazole, triazole, pyridine, isoquinoline, benzothiazole, pyridazine, pyrimidine, imidazopyridine; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

Preferably, the 5-14-membered heteroaryl within the definition of V is selected from the group consisting of furan-2-yl, furan-3-yl, thiophen-2-yl, thiophen-3-yl, pyrazol-3-yl, pyrazol-4-yl, pyrazol-5-yl, oxazol-5-yl, isoxazol-4-yl, thiazol-2-yl, thiazol-4-yl, thiazol-5-yl, 1,2,4-triazol-3-yl, 1,2,3-triazol-4-yl, pyridin-2-yl, pyridin-3-yl, pyridin-4-yl, isoquinolin-1-yl, isoquinolin-5-yl, benzo[d]thiazol-2-yl, pyridazin-3-yl, pyrimidin-5-yl, and imidazo[1,2-a]pyridin-6-yl; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

In some embodiments, the 5-14-membered heteroaryl within the definition of V is selected form the group consisting of pyrazol-3-yl, pyrazol-4-yl, thiazol-4-yl, thiazol-5-yl, pyridin-2-yl, pyridin-3-yl, and pyridine-4-yl; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

In an embodiment, the 3-14-membered cycloalkyl, saturated or unsaturated within the definition of V is cyclopropyl, cyclobutyl, cyclopentyl, cyclohexyl, cycloheptyl, cyclooctyl, cyclononyl, or cyclodecyl including unfused or unbridged, fused, or bridged cycloalkyls; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

In an embodiment, the 5-14-membered aryl within the definition of V is phenyl or another 5-14-membered aryl, unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

In further embodiments of the pyrazolo[1,5-a]pyridine derivatives according to the invention V represents 3-14-membered heterocycloalkyl, saturated or unsaturated, unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

In some embodiments, the 3-14-membered heterocycloalkyl within the definition of V is selected from azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxane, oxepane, oxetane, oxirane, piperazine, piperidine, pyrazolidine, pyrrolidine, quinuclidine, tetrahydrofurane, tetrahydropyrane, tetrahydrothiopyrane, thiazolidine, thietane, thiirane, thiolane, thiomorpholine, indoline, dihydrobenzofuran, dihydrobenzo-thiophene, 1,1-dioxothia-cyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexa-hydro-cyclopenta[c]pyrrole, octahydro-cyclopenta[c]pyrrole, and octahydropyrrolo[1,2-a]pyrazine; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

In some embodiments, the 3-14-membered heterocycloalkyl within the definition of V is oxane, oxan-4-yl, oxetane, or oxetan-3-yl; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

In another preferred embodiment of the pyrazolo[1,5-a]pyridine derivatives according to the invention V represents C₁-C₆ alkyl or C₁-C₆ heteroalkyl, saturated or unsaturated, unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

In some embodiments of the pyrazolo[1,5-a]pyridine derivative according to the invention V is unsubstituted, mono- or polysubstituted with substituents independently of one another selected from
—F, —Cl, —Br, —I, —CN, —C(═O)OH, —NH₂, —NO₂, —OH, ═O, —SF₅;
—C₁₋₆-alkyl, saturated or unsaturated, unsubstituted, mono- or polysubstituted;
—C(═O)O—C₁₋₆-alkyl, saturated or unsaturated, unsubstituted, mono- or polysubstituted;
—NHC₁₋₆-alkyl, saturated or unsaturated, unsubstituted, mono- or polysubstituted;
—N(C₁₋₆-alkyl)₂, saturated or unsaturated, unsubstituted, mono- or polysubstituted;
—O—C₁₋₆-alkyl, saturated or unsaturated, unsubstituted, mono- or polysubstituted;
—S(═O)₂—C₁₋₆-alkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted.

In some embodiments, V is unsubstituted, mono- or polysubstituted with substituents independently of one another selected from —OH, —F, —Cl, —Br, —I, —SH, —CF₃, —CHF₂, —CH₂F, —OCF₃, —OCHF₂, —OCH₂F, SF₅, —CN, —NO₂, —C(═O)OH, —NH₂, or —N(CH₃)₂;
—C₁₋₆-alkyl, saturated or unsaturated, unsubstituted, mono- or polysubstituted with substituents independently of one another, selected from the group consisting of —F, —C, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
—C₁₋₆-heteroalkyl, saturated or unsaturated, unsubstituted, mono- or polysubstituted with substituents independently of one another, selected from the group consisting of —F, —C, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
—OC₁₋₆-alkyl, unsubstituted, mono- or polysubstituted with substituents independently of one another, selected from the group consisting of —F, —C, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
—O(C═O)C₁₋₆-alkyl, unsubstituted, mono- or polysubstituted with substituents independently of one another, selected from the group consisting of —F, —C, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
—C(═O)OC₁₋₆-alkyl, unsubstituted, mono- or polysubstituted with substituents independently of one another, selected from the group consisting of —F, —C, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
3-14-membered cycloalkyl selected from the group consisting of cyclopropyl, cyclobutyl, cyclopentyl, cyclohexyl and cycloheptyl; in each case unsubstituted, mono- or polysubstituted with substituents independently of one another, selected from the group consisting of —F, —C, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
3-14-membered heterocycloalkyl selected from the group consisting of azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, tetrahydrothiopyran, thiazolidine, thietane, thiirane, thiolane, thiomorpholine, indoline, dihydrobenzofuran, dihydrobenzothiophene, 1,1-dioxothiacyclo-hexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexahydro-cyclopenta[c]pyrrole, octahydrocyclopenta[c]pyrrole, and octahydropyrrolo[1,2-a]pyrazine, in each case unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂.

In some embodiments, V is unsubstituted, mono- or polysubstituted with substituents independently of one another selected from —F, —Cl, —CN, —OH, ═O, —C₁₋₆-alkyl, methyl, ethyl, —CHF₂, —CF₃, —C₁₋₆-alkylene-NH₂, —C₁₋₆-alkylene-NHC(═O)O—C₁₋₆-alkyl, —C₁₋₆-alkylene-OH, —C₁₋₆-alkylene-NHC(═O)—O—C₁₋₆-alkyl, —C(═O)O—C₁₋₆-alkyl, —N(C₁₋₆-alkyl)₂, —OC₁₋₆-alkyl, —OCF₃, —O—C₁₋₆-alkylene-N(C₁₋₆-alkyl)₂, —S(═O)₂—C₁₋₆-alkyl, -azetidine, —C₁₋₆-alkylene-O-tetrahydropyran, or -piperazine substituted with —C₁₋₆-alkyl.

In some embodiments of the pyrazolo[1,5-a]pyridine derivative according to the invention V is
(i) unsubstituted;
(ii) monosubstituted;
(iii) disubstituted;
(iv) trisubstituted; or
(v) tetrasubstituted.

In some embodiments of the pyrazolo[1,5-a]pyridine derivatives according to the invention V is
(i) unsubstituted;
(ii) monosubstituted; or
(iii) disubstituted.

In some embodiments, V represents a 3-14-membered heterocycloalkyl (preferably 3-5-membered heterocycloalkyl), saturated or unsaturated; 5-14-membered heteroaryl (preferably 5-6-membered heteroaryl); 3-14-membered cycloalkyl, saturated or unsaturated; 5-14-membered aryl; or C₁-C₆ alkyl; in each case unsubstituted, mono- or polysubstituted; preferably a residue selected from the group consisting of:

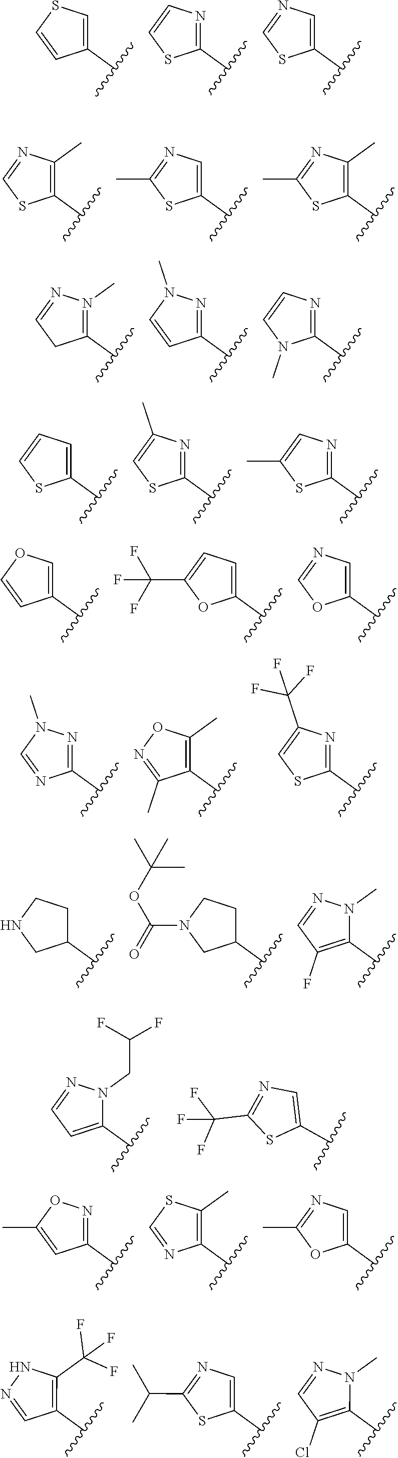

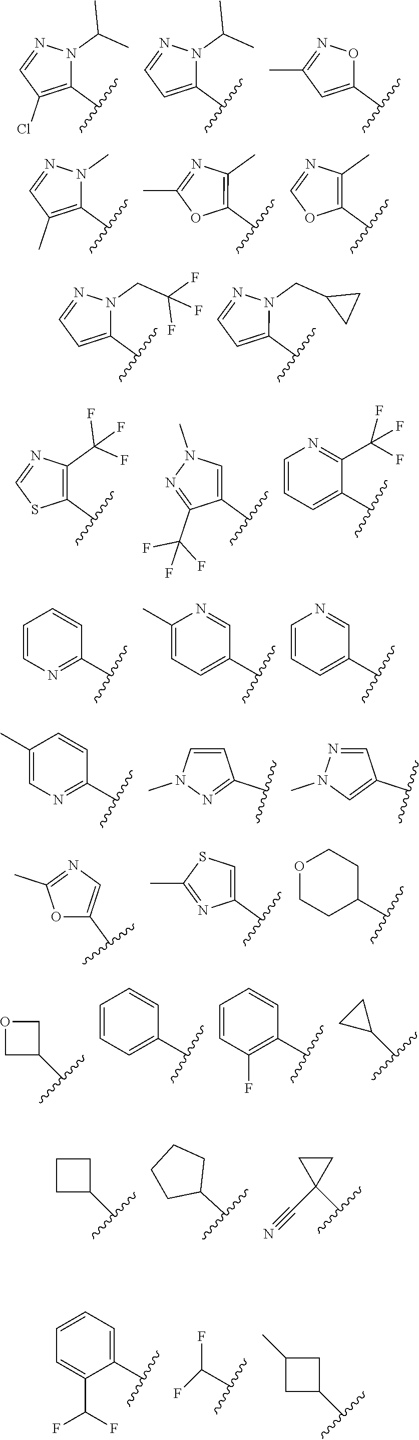

In an embodiment, V represents -oxetanyl, unsubstituted, mono- or polysubstituted; preferably

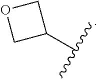 [SMILES: *C1COC1]

In some embodiments, V represents a residue according to general formula (E)

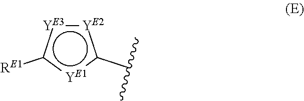

wherein
Y^{E1} represents —N═, —NR^{E2}—, S, O, or —CR^{E3}═; Y^{E2} represents —N═, —NR^{E3}—, S, O, or —CR^{E4}═; and Y^{E3} represent —N═, —NR^{E4}—, S, O, or —CR^{E5}═; with the proviso that at least one of Y^{E1}, Y^{E2} and Y^{E3} is not —CR^{E3}=, —CR^{E4}=, and —CR^{E5}=, respectively. In another preferred embodiment, V represents a residue according to general formula (E)
wherein YE1 represents —N═, —NRE2-, S, or -CRE3=; YE2 represents —N═, —NRE3-, S, or -CRE4=; and Y^{E3} represent —N═, —NRE4-, S, or -CRE5=; with the proviso that at least one of YE1, YE2, and YE3 is not —CRE3=, —CRE4=, and -CRE5=, respectively.

R^{E1}, R^{E2}, R^{E3}, and R^{E4} independently of one another represent —H, —CH₃, —CH₂-cyclopropyl, —CH₂CF₃, —CH₂CHF₂ or —CF₃; more in particular R^{E1}, R^{E2}, R^{E3}, and R^{E4} independently of one another represent —H, —CH₃, or —CF₃; preferably with the proviso that only one of R^{E1}, R^{E2}, R^{E3}, and R^{E4} represents a residue that is not —H.

In some embodiments, V represents 2-pyridine, unsubstituted, mono- or polysubstituted. In some embodiments, V represents a residue selected from the group consisting of:

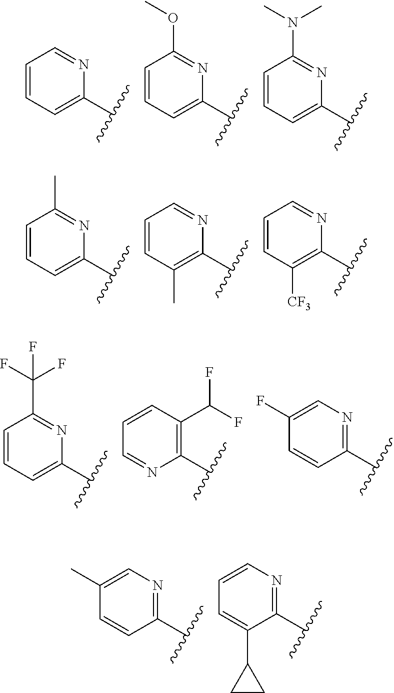

In some embodiments, V represents 3-pyridine, unsubstituted, mono- or polysubstituted. In preferred embodiments, V represents a residue selected from the group consisting of:

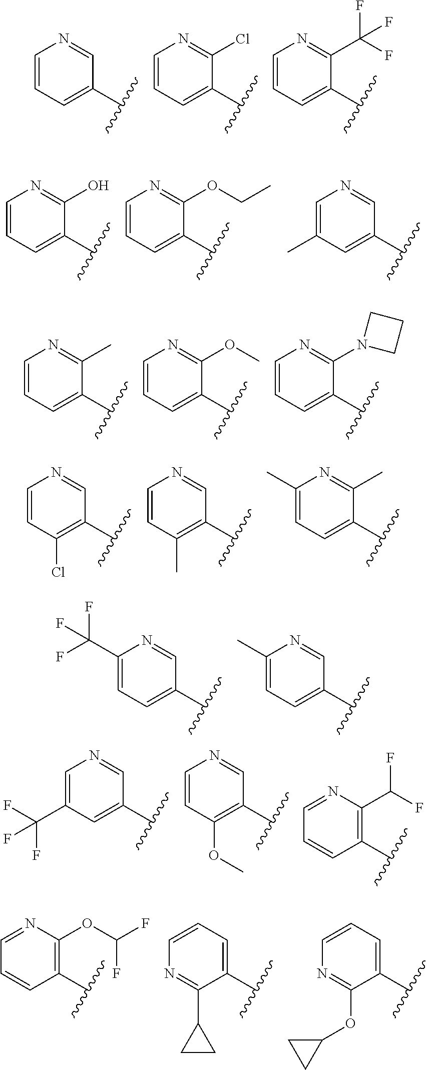

In some embodiments, V represents 4-pyridine, unsubstituted, mono- or polysubstituted. In preferred embodiments, V represents a residue selected from the group consisting of:

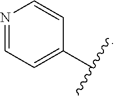 [SMILES: *c1ccncc1]

In some embodiments, optionally where U—CH₂, V represents a residue selected from the group consisting of:

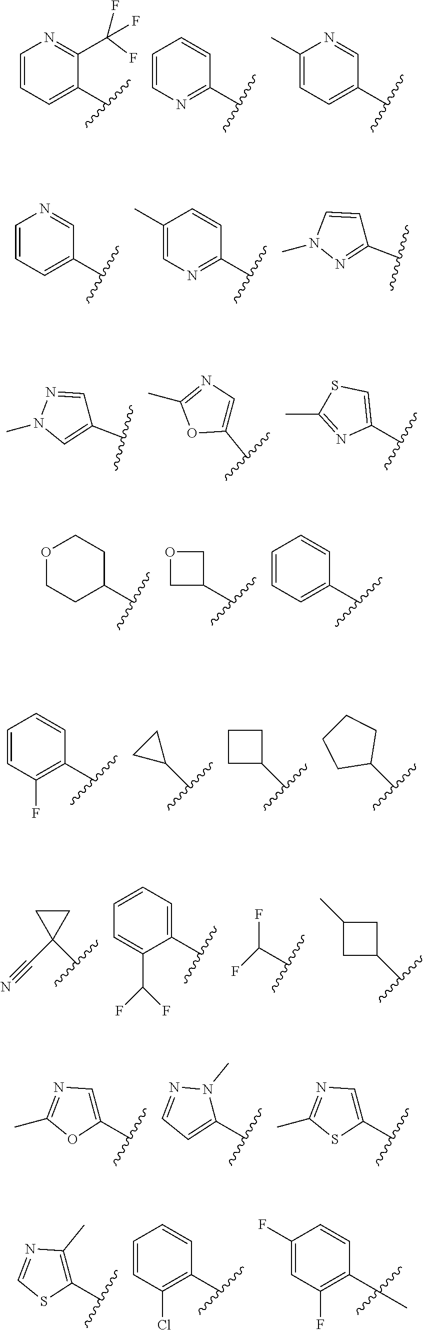

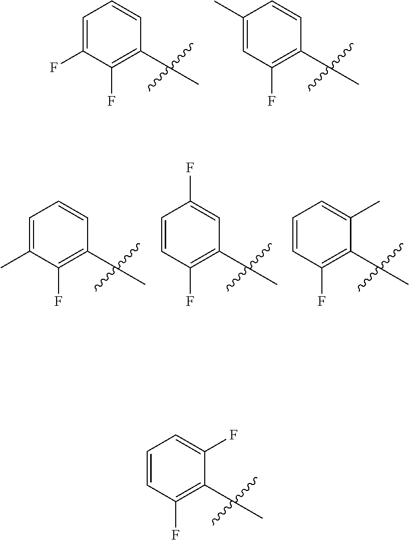

In alternative embodiments, V represents a residue selected from the group consisting of:

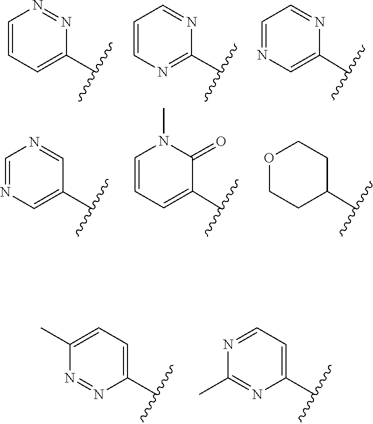

In some embodiments, V represents a bicyclic heteroaryl, unsubstituted, mono- or polysubstituted, preferably selected from the group consisting of:

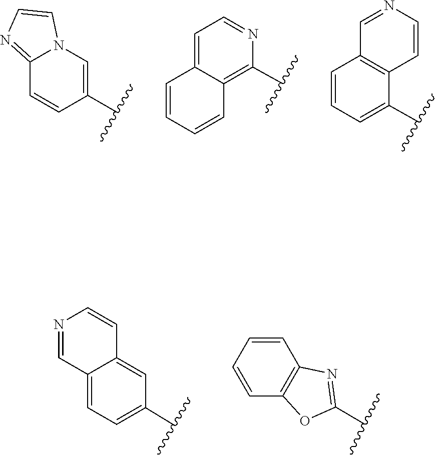 [SMILES: *c1ccc2nccn2c1 ; *c1nccc2ccccc12 ; *c1cccc2cnccc12 ; *c1ccc2cnccc2c1 ; *c1nc2ccccc2o1]

In some embodiments, V represents a residue according to general formula (F′)

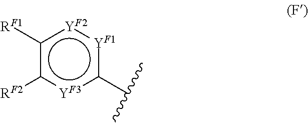

wherein
Y^{F1} represents —N═ or —CR^{F4}=; and Y^{F2} represents —N═ or —CR^{F5}=; and Y^{F3} represents —N═ or —CR^{F3}=; with the proviso that at least one of Y^{F1} and Y^{F2} is not —CR^{F4}═ and —CR^{F5}=, respectively; R^{F1}, R^{F2}, R^{F3}, R^{F4}, and R^{F5} independently of one another represent —H, —CH₃, —CF₃, —OH, —OCH₃, —OCH₂CH₃, —Cl, or -azetidinyl; preferably with the proviso that only one of R^{F1}, R^{F2}, R^{F3}, R^{F4} and R^{F5} represents a residue that is not —H.

In another embodiment, V represents a residue according to general formula (F)

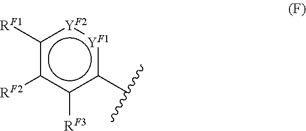

wherein
Y^{F1} represents —N═ or —CR^{F4}═; and Y^{F2} represents —N═ or —CR^{F5}═; with the proviso that at least one of Y^{F1} and Y^{F2} is not —CR^{F4}═ and —CR^{F5}═, respectively;
R^{F1}, R^{F2}, R^{F3}, R^{F4}, and R^{F5} independently of one another represent —H, —CH₃, —CF₃, —OH, —OCH₃, —OCH₂CH₃, —Cl, or -azetidinyl; preferably with the proviso that only one of R^{F1}, R^{F2}, R^{F3}, R^{F4} and R^{F5} represents a residue that is not —H.

In some embodiments, V represents a residue according to general formula (G) or (H)

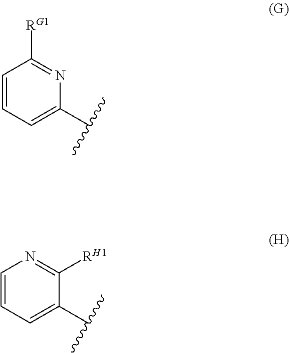

wherein R^{G1} and R^{H1} are selected from the group consisting of —H, —CH₃, —CF₃, —OH, —OCH₃, —OCH₂CH₃, —Cl, azetidinyl, -cyclopropyl, —O-cyclopropyl, and —CHF₂; or wherein R^{G1} and R^{H1} are selected from the group consisting of —H, —CH₃, —CF₃, —OH, —OCH₃, —OCH₂CH₃, —Cl, and azetidinyl.

In other embodiments, V represents a residue according to general formula (G′) or (H′)

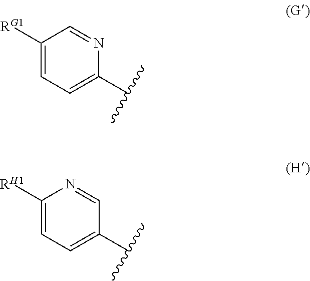

wherein R^{G1} and R^{H1} are selected from the group consisting of —H, —CH₃, —CF₃, —OH, —OCH₃, —OCH₂CH₃, —Cl, azetidinyl, -cyclopropyl, —O-cyclopropyl, and —CHF₂; or wherein R^{G1} and R^{H1} are selected from the group consisting of —H, —CH₃, —CF₃, —OH, —OCH₃, —OCH₂CH₃, —Cl, and azetidinyl;

In an embodiment of the pyrazolo[1,5-a]pyridine derivatives according to the invention R¹ represents
—H, —F, —Cl, —Br, —I, —CN;
—C₁₋₆-alkyl, saturated or unsaturated, unsubstituted, mono- or polysubstituted; —O—C₁₋₆-alkyl, saturated or unsaturated, unsubstituted, mono- or polysubstituted;
—C(═O)C₁₋₆-alkyl, saturated or unsaturated, unsubstituted, mono- or polysubstituted;
—C(═O)OC₁₋₆-alkyl, saturated or unsaturated, unsubstituted, mono- or polysubstituted;
—C(═O)NHC₁₋₆-alkyl, saturated or unsaturated, unsubstituted, mono- or polysubstituted;
—C(═O)N(C₁₋₆-alkyl)₂, saturated or unsaturated, unsubstituted, mono- or polysubstituted;
—S(═O)C₁₋₆-alkyl, saturated or unsaturated, unsubstituted, mono- or polysubstituted;
—S(═O)₂—C₁₋₆-alkyl, saturated or unsaturated, unsubstituted, mono- or polysubstituted;
—C₁-C₆-heteroalkyl, saturated or unsaturated, unsubstituted, mono- or polysubstituted; or
3-14-membered cycloalkyl, saturated or unsaturated, unsubstituted, mono- or polysubstituted;
wherein said 3-14-membered cycloalkyl is optionally connected through —C₁-C₆-alkylene- or —C₁-C₆-heteroalkylene-, in each case saturated or unsaturated, unsubstituted, mono- or polysubstituted.

In some embodiments, R¹ represents —H, —F, —Cl, —Br, —I, —C₁₋₆-alkyl, —O—C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)C₁₋₆-alkyl, —C(═O)OC₁₋₆-alkyl, —C(═O)NH₂, —C(═O)NHC₁₋₆-alkyl, —C(═O)N(C₁₋₆-alkyl)₂, —S(═O)—C₁₋₆-alkyl, —S(═O)₂—C₁₋₆-alkyl, —O—C₁₋₆-alkyl, -cyclopropyl unsubstituted, cyclobutyl unsubstituted, cyclopentyl unsubstituted or cyclohexyl unsubstituted.

In some embodiments, R¹ represents —H, —C₁₋₆-alkyl, —C₁₋₆-alkylene-O—C₁₋₆-alkyl, —CH₂F, —CHF₂, —CF₃, -cyclopentyl, unsubstituted, or -cyclopropyl. Preferably, R¹ represents —H, —C₁₋₆-alkyl, —C₁₋₆-alkylene-O—C₁₋₆-alkyl, —CH₂F, —CHF₂, —CF₃, -cyclopentyl, or unsubstituted. In some embodiments, R¹ represents —CH₃.

In some embodiments, R¹ represents —CH₂F, —CHF₂, —CH₃, or -cyclopropyl. Preferably, R¹ represents —CH₂F, —CHF₂, or —CH₃. In some embodiments, R¹ represents —C(═O)NH₂, or —CHF₂.

In some embodiments, R¹ represents —H, —C₁₋₃-alkyl, —CF₃, —CF₂H, —CFH₂, —CF₂Cl, —CFCl₂, —C₁₋₃-alkylene-CF₃, —C₁₋₃-alkylene-CF₂H, —C₁₋₃-alkylene-CFH₂, or -cyclopropyl; preferably, R¹ represents —H, —C₁₋₃-alkyl, —CF₃, —CF₂H, —CFH₂, —CF₂C₁, —CFCl₂, —C₁₋₃-alkylene-CF₃, —C₁₋₃-alkylene-CF₂H, or —C₁₋₃-alkylene-CFH₂; for example —CH₃.

In some embodiments of the pyrazolo[1,5-a]pyridine derivative according to the invention R² represents
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted;
wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted.

In some embodiments, R² represents —H, —C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, -C₁. 6-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃.

In some embodiments, R² represents —H or —C₁₋₆-alkyl.

In an embodiment of the pyrazolo[1,5-a]pyridine derivative according to the invention R³ represents
—H;
—OH;
—C₁-C₆-alkyl, saturated or unsaturated, unsubstituted, mono- or polysubstituted; or
—C₁-C₆-heteroalkyl, saturated or unsaturated, unsubstituted, mono- or polysubstituted.

In some embodiments, R³ represents —H, —OH, —C₁₋₆-alkyl, —C₁₋₆-alkylene-OH, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃.

In some embodiments, R³ represents —H, —OH, or —C₁₋₆-alkyl, saturated, unsubstituted or monosubstituted with —OH. Preferably, R³ represents —H.

In some embodiments, R³ represents —H and R⁴ represents a residue other than —H.

In an embodiment of the pyrazolo[1,5-a]pyridine derivatives according to the invention R⁴ represents
—H;
—S(═O)C₁₋₆-alkyl, saturated or unsaturated, unsubstituted, mono- or polysubstituted;
—S(═O)₂—C₁₋₆-alkyl, saturated or unsaturated, unsubstituted, mono- or polysubstituted;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted;
wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.

In some embodiments, R⁴ represents
—S(═O)₂C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, -C₁. 6-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
—S(═O)₂(3-14-membered cycloalkyl), wherein said 3-14-membered cycloalkyl is selected from the group consisting of cyclopropyl, cyclobutyl, cyclopentyl, cyclohexyl, and cycloheptyl, in each case saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
—C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁-6-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is selected from the group consisting of cyclopropyl, cyclobutyl, cyclopentyl, cyclohexyl, and cycloheptyl, in each case saturated or unsaturated, in each case unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from the group consisting of azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, tetrahydrothiopyran, thiazolidine, thietane, thiirane, thiolane, thiomorpholine, indoline, dihydrobenzofuran, dihydrobenzothiophene, 1,1-dioxothiacyclo-hexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexahydro-cyclopenta[c]pyrrole, octahydrocyclopenta[c]pyrrole, and octahydropyrrolo[1,2-a]pyrazine; in each case unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
-phenyl unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of benzimidazole, benzisoxazole, benzoxazole, benzodioxole, benzofuran, benzothiadiazole, benzothiazole, benzothiophene, carbazole, cinnoline, dibenzofuran, furane, furazane, imidazole, imidazopyridine, pyrazolo[1,5-a]pyridine, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine; in each case unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted.

In some embodiments, R⁴ represents
—H;
—S(═O)₂C₁₋₆-alkyl, saturated, unsubstituted, monosubstituted or polysubstituted with —F;
—S(═O)₂(3-14-membered cycloalkyl), saturated, unsubstituted;
—C₁₋₆-alkyl, saturated, unsubstituted, monosubstituted or disubstituted with substituents independently of one another selected from the group consisting of —OH, ═O, —NH₂, —NHC₁₋₆-alkyl, —N(C₁₋₆-alkyl)₂, —OC₁₋₆-alkyl, —C₁₋₆-alkylene-NH₂, —C₁₋₆-alkylene-NH—C₁₋₆-alkyl, —C(═O)NH₂, —C(═O)—NH—C₁₋₃-alkyl, —C(═O)—N(C₁₋₃-alkyl)₂, -phenyl unsubstituted;
3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is saturated, unsubstituted, monosubstituted or disubstituted with substituents independently of one another selected from the group consisting of —C₁₋₆-alkyl, —C₁₋₆-alkylene-NH₂, —C₁₋₆-alkylene-NH—C₁₋₆-alkylene-CF₃, —C₁₋₆-alkylene-OH, —C₁₋₆-alkylene-NHC(═O)O—C₁₋₆-alkyl, —OH, —OC₁₋₆-alkyl, —NH₂, —N(C₁₋₆-alkyl)₂, —NHC(═O)O—C₁₋₆-alkyl;
3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from azetane, 1,4-oxazepane, pyrrolidine, piperidine, azepane, diazepane, tetrahydrofuran, tetrahydropyran, oxetane, morpholine, piperazine, hexahydrocyclopenta[c]pyrrole, octahydrocyclopenta[c]pyrrole, octahydropyrrolo[1,2-a]pyrazine, 8-azabicyclo[3.2.1]octane, 9-azabicyclo[3.3.1]nonane, quinuclidine, hexahydro-11H-pyrrolizine, 2-oxaspiro[3.3]heptane, 2-azaspiro[3.3]heptane, 7-azaspiro[3.5]nonane, 1,1-dioxothiacyclohexane, in each case unsubstituted, mono- or polysubstituted with substituents independently of one another selected from the group consisting of —F, —OH, ═O, —C₁₋₆-alkyl, —C₁₋₆-alkylene-CF₃, —C₁₋₆-alkylene-OH, —C₁₋₆-alkylene-O—C₁₋₆-alkyl, —NH₂, —N(C₁₋₆-alkyl)₂, —C₁₋₆-alkylene-NH₂, —C₁₋₆-alkylene-N(C₁₋₆-alkyl)₂, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —S(═O)₂C₁₋₆-alkyl, oxetanyl, pyrimidinyl, —C₁₋₆-alkylene-phenyl;
phenyl unsubstituted;
5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of pyridine, pyridazine, pyrazine, pyrazole, isoxazole, triazole, and [1,2,4]triazolo[4,3-a]pyrimidine, in each case unsubstituted, monosubstituted or disubstituted with substituents independently of one another selected from the group consisting of —C₁₋₆-alkyl, —OH.

In an embodiment of the pyrazolo[1,5-a]pyridine derivative according to the invention R³ and R⁴ together form a 5- or 6-membered heterocycle containing 1 or 2 heteroatoms selected from N, O and S, saturated or unsaturated, unsubstituted or mono- or polysubstituted.

In some embodiments, R³ and R⁴ together form a heterocycle selected from the group consisting of pyrrolidine, piperidine, morpholine, and piperazine, in each case unsubstituted, mono- or polysubstituted with substituents independently of one another selected from the group consisting of —F, —C₁₋₆-alkyl, —NH₂, —NHCH₃, —N(CH₃)₂, —C(═O)NH—C₁₋₆-alkyl, —C(═O)N(C₁. 6-alkyl)₂, —C(═O)O—C₁₋₆-alkyl, —NHC(═O)O—C₁₋₆-alkyl, -pyridyl unsubstituted, and 1,2,4-oxadiazole unsubstituted or monosubstituted with —C₁₋₆-alkyl. In an embodiment, R³ and R⁴ together do not form morpholine unsubstituted, mono- or polysubstituted.

In some embodiments, R³ and R⁴ together form a pyrrolidine ring, unsubstituted or monosubstituted with —N(CH₃)₂;
piperidine ring, unsubstituted or monosubstituted with a substituent selected from the group consisting of —C₁₋₆-alkyl, —NH₂, —N(CH₃)₂, —C(═O)NH—C₁₋₆-alkyl, —C(═O)O—C₁₋₆-alkyl, —NHC(═O)O—C₁₋₆-alkyl, and 1,2,4-oxadiazole unsubstituted or monosubstituted with —C₁₋₆-alkyl; morpholine ring, unsubstituted; or piperazine ring, unsubstituted or N-substituted with a substituent selected from the group consisting of —C₁₋₆-alkyl and -pyridyl unsubstituted.

In some embodiments, R³ and R⁴ both do not represent —H. In some embodiments, R³ and R⁴ together with the nitrogen atom to which they are attached form a residue selected from the group consisting of:

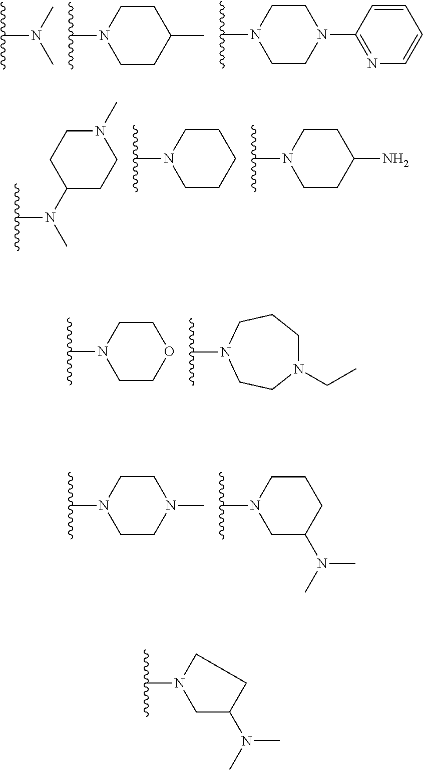

In other embodiments, R³ represents —H and R⁴ does not represent —H.

In some embodiments, R³ represents —H and R⁴ represents —C₁-C₆-alkyl, saturated or unsaturated, unsubstituted, mono- or polysubstituted. In some embodiments, R³ represents —H and R⁴ represents a residue selected from the group consisting of:

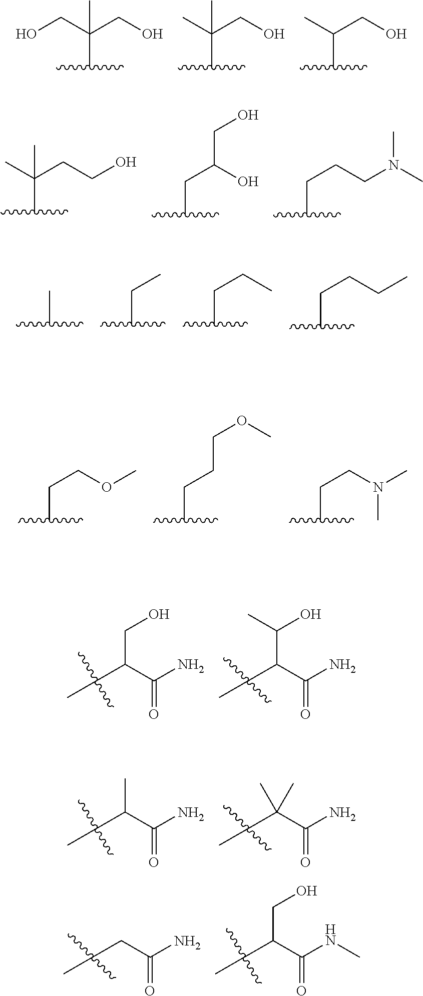

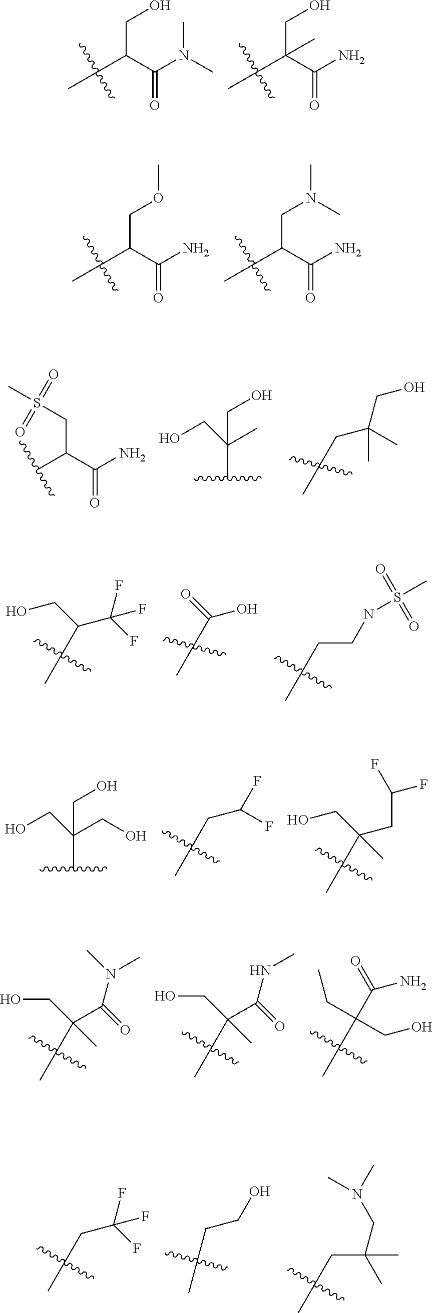

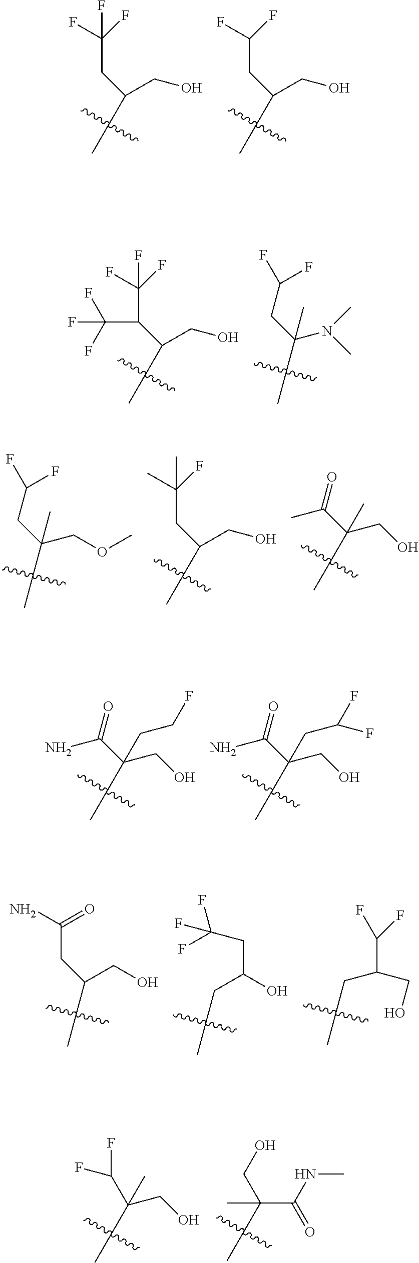

In further embodiments, R³ represents —H and R⁴ represents a residue —CR′R″—(CH₂)m-OH, wherein m is an integer of from 1 to 6, preferably from 1 to 3; and wherein R′ and R″ independently of one another represent —H, —C₁₋₃-alkyl, —CF₃, —CF₂H, —CFH₂, —C₁₋₃-alkylene-CF₃, —C₁₋₃-alkylene-CF₂H, —C₁₋₃-alkylene-CFH₂, —C₁₋₃-alkylene-O—C₁₋₃-alkyl, —C₁₋₃-alkylene-OH, —C(═O)—NH₂, or C(═O)—NH—C₁₋₃-alkyl; preferably —H, —CH₃, —C₁₋₃-alkylene-OH, —C(═O)—NH₂, or C(═O)—NH—C₁₋₃-alkyl. In an embodiment, at least R′ or R″ does not represent —H. In alternative embodiments, neither R′ nor R″ represents —H.

In further embodiments, R³ represents —H and R⁴ represents a 3-14-membered cycloalkyl, saturated or unsaturated, unsubstituted, mono- or polysubstituted; or a 3-14-membered heterocycloalkyl, saturated or unsaturated, unsubstituted, mono- or polysubstituted.

In further embodiments, R³ represents —H and R⁴ represents a 3-membered cycloalkyl, saturated or unsaturated, unsubstituted, mono- or polysubstituted; or a 3-membered heterocycloalkyl, saturated or unsaturated, unsubstituted, mono- or polysubstituted. In some embodiments, R³ represents —H and R⁴ represents a residue selected from the group consisting of:

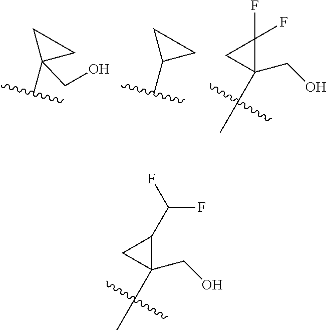

In some embodiments, R³ represents —H and R⁴ represents a 4-membered cycloalkyl, saturated or unsaturated, unsubstituted, mono- or polysubstituted; or a 3-14-membered heterocycloalkyl (preferably a 4-membered heterocycloalkyl), saturated or unsaturated, unsubstituted, mono- or polysubstituted. In some embodiments, R³ represents —H and R⁴ represents a residue selected from the group consisting of:

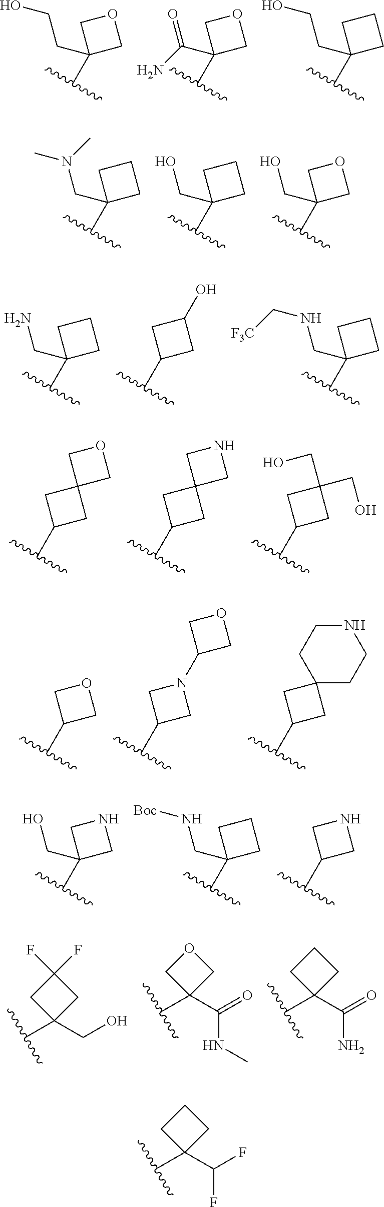

In some embodiments, R³ represents —H and R⁴ represents a residue according to general formula (A),

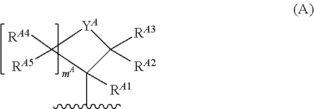

wherein
m^{A} is 0 or 1;
Y^{A} is selected from —O—, —NR^{A6}— and —CR^{A7}R^{A8}—; and
R^{A1}, R^{A2}, R^{A3}, R^{A4}, R^{A5}, R^{A6}, R^{A7}, and R^{A8} independently of one another represent —H, F, —C₁₋₃-alkyl, —C₁₋₃-alkylene-OH, —C₁₋₃-alkylene-NH₂, —C₁₋₃-alkylene-NH(C₁₋₃-alkyl), —C₁₋₃-alkylene-N(C₁-3-alkyl)₂, —C₁₋₃-alkylene-NH(C₁₋₃-alkylene-CF₃), —C₁₋₃-alkylene-C(═O)NH₂, —C₁₋₃-alkylene-NH—C(═O)OC₁₋₄-alkyl, —C(═O)NH₂, —C(═O)—NH—C₁₋₃-alkyl, —C(═O)—N(C₁₋₃-alkyl)₂, -3-oxetanyl, or —CHF₂; preferably, R^{A1}, R^{A2}, R^{A3}, R^{A4}, R^{A5}, R^{A6}, R^{A7}, and R^{A8} independently of one another represent —H, F, —C₁₋₃-alkyl, —C₁₋₃-alkylene-OH, —C₁₋₃-alkylene-NH₂, —C₁₋₃-alkylene-NH(C₁₋₃-alkyl), —C₁₋₃-alkylene-N(C₁₋₃-alkyl)₂, —C₁₋₃-alkylene-NH(C₁₋₃-alkylene-CF₃), —C₁₋₃-alkylene-C(═O)NH₂, —C₁₋₃-alkylene-NH—C(═O)OC₁₋₄-alkyl, —C(═O)NH₂, —C(═O)—NH—C₁₋₃-alkyl, —C(═O)—N(C₁₋₃-alkyl)₂, or -3-oxetanyl; or R^{A7} and R^{A8} together with the carbon atom to which they are attached form a ring and represent —CH₂OCH₂—, —CH₂OCH₂CH₂— or —CH₂CH₂OCH₂CH₂—, —CH₂NHCH₂—, —CH₂NHCH₂CH₂— or —CH₂CH₂NHCH₂CH₂—.

In some embodiments, R³ represents —H and R⁴ represents a residue according to general formula (A) as defined above, wherein
m^{A} is 0 or 1;
Y^{A} is selected from —O— and —CR^{A7}R^{A8}—; and
R^{A1}, R^{A2}, R^{A3}, R^{A4}, R^{A5}, R^{A7}, and R^{A8} independently of one another represent —H, —C₁₋₃-alkylene-OH, —C₁₋₃-alkylene-N(C₁₋₃-alkyl)₂, —C(═O)NH₂, or —CHF₂; preferably R^{A1}, R^{A2}, R^{A3}, R^{A4}, R^{A5}, R^{A7}, and R^{A8} independently of one another represent —H, —C₁₋₃-alkylene-OH, —C₁₋₃-alkylene-N(C₁₋₃-alkyl)₂, or —C(═O)NH₂; preferably with the proviso that only one of R^{A1}, R^{A2}, R^{A3}, R^{A4}, R^{A5}, R^{A7}, and R^{A8} represents a residue that is not —H.

In some embodiments, R³ represents —H and R⁴ represents a residue according to general formula (A) as defined above, wherein
m^{A} is 0 or 1;
Y^{A} is selected from —O— and —CR^{A7}R^{A8}—; and
R^{A1} represents —C₁₋₃-alkylene-OH, —C₁₋₃-alkylene-N(C₁₋₃-alkyl)₂, —C(═O)NH₂, or —CHF₂; preferably R^{A1} represents —C₁₋₃-alkylene-OH, —C₁₋₃-alkylene-N(C₁₋₃-alkyl)₂, or —C(═O)NH₂; and
R^{A2}, R^{A3}, R^{A4}, R^{A5}, R^{A7}, and R^{A8} represent —H.

In some embodiments, R³ represents —H and R⁴ represents a 3-14-membered cycloalkyl (preferably a 5-membered cycloalkyl), saturated or unsaturated, unsubstituted, mono- or polysubstituted; or a 3-14-membered heterocycloalkyl (preferably a 5-membered heterocycloalkyl), saturated or unsaturated, unsubstituted, mono- or polysubstituted; or a 5-14-membered heteroaryl (preferably a 5-membered heteroaryl), unsubstituted, mono- or polysubstituted. In preferred embodiments, R³ represents —H and R⁴ represents a residue selected from the group consisting of:

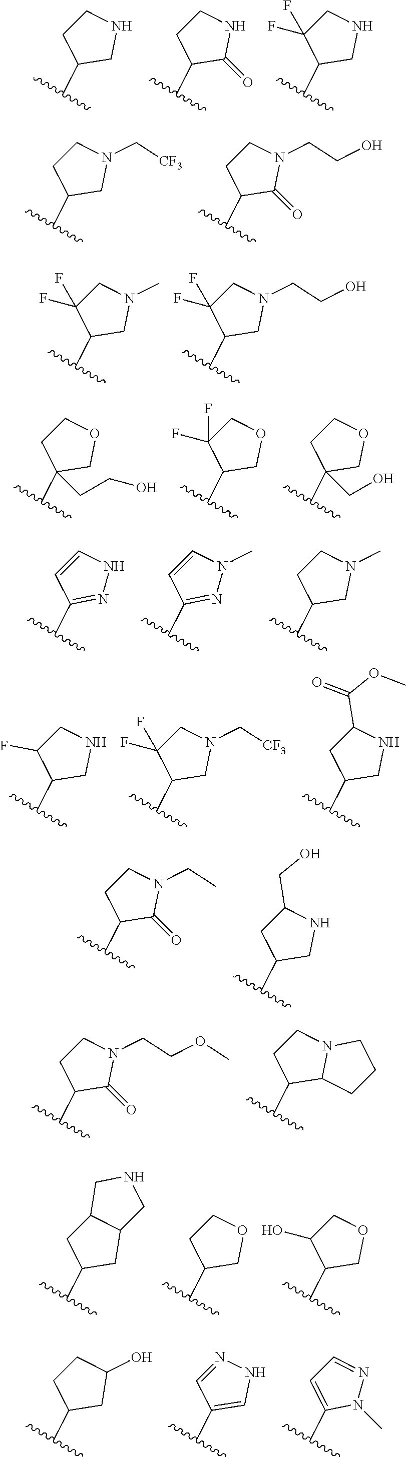

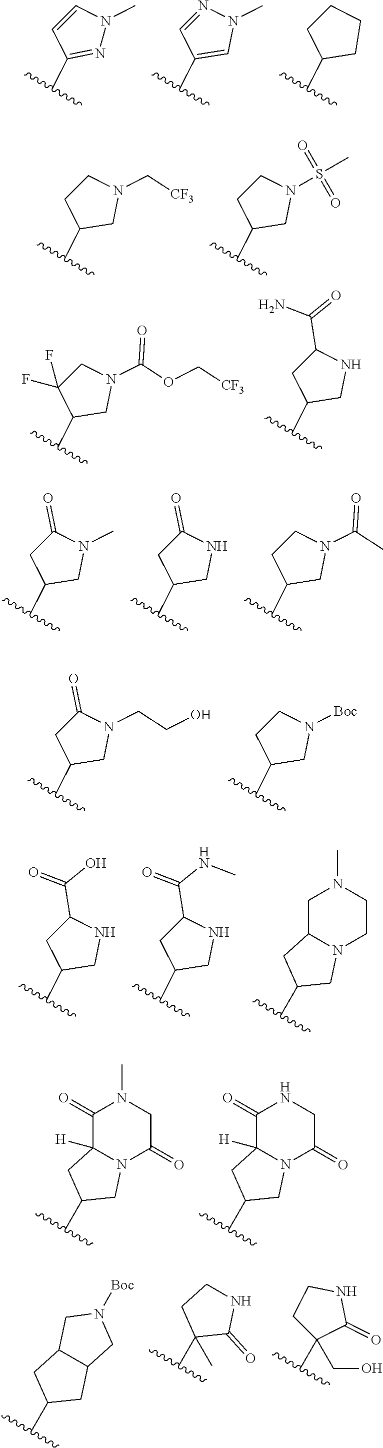

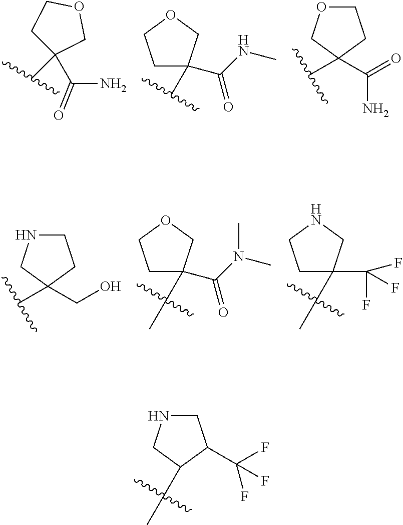

In some embodiments, R³ represents —H and R⁴ represents a residue according to general formula (B).

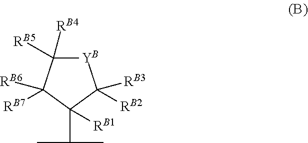

wherein
Y^{B} is selected from —O—, —NR^{B8}— and —CR^{B9}R^{B10}—; and
R^{B1}, R^{B2}, R^{B3}, R^{B4}, R^{B5}, R^{B6}, R^{B7}, R^{B8}, R^{B9} and R^{B10} independently of one another represent —H, —F, —OH, —C₁₋₃-alkyl, —C₁₋₃-alkylene-OH, —C₁₋₃-alkylene-O—C₁₋₃-alkyl, —C₁₋₃-alkylene-CF₃, —C₁₋₃-alkylene-CO₂H, —C₁₋₃-alkylene-C(═O)O—C₁₋₃-alkyl, —C(═O)NH₂, —C(═O)NH—C₁₋₃-alkyl, or —C(═O)N(C₁₋₃-alkyl)₂; or R^{B2} and R^{B3} together represent ═O; or R^{B4} and R^{B}5 together represent ═O.

In some embodiments, R³ represents —H and R⁴ represents a residue according to general formula (B) as defined above, wherein
Y^{B} is selected from —O— and —NR^{B8}—; and
R^{B}1, R^{B2}, R^{B3}, R^{B4}, R^{B5}, R^{B6}, R^{B7}, R^{B8} independently of one another represent —H, —F, —C₁₋₃-alkyl, —C₁₋₃-alkylene-OH, —C₁₋₃-alkylene-CF₃ or —C(═O)NH₂; or R^{B2} and R^{B3} together represent ═O; or
R^{B4} and R^{B5} together represent ═O; preferably with the proviso that only 1, 2 or 3 of R^{A1}, R^{A2}R^{A3}, R^{A4}, R^{A5}, R^{A7}, and R^{A8} represent a residue that is not —H; preferably with the proviso that at least one of R^{A1}, R^{A2}, R^{A3}, R^{A4}, R^{A5}, R^{A7}, and R^{A8} represent a residue that is not —H.

In some embodiments, R³ represents —H and R⁴ represents a 3-14-membered cycloalkyl (preferably a 6-membered cycloalkyl), saturated or unsaturated, unsubstituted, mono- or polysubstituted; or a 3-14-membered heterocycloalkyl (preferably a 6-membered heterocycloalkyl), saturated or unsaturated, unsubstituted, mono- or polysubstituted; or a 6-14-membered aryl (preferably a 6-membered aryl), unsubstituted, mono- or polysubstituted; or a 5-14-membered heteroaryl (preferably a 6-membered heteroaryl), unsubstituted, mono- or polysubstituted. In preferred embodiments, R³ represents —H and R⁴ represents a residue selected from the group consisting of:

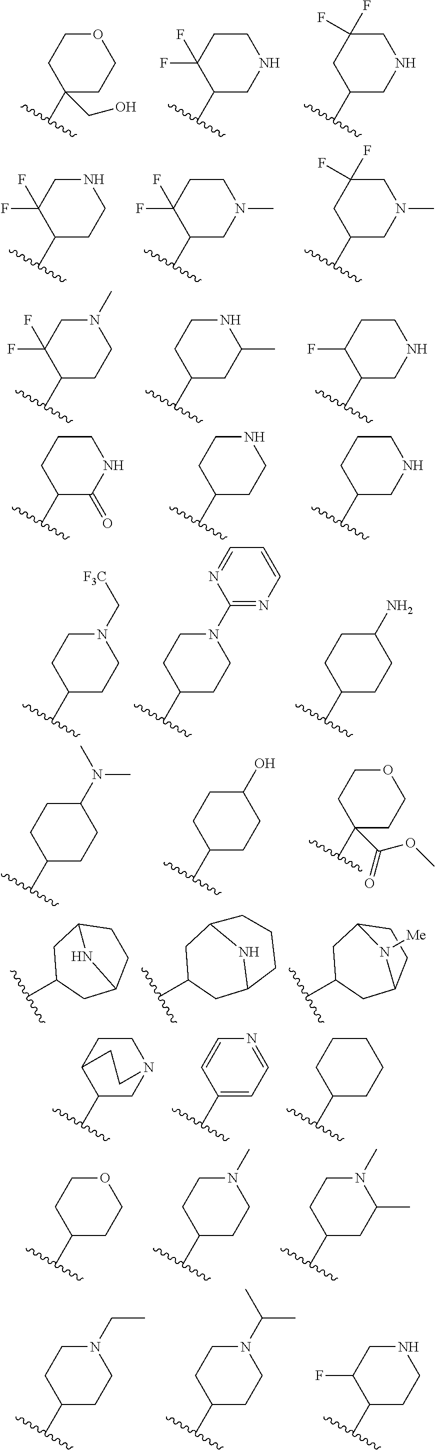

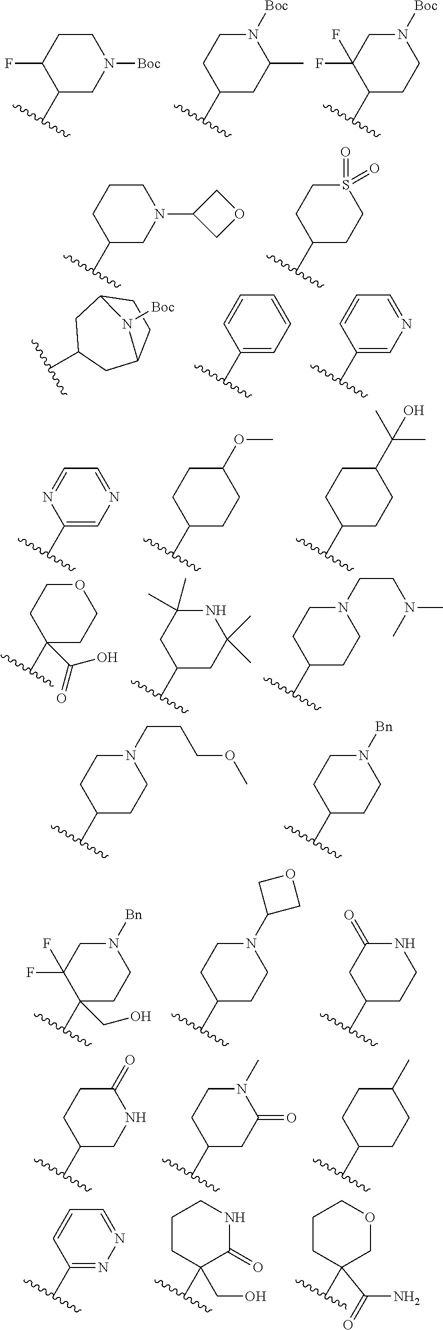

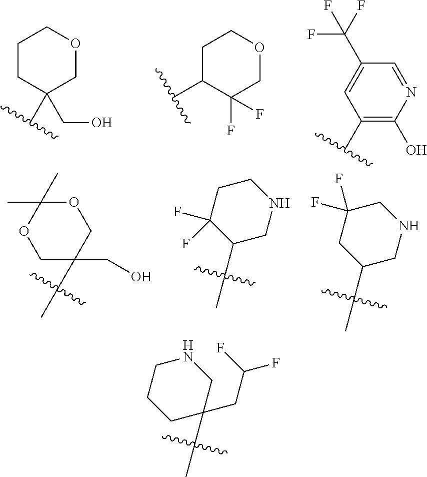

In some embodiments, R³ represents —H and R⁴ represents a residue according to general formula (C),

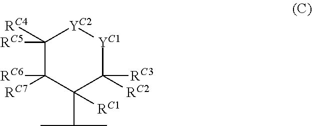

wherein
Y^{C1} is selected from —O—, —S(═O)₂—, —NR^{C8}— and —CR^{C9}R^{C10}— and Y^{C2} represents —CR^{C11}R^{C12}—; or
Y^{C1} represents —CR^{C9}R^{C10}— and Y^{C2} is selected from —O—, —S(═O)₂—, and —NR^{C8}—; R^{C1}, R^{C2}, R^{C3}, R^{C4}, R^{C5}, R^{C6}, R^{C7}, R^{C8}, R^{C9}, R^{C10}, R^{C11} and R^{C12} independently of one another represent
—H, —F, —OH, —C(═O)OC₁₋₃-alkyl, —NH₂, —NH(C₁₋₃-alkyl), —N(C₁₋₃-alkyl)₂, —C₁₋₃-alkyl, —C₁₋₃-alkylene-OH, —C₁₋₃-alkylene-, —C(═O)NH₂, —C(═O)NH—C₁₋₃-alkyl, or —C(═O)N(C₁₋₃-alkyl)₂: or R^{C2} and R^{C3} together represent ═O; or R^{C4} and R^{C5} together represent ═O; or R^{C9} and R^{C10} together represent ═O; or R^{C1} and R^{C12} together represent ═O.

In some embodiments, R³ represents —H and R⁴ represents a residue according to general formula (C) as defined above, wherein
Y^{C1} is selected from —O— or —NR^{C8}— and Y^{C2} represents —CR^{C11}R^{C12}—; or Y^{C1} represents —CR^{C9}R^{C10}— and Y^{C2} is selected from —O—, and —NR^{C8}—;
R^{C1}, R^{C2}, R^{C3}, R^{C4}, R^{C5}, R^{C6}, R^{C7}, R^{C8}, R^{C9}, R^{C10}, R^{C11} and R^{C12} independently of one another represent —H, —F, —C₁₋₃-alkyl, —C₁₋₃-alkylene-OH, or —C(═O)NH₂; preferably with the proviso that only 1, 2 or 3 of R^{C1}, R^{C2}, R^{C3}, R^{C4}, R^{C5}, R^{C6}, R^{C7}, R^{C8}, R^{C9}, R^{C10}, R^{C11} and R^{C12} represent a residue that is not —H; preferably with the proviso that at least one of R^{C1}, R^{C2}, R^{C3}, R^{C4}, R^{C5}, R^{C6}, R^{C7}, R^{C8}, R^{C9}, R^{C10}, R^{C11} and R^{C12} represent a residue that is not —H.

In some embodiments, R³ represents —H and R⁴ represents a 7-membered cycloalkyl, saturated or unsaturated, unsubstituted, mono- or polysubstituted; or a 7-membered heterocycloalkyl, saturated or unsaturated, unsubstituted, mono- or polysubstituted. In some embodiments, R³ represents —H and R⁴ represents a residue:

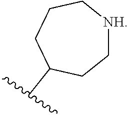 [SMILES: *C1CCCNCC1]

In some embodiments, R³ represents —H and R⁴ represents a 3-14-membered cycloalkyl (preferably a 3, 4, 5 or 6-membered cycloalkyl), saturated or unsaturated, unsubstituted, mono- or polysubstituted; wherein said 3-14-membered cycloalkyl is connected through —C₁-C₆-alkylene-, saturated or unsaturated, unsubstituted, mono- or polysubstituted; or a 3-14-membered heterocycloalkyl (preferably a 4, 5 or 6-membered heterocycloalkyl), saturated or unsaturated, unsubstituted, mono- or polysubstituted; wherein said 3-14-membered heterocycloalkyl is connected through —C₁-C₆-alkylene-, saturated or unsaturated, unsubstituted, mono- or polysubstituted; or a 6-14-membered aryl (preferably a 6-membered aryl), unsubstituted, mono- or polysubstituted; wherein said 6-14-membered aryl is connected through —C₁-C₆-alkylene-, saturated or unsaturated, unsubstituted, mono- or polysubstituted; or a 5-14-membered heteroaryl (preferably a 5 or 6-membered heteroaryl), unsubstituted, mono- or polysubstituted; wherein said 5-14-membered heteroaryl is connected through —C₁-C₆-alkylene-, saturated or unsaturated, unsubstituted, mono- or polysubstituted. In preferred embodiments, R³ represents —H and R⁴ represents a residue selected from the group consisting of:

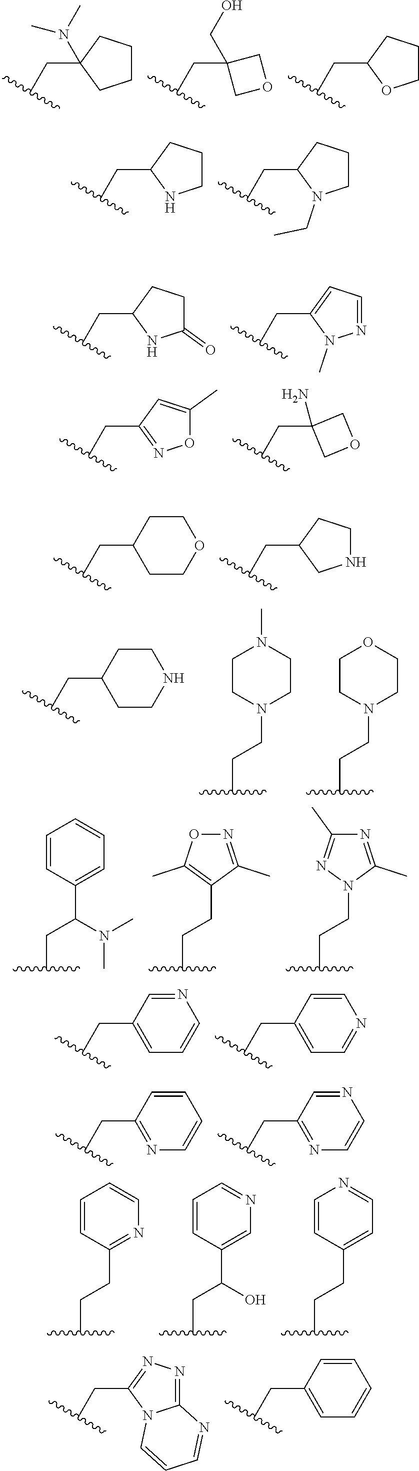

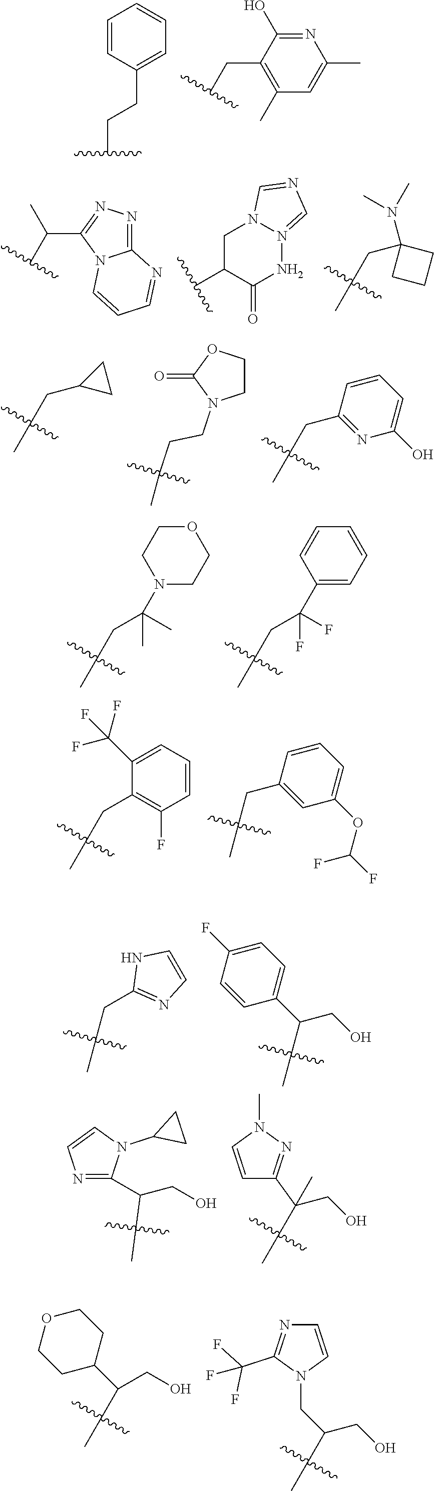

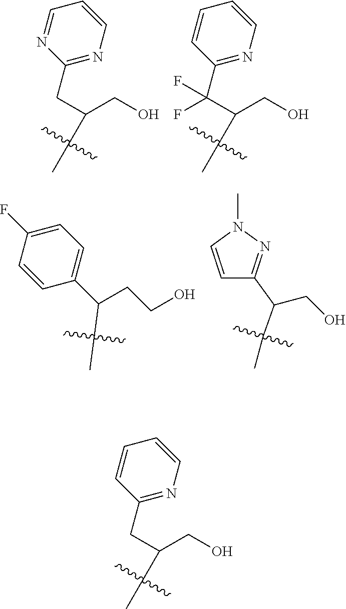

In some embodiments, R³ represents —H and R⁴ represents a 5-membered heterocycloalkyl, saturated or unsaturated, unsubstituted, mono- or polysubstituted; wherein said 5-membered heterocycloalkyl is connected through —C₁-C₆-alkylene-, saturated or unsaturated, unsubstituted, mono- or polysubstituted; or a 5-membered heteroaryl, unsubstituted, mono- or polysubstituted; wherein said 5-membered heteroaryl is connected through —C₁-C₆-alkylene-, saturated or unsaturated, unsubstituted, mono- or polysubstituted.

In some embodiments, R³ represents —H and R⁴ represents a residue selected from the group consisting of:

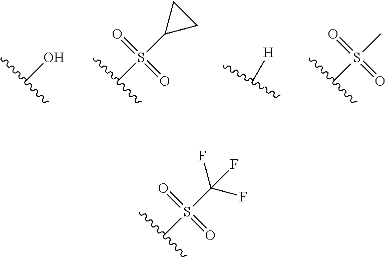

In some embodiments, R represents —H and R represents
(i) a residue —CR′R″—(CH₂)ₘ—OH, wherein m is an integer of from 1 to 6, preferably from 1 to 3; and wherein R′ and R″ independently of one another represent —H, —C₁₋₃-alkyl, —CF₃, —CF₂H, —CFH₂, —C₁₋₃-alkylene-CF₃, —C₁₋₃-alkylene-CF₂H, —C₁₋₃-alkylene-CFH₂, —C₁₋₃-alkylene-O—C₁₋₃-alkyl, or —C₁₋₃-alkylene-OH; preferably —H, —CH₃, or —C₁₋₃-alkylene-OH. In an embodiment, at least R′ or R″ does not represent —H. In an embodiment, neither R′ nor R″ represents —H; or
(ii) a residue according to general formula (D),

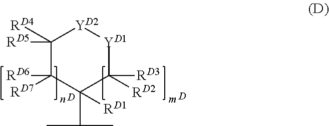

wherein
m^{D} and n^{D} independently of one another are 0, 1, 2, or 3; preferably with the proviso that m^{D}+n^{D}≤3;
Y^{D1} is selected from —O—, —S(═O)₂—, —S(═O)(═NH)—, —NR^{D8}— and —CR^{D9}R^{D10}— and Y^{D2} represents —CR^{D11}R^{D12}—; or Y^{D1} is selected from —O—, —S(═O)₂—, —NR^{D8}— and —CR^{D9}DR¹⁰ and Y^{D2} represents —CR^{D11}R^{D12}—; or Y^{D1} represents —CR^{D9}R^{D10}— and Y^{D2} is selected from —O—, —S(═O)₂—, and —NR^{D8}—;
R^{D1}, R^{D2}, R^{D3}, R^{D4}, R^{D5}, R^{D6}, R^{D7}, R^{D8}, R^{D9}, R^{D10}, R^{D11} and R^{D12} independently of one another represent —H, —F, —OH, —C₁₋₃-alkylene-OH, —C(═O)NH₂, —C₁₋₃-alkylene-C(O)NH₂, —C(═O)O—C₁₋₃-alkyl, —NH₂, —C₁₋₃-alkylene-NH₂, —NH(C₁₋₃-alkyl), —N(C₁₋₃-alkyl)₂, —NH(C₁₋₃-alkylene-CF₃), —C₁₋₃-alkylene-OCH₃, —C₁₋₃-alkyl, —C₁₋₃-alkylene-CF₃: or R^{D2} and R^{D3} together represent ═O; or R^{D4} and R^{D5} together represent ═O; or R^{D9} and R^{D10} together represent ═O; or R^{D11} and R^{D12} together represent ═O;
preferably wherein
m^{D} and n^{D} independently of one another are 0, 1, 2 or 3; preferably with the proviso that m^{D}+n^{D}≤3;
Y^{D1} is selected from —O—, —NR^{D8}— and —CR^{D9}R^{D10}— and Y^{D2} represents —CR^{D11}R^{D12}—; or Y^{D1} represents —CR^{D9}R^{D10}— and Y^{D2} is selected from —O— and —NR^{D8}—;
R^{D1}, R^{D2}, R^{D3}, R^{D4}, R^{D5}, R^{D6}, R^{D7}, R^{D8}, R^{D9}, R^{D10}, R^{D11} and R^{D12} independently of one another represent —H, —F, —OH, —C₁₋₃-alkylene-OH, —C(═O)NH₂, —CH₂NH₂, —CH₂N(CH₃)₂, —NHCH₂CF₃, —CH₃, or —CH₂CF₃: or R^{D2} and R^{D3} together represent ═O; or R^{D}a and R^{D5} together represent ═O; or R^{D9} and R^{D10} together represent ═O; or R^{D11} and R^{D12} together represent ═O; preferably with the proviso that only 1, 2 or 3 of R^{D1}, R^{D2}, R^{D3}, R^{D4}, R^{D5}, R^{D6}, R^{D7}, R^{D8}, R^{D9}, R^{D10}, R^{D11} and R^{D12} represent a residue that is not —H; preferably with the proviso that at least one of R^{D1}, R^{D2}, R^{D3}, R^{D4}, R^{D5}, R^{D6}, R^{D7}, R^{D8}, R^{D9}, R^{D10}, R^{D11} and R^{D12} represent a residue that is not —H.

In some embodiments, R³ represents —H and R⁴ represents a residue selected from the group consisting of:

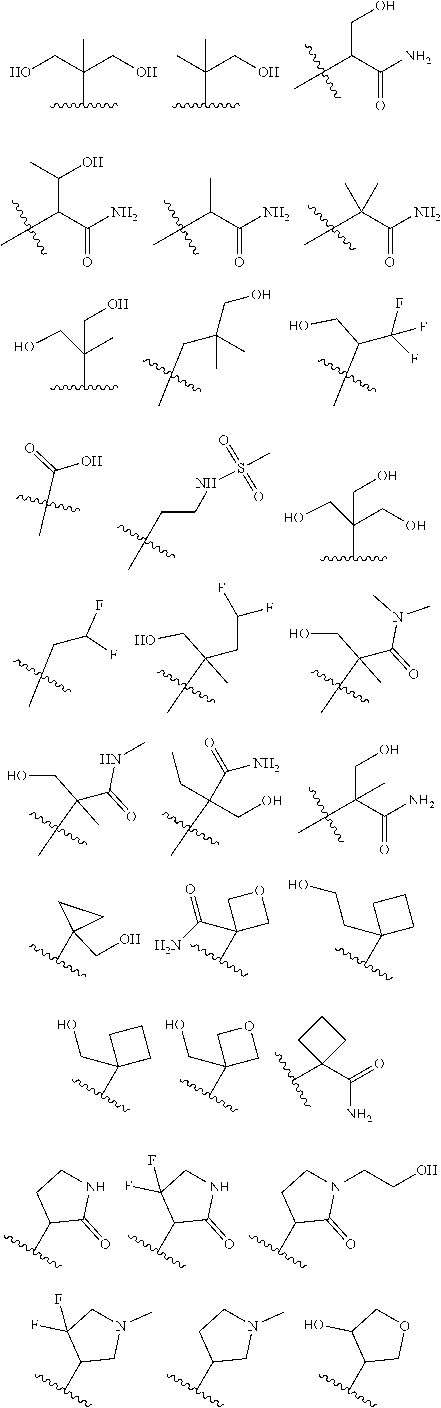

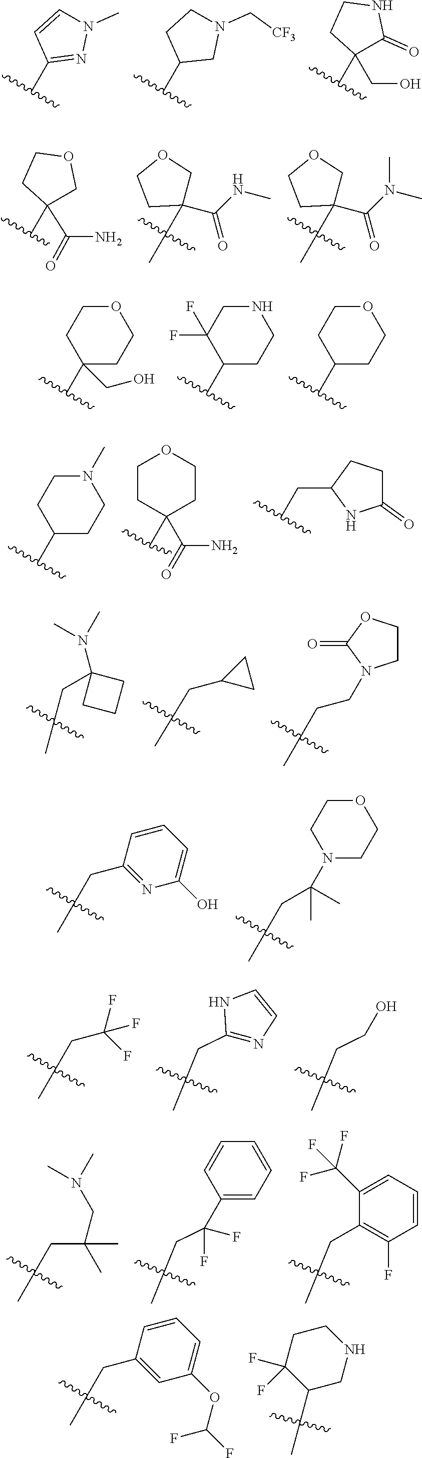

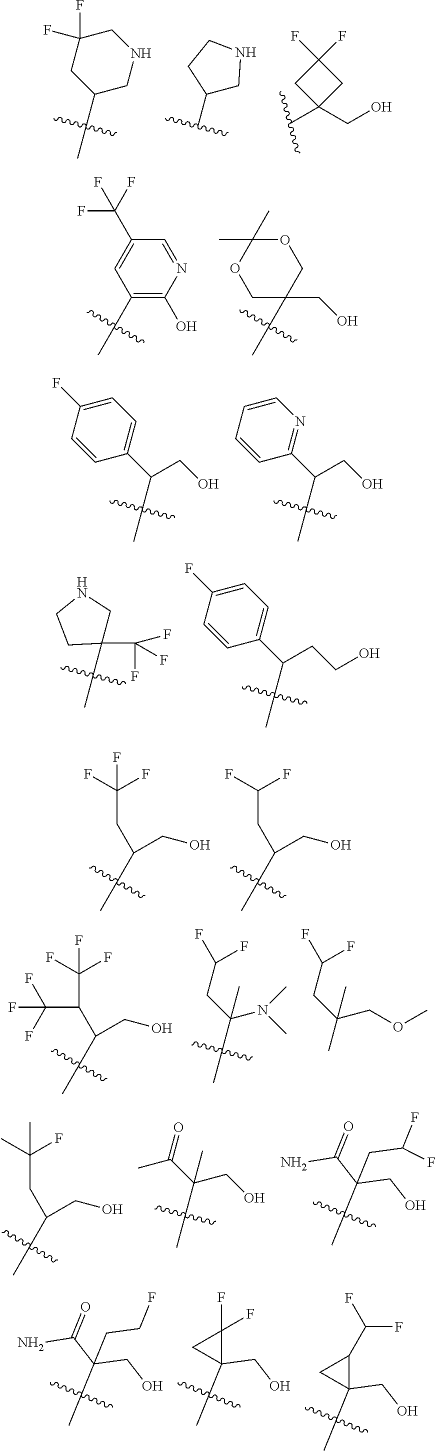

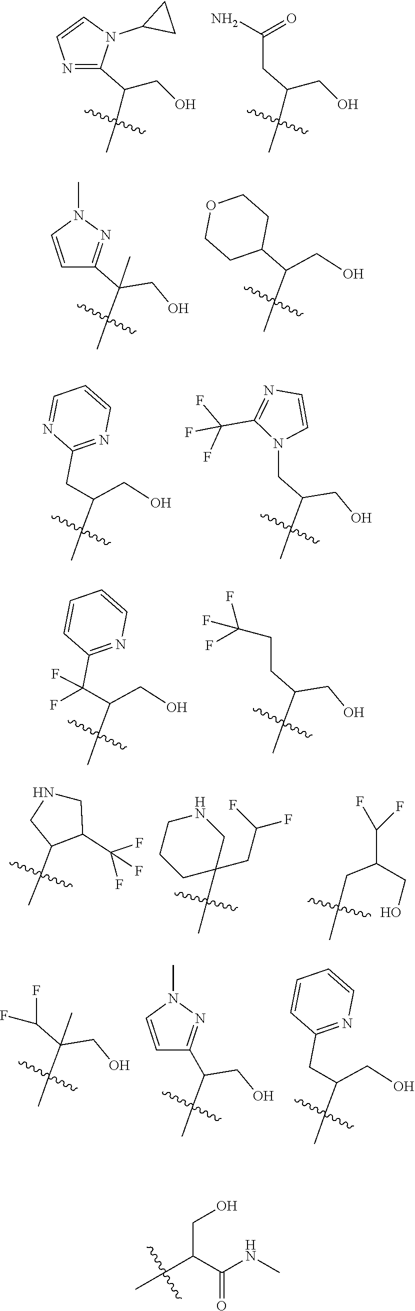

In some embodiments of the pyrazolo[1,5-a]pyridine derivative according to the invention R⁵ and R^{5′} independently of one another represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted;
wherein said 3-14-membered cycloalkyl is optionally connected through —C₁-C₆-alkylene- or —C₁-C₆-heteroalkylene-, in each case saturated or unsaturated, unsubstituted, mono- or polysubstituted.

In some embodiments, R⁵ and R^{5′} independently of one another represent —H, —C₁-C₆-alkyl, or —C₁-C₆-alkylene-N(C₁-C₆-alkyl)₂.

In some embodiments of the pyrazolo[1,5-a]pyridine derivatives according to the invention, at least one of R⁵ and R^{5′} is not —H.

In some embodiments of the pyrazolo[1,5-a]pyridine derivatives according to the invention, R⁵ and R^{5′} are both —H.

In some embodiments, T represents —O— and U represents —CR⁵R^{5′}— and the resultant moiety —O—CR⁵R^{5′}— represents a residue selected from the group consisting of:

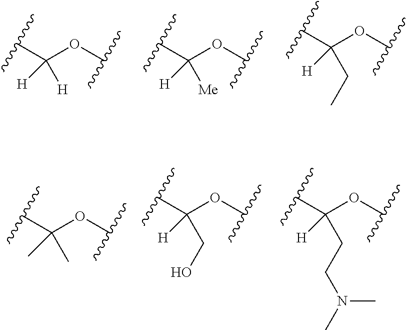

In some embodiments, T represents —CR⁵R^{5′}— and U represents —O— and the resultant moiety —CR⁵R^{5′}—O— represents a residue:

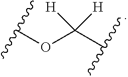 [SMILES: *CO*]

In some embodiments, R⁵ represents —H and R^{5′} represents a residue selected from the group consisting of —H, —C₁₋₃-alkyl, —CF₃, —CF₂H, —CFH₂, —C₁₋₃-alkylene-CF₃, —C₁₋₃-alkylene-CF₂H, —C₁₋₃-alkylene-CFH₂, and —C₁₋₃-alkylene-OH; preferably —H or C₁₋₃-alkyl.

In some embodiments of the pyrazolo[1,5-a]pyridine derivatives according to the invention R⁶, R⁷ and R⁸ independently of one another represent
—H; —F, —Cl, —Br, —I, —OH, —SH, —SF₅, —CN, —NO₂, —C(═O)OH, —NH₂;
—C₁₋₆-alkyl, saturated or unsaturated, unsubstituted, mono- or polysubstituted;
—O—C₁₋₆-alkyl, saturated or unsaturated, unsubstituted, mono- or polysubstituted;
—NHC₁₋₆-alkyl, saturated or unsaturated, unsubstituted, mono- or polysubstituted;
—N(C₁₋₆-alkyl)₂, saturated or unsaturated, unsubstituted, mono- or polysubstituted;
—C(═O)OC₁₋₆-alkyl, saturated or unsaturated, unsubstituted, mono- or polysubstituted;
—OC(═O)C₁₋₆-alkyl, saturated or unsaturated, unsubstituted, mono- or polysubstituted;
—C₁₋₆-heteroalkyl, saturated or unsaturated, unsubstituted, mono- or polysubstituted.

In some embodiments, R⁶, R⁷ and R⁸ independently of one another represent
—H, —F, —Cl, —Br, —I, —OH, —SH, —SF₅, —CN, —NO₂, —C(═O)OH, —NH₂,
—C₁₋₆-alkyl, —CF₃, —CHF₂, —CH₂F,
—O—C₁₋₆-alkyl, —OCF₃, —OCHF₂, —OCH₂F, —NHC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—N(C₁₋₆-alkyl)₂ unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—C(═O)OC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—OC(═O)C₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂; or —C₁₋₆-heteroalkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂.

In some embodiments, R⁶, R⁷ and R⁸ independently of one another represents a residue selected from the group consisting of —H, —F, —Cl, —Br, —I, —CN, C₁₋₃-alkyl, —CF₃, —CF₂H, and —CFH₂; preferably —H or —F.

In some embodiments of the pyrazolo[1,5-a]pyridine derivatives according to the invention R⁶ represents —H, —F, —C, —CN, or —C₁-C₆-alkyl.

In some embodiments of the pyrazolo[1,5-a]pyridine derivatives according to the invention, R⁶ does not represent —H.

In some embodiments, R⁶ represents a residue selected from the group consisting of —H, —F, —C, —CN or —CH₃; preferably —H, —F, —CN or —CH₃.

In some embodiments of the pyrazolo[1,5-a]pyridine derivatives according to the invention R¹ represents —H, —F, —C, —CN, or —C₁-C₆-alkyl.

In some embodiments of the pyrazolo[1,5-a]pyridine derivatives according to the invention R¹ does not represent —H.

In some embodiments, especially when Q represents —NR³R⁴, R¹ represents a residue selected from the group consisting of —H, —F, —C, —CN or CH₃.

In some embodiments, especially when Q represents —OR², R¹ represents a residue selected from the group consisting of —H or

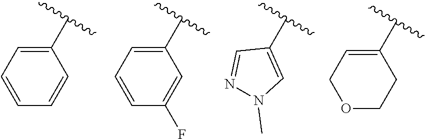

In some embodiments of the pyrazolo[1,5-a]pyridine derivatives according to the invention R⁸ represents —H, —F, —Cl, —CN, or —C₁-C₆-alkyl.

In some embodiments of the pyrazolo[1,5-a]pyridine derivatives according to the invention R⁸ does not represent —H.

In some embodiments, R⁸ represents a residue selected from the group consisting of —H, —F, —Cl, —CN or CH₃; preferably —F.

In some embodiments of the pyrazolo[1,5-a]pyridine derivatives according to the invention
(i) R⁶, R⁷ and R⁸ each represent —H; or
(ii) two of R⁶, R⁷ and R⁸ represent —H and the other of R⁶, R⁷ and R⁸ represents —F, —Cl, —CN, or —CH₃; or
(iii) one of R⁶, R⁷ and R⁸ represents —H and the other of R⁶, R⁷ and R⁸ independently of one another represent —F, —Cl, —CN, or —CH₃.

In some embodiments, the compound is according to general formula (I), wherein
R¹ represents —CH₃; and/or
R⁶, R⁷ and R⁸ each represent —H; and/or
T represents —O—; and/or
U represents —CH₂—; and/or
V represents thiazolyl, pyridyl, or pyrazolyl; wherein said thiazolyl, pyridyl, and pyrazolyl each independently from one another can be unsubstituted, monosubstituted or disubstituted with a substituent selected from the group consisting of —CH₃; —F; —CH₂CHF₂; and —CF₃; and/or
Q represents NR³R⁴; and/or
R³ represents H; and/or
R⁴ represents

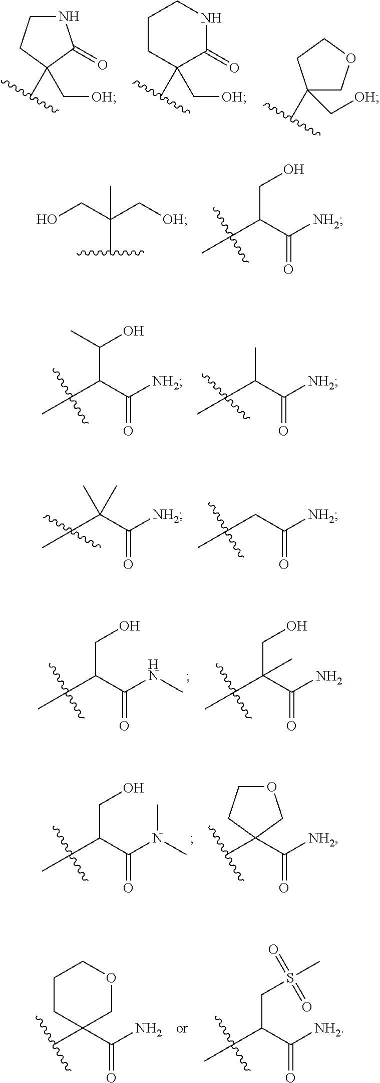

In exemplary embodiments of the invention, the pyrazolo[1,5-a]pyridine derivative is selected from the group consisting of:
Cpd 001—2-({5-[(2-fluorophenyl)methoxy]-2-methylpyrazolo[1,5-a]pyridin-3-yl}formamido)-2-(hydroxymethyl)butanamide;
Cpd 002—N-(1,3-dihydroxy-2-methylpropan-2-yl)-5-[(2-fluorophenyl)methoxy]-2-methylpyrazolo[1,5-a]pyridine-3-carboxamide;
Cpd 003—(2S)-2-({5-[(2-fluorophenyl)methoxy]-2-methylpyrazolo[1,5-a]pyridin-3-yl}formamido)-3-hydroxypropanamide;
Cpd 004—2-({5-[(2-fluorophenyl)methoxy]-2-methylpyrazolo[1,5-a]pyridin-3-yl}formamido)-2-methylpropanamide;
Cpd 005—5-[(2-fluorophenyl)methoxy]-N-(2-methanesulfonamidoethyl)-2-methylpyrazolo[1,5-a]pyridine-3-carboxamide;
Cpd 006—N-(2,2-difluoroethyl)-5-[(2-fluorophenyl)methoxy]-2-methylpyrazolo[1,5-a]pyridine-3-carboxamide;
Cpd 007—(2S,3R)-2-({5-[(2-fluorophenyl)methoxy]-2-methylpyrazolo[1,5-a]pyridin-3-yl}formamido)-3-hydroxybutanamide;
Cpd 008—N-(1,3-dihydroxy-2-methylpropan-2-yl)-2-methyl-5-[(4-methyl-1,3-thiazol-5-yl)methoxy]pyrazolo[1,5-a]pyridine-3-carboxamide;
Cpd 009—2-({5-[(2-fluorophenyl)methoxy]-2-methylpyrazolo[1,5-a]pyridin-3-yl}formamido)-3-hydroxy-N-methylpropanamide;
Cpd 010—(2S)-3-hydroxy-2-({2-methyl-5-[(4-methyl-1,3-thiazol-5-yl)methoxy]pyrazolo[1,5-a]pyridin-3-yl}formamido)propanamide;
Cpd 011—2-({5-[(2-fluorophenyl)methoxy]-2-methylpyrazolo[1,5-a]pyridin-3-yl}formamido)-3-hydroxy-N,2-dimethylpropanamide;
Cpd 012—(2S)-2-({5-[(2-chlorophenyl)methoxy]-2-methylpyrazolo[1,5-a]pyridin-3-yl}formamido)-3-hydroxypropanamide;
Cpd 013—2-({5-[(2-chlorophenyl)methoxy]-2-methylpyrazolo[1,5-a]pyridin-3-yl}formamido)-2-methylpropanamide;
Cpd 014—5-[(2-chlorophenyl)methoxy]-N-(1,3-dihydroxy-2-methylpropan-2-yl)-2-methylpyrazolo[1,5-a]pyridine-3-carboxamide;
Cpd 015—2-{[5-(2,2-difluoroethoxy)-2-methylpyrazolo[1,5-a]pyridin-3-yl]formamido}-3-hydroxy-2-methylpropanamide;
Cpd 016—2-{[5-(benzyloxy)-2-methylpyrazolo[1,5-a]pyridin-3-yl]formamido}-3-hydroxy-2-methylpropanamide;
Cpd 017—2-({5-[(2-fluorophenyl)methoxy]-2-methylpyrazolo[1,5-a]pyridin-3-yl}formamido)-3-hydroxy-N,N,2-trimethylpropanamide;
Cpd 018—5-[(2-fluorophenyl)methoxy]-N-[3-(hydroxymethyl)-2-oxopyrrolidin-3-yl]-2-methylpyrazolo[1,5-a]pyridine-3-carboxamide;
Cpd 019—2-({5-[(2-chlorophenyl)methoxy]-2-methylpyrazolo[1,5-a]pyridin-3-yl}formamido)-3-hydroxy-2-methylpropanamide;
Cpd 020—2-({5-[(2-fluorophenyl)methoxy]-2-methylpyrazolo[1,5-a]pyridin-3-yl}formamido)-3-hydroxy-2-methylpropanamide;
Cpd 021—3-hydroxy-2-methyl-2-({2-methyl-5-[(6-methylpyridin-3-yl)methoxy]pyrazolo[1,5-a]pyridin-3-yl}formamido)propanamide;
Cpd 022—2-{[5-(cyclopropylmethoxy)-2-methylpyrazolo[1,5-a]pyridin-3-yl]formamido}-3-hydroxy-2-methylpropanamide;
Cpd 023—2-[(5-{[2-(difluoromethyl)phenyl]methoxy}-2-methylpyrazolo[1,5-a]pyridin-3-yl)formamido]-3-hydroxy-2-methylpropanamide;
Cpd 024—2-(1-{5-[(2-fluorophenyl)methoxy]-2-methylpyrazolo[1,5-a]pyridin-3-yl}-N-methylformamido)-3-hydroxy-2-methylpropanamide;
Cpd 025—N-(4,4-difluoro-1-hydroxy-2-methylbutan-2-yl)-5-[(2-fluorophenyl)methoxy]-2-methylpyrazolo[1,5-a]pyridine-3-carboxamide;
Cpd 026—2-({5-[(2,4-difluorophenyl)methoxy]-2-methylpyrazolo[1,5-a]pyridin-3-yl}formamido)-3-hydroxy-2-methylpropanamide;
Cpd 027—2-({5-[(2,3-difluorophenyl)methoxy]-2-methylpyrazolo[1,5-a]pyridin-3-yl}formamido)-3-hydroxy-2-methylpropanamide;
Cpd 028—2-({5-[(2-fluoro-4-methylphenyl)methoxy]-2-methylpyrazolo[1,5-a]pyridin-3-yl}formamido)-3-hydroxy-2-methylpropanamide;
Cpd 029—2-({5-[(2-fluoro-3-methylphenyl)methoxy]-2-methylpyrazolo[1,5-a]pyridin-3-yl}formamido)-3-hydroxy-2-methylpropanamide;
Cpd 030—2-({5-[(2,5-difluorophenyl)methoxy]-2-methylpyrazolo[1,5-a]pyridin-3-yl}formamido)-3-hydroxy-2-methylpropanamide;
Cpd 031—2-({5-[(2-fluoro-6-methylphenyl)methoxy]-2-methylpyrazolo[1,5-a]pyridin-3-yl}formamido)-3-hydroxy-2-methylpropanamide;
Cpd 032—2-({5-[(2,6-difluorophenyl)methoxy]-2-methylpyrazolo[1,5-a]pyridin-3-yl}formamido)-3-hydroxy-2-methylpropanamide;
Cpd 033—5-(benzyloxy)-2-methylpyrazolo[1,5-a]pyridine-3-carboxylic acid;
Cpd 034—5-(cyclopropylmethoxy)-2-methylpyrazolo[1,5-a]pyridine-3-carboxylic acid;
Cpd 035—5-[(2-fluorophenyl)methoxy]-2-methylpyrazolo[1,5-a]pyridine-3-carboxylic acid;
Cpd 036—5-[(2-chlorophenyl)methoxy]-2-methylpyrazolo[1,5-a]pyridine-3-carboxylic acid;
Cpd 037—N-(4,4-difluoro-1-hydroxy-2-methylbutan-2-yl)-2-methyl-5-[(4-methyl-1,3-thiazol-5-yl)methoxy]pyrazolo[1,5-a]pyridine-3-carboxamide,
Cpd 038—N-(4,4-difluoro-1-hydroxy-2-methylbutan-2-yl)-2-methyl-5-[(pyridin-2-yl)methoxy]pyrazolo[1,5-a]pyridine-3-carboxamide,
Cpd 042—2-methyl-5-[(pyridin-2-yl)methoxy]-N-(2,2,2-trifluoroethyl)pyrazolo[1,5-a]pyridine-3-carboxamide,
Cpd 043—N-(2-hydroxyethyl)-2-methyl-5-[(pyridin-2-yl)methoxy]pyrazolo[1,5-a]pyridine-3-carboxamide,
Cpd 044—N-[(1H-imidazol-2-yl)methyl]-2-methyl-5-[(pyridin-2-yl)methoxy]pyrazolo[1,5-a]pyridine-3-carboxamide,
Cpd 045—N-[3-(dimethylamino)-2,2-dimethylpropyl]-2-methyl-5-[(pyridin-2-yl)methoxy]pyrazolo[1,5-a]pyridine-3-carboxamide,
Cpd 046—N-(2,2-difluoro-2-phenylethyl)-2-methyl-5-[(pyridin-2-yl)methoxy]pyrazolo[1,5-a]pyridine-3-carboxamide,
Cpd 047—N-{[2-fluoro-6-(trifluoromethyl)phenyl]methyl}-2-methyl-5-[(pyridin-2-yl)methoxy]pyrazolo[1,5-a]pyridine-3-carboxamide,
Cpd 048—N-{[3-(difluoromethoxy)phenyl]methyl}-2-methyl-5-[(pyridin-2-yl)methoxy]pyrazolo[1,5-a]pyridine-3-carboxamide,
Cpd 049—N-(4,4-difluoropiperidin-3-yl)-2-methyl-5-[(4-methyl-1,3-thiazol-5-yl)methoxy]pyrazolo[1,5-a]pyridine-3-carboxamide,
Cpd 050—N-(4,4-difluoropiperidin-3-yl)-2-methyl-5-[(pyridin-2-yl)methoxy]pyrazolo[1,5-a]pyridine-3-carboxamide,
Cpd 051—N-(5,5-difluoropiperidin-3-yl)-2-methyl-5-[(4-methyl-1,3-thiazol-5-yl)methoxy]pyrazolo[1,5-a]pyridine-3-carboxamide,
Cpd 052—N-(5,5-difluoropiperidin-3-yl)-2-methyl-5-[(pyridin-2-yl)methoxy]pyrazolo[1,5-a]pyridine-3-carboxamide,
Cpd 053—2-methyl-5-[(4-methyl-1,3-thiazol-5-yl)methoxy]-N-(pyrrolidin-3-yl)pyrazolo[1,5-a]pyridine-3-carboxamide,
Cpd 054—2-methyl-5-[(pyridin-2-yl)methoxy]-N-(pyrrolidin-3-yl)pyrazolo[1,5-a]pyridine-3-carboxamide,
Cpd 055—N-[3,3-difluoro-1-(hydroxymethyl)cyclobutyl]-2-methyl-5-[(pyridin-2-yl)methoxy]pyrazolo[1,5-a]pyridine-3-carboxamide,
Cpd 056—N-[2-hydroxy-5-(trifluoromethyl)pyridin-3-yl]-2-methyl-5-[(pyridin-2-yl)methoxy]pyrazolo[1,5-a]pyridine-3-carboxamide,
Cpd 057—N-[5-(hydroxymethyl)-2,2-dimethyl-1,3-dioxan-5-yl]-2-methyl-5-[(pyridin-2-yl)methoxy]pyrazolo[1,5-a]pyridine-3-carboxamide,
Cpd 058—N-[1-(4-fluorophenyl)-2-hydroxyethyl]-2-methyl-5-[(pyridin-2-yl)methoxy]pyrazolo[1,5-a]pyridine-3-carboxamide,
Cpd 059—N-[2-hydroxy-1-(pyridin-2-yl)ethyl]-2-methyl-5-[(pyridin-2-yl)methoxy]pyrazolo[1,5-a]pyridine-3-carboxamide,
Cpd 061—N-[1-(4-fluorophenyl)-3-hydroxypropyl]-2-methyl-5-[(pyridin-2-yl)methoxy]pyrazolo[1,5-a]pyridine-3-carboxamide,
Cpd 062—2-methyl-5-[(pyridin-2-yl)methoxy]-N-(4,4,4-trifluoro-1-hydroxybutan-2-yl)pyrazolo[1,5-a]pyridine-3-carboxamide,
Cpd 063—2-methyl-5-[(pyridin-2-yl)methoxy]-N-[4,4,4-trifluoro-1-hydroxy-3-(trifluoromethyl)butan-2-yl]pyrazolo[1,5-a]pyridine-3-carboxamide,
Cpd 064—N-(4,4-difluoro-1-hydroxybutan-2-yl)-2-methyl-5-[(pyridin-2-yl)methoxy]pyrazolo[1,5-a]pyridine-3-carboxamide,
Cpd 066—N-(4,4-difluoro-1-methoxy-2-methylbutan-2-yl)-2-methyl-5-[(pyridin-2-yl)methoxy]pyrazolo[1,5-a]pyridine-3-carboxamide,
Cpd 068—N-(1-hydroxy-2-methyl-3-oxobutan-2-yl)-2-methyl-5-[(pyridin-2-yl)methoxy]pyrazolo[1,5-a]pyridine-3-carboxamide,
Cpd 071—N-[2,2-difluoro-1-(hydroxymethyl)cyclopropyl]-2-methyl-5-[(pyridin-2-yl)methoxy]pyrazolo[1,5-a]pyridine-3-carboxamide,
Cpd 071A—N-[cis-2,2-difluoro-1-(hydroxymethyl)cyclopropyl]-2-methyl-5-[(pyridin-2-yl)methoxy]pyrazolo[1,5-a]pyridine-3-carboxamide,
Cpd 071B—N-[trans-2,2-difluoro-1-(hydroxymethyl)cyclopropyl]-2-methyl-5-[(pyridin-2-yl)methoxy]pyrazolo[1,5-a]pyridine-3-carboxamide,
Cpd 072—N-[cis-2-(difluoromethyl)-1-(hydroxymethyl)cyclopropyl]-2-methyl-5-[(pyridin-2-yl)methoxy]pyrazolo[1,5-a]pyridine-3-carboxamide,
Cpd 074—N-[1-(1-cyclopropyl-1H-imidazol-2-yl)-2-hydroxyethyl]-2-methyl-5-[(pyridin-2-yl)methoxy]pyrazolo[1,5-a]pyridine-3-carboxamide,
Cpd 075—N-[1-hydroxy-2-(1-methyl-1H-pyrazol-3-yl)propan-2-yl]-2-methyl-5-[(pyridin-2-yl)methoxy]pyrazolo[1,5-a]pyridine-3-carboxamide,
Cpd 076—(R)-4-hydroxy-3-({2-methyl-5-[(pyridin-2-yl)methoxy]pyrazolo[1,5-a]pyridin-3-yl}formamido)butanamide,
Cpd 077—N-[2-hydroxy-1-(oxan-4-yl)ethyl]-2-methyl-5-[(pyridin-2-yl)methoxy]pyrazolo[1,5-a]pyridine-3-carboxamide,
Cpd 079—N-[1-hydroxy-3-(pyrimidin-2-yl)propan-2-yl]-2-methyl-5-[(pyridin-2-yl)methoxy]pyrazolo[1,5-a]pyridine-3-carboxamide,
Cpd 079A—(R)—N-[1-hydroxy-3-(pyrimidin-2-yl)propan-2-yl]-2-methyl-5-[(pyridin-2-yl)methoxy]pyrazolo[1,5-a]pyridine-3-carboxamide,
Cpd 079B—(S)—N-[1-hydroxy-3-(pyrimidin-2-yl)propan-2-yl]-2-methyl-5-[(pyridin-2-yl)methoxy]pyrazolo[1,5-a]pyridine-3-carboxamide,
Cpd 081—2-methyl-5-[(pyridin-2-yl)methoxy]-N-(4,4,4-trifluoro-2-hydroxybutyl)pyrazolo[1,5-a]pyridine-3-carboxamide,
Cpd 082—2-methyl-5-[(pyridin-2-yl)methoxy]-N-[4-(trifluoromethyl)pyrrolidin-3-yl]pyrazolo[1,5-a]pyridine-3-carboxamide,
Cpd 082A—2-methyl-5-[(pyridin-2-yl)methoxy]-N-[cis-4-(trifluoromethyl)pyrrolidin-3-yl]pyrazolo[1,5-a]pyridine-3-carboxamide,
Cpd 082B—2-methyl-5-[(pyridin-2-yl)methoxy]-N-[trans-4-(trifluoromethyl)pyrrolidin-3-yl]pyrazolo[1,5-a]pyridine-3-carboxamide,
Cpd 084—N-[3-(2,2-difluoroethyl)piperidin-3-yl]-2-methyl-5-[(pyridin-2-yl)methoxy]pyrazolo[1,5-a]pyridine-3-carboxamide,
Cpd 087—N-[2-hydroxy-1-(1-methyl-1H-pyrazol-3-yl)ethyl]-2-methyl-5-[(pyridin-2-yl)methoxy]pyrazolo[1,5-a]pyridine-3-carboxamide,
Cpd 088—N-[1-hydroxy-3-(pyridin-2-yl)propan-2-yl]-2-methyl-5-[(pyridin-2-yl)methoxy]pyrazolo[1,5-a]pyridine-3-carboxamide,
Cpd 089—3-hydroxy-2-methyl-2-({2-methyl-5-[(pyridin-2-yl)methoxy]pyrazolo[1,5-a]pyridin-3-yl}formamido)propanamide,
Cpd 090—2-methyl-5-[(pyridin-2-yl)methoxy]-N-(1,1,1-trifluoro-3-hydroxypropan-2-yl)pyrazolo[1,5-a]pyridine-3-carboxamide,
Cpd 091—N-[3-(hydroxymethyl)-2-oxopyrrolidin-3-yl]-2-methyl-5-[(pyridin-2-yl)methoxy]pyrazolo[1,5-a]pyridine-3-carboxamide,

The pyrazolo[1,5-a]pyridine derivatives according to the invention is for use in the treatment of pain which is preferably selected from nociceptive pain, inflammatory pain, and neuropathic pain. More preferably, the pain is post-operative pain. The pyrazolo[1,5-a]pyridine derivatives according to the invention is for use in the treatment of epilepsy.

In some embodiments, the pyrazolo[1,5-a]pyridine derivatives are selected from the group consisting of compounds 001-091 shown in table 1 below, including stereoisomers and pharmaceutically acceptable salts thereof:

TABLE 1
Exemplary pyrazolo[1,5-a]pyridine derivatives
Structure and Compound
CODE 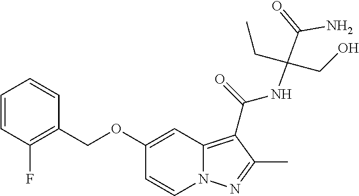 [SMILES: CCC(CO)(NC(=O)c1c(C)nn2ccc(OCc3ccccc3F)cc12)C(N)=O]
Cpd 001 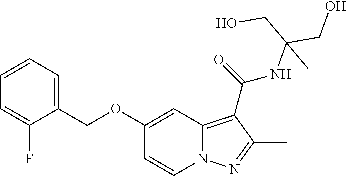 [SMILES: Cc1nn2ccc(OCc3ccccc3F)cc2c1C(=O)NC(C)(CO)CO]
Cpd 002 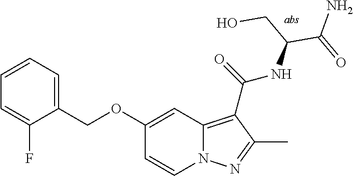 [SMILES: Cc1nn2ccc(OCc3ccccc3F)cc2c1C(=O)N[C@@H](CO)C(N)=O]
Cpd 003 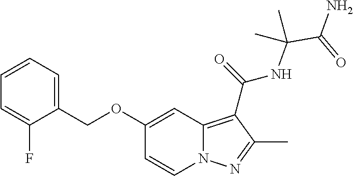 [SMILES: Cc1nn2ccc(OCc3ccccc3F)cc2c1C(=O)NC(C)(C)C(N)=O]
Cpd 004 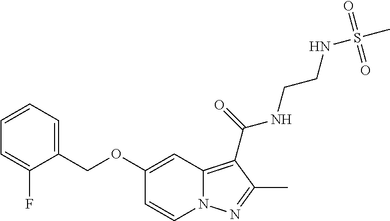 [SMILES: Cc1nn2ccc(OCc3ccccc3F)cc2c1C(=O)NCCNS(C)(=O)=O]
Cpd 005 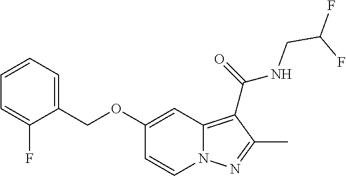 [SMILES: Cc1nn2ccc(OCc3ccccc3F)cc2c1C(=O)NCC(F)F]
Cpd 006 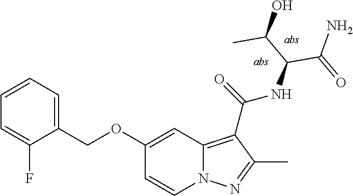 [SMILES: Cc1nn2ccc(OCc3ccccc3F)cc2c1C(=O)N[C@H](C(N)=O)[C@@H](C)O]
Cpd 007 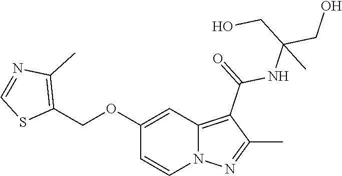 [SMILES: Cc1ncsc1COc1ccn2nc(C)c(C(=O)NC(C)(CO)CO)c2c1]
Cpd 008 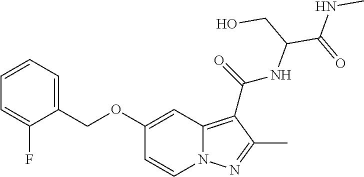 [SMILES: CNC(=O)C(CO)NC(=O)c1c(C)nn2ccc(OCc3ccccc3F)cc12]
Cpd 009 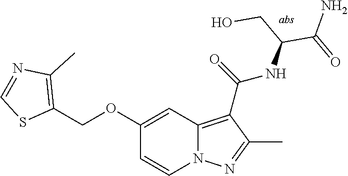 [SMILES: Cc1ncsc1COc1ccn2nc(C)c(C(=O)N[C@@H](CO)C(N)=O)c2c1]
Cpd 010 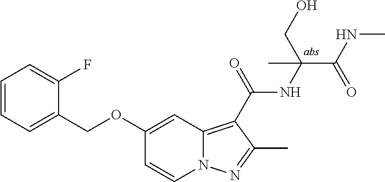 [SMILES: CNC(=O)C(C)(CO)NC(=O)c1c(C)nn2ccc(OCc3ccccc3F)cc12]
Cpd 011 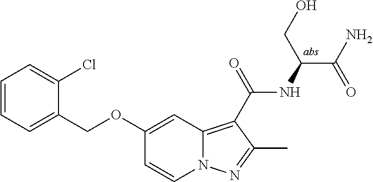 [SMILES: Cc1nn2ccc(OCc3ccccc3Cl)cc2c1C(=O)N[C@@H](CO)C(N)=O]
Cpd 012 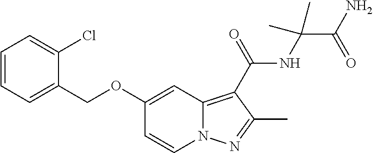 [SMILES: Cc1nn2ccc(OCc3ccccc3Cl)cc2c1C(=O)NC(C)(C)C(N)=O]
Cpd 013 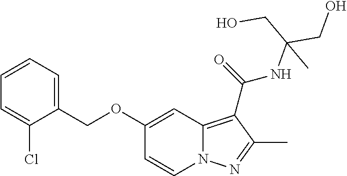 [SMILES: Cc1nn2ccc(OCc3ccccc3Cl)cc2c1C(=O)NC(C)(CO)CO]
Cpd 014 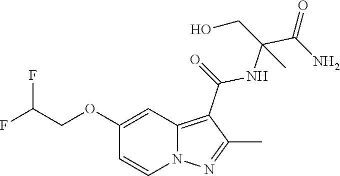 [SMILES: Cc1nn2ccc(OCC(F)F)cc2c1C(=O)NC(C)(CO)C(N)=O]
Cpd 015 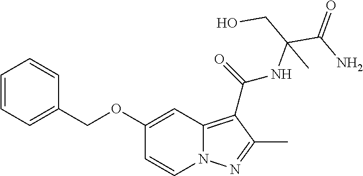 [SMILES: Cc1nn2ccc(OCc3ccccc3)cc2c1C(=O)NC(C)(CO)C(N)=O]
Cpd 016 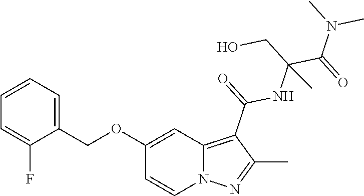 [SMILES: Cc1nn2ccc(OCc3ccccc3F)cc2c1C(=O)NC(C)(CO)C(=O)N(C)C]
Cpd 017 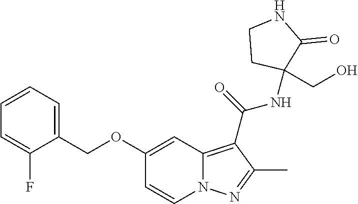 [SMILES: Cc1nn2ccc(OCc3ccccc3F)cc2c1C(=O)NC1(CO)CCNC1=O]
Cpd 018 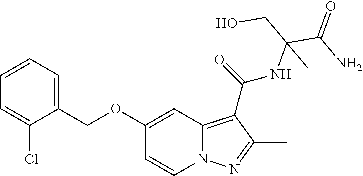 [SMILES: Cc1nn2ccc(OCc3ccccc3Cl)cc2c1C(=O)NC(C)(CO)C(N)=O]
Cpd 019 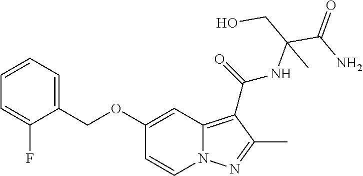 [SMILES: Cc1nn2ccc(OCc3ccccc3F)cc2c1C(=O)NC(C)(CO)C(N)=O]
Cpd 020 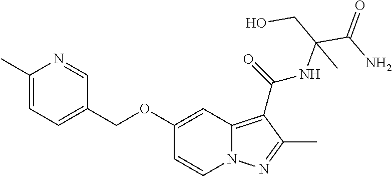 [SMILES: Cc1ccc(COc2ccn3nc(C)c(C(=O)NC(C)(CO)C(N)=O)c3c2)cn1]
Cpd 021 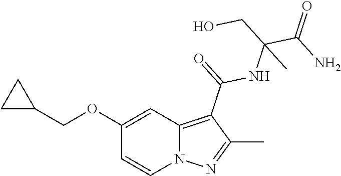 [SMILES: Cc1nn2ccc(OCC3CC3)cc2c1C(=O)NC(C)(CO)C(N)=O]
Cpd 022 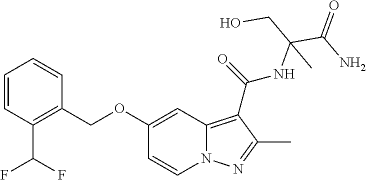 [SMILES: Cc1nn2ccc(OCc3ccccc3C(F)F)cc2c1C(=O)NC(C)(CO)C(N)=O]
Cpd 023 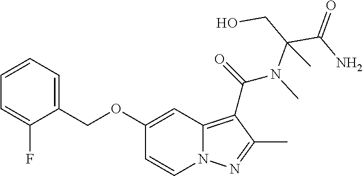 [SMILES: Cc1nn2ccc(OCc3ccccc3F)cc2c1C(=O)N(C)C(C)(CO)C(N)=O]
Cpd 024 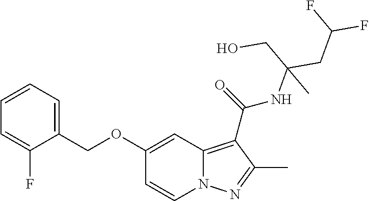 [SMILES: Cc1nn2ccc(OCc3ccccc3F)cc2c1C(=O)NC(C)(CO)CC(F)F]
Cpd 025 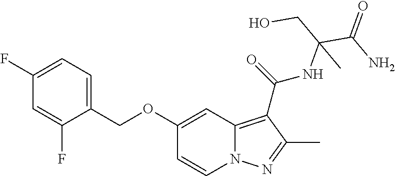 [SMILES: Cc1nn2ccc(OCc3ccc(F)cc3F)cc2c1C(=O)NC(C)(CO)C(N)=O]
Cpd 026 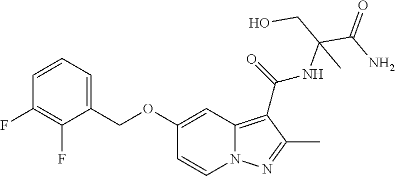 [SMILES: Cc1nn2ccc(OCc3cccc(F)c3F)cc2c1C(=O)NC(C)(CO)C(N)=O]
Cpd 027 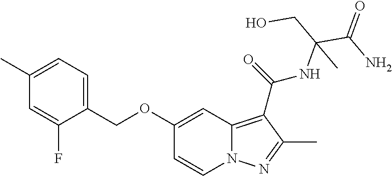 [SMILES: Cc1ccc(COc2ccn3nc(C)c(C(=O)NC(C)(CO)C(N)=O)c3c2)c(F)c1]
Cpd 028 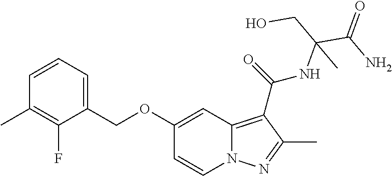 [SMILES: Cc1cccc(COc2ccn3nc(C)c(C(=O)NC(C)(CO)C(N)=O)c3c2)c1F]
Cpd 029 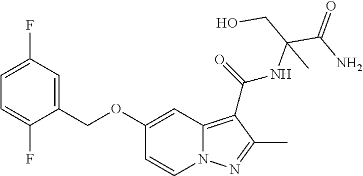 [SMILES: Cc1nn2ccc(OCc3cc(F)ccc3F)cc2c1C(=O)NC(C)(CO)C(N)=O]
Cpd 030 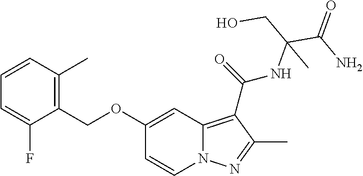 [SMILES: Cc1cccc(F)c1COc1ccn2nc(C)c(C(=O)NC(C)(CO)C(N)=O)c2c1]
Cpd 031 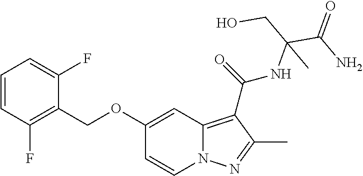 [SMILES: Cc1nn2ccc(OCc3c(F)cccc3F)cc2c1C(=O)NC(C)(CO)C(N)=O]
Cpd 032 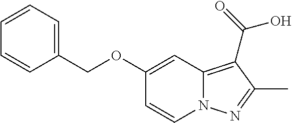 [SMILES: Cc1nn2ccc(OCc3ccccc3)cc2c1C(=O)O]
Cpd 033 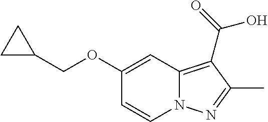 [SMILES: Cc1nn2ccc(OCC3CC3)cc2c1C(=O)O]
Cpd 034 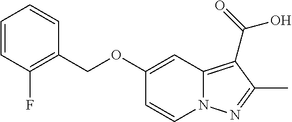 [SMILES: Cc1nn2ccc(OCc3ccccc3F)cc2c1C(=O)O]
Cpd 035 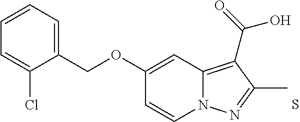
Cpd 036

In some embodiments, the pyrazolo[1,5-a]pyridine derivatives are selected from the group consisting of compounds 037-088 shown in table 2 below, including stereoisomers and pharmaceutically acceptable salts thereof:

TABLE 2
Exemplary Pyrazolo[1,5-a]pyridine derivatives
Structure and Compound
CODE 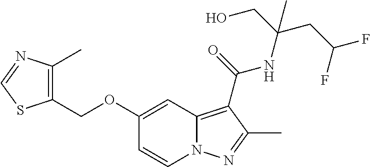 [SMILES: Cc1ncsc1COc1ccn2nc(C)c(C(=O)NC(C)(CO)CC(F)F)c2c1]
Cpd 037 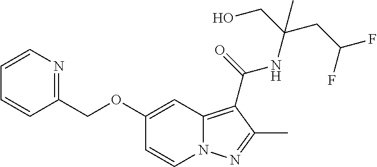 [SMILES: Cc1nn2ccc(OCc3ccccn3)cc2c1C(=O)NC(C)(CO)CC(F)F]
Cpd 038 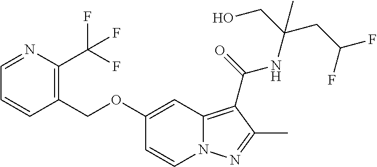 [SMILES: Cc1nn2ccc(OCc3cccnc3C(F)(F)F)cc2c1C(=O)NC(C)(CO)CC(F)F]
Cpd 039 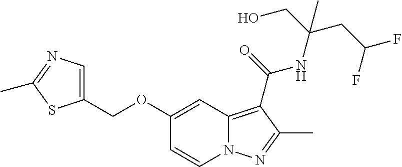 [SMILES: Cc1ncc(COc2ccn3nc(C)c(C(=O)NC(C)(CO)CC(F)F)c3c2)s1]
Cpd 040 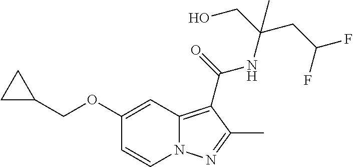 [SMILES: Cc1nn2ccc(OCC3CC3)cc2c1C(=O)NC(C)(CO)CC(F)F]
Cpd 041 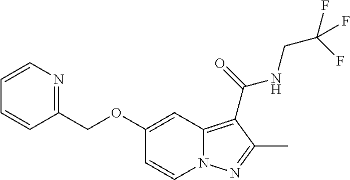 [SMILES: Cc1nn2ccc(OCc3ccccn3)cc2c1C(=O)NCC(F)(F)F]
Cpd 042 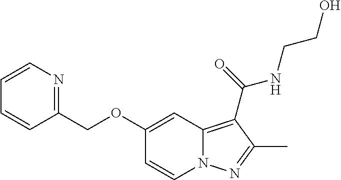 [SMILES: Cc1nn2ccc(OCc3ccccn3)cc2c1C(=O)NCCO]
Cpd 043 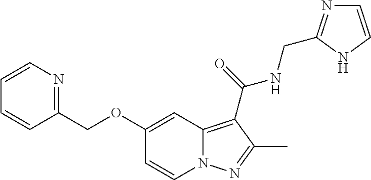 [SMILES: Cc1nn2ccc(OCc3ccccn3)cc2c1C(=O)NCc1ncc[nH]1]
Cpd 044 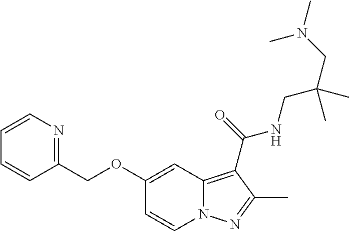 [SMILES: Cc1nn2ccc(OCc3ccccn3)cc2c1C(=O)NCC(C)(C)CN(C)C]
Cpd 045 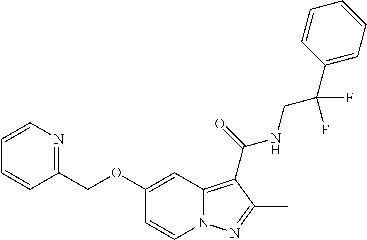 [SMILES: Cc1nn2ccc(OCc3ccccn3)cc2c1C(=O)NCC(F)(F)c1ccccc1]
Cpd 046 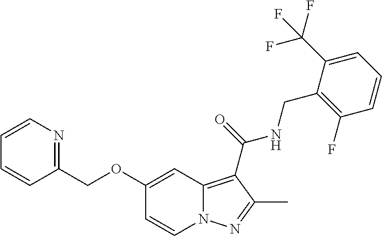 [SMILES: Cc1nn2ccc(OCc3ccccn3)cc2c1C(=O)NCc1c(F)cccc1C(F)(F)F]
Cpd 047 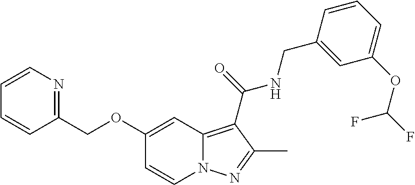 [SMILES: Cc1nn2ccc(OCc3ccccn3)cc2c1C(=O)NCc1cccc(OC(F)F)c1]
Cpd 048 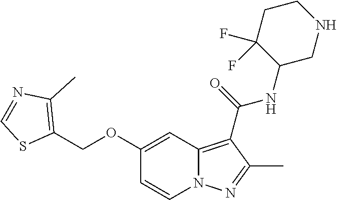 [SMILES: Cc1ncsc1COc1ccn2nc(C)c(C(=O)NC3CNCCC3(F)F)c2c1]
Cpd 049 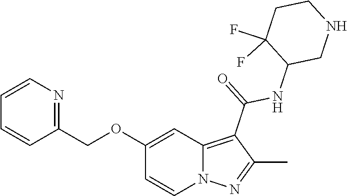 [SMILES: Cc1nn2ccc(OCc3ccccn3)cc2c1C(=O)NC1CNCCC1(F)F]
Cpd 050 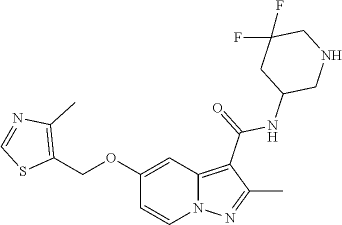 [SMILES: Cc1ncsc1COc1ccn2nc(C)c(C(=O)NC3CNCC(F)(F)C3)c2c1]
Cpd 051 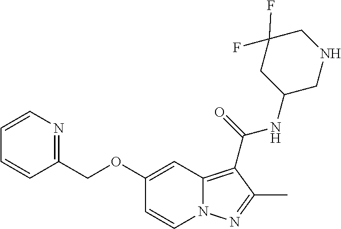 [SMILES: Cc1nn2ccc(OCc3ccccn3)cc2c1C(=O)NC1CNCC(F)(F)C1]
Cpd 052 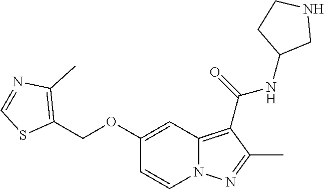 [SMILES: Cc1ncsc1COc1ccn2nc(C)c(C(=O)NC3CCNC3)c2c1]
Cpd 053 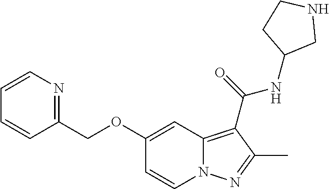 [SMILES: Cc1nn2ccc(OCc3ccccn3)cc2c1C(=O)NC1CCNC1]
Cpd 054 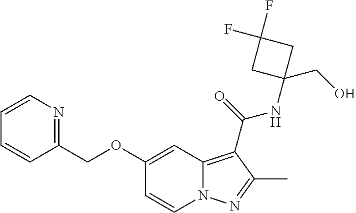 [SMILES: Cc1nn2ccc(OCc3ccccn3)cc2c1C(=O)NC1(CO)CC(F)(F)C1]
Cpd 055 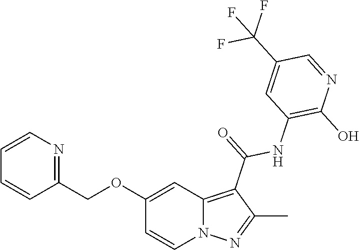 [SMILES: Cc1nn2ccc(OCc3ccccn3)cc2c1C(=O)Nc1cc(C(F)(F)F)cnc1O]
Cpd 056 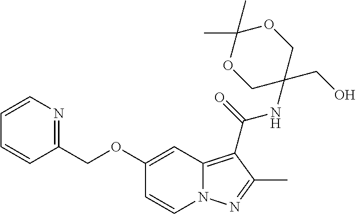 [SMILES: Cc1nn2ccc(OCc3ccccn3)cc2c1C(=O)NC1(CO)COC(C)(C)OC1]
Cpd 057 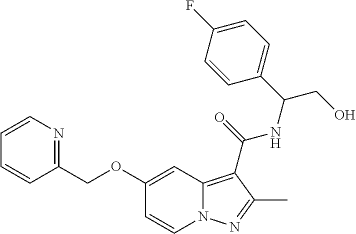 [SMILES: Cc1nn2ccc(OCc3ccccn3)cc2c1C(=O)NC(CO)c1ccc(F)cc1]
Cpd 058 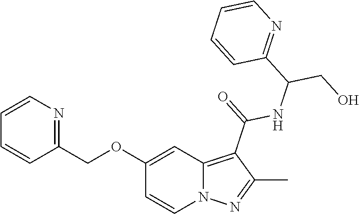 [SMILES: Cc1nn2ccc(OCc3ccccn3)cc2c1C(=O)NC(CO)c1ccccn1]
Cpd 059 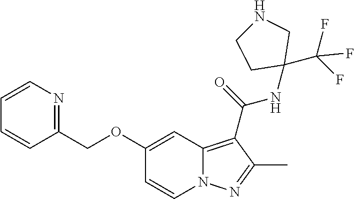 [SMILES: Cc1nn2ccc(OCc3ccccn3)cc2c1C(=O)NC1(C(F)(F)F)CCNC1]
Cpd 060 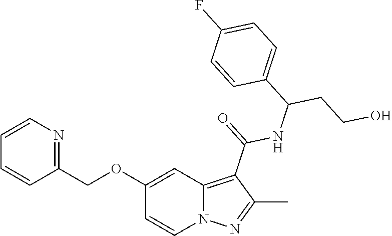 [SMILES: Cc1nn2ccc(OCc3ccccn3)cc2c1C(=O)NC(CCO)c1ccc(F)cc1]
Cpd 061 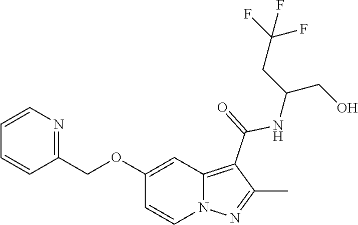 [SMILES: Cc1nn2ccc(OCc3ccccn3)cc2c1C(=O)NC(CO)CC(F)(F)F]
Cpd 062 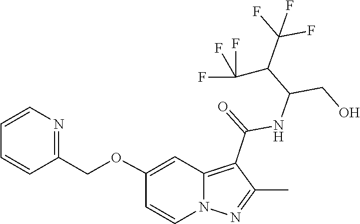 [SMILES: Cc1nn2ccc(OCc3ccccn3)cc2c1C(=O)NC(CO)C(C(F)(F)F)C(F)(F)F]
Cpd 063 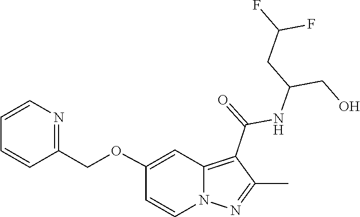 [SMILES: Cc1nn2ccc(OCc3ccccn3)cc2c1C(=O)NC(CO)CC(F)F]
Cpd 064 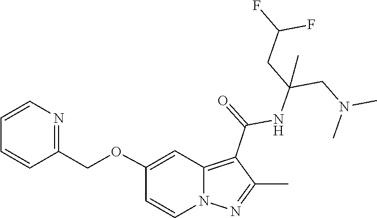 [SMILES: Cc1nn2ccc(OCc3ccccn3)cc2c1C(=O)NC(C)(CC(F)F)CN(C)C]
Cpd 065 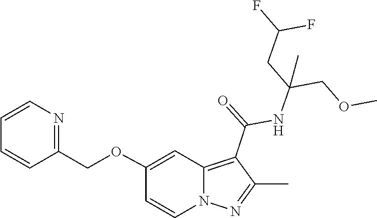 [SMILES: COCC(C)(CC(F)F)NC(=O)c1c(C)nn2ccc(OCc3ccccn3)cc12]
Cpd 066 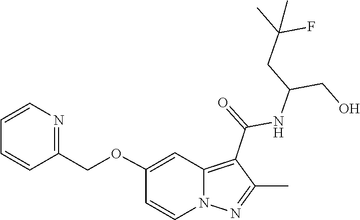 [SMILES: Cc1nn2ccc(OCc3ccccn3)cc2c1C(=O)NC(CO)CC(C)(C)F]
Cpd 067 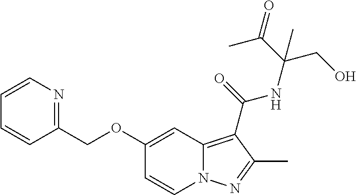 [SMILES: CC(=O)C(C)(CO)NC(=O)c1c(C)nn2ccc(OCc3ccccn3)cc12]
Cpd 068 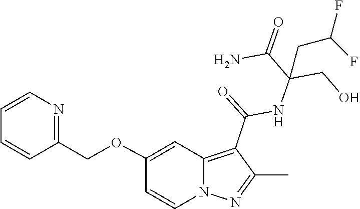 [SMILES: Cc1nn2ccc(OCc3ccccn3)cc2c1C(=O)NC(CO)(CC(F)F)C(N)=O]
Cpd 069 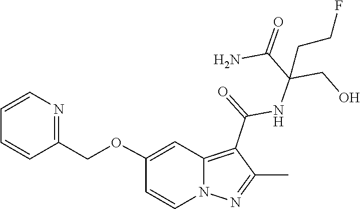 [SMILES: Cc1nn2ccc(OCc3ccccn3)cc2c1C(=O)NC(CO)(CCF)C(N)=O]
Cpd 070 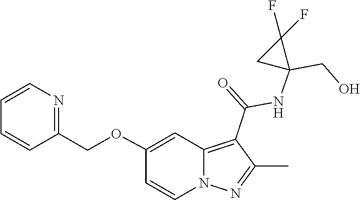 [SMILES: Cc1nn2ccc(OCc3ccccn3)cc2c1C(=O)NC1(CO)CC1(F)F]
Cpd 071 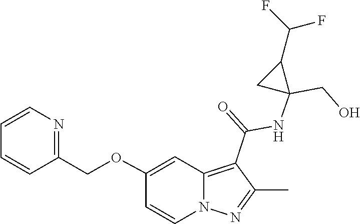 [SMILES: Cc1nn2ccc(OCc3ccccn3)cc2c1C(=O)NC1(CO)CC1C(F)F]
Cpd 072 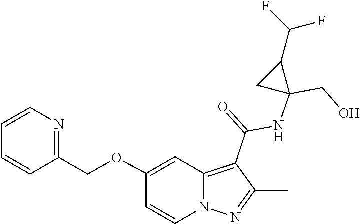 [SMILES: Cc1nn2ccc(OCc3ccccn3)cc2c1C(=O)NC1(CO)CC1C(F)F]
Cpd 073 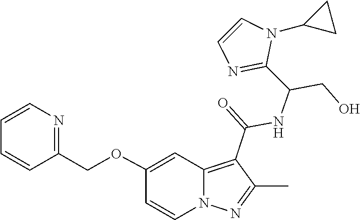 [SMILES: Cc1nn2ccc(OCc3ccccn3)cc2c1C(=O)NC(CO)c1nccn1C1CC1]
Cpd 074 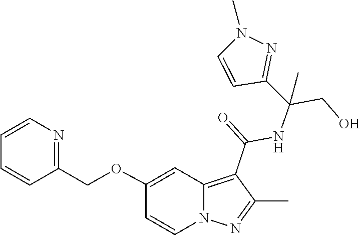 [SMILES: Cc1nn2ccc(OCc3ccccn3)cc2c1C(=O)NC(C)(CO)c1ccn(C)n1]
Cpd 075 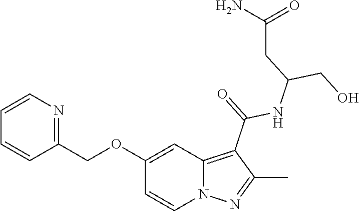 [SMILES: Cc1nn2ccc(OCc3ccccn3)cc2c1C(=O)NC(CO)CC(N)=O]
Cpd 076 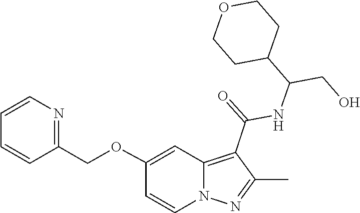 [SMILES: Cc1nn2ccc(OCc3ccccn3)cc2c1C(=O)NC(CO)C1CCOCC1]
Cpd 077 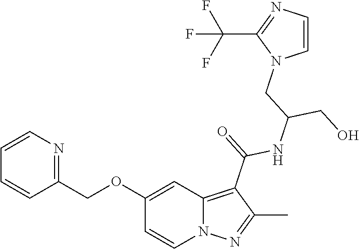 [SMILES: Cc1nn2ccc(OCc3ccccn3)cc2c1C(=O)NC(CO)Cn1ccnc1C(F)(F)F]
Cpd 078 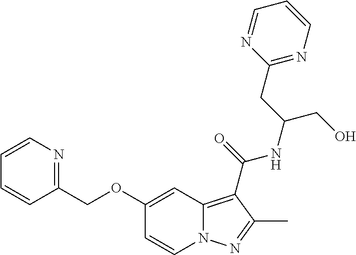 [SMILES: Cc1nn2ccc(OCc3ccccn3)cc2c1C(=O)NC(CO)Cc1ncccn1]
Cpd 079 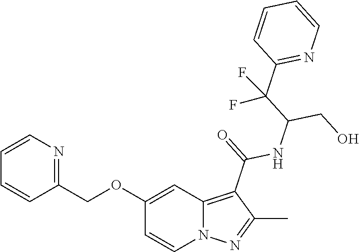 [SMILES: Cc1nn2ccc(OCc3ccccn3)cc2c1C(=O)NC(CO)C(F)(F)c1ccccn1]
Cpd 080 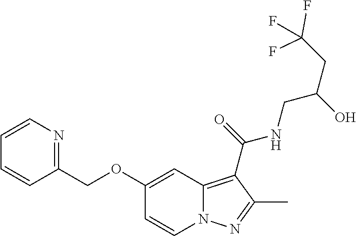 [SMILES: Cc1nn2ccc(OCc3ccccn3)cc2c1C(=O)NCC(O)CC(F)(F)F]
Cpd 081 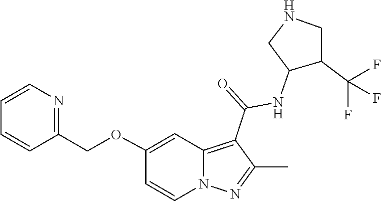 [SMILES: Cc1nn2ccc(OCc3ccccn3)cc2c1C(=O)NC1CNCC1C(F)(F)F]
Cpd 082 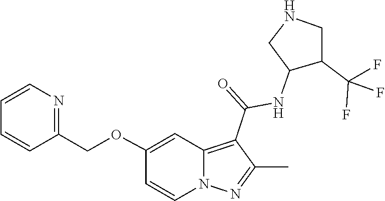 [SMILES: Cc1nn2ccc(OCc3ccccn3)cc2c1C(=O)NC1CNCC1C(F)(F)F]
Cpd 083 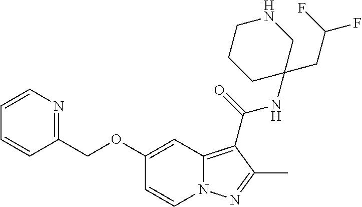 [SMILES: Cc1nn2ccc(OCc3ccccn3)cc2c1C(=O)NC1(CC(F)F)CCCNC1]
Cpd 084 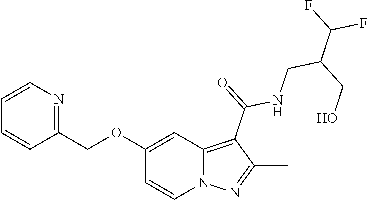 [SMILES: Cc1nn2ccc(OCc3ccccn3)cc2c1C(=O)NCC(CO)C(F)F]
Cpd 085 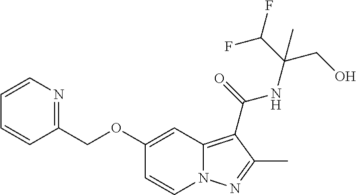 [SMILES: Cc1nn2ccc(OCc3ccccn3)cc2c1C(=O)NC(C)(CO)C(F)F]
Cpd 086 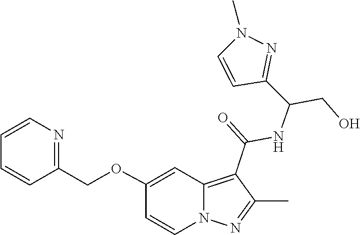 [SMILES: Cc1nn2ccc(OCc3ccccn3)cc2c1C(=O)NC(CO)c1ccn(C)n1]
Cpd 087 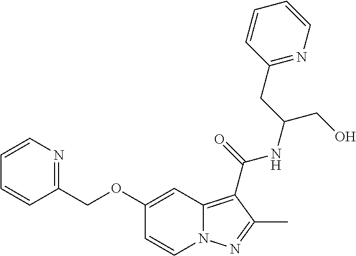 [SMILES: Cc1nn2ccc(OCc3ccccn3)cc2c1C(=O)NC(CO)Cc1ccccn1]
Cpd 088 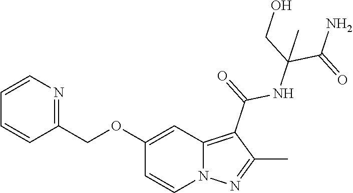 [SMILES: Cc1nn2ccc(OCc3ccccn3)cc2c1C(=O)NC(C)(CO)C(N)=O]
Cpd 089 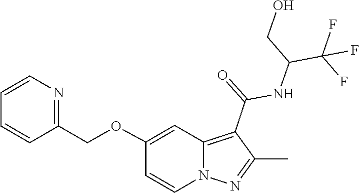 [SMILES: Cc1nn2ccc(OCc3ccccn3)cc2c1C(=O)NC(CO)C(F)(F)F]
Cpd 090 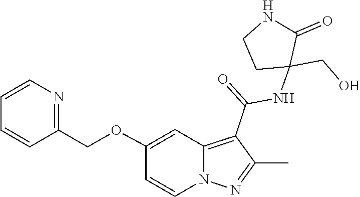 [SMILES: Cc1nn2ccc(OCc3ccccn3)cc2c1C(=O)NC1(CO)CCNC1=O]
Cpd 091

All definitions, embodiments and meanings of Q, T, U, V, R¹, R², R³, R⁴, R⁵, R^{5′}, R⁶, R⁷, and R⁸ including the disclosed substituents also analogously apply the pyrazolo[1,5-a]pyridine derivatives according to the invention, including but not limited to (a-1), (a-2), (a-3), (b-1), (b-2), and (b-3), which are not necessarily restricted for use in the treatment of pain. Thus, this aspect of the invention relates to the pyrazolo[1,5-a]pyridine derivatives as such, compositions comprising the pyrazolo[1,5-a]pyridine derivatives, medicaments comprising the pyrazolo[1,5-a]pyridine derivatives, and the pyrazolo[1,5-a]pyridine derivatives for use in the prevention and/or treatment of TRPM3 mediated disorders such as pain and/or inflammatory hypersensitivity and/or epilepsy; and/or for counteracting pain and/or inflammatory hypersensitivity and/or epilepsy. Preferably, the pain is selected from nociceptive pain, inflammatory pain, and neuropathic pain. More preferably, the pain is post-operative pain.

In some embodiments of the invention, the pyrazolo[1,5-a]pyridine derivative is selected from the group consisting of cpd 001 to cpd 036 as mentioned above and the physiologically acceptable salts thereof.

In some embodiments of the invention, the pyrazolo[1,5-a]pyridine derivative is selected from the group consisting of cpd 037 to cpd 091 as mentioned above and the physiologically acceptable salts thereof.

Another aspect of the invention relates to a pharmaceutical composition or a medicament comprising a compound according to the invention as described above.

Reference throughout this specification to “one embodiment” or “an embodiment” means that a particular feature, structure or characteristic described in connection with the embodiment is included in at least one embodiment of the invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments. Also, embodiments described for an aspect of the invention may be used for another aspect of the invention and can be combined. Where an indefinite or definite article is used when referring to a singular noun e.g., “a” or “an”, “the”, this includes a plural of that noun unless something else is specifically stated.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects.

In each of the following definitions, the number of carbon atoms represents the maximum number of carbon atoms generally optimally present in the substituent or linker; it is understood that where otherwise indicated in the present application, the number of carbon atoms represents the optimal maximum number of carbon atoms for that particular substituent or linker.

The term “leaving group” or “LG” as used herein means a chemical group which is susceptible to be displaced by a nucleophile or cleaved off or hydrolyzed in basic or acidic conditions. In a particular embodiment, a leaving group is selected from a halogen atom (e.g., Cl, Br, I) or a sulfonate (e.g., mesylate, tosylate, triflate).

The term “protecting group” refers to a moiety of a compound that masks or alters the properties of a functional group or the properties of the compound as a whole. The chemical substructure of a protecting group varies widely. One function of a protecting group is to serve as intermediates in the synthesis of the parental drug substance. Chemical protecting groups and strategies for protection/deprotection are well known in the art. See: “Protective Groups in Organic Chemistry”, Theodora W. Greene (John Wiley & Sons, Inc., New York, 1991.

Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug.

Protecting groups are removed either in vitro, in the instance of chemical intermediates, or in vivo, in the case of prodrugs. With chemical intermediates, it is not particularly important that the resulting products after deprotection, e.g., alcohols, be physiologically acceptable, although in general it is more desirable if the products are pharmacologically innocuous.

The term “heteroatom(s)” as used herein means an atom selected from nitrogen, which can be quaternized or present as an oxide; oxygen; and sulfur, including oxidized sulfurs including, sulfoxide and sulfone, and in some cases sulfonate. In certain instances, the compounds and/or synthetic intermediates may include heteroatoms such as boron, phosphorous, and silicon.

The term “alkyl, saturated or unsaturated” as used herein encompasses saturated alkyl as well as unsaturated alkyl such as alkenyl, alkynyl, and the like. The term “alkyl” as used herein means normal, secondary, or tertiary, linear or branched hydrocarbon with no site of unsaturation. Examples are methyl, ethyl, 1-propyl (n-propyl), 2-propyl (iPr), 1-butyl, 2-methyl-1-propyl(i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. The term “alkenyl” as used herein means normal, secondary or tertiary, linear or branched hydrocarbon with at least one site (usually 1 to 3, preferably 1) of unsaturation, namely a carbon-carbon, sp2 double bond. Examples include, but are not limited to: ethylene or vinyl (—CH═CH₂), allyl (—CH₂CH═CH₂), and 5-hexenyl (—CH₂CH₂CH₂CH₂CH═CH₂). The double bond may be in the cis or trans configuration. The term “alkynyl” as used herein means normal, secondary, tertiary, linear or branched hydrocarbon with at least one site (usually 1 to 3, preferably 1) of unsaturation, namely a carbon-carbon, sp triple bond. Examples include, but are not limited to: ethynyl (—C═CH), and 1-propynyl (propargyl, —CH₂C═CH).

The term “alkylene, saturated or unsaturated” as used herein encompasses saturated alkylene as well as unsaturated alkylene such as alkenylene, alkynylene, alkenynylene and the like. The term “alkylene” as used herein means saturated, linear or branched chain hydrocarbon radical having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like. The term “alkenylene” as used herein means linear or branched chain hydrocarbon radical with at least one site (usually 1 to 3, preferably 1) of unsaturation, namely a carbon-carbon, sp2 double bond, and having two monovalent radical centers derived by the removal of two hydrogen atoms from the same or two different carbon atoms of a parent alkene. The term “alkynylene” as used herein means linear or branched chain hydrocarbon radical with at least one site (usually 1 to 3, preferably 1) of unsaturation, namely a carbon-carbon, sp triple bond, and having two monovalent radical centers derived by the removal of two hydrogen atoms from the same or two different carbon atoms of a parent alkyne.

The term “heteroalkyl, saturated or unsaturated” as used herein encompasses saturated heteroalkyl as well as unsaturated heteroalkyl such as heteroalkenyl, heteroalkynyl, heteroalkenynyl and the like. The term “heteroalkyl” as used herein means linear or branched chain alkyl wherein one or more carbon atoms (usually 1, 2 or 3) are replaced by a heteroatom, i.e., an oxygen, nitrogen or sulfur atom, with the proviso that said chain may not contain two adjacent O atoms or two adjacent S atoms. This means that one or more —CH₃ of said alkyl can be replaced by —NH₂ and/or that one or more —CH₂— of said alkyl can be replaced by —NH—, —O— or —S—. The S atoms in said chains may be optionally oxidized with one or two oxygen atoms, to afford sulfoxides and sulfones, respectively. Furthermore, the heteroalkyl groups in the pyrazolo[1,5-a]pyridine derivatives of the invention can contain an oxo or thio group at any carbon or heteroatom that will result in a stable compound. Exemplary heteroalkyl groups include, but are not limited to, alcohols, alkyl ethers (such as for example -methoxy, -ethoxy, -butoxy . . . ), primary, secondary, and tertiary alkyl amines, amides, ketones, esters, alkyl sulfides, and alkyl sulfones. The term “heteroalkenyl” means linear or branched chain alkenyl wherein one or more carbon atoms (usually 1, 2 or 3) are replaced by an oxygen, nitrogen or sulfur atom, with the proviso that said chain may not contain two adjacent O atoms or two adjacent S atoms. The term heteroalkenyl thus comprises imines, —O-alkenyl, —NH-alkenyl, —N(alkenyl)₂, —N(alkyl)(alkenyl), and —S-alkenyl. The term “heteroalkynyl” as used herein means linear or branched chain alkynyl wherein one or more carbon atoms (usually 1, 2 or 3) are replaced by an oxygen, nitrogen or sulfur atom, with the proviso that said chain may not contain two adjacent O atoms or two adjacent S atoms. The term heteroalkynyl thus comprises -cyano, —O-alkynyl, —NH-alkynyl, —N(alkynyl)₂, —N(alkyl)(alkynyl), —N(alkenyl)(alkynyl), and —S-alkynyl.

The term “heteroalkylene, saturated or unsaturated” as used herein encompasses saturated heteroalkylene as well as unsaturated heteroalkylene such as heteroalkenylene, heteroalkynylene, heteroalkenynylene and the like. The term “heteroalkylene” as used herein means linear or branched chain alkylene wherein one or more carbon atoms (usually 1, 2 or 3) are replaced by a heteroatom, i.e., an oxygen, nitrogen or sulfur atom, with the proviso that said chain may not contain two adjacent O atoms or two adjacent S atoms. The term “heteroalkenylene” as used herein means linear or branched chain alkenylene wherein one or more carbon atoms (usually 1, 2 or 3) are replaced by an oxygen, nitrogen or sulfur atom, with the proviso that said chain may not contain two adjacent O atoms or two adjacent S atoms. The term “heteroalkynylene” as used herein means linear or branched chain alkynylene wherein one or more carbon atoms (usually 1, 2 or 3) are replaced by an oxygen, nitrogen or sulfur atom, with the proviso that said chain may not contain two adjacent O atoms or two adjacent S atoms.

The term “cycloalkyl, saturated or unsaturated” as used herein encompasses saturated cycloalkyl as well as unsaturated cycloalkyl such as cycloalkenyl, cycloalkynyl and the like. The term “cycloalkyl” as used herein and unless otherwise stated means a saturated cyclic hydrocarbon radical, such as for instance cyclopropyl, cyclobutyl, cyclopentyl, cyclohexyl, cycloheptyl, cyclooctyl, norbornyl, fenchyl, decalinyl, adamantyl and the like. The term “cycloalkenyl” as used herein means a non-aromatic cyclic hydrocarbon radical with at least one site (usually 1 to 3, preferably 1) of unsaturation, namely a carbon-carbon, sp2 double bond. Examples include, but are not limited to cyclopentenyl and cyclohexenyl. The double bond may be in the cis or trans configuration. The term “cycloalkynyl” as used herein means a non-aromatic cyclic hydrocarbon radical with at least one site (usually 1 to 3, preferably 1) of unsaturation, namely a carbon-carbon, sp triple. An example is cyclohept-1-yne. Fused systems of a cycloalkyl ring with a heterocycloalkyl ring are considered as heterocycloalkyl irrespective of the ring that is bound to the core structure. Fused systems of a cycloalkyl ring with an aryl ring are considered as aryl irrespective of the ring that is bound to the core structure. Fused systems of a cycloalkyl ring with a heteroaryl ring are considered as heteroaryl irrespective of the ring that is bound to the core structure.

The term “heterocycloalkyl, saturated or unsaturated” as used herein encompasses saturated heterocycloalkyl as well as unsaturated non-aromatic heterocycloalkyl including at least one heteroatom, i.e., an N, O, or S as ring member. The term “heterocycloalkyl” as used herein and unless otherwise stated means “cycloalkyl” wherein one or more carbon atoms (usually 1, 2 or 3) are replaced by an oxygen, nitrogen or sulfur atom, with the proviso that said chain may not contain two adjacent O atoms or two adjacent S atoms. The term “heterocycloalkenyl” as used herein and unless otherwise stated means “cycloalkenyl” wherein one or more carbon atoms (usually 1, 2 or 3) are replaced by an oxygen, nitrogen or sulfur atom, with the proviso that said chain may not contain two adjacent O atoms or two adjacent S atoms. The term “heterocycloalkynyl” as used herein and unless otherwise stated means “cycloalkynyl” wherein one or more carbon atoms (usually 1, 2 or 3) are replaced by an oxygen, nitrogen or sulfur atom, with the proviso that said chain may not contain two adjacent O atoms or two adjacent S atoms. Examples of saturated and unsaturated heterocycloalkyl include but are not limited to azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, tetrahydrothiopyran, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexahydrocyclopenta[c]pyrrole, octahydrocyclopenta[c]pyrrole, and octahydropyrrolo[1,2-a]pyrazine. Further heterocycloalkyls in the meaning of the invention are described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566. When the heterocycloalkyl contains no nitrogen as ring member, it is typically bonded through carbon. When the heterocycloalkyl contains nitrogen as ring member, it may be bonded through nitrogen or carbon. Fused systems of heterocycloalkyl ring with a cycloalkyl ring are considered as heterocycloalkyl irrespective of the ring that is bound to the core structure. Fused systems of a heterocycloalkyl ring with an aryl ring are considered as heterocycloalkyl irrespective of the ring that is bound to the core structure. Fused systems of a heterocycloalkyl ring with a heteroaryl ring are considered as heteroaryl irrespective of the ring that is bound to the core structure.

The term “aryl” as used herein means an aromatic hydrocarbon. Typical aryl groups include, but are not limited to 1 ring, or 2 or 3 rings fused together, radicals derived from benzene, naphthalene, anthracene, biphenyl, and the like. Fused systems of an aryl ring with a cycloalkyl ring are considered as aryl irrespective of the ring that is bound to the core structure. Fused systems of an aryl ring with a heterocycloalkyl ring are considered as heterocycloalkyl irrespective of the ring that is bound to the core structure. Thus, indoline, dihydrobenzofuran, dihydrobenzothiophene and the like are considered as heterocycloalkyl according to the invention. Fused systems of an aryl ring with a heteroaryl ring are considered as heteroaryl irrespective of the ring that is bound to the core structure.

The term “heteroaryl” as used herein means an aromatic ring system including at least one heteroatom, i.e., N, O, or S as ring member of the aromatic ring system. Examples of heteroaryl include but are not limited to benzimidazole, benzisoxazole, benzoxazole, benzodioxole, benzofuran, benzothiadiazole, benzothiazole, benzothiophene, carbazole, cinnoline, dibenzofuran, furane, furazane, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine.

By further way of example, carbon bonded heterocyclic rings are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

Carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl. By way of example, nitrogen bonded heterocyclic rings are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of an isoindole, or isoindoline, position 4 of a morpholine, and position 9 of a carbazole, or R-carboline. Nitrogen bonded heterocycles include 1-aziridyl, 1-azetedyl, 1-pyrrolyl, 1-imidazolyl, 1-pyrazolyl, and 1-piperidinyl. Further heteroaryls in the meaning of the invention are described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566.

As used herein with respect to a substituting group, and unless otherwise stated, the terms “monosubstituted”, “disubstituted”, “trisubstituted”, “polysubstituted” and the like means chemical structures defined herein, wherein the respective moiety is substituted with one or more substituents, meaning that one or more hydrogen atoms of said moiety are each independently replaced with a substituent. For example, —C₁₋₆-alkyl that may be polysubstituted with —F includes —CH₂F, —CHF₂, —CF₃, —CH₂CF₃, CF₂CF₃, and the like. Likewise, —C₁₋₆-alkyl that may be polysubstituted with substituents independently of one another selected from —F and —Cl includes —CH₂F, —CHF₂, —CF₃, —CH₂CF₃, CF₂CF₃, —CH₂Cl, —CHCl₂, —CCl₃, —CH₂CCl₃, CCl₂CCl₃, —CHClF, —CClF₂, —CCl₂CF₃, —CF₂CCl₃, —CClFCCl₂F, and the like. Any substituent designation that is found in more than one site in a compound of this invention shall be independently selected.

As used herein and unless otherwise stated, the term “solvate” includes any combination which may be formed by a derivative of this invention with a suitable inorganic solvent (e.g., hydrates) or organic solvent, such as but not limited to alcohols, ketones, esters, ethers, nitriles and the like.

The term “subject” as used herein, refers to an animal including humans, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation or partial alleviation of the symptoms of the disease or disorder being treated.

The term “composition” as used herein is intended to encompass a product comprising the specified ingredients in the therapeutically effective amounts, as well as any product which results, directly or indirectly, from combinations of the specified ingredients in the specified amounts.

The term “antagonist” or “inhibitor” as used herein refers to a compound capable of producing, depending on the circumstance, a functional antagonism of the TRPM3 ion channel, including competitive antagonists, non-competitive antagonists, desensitizing agonists, and partial agonists. In general, “antagonists” and “inhibitors” can be understood to modulate TRPM3.

For purposes of the invention, the term “TRPM3-modulated” is used to refer to the condition of being affected by the modulation of the TRPM3 ion channel, including the state of being mediated by the TRPM3 ion channel.

The term “TRPM3 mediated disorder” as used herein refers to disorders or diseases for which the use of an antagonist or modulator of TRPM3 would prevent, treat, (partially) alleviate or improve the symptoms and consist of pain, inflammatory hypersensitivity condition and epilepsy. According to the International Association for the Study of Pain and for the purpose of the invention, pain is an unpleasant sensory and emotional experience associated with actual or potential tissue damage, or described in terms of such damage. Preferably, the TRPM3 mediated disorder is pain which is preferably selected from nociceptive pain, inflammatory pain, and neuropathic pain. More preferably, the pain is post-operative pain. For the purpose of the invention, the term “inflammatory hypersensitivity” is used to refer to a condition that is characterized by one or more hallmarks of inflammation, including edema, erythema, hyperthermia and pain, and/or by an exaggerated physiologic or pathophysiologic response to one or more than one type of stimulation, including thermal, mechanical and/or chemical stimulation.

The pyrazolo[1,5-a]pyridine derivatives of the invention have been shown to be or are understood to be antagonists or modulators of TRPM3 and the invention therefore provides the compounds as such, the compounds for use as a medicine, more specifically for use as a medicine in the prevention or treatment of TRPM3 mediated disorders in a subject with a therapeutically effective amount of a pyrazolo[1,5-a]pyridine derivative of the invention.

In an embodiment of the invention, the pyrazolo[1,5-a]pyridine derivative of the invention is the sole pharmacologically active compound to be administered for therapy. In another embodiment of the invention, the pyrazolo[1,5-a]pyridine derivative of the invention may be employed in combination with other therapeutic agents for the treatment or prophylaxis of TRPM3 mediated disorders. The invention therefore also relates to the use of a composition comprising:
one or more compounds of the formulae and embodiments herein, and
one or more further therapeutic or preventive agents that are used for the prevention or treatment of TRPM3 mediated disorders as biologically active agents in the form of a combined preparation for simultaneous, separate or sequential use.

The pharmaceutical composition or combined preparation according to this invention may contain pyrazolo[1,5-a]pyridine derivatives of the invention over a broad content range depending on the contemplated use and the expected effect of the preparation. Generally, the content of the pyrazolo[1,5-a]pyridine derivatives of the invention of the combined preparation is within the range of 0.1 to 99.9% by weight, preferably from 1 to 99% by weight, more preferably from 5 to 95% by weight.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g., one of them may be in the form of an oral or parenteral formulation whereas the other is in the form of an ampoule for intravenous injection or an aerosol.

Those of skill in the art will also recognize that the pyrazolo[1,5-a]pyridine derivatives of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The terms “pharmaceutically acceptable salts” or “physiologically acceptable salts” as used herein means the therapeutically active non-toxic salt forms which the compounds of formulae herein are able to form. Therefore, the compounds of this invention optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The pyrazolo[1,5-a]pyridine derivatives of the invention may bear multiple positive or negative charges. The net charge of the pyrazolo[1,5-a]pyridine derivatives of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li*, Na*, and K*. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalogen acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which the compounds of formulae herein as well as their salts are able to form, such as for example hydrates, alcoholates and the like. Finally, it is to be understood that the compositions herein comprise pyrazolo[1,5-a]pyridine derivatives of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The pyrazolo[1,5-a]pyridine derivatives of the invention also include physiologically acceptable salts thereof. Examples of physiologically acceptable salts of the pyrazolo[1,5-a]pyridine derivatives of the invention include salts derived from an appropriate base, such as an alkali metal (for example, sodium), an alkaline earth (for example, magnesium), ammonium and NX4⁺ (wherein X is —C₁₋₆-alkyl). Physiologically acceptable salts of a hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX4⁺ (wherein X typically is independently selected from —H or a —C₁₋₄-alkyl group). However, salts of acids or bases which are not physiologically acceptable may also find use, for example, in the preparation or purification of a physiologically acceptable compound. All salts, whether or not derived form a physiologically acceptable acid or base, are within the scope of the invention.

As used herein and unless otherwise stated, the term “enantiomer” means each individual optically active form of an pyrazolo[1,5-a]pyridine derivative of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “isomers” as used herein means all possible isomeric forms, including tautomeric and stereochemical forms, which the compounds of formulae herein may possess, but not including position isomers. Typically, the structures shown herein exemplify only one tautomeric or resonance form of the compounds, but the corresponding alternative configurations are contemplated as well. Unless otherwise stated, the chemical designation of compounds denotes the mixture of all possible stereochemically isomeric forms, said mixtures containing all diastereomers and enantiomers (since the compounds of formulae herein may have at least one chiral center) of the basic molecular structure, as well as the stereochemically pure or enriched compounds. More particularly, stereogenic centers may have either the R- or S-configuration, and multiple bonds may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e., at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question.

Separation of stereoisomers is accomplished by standard methods known to those in the art. One enantiomer of an pyrazolo[1,5-a]pyridine derivative of the invention can be separated substantially free of its opposing enantiomer by a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched compound. A method of determining optical purity involves making chiral esters, such as a menthyl ester or Mosher ester, a-methoxy-a-(trifluoromethyl)phenyl acetate (Jacob Ill. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111).Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCel® CA, OA, OB5, OC5, OD, OF, OG, OJ and OK, and Chiralpak® AD, AS, OP(+) and OT(+). Appropriate eluents or mobile phases for use in combination with said polysaccharide chiral stationary phases are hexane and the like, modified with an alcohol such as ethanol, isopropanol and the like. (“Chiral Liquid Chromatography” (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

The terms cis and trans are used herein in accordance with Chemical Abstracts nomenclature and include reference to the position of the substituents on a ring moiety. The absolute stereochemical configuration of the compounds of the formulae described herein may easily be determined by those skilled in the art while using well-known methods such as, for example, X-ray diffraction.

When a compound is crystallized from a solution or slurry, it can be crystallized in a different arrangement lattice of spaces (this property is called “polymorphism”) to form crystals with different crystalline forms, each of which is known as “polymorphs”. The term “Polymorph” as used herein, therefore, refers to a crystal form of a compound of Formula (I), where the molecules are localized in the three-dimensional lattice sites. Different polymorphs of the compound of Formula (I) may be different from each other in one or more physical properties, such as solubility and dissolution rate, true specific gravity, crystal form, accumulation mode, flowability and/or solid state stability. etc.

Pyrazolo[1,5-a]pyridine derivatives of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intranasal, intravenous, intraarterial, intradermal, intrathecal and epidural). The preferred route of administration may vary with for example the condition of the recipient.

The therapeutically effective amount of the preparation of the compound(s), especially for the treatment of TRPM3 mediated disorders in humans and other mammals or in animals, preferably is a TRPM3 ion channel inhibiting amount of the compounds as defined herein and corresponds to an amount which ensures a plasma level of between 1 μg/ml and 100 mg/ml.

Suitable dosages of the compounds or compositions of the invention should be used to treat or prevent the TRPM3 mediated disorders in a subject. Depending upon the pathologic condition to be treated and the patient's condition, the said effective amount may be divided into several sub-units per day or may be administered at more than one day intervals.

The invention further provides (pharmaceutical) compositions comprising one or more pyrazolo[1,5-a]pyridine derivatives of the invention, more in particular of all the Formula (I) and other formulas and embodiments described herein and the more particular aspects or embodiments thereof. Furthermore, the invention provides the compounds or (pharmaceutical) compositions of the invention, more in particular of all the Formula (I) and other formulas and embodiments described herein and the more particular aspects or embodiments thereof, for use as a medicine, more in particular for use in the treatment of pain. The TRPM3 mediated disorders are selected from pain and an inflammatory hypersensitivity condition and epilepsy.

The pyrazolo[1,5-a]pyridine derivatives of the invention may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986).

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e., the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the invention. They may also include additives such as wetting agents, dispersing agents, stickers, adhesives, emulsifying agents, surface-active agents, solvents, coatings, antibacterial and antifungal agents, isotonic agents and the like, provided the same are consistent with pharmaceutical practice, i.e., carriers and additives which do not create permanent damage to mammals. The pharmaceutical compositions of the invention may be prepared in any known manner, for instance by homogeneously mixing, coating and/or grinding the active ingredients, in a one-step or multi-steps procedure, with the selected carrier material and, where appropriate, the other additives such as surface-active agents. may also be prepared by micronisation, for instance in view to obtain them in the form of microspheres usually having a diameter of about 1 to 10 gm, namely for the manufacture of microcapsules for controlled or sustained release of the active ingredients.

While it is possible for the pyrazolo[1,5-a]pyridine derivatives to be administered alone it is preferable to present them as pharmaceutical formulations. The formulations, both for veterinary and for human use, of the invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations of the invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent.

Molded tablets may be made by molding in a suitable machine a mixture of the powdered compound moistened with an inert liquid diluent. The tablets may optionally be coated or scored and may be formulated so as to provide slow or controlled release of the active ingredient therein. For infections of the eye or other external tissues e.g., mouth and skin, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus, the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously 0.5 to 10% particularly about 1.5% w/w. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc.), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Pyrazolo[1,5-a]pyridine derivatives of the invention can be used to provide controlled release pharmaceutical formulations containing as active ingredient one or more pyrazolo[1,5-a]pyridine derivatives of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more pyrazolo[1,5-a]pyridine derivatives of the invention can be prepared according to conventional methods.

Another embodiment of this invention relates to various precursor or “prodrug” forms of the pyrazolo[1,5-a]pyridine derivatives of the invention. It may be desirable to formulate the pyrazolo[1,5-a]pyridine derivatives of the invention in the form of a chemical species which itself is not significantly biologically-active, but which when delivered to the animal, mammal or human will undergo a chemical reaction catalyzed by the normal function of the body, inter alia, enzymes present in the stomach or in blood serum, said chemical reaction having the effect of releasing a compound as defined herein. The term “prodrug” thus relates to these species which are converted in vivo into the active pharmaceutical ingredient.

The prodrugs of the pyrazolo[1,5-a]pyridine derivatives of the invention can have any form suitable to the formulator, for example, esters are non-limiting common pro-drug forms. In the present case, however, the pro-drug may necessarily exist in a form wherein a covalent bond is cleaved by the action of an enzyme present at the target locus. For example, a C—C covalent bond may be selectively cleaved by one or more enzymes at said target locus and, therefore, a pro-drug in a form other than an easily hydrolysable precursor, inter alia an ester, an amide, and the like, may be used. The counterpart of the active pharmaceutical ingredient in the pro-drug can have different structures such as an amino acid or peptide structure, alkyl chains, sugar moieties and others as known in the art.

For the purpose of the invention the term “therapeutically suitable pro-drug” is defined herein as “a compound modified in such a way as to be transformed in vivo to the therapeutically active form, whether by way of a single or by multiple biological transformations, when in contact with the tissues of the animal, mammal or human to which the pro-drug has been administered, and without undue toxicity, irritation, or allergic response, and achieving the intended therapeutic outcome”.

More specifically the term “prodrug”, as used herein, relates to an inactive or significantly less active derivative of a compound such as represented by the structural formulae herein described, which undergoes spontaneous or enzymatic transformation within the body in order to release the pharmacologically active form of the compound. For a comprehensive review, reference is made to Rautio J. et al. (“Prodrugs: design and clinical applications” Nature Reviews Drug Discovery, 2008, doi: 10.1038/nrd2468).

Representative pyrazolo[1,5-a]pyridine derivatives of the invention can be synthesized in accordance with the general synthetic methods described below and illustrated in the schemes that follow. Since the schemes are an illustration, the invention should not be construed as being limited by the specific chemical reaction and specific conditions described in the schemes and examples. The various starting material used in the schemes are commercially available or may be prepared by methods well within the skill persons versed in the art. The variables are as defined herein and within the skill of persons verses in the art.

Exemplary embodiments of the invention are summarized as Clauses 1 to 51 hereinafter:
1. A compound of formula (I), a stereo-isomeric form, a physiologically acceptable salt, solvate and/or polymorph thereof

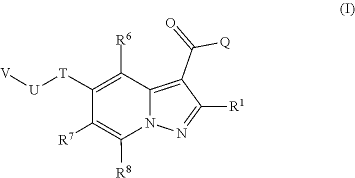

preferably, the compound of formula (I), a stereo-isomeric form, a physiologically acceptable salt, solvate and/or polymorph thereof, optionally for use in the treatment of pain or epilepsy or in methods of treating pain or epilepsy;
wherein
R¹ represents —F, —Cl, —Br, —I, —CN, —R^{W}, —OR^{W}, —OC(═O)R^{W}, —NR^{W}R^{X}, —NR^{W}C(═O)R^{X}, —SR^{W}, —S(═O)R^{W}, —S(═O)₂R^{W}, —C(═O)R^{W}, —C(═O)OR^{W}, or —C(═O)NR^{W}R^{X};
Q represents —OR² or —NR³R⁴;
R² represents —R^{Y};
R³ represents —OH or —R^{Y};
R⁴ represents —R^{Y} or —S(═O)₂R^{Y};
   or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —CR⁵R^{5′}—; or T represents —CR⁵R^{5′}— and U represents
—O—;
R⁵ and R^{5′} independently of one another represent —R^{Y};
R⁶, R⁷ and R⁸ independently of one another represent —F, —Cl, —Br, —I, —CN, —NO₂, —SF₅, —R^{W}, —OR^{W}, —OC(═O)R^{W}, —NR^{W}R^{X}, —NR^{W}C(═O)R^{X}, —SR^{W}, —S(═O)R^{W}, —S(═O)₂R^{W}, —C(═O)R^{W}, —C(═O)OR^{W}, or —C(═O)NR^{W}R^{X};
V represents 3-14-membered heterocycloalkyl, saturated or unsaturated; 3-14-membered cycloalkyl, saturated or unsaturated; 5-14-membered aryl, C₁-C₆ alkyl; or 5-14-membered heteroaryl; in each case unsubstituted, mono- or polysubstituted with substituents independently of one another selected from —F, —Cl, —Br, —I, —CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}; wherein
R^{W} and R^{X} independently of one another in each case independently represent
   —H;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R^{Y} and R^{Z} independently of one another in each case independently represent
   —H;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
   6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
or R^{Y} and R^{Z} together form a 4, 5, 6, 7 or 8 membered heterocycle containing 1 to 3 heteroatoms selected from N, O and S, saturated or unsaturated, unsubstituted or mono- or polysubstituted;
and wherein “mono- or polysubstituted” in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-O—CF₃, —C₁₋₆-alkylene-O—CF₂H, —C₁₋₆-alkylene-O—CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —N(3-14-membered cycloalkyl)(C₁₋₆-alkyl), —N(C₁₋₆-alkyl)-C₁₋₆-alkylene-OH, —N(H)—C₁₋₆-alkylene-OH, —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered hetero-cycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, —S(═O)₂-(5 to 14-membered heteroaryl).
2. The compound per se, or for use according to Clause 1, wherein T represents —O— and U represents —CR⁵R^{5′}—.
3. The compound per se, or for use according to Clause 1, wherein T represents —CR⁵R^{5′}- and U represents —O—.
4. The compound per se, or for use according to any one of Clauses 1 to 3, wherein Q represents —NR³R⁴.
5. The compound per se, or for use according to any one of Clauses 1 to 3, wherein Q represents —OR².
6. The compound per se, or for use according to any one of Clauses 1 to 5, wherein V represents 3-14-membered heterocycloalkyl, saturated or unsaturated; 3-14-membered cycloalkyl, saturated or unsaturated; 5-14-membered aryl; C₁-C₆ alkyl; or 5-14-membered heteroaryl; in each case unsubstituted, mono- or polysubstituted with substituents independently of one another selected from —F, —Cl, —Br, —I, —CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
7. The compound per se, or for use according to Clause 6, wherein the 5-14-membered heteroaryl is mono- or poly-substituted.
8. The compound per se, or for use according to Clause 6 or 7, wherein the 5-14-membered heteroaryl is selected from benzimidazole, benzisoxazole, benzoxazole, benzodioxole, benzofuran, benzothiadiazole, benzothiazole, benzothiophene, carbazole, cinnoline, dibenzofuran, furane, furazane, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
9. The compound per se, or for use according to any one of Clauses 6 to 8, wherein the 5-14-membered heteroaryl is selected from the group consisting of furane, thiophene, imidazole, pyrazole, oxazole, isoxazole, thiazole, triazole, pyridine, isoquinoline, benzothiazole, pyridazine, pyrimidine, imidazopyridine; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
10. The compound per se, or for use according to any one of Clauses 6 to 9, wherein the 5-14-membered heteroaryl is selected from the group consisting of furan-2-yl, furan-3-yl, thiophen-2-yl, thiophen-3-yl, pyrazol-3-yl, pyrazol-4-yl, pyrazol-5-yl, oxazol-5-yl, isoxazol-4-yl, thiazol-2-yl, thiazol-4-yl, thiazol-5-yl, 1,2,4-triazol-3-yl, 1,2,3-triazol-4-yl, pyridin-2-yl, pyridin-3-yl, pyridin-4-yl, isoquinolin-1-yl, isoquinolin-5-yl, benzo[d]thiazol-2-yl, pyridazin-3-yl, pyrimidin-5-yl, and imidazo[1,2-a]pyridin-6-yl; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
11. The compound per se, or for use according to any one of Clauses 1 to 5, wherein V represents 3-14-membered heterocycloalkyl, saturated or unsaturated, unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
12. The compound per se, or for use according to Clause 11, wherein the 3-14-membered heterocycloalkyl is selected from azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, tetrahydrothiopyran, thiazolidine, thietane, thiirane, thiolane, thiomorpholine, indoline, dihydrobenzofuran, dihydrobenzothiophene, 1,1-dioxothia-cyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexa-hydrocyclopenta[c]pyrrole, octahydrocyclopenta[c]pyrrole, and octahydropyrrolo[1,2-a]pyrazine; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
13. The compound per se, or for use according to Clause 11 or 12, wherein the 3-14-membered heterocycloalkyl is oxane, or oxetanyl; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
14. The compound per se, or for use according to any one of Clauses 11 to 13, wherein the 3-14-membered heterocycloalkyl is oxan-4-yl or oxetan-3-yl; in each case unsubstituted, mono- or polysubstituted with substituents independently of one another selected from —F, —Cl, —Br, —I, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
15. The compound per se, or for use according to any one of clauses 1 to 5, wherein the 3-14-membered cycloalkyl, saturated or unsaturated, is cyclopropyl, cyclobutyl, cyclopentyl, cyclohexyl, cycloheptyl, cyclooctyl, cyclononyl, or cyclodecyl including unfused or unbridged, fused, or bridged cycloalkyls; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
16. The compound per se, or for use according to any one of clauses 1 to 5, wherein the 5-14-membered aryl is phenyl or another 5-14 membered aryl unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
17. The compound per se, or for use according to any one of clauses 1 to 6, wherein the C₁-C₆ alkyl or C₁-C₆ heteroalkyl representing V is saturated or unsaturated, unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
18. The compound per se, or for use according to any one of the preceding Clauses, wherein V is unsubstituted, mono- or polysubstituted with substituents independently of one another selected from
   —F, —Cl, —Br, —I, CF₃, —CF₂H, —CN, —C(═O)OH, —NH₂, —NO₂, —OH, ═O, —SF₅;
   —C₁₋₆-alkyl, saturated or unsaturated, unsubstituted, mono- or polysubstituted;
   —C(═O)O—C₁₋₆-alkyl, saturated or unsaturated, unsubstituted, mono- or polysubstituted;
   —NHC₁₋₆-alkyl, saturated or unsaturated, unsubstituted, mono- or polysubstituted;
   —N(C₁₋₆-alkyl)₂, saturated or unsaturated, unsubstituted, mono- or polysubstituted;
   —O—C₁₋₆-alkyl, saturated or unsaturated, unsubstituted, mono- or polysubstituted;
   —S(═O)₂—C₁₋₆-alkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted.
19. The compound per se, or for use according to any one of the preceding Clauses, wherein V is unsubstituted, mono- or polysubstituted with substituents independently of one another selected from
   —OH, —F, —Cl, —Br, —I, —SH, —CF₃, —CHF₂, —CH₂F, —OCF₃, —OCHF₂, —OCH₂F, SF₅, —CN, —NO₂, —C(═O)OH, —NH₂, —N(CH₃)₂, -cyclopropyl, or —O-cyclopropyl; preferably selected from —OH, —F, —Cl, —Br, —I, —SH, —CF₃, —CHF₂, —CH₂F, —OCF₃, —OCHF₂, —OCH₂F, SF₅, —CN, —NO₂, —C(═O)OH, —NH₂, or —N(CH₃)₂;
   —C₁₋₆-alkyl, saturated or unsaturated, unsubstituted, mono- or polysubstituted with substituents independently of one another, selected from the group consisting of —F, —C₁, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, —C(═O)NH₂, and -cyclopropyl; preferably selected from the group consisting of —F, —C, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   —C₁₋₆-heteroalkyl, saturated or unsaturated, unsubstituted, mono- or polysubstituted with substituents independently of one another, selected from the group consisting of —F, —C₁, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   —OC₁₋₆-alkyl, unsubstituted, mono- or polysubstituted with substituents independently of one another, selected from the group consisting of —F, —Cl, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   —O(C═O)C₁₋₆-alkyl, unsubstituted, mono- or polysubstituted with substituents independently of one another, selected from the group consisting of —F, —Cl, —Br, —I, —C₁-6-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   —C(═O)OC₁₋₆-alkyl, unsubstituted, mono- or polysubstituted with substituents independently of one another, selected from the group consisting of —F, —Cl, —Br, —I, -C₁. 6-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   3-14-membered cycloalkyl selected from the group consisting of cyclopropyl, cyclobutyl, cyclopentyl, cyclohexyl and cycloheptyl; in each case unsubstituted, mono- or polysubstituted with substituents independently of one another, selected from the group consisting of —F, —C, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂;
   3-14-membered heterocycloalkyl selected from the group consisting of azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, tetrahydrothiopyran, thiazolidine, thietane, thiirane, thiolane, thiomorpholine, indoline, dihydrobenzofuran, dihydrobenzothiophene, 1,1-dioxothiacyclohexane, 2-azaspiro[3.3]heptane, 2-oxaspiro[3.3]heptane, 7-azaspiro[3.5]-nonane, 8-azabicyclo[3.2.1]octane, 9-azabicyclo[3.3.1]nonane, hexahydro-1H-pyrrolizine, hexahydrocyclopenta[c]pyrrole, octahydrocyclopenta[c]pyrrole, and octahydropyrrolo[1,2-a]pyrazine, in each case unsubstituted, mono- or polysubstituted with substituents independently of one another, selected from the group consisting of —F, —C, —Br, —I, —C₁₋₆-alkyl, C₂₋₆-alkenyl, —C₂₋₆-alkynyl, —OH, ═O, —SH, ═S, —CN, —CF₃, —CHF₂, —CH₂F, —OCF₃, —OCHF₂, —OCH₂F, SF₅, —NO₂, —C(═O)OH, —NH₂, C(═O)CHF₂, and —C(═O)NH₂.
20. The compound per se, or for use according to any one of the preceding Clauses, wherein V is unsubstituted, mono- or polysubstituted with substituents independently of one another selected from —F, —Cl, —CN, —OH, ═O, —C₁₋₆-alkyl, —CHF₂, —CF₃, —C₁₋₆-alkylene-NH₂, —C₁₋₆-alkylene-NHC(═O)O—C₁₋₆-alkyl, —C₁₋₆-alkylene-OH, —C₁₋₆-alkylene-NHC(═O)—O—C₁₋₆-alkyl, —C(═O)O—C₁₋₆-alkyl, —N(C₁₋₆-alkyl)₂, —OC₁₋₆-alkyl, —OCF₃, —O—C₁₋₆-alkylene-N(C₁₋₆-alkyl)₂, —S(═O)₂—C₁₋₆-alkyl, -azetidine, —C₁₋₆-alkylene-O-tetrahydropyran, or -piperazine substituted with —C₁₋₆-alkyl; or represents oxetanyl, unsubstituted, mono- or polysubstituted.
21. The compound per se, or for use according to any one of the preceding Clauses, wherein V is
   (i) unsubstituted;
   (ii) monosubstituted;
   (iii) disubstituted;
   (iv) trisubstituted; or
   (v) tetrasubstituted.
22. The compound per se, or for use according to any one of the preceding Clauses, wherein R¹ represents
   —H, —F, —Cl, —Br, —I;
   —C₁₋₆-alkyl, saturated or unsaturated, unsubstituted, mono- or polysubstituted; —O—C₁₋₆-alkyl, saturated or unsaturated, unsubstituted, mono- or polysubstituted;
   —C(═O)C₁₋₆-alkyl, saturated or unsaturated, unsubstituted, mono- or polysubstituted;
   —C(═O)OC₁₋₆-alkyl, saturated or unsaturated, unsubstituted, mono- or polysubstituted;
   —C(═O)NHC₁₋₆-alkyl, saturated or unsaturated, unsubstituted, mono- or polysubstituted;
   —C(═O)N(C₁₋₆-alkyl)₂, saturated or unsaturated, unsubstituted, mono- or polysubstituted;
   —S(═O)C₁₋₆-alkyl, saturated or unsaturated, unsubstituted, mono- or polysubstituted;
   —S(═O)₂—C₁₋₆-alkyl, saturated or unsaturated, unsubstituted, mono- or polysubstituted;
   —C₁-C₆-heteroalkyl, saturated or unsaturated, unsubstituted, mono- or polysubstituted; or
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.
23. The compound per se, or for use according to any one of the preceding Clauses, wherein R¹ represents —H, —F, —Cl, —Br, —I, —C₁₋₆-alkyl, —O—C₁₋₆-alkyl, —C₁₋₆-alkylene-O—C₁₋₆-alkyl, -C₁. 6-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)C₁₋₆-alkyl, —C(═O)OC₁₋₆-alkyl, —C(═O)NHC₁₋₆-alkyl, —C(═O)N(C₁₋₆-alkyl)₂, —S(═O)—C₁₋₆-alkyl, —S(═O)₂—C₁₋₆-alkyl, —O—C₁₋₆-alkyl, -cyclopropyl unsubstituted, cyclobutyl unsubstituted, cyclopentyl unsubstituted or cyclohexyl unsubstituted.
24. The compound per se, or for use according to any one of the preceding Clauses, wherein R¹ represents —H, —C₁₋₆-alkyl, —C₁₋₆-alkylene-O—C₁₋₆-alkyl, —CH₂F, —CHF₂, —CF₃, -cyclopentyl, unsubstituted, or -cyclopropyl, unsubstituted; preferably wherein R′ represents —H, —C₁₋₆-alkyl, —C₁₋₆-alkylene-O—C₁₋₆-alkyl, —CH₂F, —CHF₂, —CF₃, or -cyclopentyl, unsubstituted.
25. The compound per se, or for use according to any one of the preceding Clauses, wherein R¹ represents —CH₂F, —CHF₂, —CH₃, or -cyclopropyl, unsubstituted; preferably wherein R′ represents —CH₂F, —CHF₂, —CH₃, or —CH₂CH₃.
26. The compound per se, or for use according to any one of the preceding Clauses, wherein R² represents
   —H;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted.
27. The compound per se, or for use according to any one of the preceding Clauses, wherein R² represents —H, —C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃.
28. The compound per se, or for use according to any one of the preceding Clauses, wherein R² represents —H or —C₁₋₆-alkyl.
29. The compound per se, or for use according to any one of the preceding Clauses, wherein R³ represents
   —H;
   —OH;
   —C₁-C₆-alkyl, saturated or unsaturated, unsubstituted, mono- or polysubstituted; or
   —C₁-C₆-heteroalkyl, saturated or unsaturated, unsubstituted, mono- or polysubstituted.
30. The compound per se, or for use according to any one of the preceding Clauses, wherein R³ represents —H, —OH, —C₁₋₆-alkyl, —C₁₋₆-alkylene-OH, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, -C₁. 6-alkylene-NH—C₁₋₆-alkylene-CF₃, or —C₁₋₆-alkylene-N(C₁₋₆-alkyl)-C₁₋₆-alkylene-CF₃.
31. The compound per se, or for use according to any one of the preceding Clauses, wherein R³ represents —H, —OH, or —C₁₋₆-alkyl, saturated, unsubstituted or monosubstituted with —OH.
32. The compound per se, or for use according to any one of the preceding Clauses, wherein R⁴ represents
   —H;
   —S(═O)C₁₋₆-alkyl, saturated or unsaturated, unsubstituted, mono- or polysubstituted;
   —S(═O)₂—C₁₋₆-alkyl, saturated or unsaturated, unsubstituted, mono- or polysubstituted;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
   6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted.
33. The compound per se, or for use according to any one of the preceding Clauses, wherein R⁴ represents
   —S(═O)₂C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   —S(═O)₂(3-14-membered cycloalkyl), wherein said 3-14-membered cycloalkyl is selected from the group consisting of cyclopropyl, cyclobutyl, cyclopentyl, cyclohexyl, and cycloheptyl, in each case saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   —C₁₋₆-alkyl, saturated or unsaturated, unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is selected from the group consisting of cyclopropyl, cyclobutyl, cyclopentyl, cyclohexyl, and cycloheptyl, in each case saturated or unsaturated, in each case unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from the group consisting of azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxepane, oxetane, oxirane, piperazine, piperidine, pyrazolidine, pyrrolidine, quinuclidine, tetrahydrofuran, tetrahydropyran, tetrahydrothiopyran, thiazolidine, thietane, thiirane, thiolane, thiomorpholine, indoline, dihydrobenzofuran, dihydrobenzo-thiophene, 1,1-dioxothiacyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexahydrocyclopenta[c]pyrrole, octahydro-cyclopenta[c]pyrrole, and octahydropyrrolo[1,2-a]pyrazine; in each case unsubstituted, mono- or polysubstituted with substituents independently of one another selected from the group consisting of —F, —Cl, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted; -phenyl unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted;
   5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of benzimidazole, benzisoxazole, benzoxazole, benzodioxole, benzofuran, benzothiadiazole, benzothiazole, benzothiophene, carbazole, cinnoline, dibenzofuran, furane, furazane, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine; in each case unsubstituted, mono- or polysubstituted with substituents independently of one another selected from the group consisting of —F, —Cl, —CN, —C₁₋₆-alkyl, —C₁₋₆-alkylene-CF₃, —OH, ═O, —OC₁₋₆-alkyl, —C₁₋₆-alkylene-OH, —C₁₋₆-alkylene-O—C₁₋₆-alkyl, —NH₂, —NHC₁₋₆-alkyl, —N(C₁₋₆-alkyl)₂, —NHC(═O)O—C₁₋₆-alkyl, —N(C₁₋₆-alkyl)C(═O)O—C₁₋₆-alkyl, —C₁₋₆-alkylene-NHC(═O)O—C₁₋₆-alkyl, —C₁₋₆-alkylene-NH₂, —C₁₋₆-alkylene-NH—C₁₋₆-alkyl, —C₁₋₆-alkylene-N(C₁₋₆-alkyl)₂, —C₁₋₆-alkylene-NH—C₁₋₆-alkylene-CF₃, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —C(═O)N(C₁₋₆-alkyl)₂, —S(═O)₂C₁₋₆-alkyl, -phenyl, —C₁₋₆-alkylene-phenyl, 3-14-membered heterocycloalkyl, saturated or unsaturated, unsubstituted; and 5-14-membered heteroaryl, unsubstituted.
34. The compound per se, or for use according to any one of the preceding Clauses, wherein R⁴ represents
   —H;
   —S(═O)₂C₁₋₆-alkyl, saturated, unsubstituted, monosubstituted or polysubstituted with —F;
   —S(═O)₂(3-14-membered cycloalkyl), saturated, unsubstituted;
   —C₁₋₆-alkyl, saturated, unsubstituted, monosubstituted or disubstituted with substituents independently of one another selected from the group consisting of —OH, —OC₁₋₆-alkyl, —N(C₁₋₆-alkyl)₂, —C₁₋₆-alkylene-NH₂, —C₁₋₆-alkylene-NH—C₁₋₆-alkyl, -phenyl unsubstituted;
   3-14-membered cycloalkyl or —C₁₋₆-alkylene-(3-14-membered cycloalkyl), wherein —C₁₋₆-alkylene- is unsubstituted or monosubstituted with —OH, wherein said 3-14-membered cycloalkyl is saturated, unsubstituted, monosubstituted or disubstituted with substituents independently of one another selected from the group consisting of —C₁₋₆-alkyl, —C₁₋₆-alkylene-NH₂, —C₁₋₆-alkylene-NH—C₁₋₆-alkylene-CF₃, —C₁₋₆-alkylene-OH, —C₁₋₆-alkylene-NHC(═O)O—C₁₋₆-alkyl, —OH, —OC₁₋₆-alkyl, —NH₂, —N(C₁₋₆-alkyl)₂, —NHC(═O)O—C₁₋₆-alkyl;
   3-14-membered heterocycloalkyl or —C₁₋₆-alkylene-(3-14-membered heterocycloalkyl), wherein —C₁₋₆-alkylene- is unsubstituted or monosubstituted with —OH, wherein said 3-14-membered heterocycloalkyl in each case is selected from azetane, 1,4-oxazepane, pyrrolidine, piperidine, azepane, diazepane, tetrahydrofuran, tetrahydropyran, oxetane, morpholine, piperazine, hexahydrocyclopenta[c]pyrrole, octahydrocyclopenta[c]pyrrole, octahydropyrrolo[1,2-a]pyrazine, 8-azabicyclo[3.2.1]octane, 9-azabicyclo[3.3.1]nonane, quinuclidine, hexahydro-1H-pyrrolizine, 2-oxaspiro[3.3]heptane, 2-azaspiro[3.3]heptane, 7-azaspiro[3.5]nonane, 1,1-dioxothiacyclohexane, in each case unsubstituted, mono- or polysubstituted with substituents independently of one another selected from the group consisting of —F, —OH, ═O, —C₁₋₆-alkyl, —C₁₋₆-alkylene-CF₃, —C₁₋₆-alkylene-OH, —C₁₋₆-alkylene-O—C₁₋₆-alkyl, —NH₂, —N(C₁₋₆-alkyl)₂, —C₁₋₆-alkylene-NH₂, —C₁₋₆-alkylene-N(C₁₋₆-alkyl)₂, —C(═O)—C₁₋₆-alkyl, —C(═O)OH, —C(═O)O—C₁₋₆-alkyl, —C(═O)O—C₁₋₆-alkylene-CF₃, —C(═O)NH₂, —C(═O)NH(C₁₋₆-alkyl), —S(═O)₂C₁₋₆-alkyl, oxetanyl, pyrimidinyl, —C₁₋₆-alkylene-phenyl;
   -phenyl unsubstituted;
   5-14-membered heteroaryl or —C₁₋₆-alkylene-(5-14-membered heteroaryl), wherein —C₁₋₆-alkylene- is unsubstituted or monosubstituted with —OH, wherein said 5-14-membered heteroaryl in each case is selected from the group consisting of pyridine, pyridazine, pyrazine, pyrazole, isoxazole, triazole, and [1,2,4]triazolo[4,3-a]pyrimidine, in each case unsubstituted, monosubstituted or disubstituted with substituents independently of one another selected from the group consisting of —C₁₋₆-alkyl, —OH.
35. The compound per se, or for use according to any one of the preceding Clauses, wherein R³ and R⁴ together form a 5- or 6-membered heterocycle containing 1 or 2 heteroatoms selected from N, O and S, saturated or unsaturated, unsubstituted or mono- or polysubstituted.
36. The compound per se, or for use according to any one of the preceding Clauses, wherein R³ and R⁴ together form a heterocycle selected from the group consisting of pyrrolidine, piperidine, morpholine, and piperazine, in each case unsubstituted, mono- or polysubstituted with substituents independently of one another selected from the group consisting of —C₁₋₆-alkyl, —NH₂, —NHCH₃, —N(CH₃)₂, —C(═O)NH—C₁₋₆-alkyl, —C(═O)N(C₁₋₆-alkyl)₂, —C(═O)O—C₁₋₆-alkyl, —NHC(═O)O—C₁₋₆-alkyl, -pyridyl unsubstituted, and 1,2,4-oxadiazole unsubstituted or monosubstituted with —C₁₋₆-alkyl.
37. The compound per se, or for use according to any one of the preceding Clauses, wherein R³ and R⁴ together form a pyrrolidine ring, unsubstituted or monosubstituted with —N(CH₃)₂;
   piperidine ring, unsubstituted or monosubstituted with a substituent selected from the group consisting of —C₁₋₆-alkyl, —NH₂, —N(CH₃)₂, —C(═O)NH—C₁₋₆-alkyl, —C(═O)O—C₁₋₆-alkyl, —NHC(═O)O—C₁₋₆-alkyl, and 1,2,4-oxadiazole unsubstituted or monosubstituted with -C₁. 6-alkyl;
   morpholine ring, unsubstituted; or
   piperazine ring, unsubstituted or N-substituted with a substituent selected from the group consisting of —C₁₋₆-alkyl and -pyridyl unsubstituted.
38. The compound per se, or for use according to one any one of the preceding Clauses, wherein R⁵ and R^{5′} independently of one another represent
   —H;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.
39. The compound per se, or for use according to any one of the preceding Clauses, wherein R⁵ and R^{5′} independently of one another represent —H, —C₁-C₆-alkyl, or —C₁-C₆-alkylene-N(C₁-C₆-alkyl)₂.
40. The compound per se, or for use according to any one of the preceding Clauses, wherein at least one of R⁵ and R^{5′} does not represent —H.
41. The compound per se, or for use according to any one of the preceding Clauses, wherein R⁶, R⁷ and R⁸ independently of one another represent
   —H;
   —F, —Cl, —Br, —I, —OH, —SH, —SF₅, —CN, —NO₂, —C(═O)OH, —NH₂;
   —C₁₋₆-alkyl, saturated or unsaturated, unsubstituted, mono- or polysubstituted;
   —O—C₁₋₆-alkyl, saturated or unsaturated, unsubstituted, mono- or polysubstituted;
   —NHC₁₋₆-alkyl, saturated or unsaturated, unsubstituted, mono- or polysubstituted;
   —N(C₁₋₆-alkyl)₂, saturated or unsaturated, unsubstituted, mono- or polysubstituted;
   —C(═O)OC₁₋₆-alkyl, saturated or unsaturated, unsubstituted, mono- or polysubstituted;
   —OC(═O)C₁₋₆-alkyl, saturated or unsaturated, unsubstituted, mono- or polysubstituted;
   —C₁₋₆-heteroalkyl, saturated or unsaturated, unsubstituted, mono- or polysubstituted.
42. The compound per se, or for use according to any one of the preceding Clauses, wherein R⁶, R⁷ and R⁸ independently of one another represent
   —H, —F, —Cl, —Br, —I, —OH, —SH, —SF₅, —CN, —NO₂, —C(═O)OH, —NH₂,
   —C₁₋₆-alkyl, —CF₃, —CHF₂, —CH₂F,
   —O—C₁₋₆-alkyl, —OCF₃, —OCHF₂, —OCH₂F,
   —NHC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
   —N(C₁₋₆-alkyl)₂ unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
   —C(═O)OC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
   —OC(═O)C₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂; or
   —C₁₋₆-heteroalkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂.
43. The compound per se, or for use according to any one of the preceding Clauses, wherein R⁶ represents —H, —F, —Cl, —CN, or —C₁-C₆-alkyl.
44. The compound per se, or for use according to any one of the preceding Clauses, wherein R⁶ does not represent —H.
45. The compound per se, or for use according to any one of the preceding Clauses, wherein R¹ represents —H, —F, —C, —CN, or —C₁-C₆-alkyl.
46. The compound per se, or for use according to any one of the preceding Clauses, wherein R¹ does not represent —H.
47. The compound per se, or for use according to any one of the preceding Clauses, wherein R⁸ represents —H, —F, —C, —CN, or —C₁-C₆-alkyl.
48. The compound per se, or for use according to any one of the preceding Clauses, wherein R⁸ does not represent —H.
49. The compound per se, or for use according to any one of the preceding Clauses, wherein
   (i) R⁶, R⁷ and R⁸ each represent —H; or
   (ii) two of R⁶, R⁷ and R⁸ represent —H and the other of R⁶, R⁷ and R⁸ represents —F, —C, —CN, or —CH₃; or
   (iii) one of R⁶, R⁷ and R⁸ represents —H and the other of R⁶, R⁷ and R⁸ independently of one another represent —F, —C, —CN, or —CH₃.
50. The compound per se, or for use according to any one of the preceding Clauses, which is selected from the group consisting of cpd 001 to cpd 036, or cpd 037 to 091, as mentioned above and the physiologically acceptable salts thereof.
51. The compound per se, or for use according to any one of the preceding Clauses, wherein the pain is selected from nociceptive pain, inflammatory pain, and neuropathic pain.
52. The compound per se, or for use according to any one of the preceding Clauses, wherein the pain is post-operative pain.
53. A compound of formula (I), a stereo-isomeric form, a physiologically acceptable salt, solvate and/or polymorph thereof, as defined in any one of the preceding Clauses, wherein
   (a-1) Q represents —NR³R⁴; R¹ represents R^{W}; and R^{W} represents —C₁-C₆-alkyl - and/or
   (a-2) Q represents —NR³R⁴; and at least one of R¹ and R^{5′} represents —H; and/or
   (a-3) Q represents —NR³R⁴; and R⁶ represents —H; and/or
   (a-4) Q represents —NR³R⁴; and R⁸ represents —H; or
   (b-1)
   (b-1) Q represents —NR³R⁴; and R¹ represents —CH₂F, —CHF₂, —CF₃, —CN, -methyl, -ethyl, -propyl or -cyclopropyl; and/or
   (b-2) Q represents —NR³R⁴; and at least one of R⁵ and R^{5′} does not represent —H; and/or
   (b-3) Q represents —NR³R⁴; and R³ represents —H.
54. A pharmaceutical composition or a medicament comprising a compound according to any one of the preceding Clauses.

Further exemplary embodiments of the invention are summarized as items 1 to 68 hereinafter:
1. A compound of formula (I), a stereo-isomeric form, a physiologically acceptable salt, solvate and/or polymorph thereof

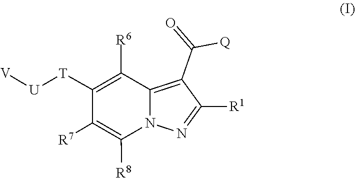

wherein
R¹ represents —F, —Cl, —Br, —I, —CN, —R^{W}, —OR^{W}, —OC(═O)R^{W}, —NR^{W}R^{X}, —NR^{W}C(═O)R^{W}, —SR^{W}, —S(═O)R^{W}, —S(═O)₂R^{W}, —C(═O)R^{W}, —C(═O)OR^{W}, or —C(═O)NR^{W}R^{X};
Q represents —OR² or —NR³R⁴;
R² represents —R^{Y};
R³ represents —OH or —R^{Y};
R⁴ represents —R^{Y} or —S(═O)₂R^{Y};
   or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —CR⁵R^{5′}—; or T represents —CR⁵R^{5′}— and U represents
—O—;
R⁵ and R^{5′} independently of one another represent —R^{Y};
R⁶, R⁷ and R⁸ independently of one another represent —F, —Cl, —Br, —I, —CN, —NO₂, —SF₅, -R^{W}, —OR^{W}, —OC(═O)R^{W}, —NR^{W}R^{X}, —NR^{W}C(═O)R*, —SR^{W}, —S(═O)R^{W}, —S(═O)₂R^{W}, —C(═O)R^{W}, —C(═O)OR^{W}, or —C(═O)NR^{W}R^{X};
V represents 3-14-membered heterocycloalkyl, saturated or unsaturated; 3-14-membered cycloalkyl, saturated or unsaturated; 5-14-membered aryl; C₁-C₆ alkyl or 5-14-membered heteroaryl; in each case unsubstituted, mono- or polysubstituted with substituents independently of one another selected from —F, —Cl, —Br, —I, —CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z};
wherein
R^{W} and R^{X} independently of one another and in each case independently represent
   —H;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R^{Y} and R^{Z} independently of one another and in each case independently represent
   —H;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
   3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
   6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
   5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
   or R^{Y} and R^{Z} together form a 4, 5, 6, 7 or 8 membered heterocycle containing 1 to 3 heteroatoms selected from N, O and S, saturated or unsaturated, unsubstituted or mono- or polysubstituted;
and wherein “mono- or polysubstituted” in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-O—CF₃, —C₁₋₆-alkylene-O—CF₂H, —C₁₋₆-alkylene-O—CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —N(3-14-membered cycloalkyl)(C₁₋₆-alkyl), —N(C₁₋₆-alkyl)-C₁₋₆-alkylene-OH, —N(H)—C₁₋₆-alkylene-OH, —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered hetero-cycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, —S(═O)₂-(5 to 14-membered heteroaryl).
2. The compound of formula (I), a stereo-isomeric form, a physiologically acceptable salt, solvate and/or polymorph thereof, preferably according to item 1,

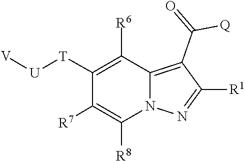

wherein
Q represents —OR² or —NR³R⁴;
T represents —O—;
U represents —CR⁵R^{5′}—;
V represents H or R⁴
R¹ represents —H, R⁹, —O—C₁₋₆-alkyl, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —S(═O)—C₁₋₆-alkyl, —S(═O)₂—C₁₋₆-alkyl, cyclopropyl unsubstituted, cyclobutyl unsubstituted, cyclopentyl unsubstituted or cyclohexyl unsubstituted;
R² represents hydrogen or R⁹;
R³ represents —H, R⁹, —OH, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃;
R⁴ represents R^{4′} or —SO₂—R^{4′}, wherein
R^{4′} represents —R⁹, —R^{cycl} or —R¹⁰-R^{cycl}; and
R^{cycl} represents
   3-14-membered cycloalkyl, saturated or unsaturated
   4-14-membered heterocycloalkyl, saturated or unsaturated, with one or more N, O or S atom in the heterocycloalkyl ring
   6-14-membered aryl, or
   5-14-membered heteroaryl with one or more N, O or S in the heteroaryl ring;
R¹ and R³ together may form a group —R¹⁰—; or
R³ and R⁴ together may form a group —R¹⁰—;
R⁵, R^{5′}, R⁶, R⁷ and R⁸ independently of each other represent hydrogen or R⁹; and
   R^{cycl} may be substituted by R⁹ and the cycloalkyl rings and the heterocycloalkyl rings of R^{cycl} independently of each other may be substituted by ═O or ═S; and
   R⁹ represents —C₁₋₆-alkyl or —C₁₋₆-heteroalkyl with one or more N, O or S in the chain; and
   R¹⁰ represents —C₁₋₆-alkylen- or —C₁₋₆-heteroalkylen- with one or more N, O or S in the chain; and
wherein R⁹ and R¹⁰ may be straight or branched, saturated or unsaturated; and
   R⁹, R¹⁰ and R^{cycl} may be substituted by R¹¹; and
   R¹¹ is selected from the list consisting of a —OR, —NR₂, halogen, —CN, —COOR, —CO—NR₂, —CONR(OR), —O—CO—O—C₁₋₆-alkyl, —NR—C(═O)—R, —NR—C(═O)—O—R, —NR—C(═O)—N(R)₂, —O—S(═O)₂—NR₂, —NR—SO₂—R, —N(R)—S(═O)₂OR, —N(R)—S(═O)₂NR₂, —SR, —S(═O)—R, —S(═O)₂—OR, —S(═O)₂—NR₂, —NO, —NO₂, —C₁₋₆-alkylen-OR, —C₁₋₆-alkylen-NR₂, =0 or ═S or 3-7-membered cycloalkyl, saturated or unsaturated wherein the residues R may be independently of each other selected from the group consisting of H or —C₁₋₆-alkyl, which may be substituted by halogen.
3. The compound of item 1, wherein R¹ represents a straight or branched alkyl with 1 to 6 carbon atoms, which may be substituted by halogen, —OR, —CONR₂ or —NR₂, wherein the residues R independently of each other represent H or C₁₋₆-alkyl, which may be substituted by halogen.
4. The compound of any one of the preceding items, wherein R³ represents hydrogen or R⁹.
5. The compound of any one of the preceding items, wherein
Q represents OR² or —NR³R⁴;
T represents —O—;
U represents —CR⁵R^{5′}—;
V represents H or R⁴
R¹ represents a straight or branched alkyl with 1 to 6 carbon atoms, which may be substituted by halogen, —OR, —CONR₂ or —NR₂, wherein the residues R independently of each other represent H or C₁₋₆-alkyl, which may be substituted by halogen;
R² and R³ represent hydrogen or R⁹;
   or R¹ and R³ together may form a group —R¹⁰—;
R⁴ represents R^{4′} or —SO₂—R^{4′}, wherein
R^{4′} represents —R⁹, —R^{cycl} or —R¹⁰-R^{cycl}; and R^{cycl} represents
   3-14-membered cycloalkyl, saturated or unsaturated
   4-14-membered heterocycloalkyl with one or more N, O or S atom in the heterocycloalkyl ring, saturated or unsaturated
   6-14-membered aryl, or
   5-14-membered heteroaryl with one or more N, O or S in the heteroaryl ring; R⁵, R^{5′}, R⁶, R⁷ and R⁸ independently of each other represent hydrogen or R⁹; and
   R^{cycl} may be substituted by R⁹ and the cycloalkyl rings and the heterocycloalkyl rings of R^{cycl} independently of each other may be substituted by ═O or ═S; and
   R⁹ represents —C₁₋₆-alkyl or —C₁₋₆-heteroalkyl with one or more N, O or S in the chain; and
   R¹⁰ represents —C₁₋₆-alkylen- or —C₁₋₆-heteroalkylen- with one or more N, O or S in the chain; and
wherein R⁹ and R¹⁰ may be straight or branched, saturated or unsaturated; and
   R⁹, R¹⁰ and R^{cycl} may be substituted by R¹¹; and
   R¹¹ is selected from the list consisting of a —OR, —NR₂, halogen, —CN, —COOR, —CO—NR₂, —CONR(OR), —O—CO—O—C₁₋₆-alkyl, —NR—C(═O)—R, —NR—C(═O)—O—R, —NR—C(═O)—N(R)₂, —O—S(═O)₂—NR₂, —NR—SO₂—R, —N(R)—S(═O)₂OR, —N(R)—S(═O)₂NR₂, —SR, —S(═O)—R, —S(═O)₂—OR, —S(═O)₂—NR₂, —NO, —NO₂, —C₁₋₆-alkylen-OR, —C₁₋₆-alkylen-NR₂ or 3-7-membered cycloalkyl, saturated or unsaturated wherein the residues R may be independently of each other H or —C₁₋₆-alkyl, which may be substituted by halogen.
6. The compound of any one of the preceding items, wherein V represents
   H,
   —R⁹,
   3-14 membered cycloalkyl, saturated or unsaturated,
   4-14-membered heterocycloalkyl, saturated or unsaturated, with one or more N, O or S atom in the heterocycloalkyl ring,
   6-14-membered aryl,
   5-14-membered heteroaryl with one or more N, O or S atom in the heteroaryl ring, wherein the cyclic groups may be substituted by R⁹.
7. The compound of any one of the preceding items, wherein R⁵, R^{5′}, R⁶, R⁷ and R⁸ independently of each other represent hydrogen or C₁₋₃-alkyl.
8. The compound of any one of the preceding items, wherein R⁵, R^{5′}, R⁶, R⁷ and R⁸ independently of each other represent hydrogen or methyl.
9. The compound of any one of the preceding items, wherein
Q represents OR² or —NR³R⁴;
T represents —O—;
U represents —CR⁵R^{5′}—;
V represents H, —R⁹ or —R^{cycl};
R¹ represents —C₁₋₆-alkyl, which may be substituted by halogen, —OH, OR, —CONR₂ or —NR₂, wherein the residues R independently of each other represent H or —C₁₋₆-alkyl, which may be substituted by halogen;
R² and R³ represent hydrogen or methyl;
   or R¹ and R³ together may form a group —R¹⁰—;
R⁴ represents R^{4′} or —SO₂—R^{4′}, wherein
R^{4′} represents —R⁹, —R^{cycl} or —R¹⁰-R^{cycl}; and R^{cycl} represents
   3-14-membered cycloalkyl, saturated or unsaturated
   4-14-membered heterocycloalkyl, saturated or unsaturated, with one or more N, O or S atom in the heterocycloalkyl ring
   6-14-membered aryl, or
   5-14-membered heteroaryl with one or more N, O or S in the heteroaryl ring;
   R⁵, R^{5′}, R⁶, R⁷ and R⁸ independently of each other represent hydrogen or methyl; and
   R^{cycl} may be substituted by R⁹ and the cycloalkyl rings and the heterocycloalkyl rings of R^{cycl} independently of each other may be substituted by ═O or ═S; and
   R⁹ represents —C₁₋₆-alkyl or —C₁₋₆-heteroalkyl with one or more N, O or S in the chain; and
   R¹⁰ represents —C₁₋₆-alkylen- or a —C₁₋₆-heteroalkylen- with one or more N, O or S in the chain; and
wherein R⁹ and R¹⁰ may be straight or branched, saturated or unsaturated; and
   R⁹, R¹⁰ and R^{cycl} may be substituted by R¹¹; and
   R¹¹ is selected from the list consisting of a —OR, —NR₂, halogen, —CN, —COOR, —CO—NR₂, —CONR(OR), —O—CO—O—C₁₋₆-alkyl, —NR—C(═O)—R, —NR—C(═O)—O—R, —NR—C(═O)—N(R)₂, —O—S(═O)₂—NR₂, —NR—SO₂—R, —N(R)—S(═O)₂OR, —N(R)—S(═O)₂NR₂, —SR, —S(═O)—R, —S(═O)₂—OR, —S(═O)₂—NR₂, —NO, —NO₂, —C₁₋₆-alkylen-OR, —C₁₋₆-alkylen-NR₂ or 3-7-membered cycloalkyl, saturated or unsaturated wherein the residues R may be independently of each other H or —C₁₋₆-alkyl, which may be substituted by halogen.
10. The compound of any one of the preceding items, wherein R⁵ and R^{5′} represent hydrogen.
11. The compound of any one of the preceding items, wherein R¹ and R³ together form a group —R¹⁰—.
12. The compound of any one of the preceding items, wherein R¹ and R³ together form a straight or branched alkanediyl or alkenediyl with 1 to 4 carbon atoms, which may not be substituted or may be substituted by Halogen, C₁₋₆-alkyl or CONR₂, wherein the residues R may be independently of each other H or a C₁₋₆-alkyl.
13. The compound of any one of the preceding items, wherein R¹ and R³ together form a straight or branched alkanediyl or alkenediyl with 1 to 3 carbon atoms, which may be not substituted or substituted by Halogen, C₁₋₆-alkyl or CONR₂, wherein the residues R may be independently of each other H or a C₁₋₆-alkyl.
14. The compound of any one of items 1 to 13, wherein R¹ and R³ are not connected to form a heterocycle.
15. The compound of any one of the preceding items, wherein R⁴ represents R^{4′} or —SO₂—C₁₋₆-alkyl.
16. The compound of any one of the preceding items, wherein V represents H, C₁₋₆-alkyl or —R^{cycl}.
17. The compound of any one of the preceding items, wherein
Q represents OR² or —NR³R⁴;
T represents —O—;
U represents —CH₂—;
V represents H, C₁₋₆-alkyl or —R^{cycl}; and wherein the cyclic groups in the group V may be substituted by at least one group selected from the list consisting of —COO—C₁₋₆-alkyl, —CO—NR₂, —CN, halogen or a C₁₋₆-alkyl, wherein R represents independently of each other H or —C₁₋₆-alkyl and the alkyl groups in the group V may be substituted by one or more halogen atoms;
R¹ represents —C₁₋₆-alkyl, which may be substituted by halogen, —OH, —CONR₂ or —NR₂, wherein the residues R independently of each other represent H or —C₁₋₆-alkyl, which may be substituted by halogen;
R² and R³ represent hydrogen or methyl;
R⁴ represents —SO₂—C₁₋₆-alkyl, —R⁹, —R^{cycl} or —R¹-R^{cycl}; and R^{cycl} represents
   3-14-membered cycloalkyl, saturated or unsaturated
   4-14-membered heterocycloalkyl, saturated or unsaturated, with one or more N, O or S atom in the heterocycloalkyl ring
   6-14-membered aryl, or
   5-14-membered heteroaryl with one or more N, O or S in the heteroaryl ring; and R⁶, R⁷ and R⁸ independently of each other represent hydrogen or methyl; and
   R^{cycl} may be substituted by R⁹ and the cycloalkyl rings and the heterocycloalkyl rings of R^{cycl} independently of each other may be substituted by ═O or ═S; and
   R⁹ represents —C₁₋₆-alkyl or a —C₁₋₆-heteroalkyl with one or more N, O or S in the chain; and
   R¹⁰ represents —C₁₋₆-alkylen- or a —C₁₋₆-heteroalkylen- with one or more N, O or S in the chain; and
wherein R⁹ and R¹⁰ may be straight or branched, saturated or unsaturated; and
   R⁹, R¹⁰ and R^{cycl} of R⁴ may be substituted by R¹¹; and
R¹¹ is selected from the list consisting of a —OR, —NR₂, halogen, —CN, —COOR, —CO—NR₂, —CONR(OR), —O—CO—O—C₁₋₆-alkyl, —NR—C(═O)—R, —NR—C(═O)—O—R, —NR—C(═O)—N(R)₂, —O—S(═O)₂—NR₂, —NR—SO₂—R, —N(R)—S(═O)₂OR, —N(R)—S(═O)₂NR₂, —SR, —S(═O)—R, —S(═O)₂—OR, —S(═O)₂—NR₂, —NO, —NO₂, —C₁₋₆-alkylen-OR, —C₁₋₆-alkylen-NR₂ or 3-7-membered cycloalkyl, saturated or unsaturated wherein the residues R may be independently of each other H or —C₁₋₆-alkyl, which may be substituted by halogen.
18. The compound of any one of the preceding items, wherein R⁶, R⁷ and R⁸ independently of each other represent hydrogen.
19. The compound of any one of the preceding items, wherein R² represents hydrogen.
20. The compound of any one of the preceding items, wherein R³ represents hydrogen.
21. The compound of any one of the preceding items, wherein R² and R³ represent hydrogen.
22. The compound of any one of the preceding items, wherein R², R³, R⁵, R^{5′}, R⁶, R⁷ and R⁸ independently of each other represent hydrogen.
23. The compound of any one of the preceding items, wherein R¹ represents —C₁₋₆-alkyl.
24. The compound of any one of the preceding items, wherein
Q represents OR² or —NR³R⁴;
T represents —O—;
U represents —CH₂—;
V represents H, C₁₋₆-alkyl or —R^{cycl}; and wherein R^{cycl} in group V may be substituted by at least one group selected from the list consisting of —COO—C₁₋₆-alkyl, —CO—NR₂, —CN, halogen or a C₁₋₆-alkyl, wherein R represents independently of each other H or —C₁₋₆-alkyl and the alkyl groups in the group V may be substituted by one or more halogen atoms;
R¹ represents —C₁₋₆-alkyl;
R² represents hydrogen;
R³ represents hydrogen;
R⁴ represents —SO₂—C₁₋₆-alkyl, —R⁹, —R^{cycl} or —R¹⁰-R^{cycl}; and R^{cycl} represents
   3-6-membered cycloalkyl, saturated or unsaturated
   4-7-membered heterocycloalkyl, saturated or unsaturated, with one or more N, O or S atom in the heterocycloalkyl ring
   6-membered aryl, or
   6-membered heteroaryl with one or more N, O or S in the heteroaryl ring; and R⁶, R⁷ and R⁸ independently of each other represent hydrogen or methyl; and
   R^{cycl} may be substituted by R⁹ and the cycloalkyl rings and the heterocycloalkyl rings of R^{cycl} independently of each other may be substituted by ═O or ═S; and
   R⁹ represents —C₁₋₆-alkyl or a —C₁₋₆-heteroalkyl with one or more N, O or S in the chain; and
   R¹⁰ represents —C₁₋₆-alkylen- or a —C₁₋₆-heteroalkylen- with one or more N, O or S in the chain; and
   wherein R⁹ and R¹⁰ may be straight or branched, saturated or unsaturated; and R⁹, R¹⁰ and R^{cycl} of R⁴ may be substituted by R¹¹; and
      R¹¹ is selected from the list consisting of a —OR, —NR₂, halogen, —CN, —COOR, —CO—NR₂, —CONR(OR), —O—CO—O—C₁₋₆-alkyl, —NR—C(═O)—R, —NR—C(═O)—O—R, —NR—C(═O)—N(R)₂, —O—S(═O)₂—NR₂, —NR—SO₂—R, —N(R)—S(═O)₂OR, —N(R)—S(═O)₂NR₂, —SR, —S(═O)—R, —S(═O)₂—OR, —S(═O)₂—NR₂, —NO, —NO₂, —C₁₋₆-alkylen-OR, —C₁₋₆-alkylen-NR₂ or 3-7-membered cycloalkyl, saturated or unsaturated wherein the residues R may be independently of each other H or —C₁₋₆-alkyl, which may be substituted by halogen.
25. The compound of any one of the preceding items, wherein R⁹ represents —C₁₋₆-alkyl, straight or branched.
26. The compound of any one of the preceding items, wherein R¹⁰ represents —C₁₋₆-alkylen-, straight or branched.
27. The compound of any one of the preceding items, wherein R⁹ represents —C₁₋₆-alkyl, straight or branched and R¹⁰ represents —C₁₋₆-alkylen-, straight or branched.
28. The compound of any one of the preceding items, wherein R⁹ represents —C₁₋₃-alkyl, straight or branched.
29. The compound of any one of the preceding items, wherein R¹⁰ represents —C₁₋₃-alkylen-straight or branched.
30. The compound of any one of the preceding items, wherein R⁹ represents —C₁₋₃-alkyl, straight or branched and R¹⁰ represents —C₁₋₃-alkylen-, straight or branched.
31. The compound of any one of the preceding items, wherein the substituents of the cyclic groups in the residue R⁴ are bonded directly to the cyclic groups.
32. The compound of any one of the preceding items, wherein
Q represents OR² or —NR³R⁴;
T represents —O—;
U represents —CH₂—;
V represents H, C₁₋₆-alkyl or —R^{cycl}; and wherein R^{cycl} in group V may be substituted by at least one group selected from the list consisting of —CN, halogen or a C₁₋₃-alkyl and the alkyl groups in the group V may be substituted by one or more halogen atoms;
R¹ represents —C₁₋₃-alkyl;
R² represents hydrogen;
R³ represents hydrogen;
R⁴ represents —SO₂—C₁₋₆-alkyl, —R⁹, —R^{cycl} or —R¹-R^{cycl}; and
R^{cycl} represents
   3-6-membered cycloalkyl, saturated or unsaturated
   4-7-membered heterocycloalkyl, saturated or unsaturated, with one or more N, O or S atom in the heterocycloalkyl ring
   6-membered aryl, or
   5-6-membered heteroaryl with one or more N, O or S in the heteroaryl ring; and R⁶, R⁷ and R⁸ represent hydrogen; and
   R^{cycl} may be substituted by R⁹ and the cycloalkyl rings and the heterocycloalkyl rings of R^{cycl} independently of each other may be substituted by ═O or ═S; and
   R⁹ represents C₁₋₆ alkyl; and
   R¹⁰ represents —C₁₋₃-alkylen; and
   wherein R⁹ and R¹⁰ may be straight or branched, saturated or unsaturated; and
   R⁹, R¹⁰ and R^{cycl} of R⁴ may be substituted R¹¹; and
   R¹¹ selected from the list consisting of OH, halogen, a C₁₋₃-alkyl group, —NR—SO₂—R or a —CONR₂ group, wherein R may be independently of each other H or a C₁₋₃-alkyl group, which may be substituted by halogen.
33. The compound of any one of the preceding items, wherein R^{cycl} comprises 1, 2 or 3 heteroatoms.
34. The compound of any one of the preceding items, wherein R^{cycl} comprises 1 or 2 heteroatoms.
35. The compound of any one of the preceding items, wherein R^{cycl} comprises 1 heteroatom.
36. The compound of any one of the preceding items, wherein
Q represents OR² or —NR³R⁴;
T represents —O—;
U represents —CH₂—;
V represents
   H;
   C₁₋₃-alkyl which may be substituted with at least one halogen;
   3-6 membered cycloalkyl, which may be substituted with a —CN group;
   4-6-membered heterocycloalkyl, comprising one oxygen atom in the heterocycloalkyl ring;
   6-membered aryl, which may be substituted with at least one halogen or with a C₁₋₃-alkyl group, wherein the C₁₋₃-alkyl group may be substituted by at least one halogen atom;
   5-6-membered heteroaryl with one or more N, O or S atom in the heteroaryl ring, wherein the heteroaryl ring may be substituted with halogen or a C₁₋₃-alkyl group which may be substituted by at least one halogen atom;
R¹ represents —C₁₋₃-alkyl;
R² represents hydrogen;
R³ represents hydrogen;
R⁴ represents
   —SO₂—C₁₋₃-alkyl;
   C₁₋₆ alkyl which may be substituted by OH, F, —NR₂, —NR—SO₂—R or —CONR₂, wherein residues R are independently from each other hydrogen or C₁₋₃ alkyl;
   3-6-membered cycloalkyl which may be substituted by halogen, C₁₋₃-alkyl, which may be substituted by halogen or —OH, ═O or —CONH₂;
   C₁₋₃-alkylene-3-6-membered cycloalkyl which may be substituted by a C₁₋₃-alkyl or a —N (C₁₋₆ alkyl)₂ group;
   4-7-membered heterocycloalkyl with one or more N or O atom in the heterocycloalkyl ring and wherein the heterocycloalkyl ring may be substituted by ═O, halogen or —CONR₂ wherein the residues R independently of each other represent H or C₁₋₃-alkyl or the heterocycloalkyl ring may be substituted by a C₁₋₃ alkyl group, which may be substituted by OH or halogen;
   C₁₋₄-alkylene-4-7-membered heterocycloalkyl with one or more N, O in the heterocycloalkyl ring, wherein the heterocycloalkyl group may be substituted by C₁₋₃ alkyl, which may be substituted by halogen or ═O;
   6-membered aryl which may be substituted by halogen, C₁₋₃ alkyl or —O—C₁₋₃ alkyl wherein the C₁₋₃ alkyl and —O—C₁₋₃ alkyl groups may be substituted by halogen; or
   5-7-membered heteroaryl with one or more N, O or S in the heteroaryl ring, wherein the heteroaryl ring may be substituted by C₁₋₃ alkyl, which may be substituted by halogen or the heteroaryl ring may be substituted by OH; or a
   C₁₋₃-alkylene-5-6-membered heteroaryl, with one or more N or O in the heteroaryl ring wherein the C₁₋₃-alkylene group may be substituted by halogen or OH and the heteroaryl ring may be substituted by OH or a C₁₋₃ alkyl group, which may be substituted by halogen; and R⁶, R⁷ and R⁸ represent hydrogen.
37. The compound of any one of the preceding items, wherein
Q represents OR² or —NR³R⁴;
T represents —O—;
U represents —CH₂—;
V represents
   H;
   C₁₋₃-alkyl which may be straight or branched and which may be substituted with at least one fluorine atom;
   3-6 membered cycloalkyl;
   4-6-membered heterocycloalkyl, comprising one oxygen atom in the heterocycloalkyl ring;
   6-membered aryl, which may be substituted with at least one fluorine atom, with at least one chlorine atom or with at least one C₁₋₃-alkyl group, wherein the C₁₋₃-alkyl group may be substituted by at least one fluorine atom;
   5-6-membered heteroaryl with one or more N, O or S atom in the heteroaryl ring, wherein the heteroaryl ring may be substituted with a C₁₋₃-alkyl group which may be substituted by at least one fluorine atom;
R¹ represents —C₁₋₃-alkyl;
R² represents hydrogen;
R³ represents hydrogen;
R⁴ represents
   C₁₋₆ alkyl which may be straight or branched and which may be substituted by OH, F, ═O, —NR₂, —NR—SO₂-Me or —CONR₂, wherein residues R are independently from each other hydrogen or methyl;
   3-5-membered cycloalkyl which may be substituted by halogen, C₁₋₃-alkyl, which may be substituted by halogen or —OH;
   4-7-membered heterocycloalkyl with one or more N or O atom in the heterocycloalkyl ring and wherein the heterocycloalkyl ring may be substituted by halogen, ═O or —CONR₂ wherein the residues R independently of each other represent H or C₁₋₃-alkyl or the heterocycloalkyl ring may be substituted by a C₁₋₃ alkyl group, which may be substituted by OH or halogen;
   C₁₋₄-alkylene-5-6-membered heterocycloalkyl with one or more N or O in the heterocycloalkyl ring, wherein the C₁₋₄-alkylene groups may be straight or branched and may substituted by OH; or
   6-membered aryl which may be substituted by halogen or straight or branched C₁₋₃ alkyl; or
   C₁₋₄-alkylene-6-membered aryl, wherein the C₁₋₄-alkylene groups may be straight or branched and may substituted by halogen or OH and the aryl groups may be substituted by halogen C₁₋₃-alkyl or —O—C₁₋₃-alkyl, wherein the C₁₋₃-alkyl and —O—C₁₋₃-alkyl groups may be independently of each other substituted by halogen; or
   5-7-membered heteroaryl with one or more N, O or S in the heteroaryl ring, wherein the heteroaryl ring may be substituted by C₁₋₆ alkyl, which may be substituted by halogen or the heteroaryl ring may be substituted by OH; or
   C₁₋₃-alkylene-5-6-membered heteroaryl, with one or more N or O in the heteroaryl ring wherein the C₁₋₃-alkylene group may be substituted by halogen or OH and the heteroaryl ring may be substituted by OH, cyclopropyl, cyclobutyl or a C₁₋₃ alkyl group, which may be substituted by halogen;
and R⁶, R⁷ and R⁸ represent hydrogen.
38. The compound of any one of the preceding items, wherein
Q represents OR² or —NR³R⁴;
T represents —O—;
U represents —CH₂—;
V represents
   methyl which may be substituted with at least one fluorine atom;
   3-5-membered cycloalkyl;
   phenyl, which may be substituted with at least one fluorine atom, with at least one chlorine atom or with at least one methyl group, which may be substituted with fluorine;
   1,3-thiazole which may be substituted with a methyl group; or pyridine, which may be substituted with a methyl group, which may be substituted with fluorine;
R¹ represents —C₁₋₃-alkyl;
R² represents hydrogen;
R³ represents hydrogen;
R⁴ represents
   C₁₋₆ alkyl which may be straight or branched and which may be substituted by OH, F, ═O, —NMe₂, —NR—SO₂-Me or —CONR₂, wherein residues R are independently from each other hydrogen or methyl;
   3-4-membered cycloalkyl which may be substituted by fluor or C₁₋₃-alkyl, which may be substituted with —OH;
   4-6-membered heterocycloalkyl with one or two N or O atom in the heterocycloalkyl ring and wherein the heterocycloalkyl ring may be substituted by halogen or ═O or C₁₋₃ alkyl group, which may be substituted by OH or fluor;
   C₁₋₄-alkylene-5-6-membered heterocycloalkyl with one or two 0 atoms in the heterocycloalkyl ring, wherein the C₁₋₄-alkylene group may be substituted with OH;
   C₁₋₄-alkylene-6-membered aryl, wherein the C₁₋₄-alkylene groups may be straight or branched and may be substituted by fluor or OH and the aryl groups may be substituted by fluor or methoxy, wherein the methoxy groups may be substituted by fluor; or
   5-6-membered heteroaryl with one or two nitrogen atoms in the heteroaryl ring, wherein the heteroaryl ring may be substituted with OH or C₁₋₃ alkyl, which may be substituted by fluor; or
   C₁₋₃-alkylene-5-6-membered heteroaryl, with one or two nitrogen atoms in the heteroaryl ring wherein the C₁₋₃-alkylene group may be substituted by fluor or OH and the heteroaryl ring may be substituted by OH, cyclopropyl, cyclobutyl or a C₁₋₃ alkyl group, which may be substituted by halogen; and R⁶, R⁷ and R⁸ represent hydrogen.
39. The compound of any one of the preceding items, wherein
Q represents OR² or —NR³R⁴;
T represents —O—;
U represents —CH₂—;
V represents
   methyl which is substituted with two fluorine atoms; cyclopropyl;
   phenyl, which may be substituted with one or two fluorine atoms or chlorine atoms or with a fluorine atom and a methyl group, which is substituted with 1, 2 or 3 fluorine atoms;
   1,3-thiazole which is substituted with a methyl group; or pyridine, which is substituted with a methyl group;
R¹ represents —C₁₋₃-alkyl;
R² represents hydrogen;
R³ represents hydrogen;
R⁴ represents
   C₁₋₅ alkyl which may be substituted by up to two OH, up to two F, —CONR₂, or —NHS(═O)₂-Me, wherein residues R are independently from each other hydrogen or methyl; or a
   3-4-membered cycloalkyl which may be substituted by fluor or C₁₋₃-alkyl, which may be substituted with fluor or —OH;
   5-6-membered heterocycloalkyl with one or two N or O atoms in the heterocycloalkyl ring and wherein the heterocycloalkyl ring may be substituted by halogen, ═O or C₁₋₂ alkyl group, which may be substituted by OH or fluor;
   C₁₋₃-alkylene-tetrahydropyran rest, wherein the C₁₋₄-alkylene group is substituted by OH;
   C₁₋₂-alkylene-6-membered aryl, wherein the C₁₋₂-alkylene groups may be straight or branched and may be substituted by fluor or OH and the aryl groups may be substituted by fluor, methyl or methoxy, wherein the methyl or methoxy groups may be substituted by fluor; or pyridine ring substituted with OH and trifluoromethyl; or
   C₁₋₃-alkylene-5-6-membered heteroaryl, with one or two nitrogen atoms in the heteroaryl ring wherein the C₁₋₃-alkylene group may be substituted by fluor or OH and the heteroaryl ring may be substituted by OH, cyclopropyl, cyclobutyl or methyl, which may be substituted by fluor; and R⁶, R⁷ and R⁸ represent hydrogen.
40. The compound of any one of the preceding items, wherein
Q represents OR² or —NR³R⁴;
T represents —O—;
U represents —CH₂—;
V represents
   H;
   C₁₋₃-alkyl which may be substituted with at least one halogen;
   3-6 membered cycloalkyl, which may be substituted with a —CN group;
   4-6-membered heterocycloalkyl, comprising one oxygen atom in the heterocycloalkyl ring;
   6-membered aryl, which may be substituted with at least one halogen or with a C₁₋₃-alkyl group, wherein the C₁₋₃-alkyl group may be substituted by at least one halogen atom;
   5-6-membered heteroaryl with one or more N, O or S atom in the heteroaryl ring, wherein the heteroaryl ring may be substituted with halogen or a C₁₋₃-alkyl group which may be substituted by at least one halogen atom;
R¹ represents —C₁₋₃-alkyl;
R² represents hydrogen;
R³ represents hydrogen;
R⁴ represents
   —SO₂—C₁₋₃-alkyl;
   C₁₋₆ alkyl which may be substituted by OH, F, —NR—SO₂—R or —CONR₂, wherein residues R are independently from each other hydrogen or C₁₋₃ alkyl;
   3-6-membered cycloalkyl which may be substituted by halogen, C₁₋₃-alkylene-OH, ═O or —CONH₂;
   C₁₋₃-alkylene-3-6-membered cycloalkyl which may be substituted by a C₁₋₃-alkyl or a —N (C₁₋₆ alkyl)₂ group;
   4-7-membered heterocycloalkyl with one or more N or O atom in the heterocycloalkyl ring and wherein the heterocycloalkyl ring may be substituted by ═O, halogen or —CONR₂ wherein the residues R independently of each other represent H or C₁₋₃-alkyl or the cycloalkyl ring may be substituted by a C₁₋₃ alkyl group, which may be substituted by at least one of OH or halogen;
   C₁₋₄-alkylene-4-7-membered heterocycloalkyl with one or more N, O in the heterocycloalkyl ring, wherein the heterocycloalkyl group may be substituted by C₁₋₃ alkyl or ═O;
   5-7-membered heteroaryl with one or more N, O or S in the heteroaryl ring, wherein the heteroaryl ring may be substituted by C₁₋₆ alkyl or OH; or a
   C₁₋₃-alkylene-5-6-membered heteroaryl, with one or more N, O or S in the heteroaryl ring wherein the heteroaryl ring may be substituted by OH; and R⁶, R⁷ and R⁸ represent hydrogen.
41. The compound of any one of the preceding items, wherein
Q represents OR² or —NR³R⁴;
T represents —O—;
U represents —CH₂—;
V represents
   H;
   C₁₋₃-alkyl which may be substituted with at least one fluorine atom;
   3-6 membered cycloalkyl, which may be substituted with a —CN group;
   4-6-membered heterocycloalkyl, comprising one oxygen atom in the heterocycloalkyl ring;
   6-membered aryl, which may be substituted with at least one fluorine atom, with at least one chlorine atom or with at least one C₁₋₃-alkyl group, wherein the C₁₋₃-alkyl group may be substituted by at least one fluorine atom;
   5-6-membered heteroaryl with one or more N, O or S atom in the heteroaryl ring, wherein the heteroaryl ring may be substituted with a C₁₋₃-alkyl group which may be substituted by at least one fluorine atom;
R¹ represents —C₁₋₃-alkyl;
R² represents hydrogen;
R³ represents hydrogen;
R⁴ represents
   C₁₋₆ alkyl which may be substituted by OH, F, —NH—SO₂—C₁₋₃-alkyl or —CONR₂, wherein residues R are independently from each other hydrogen or methyl;
   4-7-membered heterocycloalkyl with one or more N or O atom in the heterocycloalkyl ring and wherein the heterocycloalkyl ring may be substituted by ═O or by C₁₋₃-alkylene-OH; and R⁶, R⁷ and R⁸ represent hydrogen.
42. The compound of any one of the preceding items, wherein
Q represents OR² or —NR³R⁴;
T represents —O—;
U represents —CH₂—;
V represents
   methyl which may be substituted with at least one fluorine atom;
   3-5-membered cycloalkyl;
   phenyl, which may be substituted with at least one fluorine atom, with at least one chlorine atom or with at least one methyl group;
   1,3-thiazole which may be substituted with a methyl group; or pyridine, which may be substituted with a methyl group;
R¹ represents —C₁₋₃-alkyl;
R² represents hydrogen;
R³ represents hydrogen;
R⁴ represents
   C₁₋₅ alkyl which may be substituted by OH, F or —CONR₂, or —NH—S(═O)₂-Me, wherein the residues R are independently from each other hydrogen or methyl;
   4-6-membered heterocycloalkyl with one N atom in the heterocycloalkyl ring which may be a lactam ring and wherein the heterocycloalkyl ring may be substituted by a C₁₋₂-alkylene-OH;
and R⁶, R⁷ and R⁸ represent hydrogen.
43. The compound of any one of the preceding items, wherein
Q represents OR² or —NR³R⁴;
T represents —O—;
U represents —CH₂—;
V represents
   methyl which is substituted with two fluorine atoms;
   3-4-membered cycloalkyl;
   phenyl, which may be substituted with one or two fluorine atoms or with a fluorine atom and a methyl group;
   1,3-thiazole which is substituted with a methyl group; or
   pyridine, which is substituted with a methyl group;
R¹ represents —C₁₋₃-alkyl;
R² represents hydrogen;
R³ represents hydrogen;
R⁴ represents
   C₁₋₅ alkyl which may be substituted by up to two OH, up to two F, —CONR₂, or —NHS(═O)₂-Me, wherein residues R are independently from each other hydrogen or methyl; or a
   2-pyrrolidone ring with a C₁₋₂-alkylene-OH;
   and R⁶, R⁷ and R⁸ represent hydrogen.
44. The compound of item 1, wherein R³ represents —H.
45. The compound of item 2, wherein R⁴ represents a residue other than —H.
46. The compound of any one of items 44 to 45, wherein R¹ represents -methyl or ethyl.
47. The compound of any one of items 44 to 46, wherein T represents —O— and U represents —CR⁵R^{5′}—.
48. The compound of one of the preceding items, wherein V represents (i) 5-14-membered heteroaryl selected from benzimidazole, benzisoxazole, benzoxazole, benzodioxole, benzofuran, benzothiadiazole, benzothiazole, benzothiophene, carbazole, cinnoline, dibenzofuran, furane, furazane, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine; in each case unsubstituted, mono- or polysubstituted with substituents independently of one another selected from —F, —Cl, —CN, —OH, ═O, —C₁₋₆-alkyl, —CHF₂, —CF₃, —C₁₋₆-alkylene-NH₂, —C₁₋₆-alkylene-NHC(═O)O—C₁₋₆-alkyl, —C₁₋₆-alkylene-OH, —C₁₋₆-alkylene-CHF₂, —C₁₋₆-alkylene-CF₃, —C₁₋₆-alkylene-cyclopropyl, -cyclopropyl, —O-cyclopropyl, —C₁₋₆-alkylene-NHC(═O)—O—C₁₋₆-alkyl, —C(═O)O—C₁₋₆-alkyl, —N(C₁₋₆-alkyl)₂, —OC₁₋₆-alkyl, —OCF₃, —O—C₁₋₆-alkylene-N(C₁₋₆-alkyl)₂, —S(═O)₂—C₁₋₆-alkyl, -azetidine, —C₁₋₆-alkylene-O-tetrahydropyran, or -piperazine substituted with —C₁₋₆-alkyl; particularly in each case unsubstituted, mono- or polysubstituted with substituents independently of one another selected from —F, —Cl, —CN, —OH, ═O, —C₁₋₆-alkyl, —CHF₂, —CF₃, —C₁₋₆-alkylene-NH₂, —C₁₋₆-alkylene-NHC(═O)O—C₁. 6-alkyl, —C₁₋₆-alkylene-OH, —C₁₋₆-alkylene-NHC(═O)—O—C₁₋₆-alkyl, —C(═O)O—C₁₋₆-alkyl, —N(C₁₋₆-alkyl)₂, —OC₁₋₆-alkyl, —OCF₃, —O—C₁₋₆-alkylene-N(C₁₋₆-alkyl)₂, —S(═O)₂—C₁₋₆-alkyl, -azetidine, —C₁₋₆-alkylene-O-tetrahydropyran, or -piperazine substituted with —C₁₋₆-alkyl; or represents (ii) -oxetanyl, unsubstituted, mono- or polysubstituted.
49. The compound of any one of items 1 to 47, wherein the 3-14-membered cycloalkyl, saturated or unsaturated within the definition of V is cyclopropyl, cyclobutyl, cyclopentyl, cyclohexyl, cycloheptyl, cyclooctyl, cyclononyl, or cyclodecyl including unfused or unbridged, fused, or bridged cycloalkyls; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
50. The compound of any one of items 1 to 47 wherein the 5-14-membered aryl within the definition of V is phenyl or another 5-14-membered aryl, unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
51. The compound of any one of items 1 to 47 wherein the 3-14-membered heterocycloalkyl within the definition of V is selected from azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxane, oxepane, oxetane, oxirane, piperazine, piperidine, pyrazolidine, pyrrolidine, quinuclidine, tetrahydrofurane, tetrahydropyrane, tetrahydrothiopyrane, thiazolidine, thietane, thiirane, thiolane, thiomorpholine, indoline, dihydrobenzofuran, dihydrobenzo-thiophene, 1,1-dioxothia-cyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexa-hydro-cyclopenta[c]pyrrole, octahydro-cyclopenta[c]pyrrole, and octahydro-pyrrolo[1,2-a]pyrazine; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
52. The compound of any one of items 1 to 47 wherein V represents C₁-C₆ alkyl or C₁-C₆ heteroalkyl, saturated or unsaturated, unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.
53. The compound of any one of 1 to 47 wherein V is a residue selected from the group consisting of:

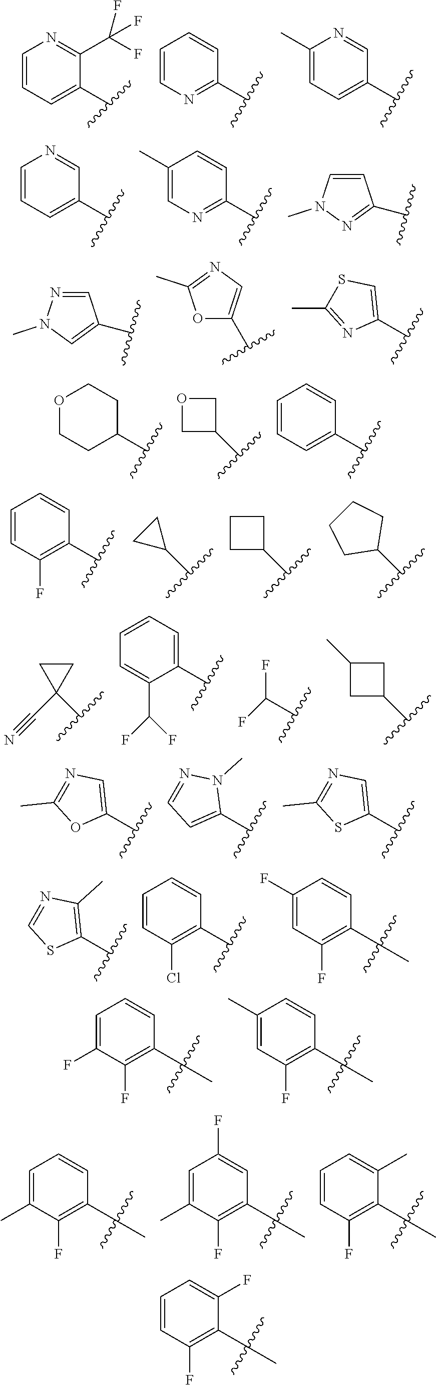

54. The compound according to any of the preceding items, wherein R¹ represents —H, —F, —Cl, —Br, —I, —C₁₋₆-alkyl, —O—C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)C₁₋₆-alkyl, —C(═O)OC₁₋₆-alkyl, —C(═O)NH₂, —C(═O)NHC₁₋₆-alkyl, —C(═O)N(C₁₋₆-alkyl)₂, —S(═O)—C₁₋₆-alkyl, —S(═O)₂—C₁₋₆-alkyl, —O—C₁₋₆-alkyl, -cyclopropyl unsubstituted, cyclobutyl unsubstituted, cyclopentyl unsubstituted or cyclohexyl unsubstituted.
55. The compound according to any of the preceding items, wherein R³ represents —H, —OH, —C₁₋₆-alkyl, —C₁₋₆-alkylene-OH, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃. 56. The compound according to any one of the preceding items, wherein R⁴ represents
   —H;
   —S(═O)₂C₁₋₆-alkyl, saturated, unsubstituted, monosubstituted or polysubstituted with —F;
   —S(═O)₂(3-14-membered cycloalkyl), saturated, unsubstituted;
   —C₁₋₆-alkyl, saturated, unsubstituted mono- or polysubstituted;
   3-14-membered cycloalkyl or —C₁₋₆-alkylene-(3-14-membered cycloalkyl), each unsubstituted, mono- or polysubstituted;
   3-14-membered heterocycloalkyl or —C₁₋₆-alkylene-(3-14-membered heterocycloalkyl), unsubstituted, mono- or polysubstituted;
   -phenyl, or —C₁₋₆-alkylene-phenyl, each unsubstituted, mono- or polysubstituted; or
   5-14-membered heteroaryl or —C₁₋₆-alkylene-(5-14-membered heteroaryl), each unsubstituted, mono- or polysubstituted.
57. The compound according to any one of claims 1 to 55 wherein R⁴ represents a 3-14-membered cycloalkyl (preferably a 3, 4, 5 or 6-membered cycloalkyl), saturated or unsaturated, unsubstituted, mono- or polysubstituted; wherein said 3-14-membered cycloalkyl is connected through —C₁-C₆-alkylene-, saturated or unsaturated, unsubstituted, mono- or polysubstituted; or a 3-14-membered heterocycloalkyl (preferably a 4, 5 or 6-membered heterocycloalkyl), saturated or unsaturated, unsubstituted, mono- or polysubstituted; wherein said 3-14-membered heterocycloalkyl is connected through —C₁-C₆-alkylene-, saturated or unsaturated, unsubstituted, mono- or polysubstituted; or a 6-14-membered aryl (preferably a 6-membered aryl), unsubstituted, mono- or polysubstituted; wherein said 6-14-membered aryl is connected through —C₁-C₆-alkylene-, saturated or unsaturated, unsubstituted, mono- or polysubstituted; or a 5-14-membered heteroaryl (preferably a 5 or 6-membered heteroaryl), unsubstituted, mono- or polysubstituted; wherein said 5-14-membered heteroaryl is connected through —C₁-C₆-alkylene-, saturated or unsaturated, unsubstituted, mono- or polysubstituted.
58. The compound according to any one of the preceding items wherein R³ is H and R⁴ is a residue selected from the group consisting of:

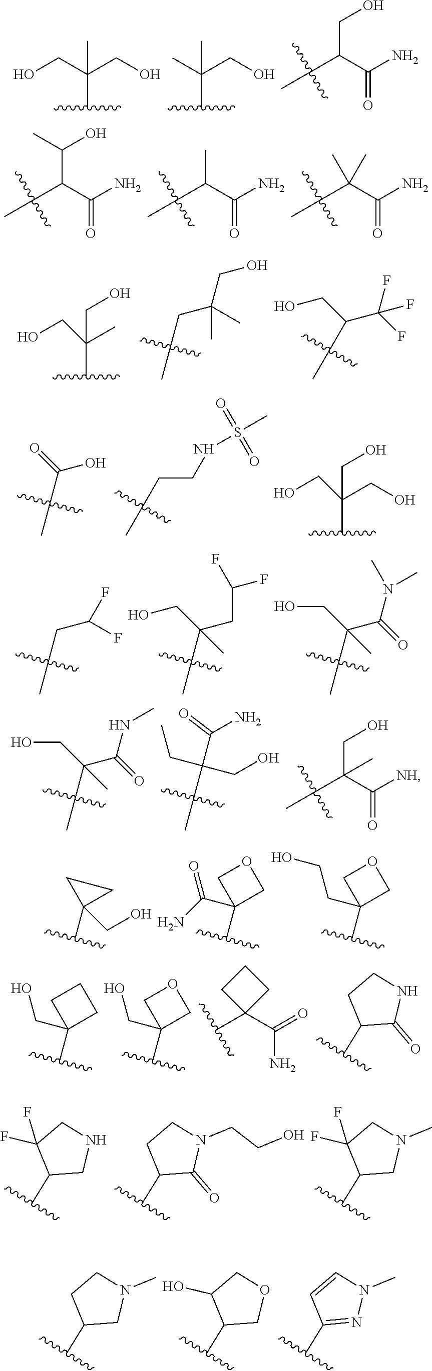

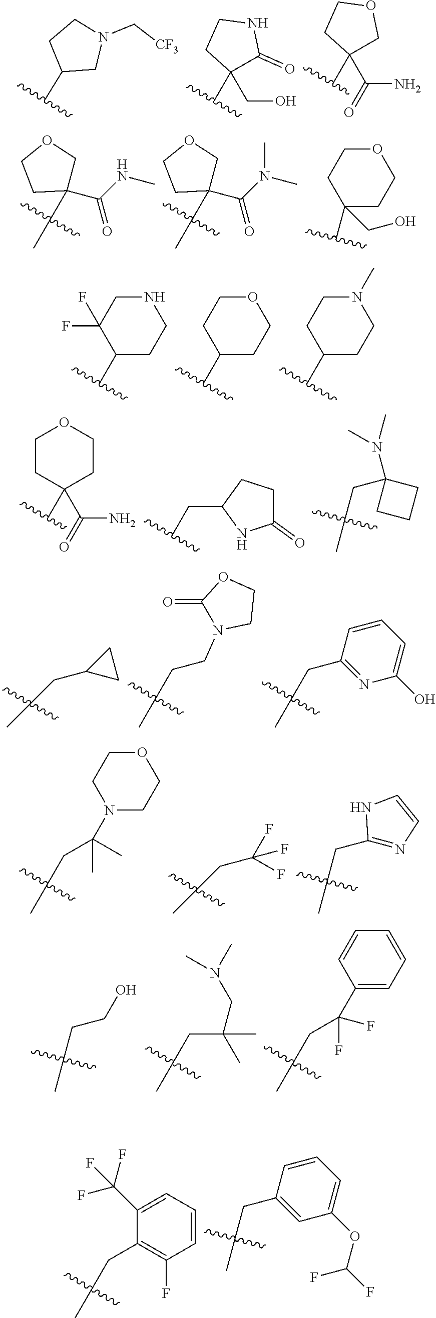

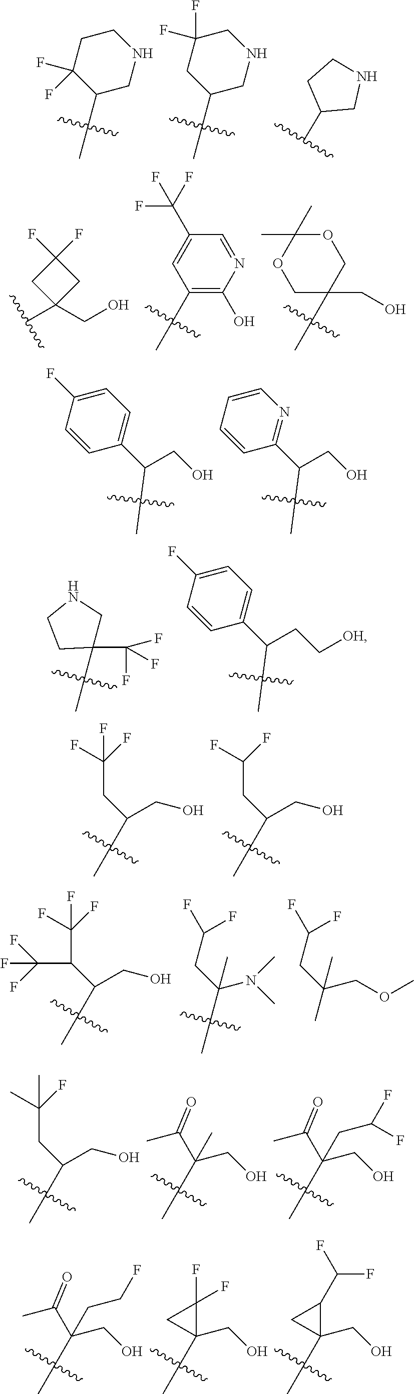

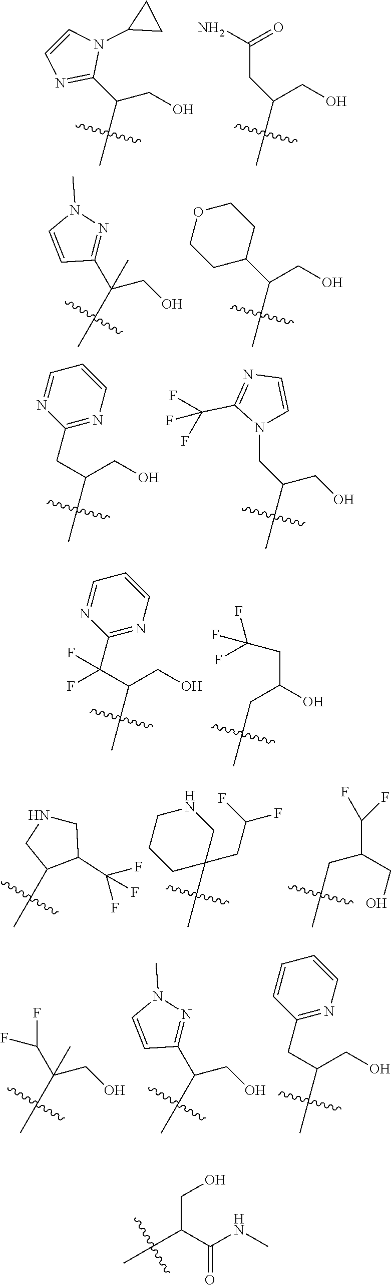

59. The compound of any one of the preceding items wherein R³ and R⁴ together form a heterocycle selected from the group consisting of pyrrolidine, piperidine, morpholine, and piperazine, in each case unsubstituted, mono- or polysubstituted with substituents independently of one another selected from the group consisting of —F, —C₁₋₆-alkyl, —NH₂, —NHCH₃, —N(CH₃)₂, —C(═O)NH—C₁₋₆-alkyl, —C(═O)N(C₁₋₆-alkyl)₂, —C(═O)O—C₁₋₆-alkyl, —NHC(═O)O—C₁₋₆-alkyl, -pyridyl unsubstituted, and 1,2,4-oxadiazole unsubstituted or monosubstituted with —C₁₋₆-alkyl.
60. The compound of any one of the preceding items, wherein R⁵ and R^{5′} independently of one another represent
   —H;
   —C₁-C₆-alkyl, saturated or unsaturated, unsubstituted, mono- or polysubstituted;
   —C₁-C₆-heteroalkyl, saturated or unsaturated, unsubstituted, mono- or polysubstituted;
   3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.
61. The compound according to any one of the preceding items, wherein R⁶, R⁷ and R⁸ independently of one another represent
   —H, —F, —Cl, —Br, —I, —OH, —SH, —SF₅, —CN, —NO₂, —C(═O)OH, —NH₂,
   —C₁₋₆-alkyl, —CF₃, —CHF₂, —CH₂F,
   —O—C₁₋₆-alkyl, —OCF₃, —OCHF₂, —OCH₂F,
   —NHC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
   —N(C₁₋₆-alkyl)₂ unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
   —C(═O)OC₁₋₆-alkyl unsubstituted or substituted with one or more substituents independently of one another selected from —OH, ═O, —F, —Cl, —Br, —I, —SH, ═S, —ON, —CF₃, —CHF₂, —CH₂F, —OCF₃, —OCHF₂, —OCH₂F, SF₅, —NO₂, —C(═O)OH, —NH₂, and —C(═O)NH₂;
   —OC(═O)C₁₋₆-alkyl unsubstituted or substituted with one or more substituents independently of one another selected from —OH, ═O, —F, —Cl, —Br, —I, —SH, ═S, —ON, —OF₃, —CHF₂, —CH₂F, —OCF₃, —OCHF₂, —OCH₂F, SF₅, —NO₂, —C(═O)OH, —NH₂, and —C(═O)NH₂; or
   —C₁₋₆-heteroalkyl unsubstituted or substituted with one or more substituents independently of one another selected from —OH, ═O, —F, —Cl, —Br, —I, —SH, ═S, —ON, —OF₃, —CHF₂, —CH₂F, —OCF₃, —OCHF₂, —OCH₂F, SF₅, —NO₂, —C(═O)OH, —NH₂, and —C(═O)NH₂.
62. The compound of any one of the preceding items, which is selected from the group consisting of compounds 001-036 as shown in the table below:

Structure and Compound CODE

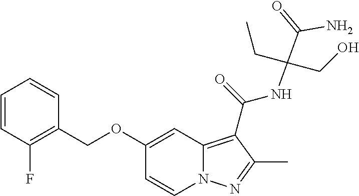 [SMILES: CCC(CO)(NC(=O)c1c(C)nn2ccc(OCc3ccccc3F)cc12)C(N)=O]

Cpd 001

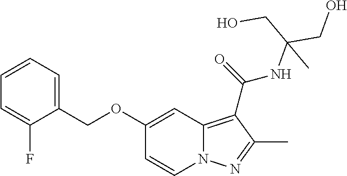 [SMILES: Cc1nn2ccc(OCc3ccccc3F)cc2c1C(=O)NC(C)(CO)CO]

Cpd 002

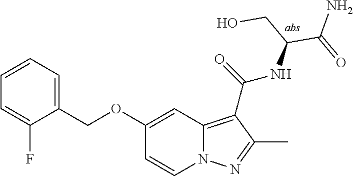 [SMILES: Cc1nn2ccc(OCc3ccccc3F)cc2c1C(=O)N[C@@H](CO)C(N)=O]

Cpd 003

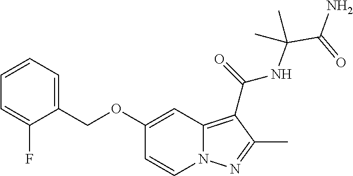 [SMILES: Cc1nn2ccc(OCc3ccccc3F)cc2c1C(=O)NC(C)(C)C(N)=O]

Cpd 004

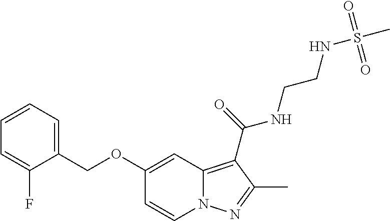 [SMILES: Cc1nn2ccc(OCc3ccccc3F)cc2c1C(=O)NCCNS(C)(=O)=O]

Cpd 005

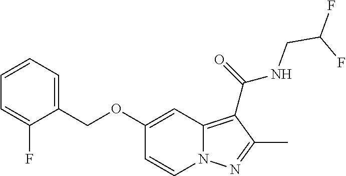 [SMILES: Cc1nn2ccc(OCc3ccccc3F)cc2c1C(=O)NCC(F)F]

Cpd 006

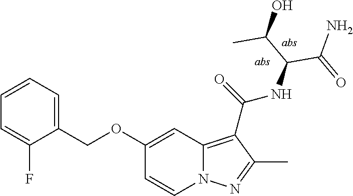 [SMILES: Cc1nn2ccc(OCc3ccccc3F)cc2c1C(=O)N[C@H](C(N)=O)[C@@H](C)O]

Cpd 007

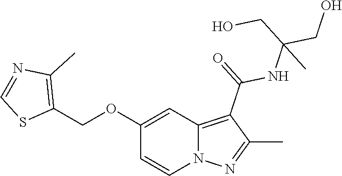 [SMILES: Cc1ncsc1COc1ccn2nc(C)c(C(=O)NC(C)(CO)CO)c2c1]

Cpd 008

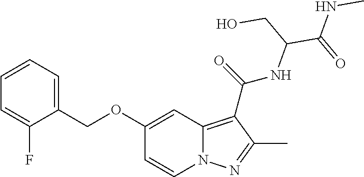 [SMILES: CNC(=O)C(CO)NC(=O)c1c(C)nn2ccc(OCc3ccccc3F)cc12]

Cpd 009

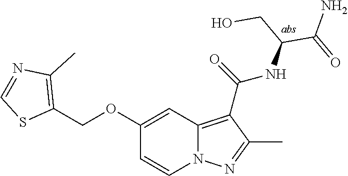 [SMILES: Cc1ncsc1COc1ccn2nc(C)c(C(=O)N[C@@H](CO)C(N)=O)c2c1]

Cpd 010

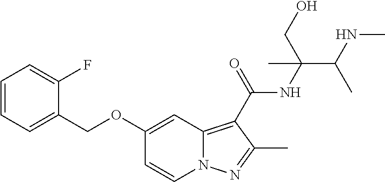 [SMILES: CNC(C)C(C)(CO)NC(=O)c1c(C)nn2ccc(OCc3ccccc3F)cc12]

Cpd 011

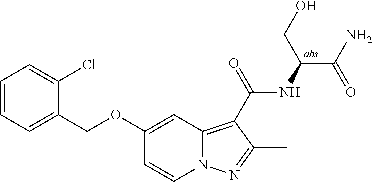 [SMILES: Cc1nn2ccc(OCc3ccccc3Cl)cc2c1C(=O)N[C@@H](CO)C(N)=O]

Cpd 012

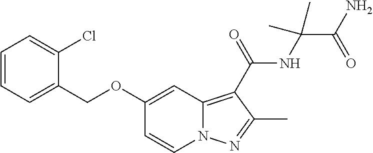 [SMILES: Cc1nn2ccc(OCc3ccccc3Cl)cc2c1C(=O)NC(C)(C)C(N)=O]

Cpd 013

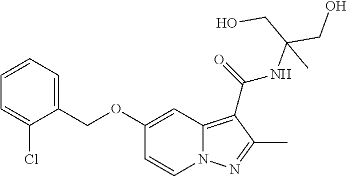 [SMILES: Cc1nn2ccc(OCc3ccccc3Cl)cc2c1C(=O)NC(C)(CO)CO]

Cpd 014

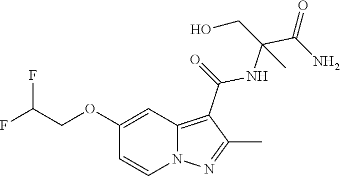 [SMILES: Cc1nn2ccc(OCC(F)F)cc2c1C(=O)NC(C)(CO)C(N)=O]

Cpd 015

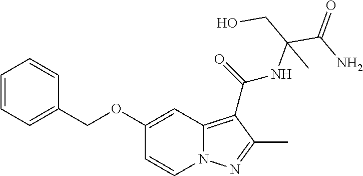 [SMILES: Cc1nn2ccc(OCc3ccccc3)cc2c1C(=O)NC(C)(CO)C(N)=O]

Cpd 016

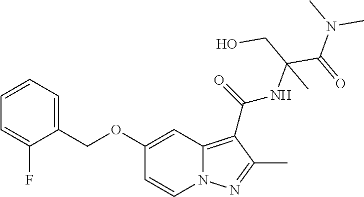 [SMILES: Cc1nn2ccc(OCc3ccccc3F)cc2c1C(=O)NC(C)(CO)C(=O)N(C)C]

Cpd 017

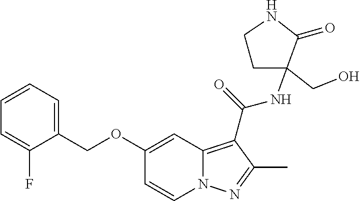 [SMILES: Cc1nn2ccc(OCc3ccccc3F)cc2c1C(=O)NC1(CO)CCNC1=O]

Cpd 018

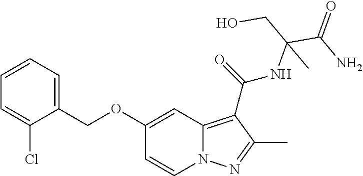 [SMILES: Cc1nn2ccc(OCc3ccccc3Cl)cc2c1C(=O)NC(C)(CO)C(N)=O]

Cpd 019

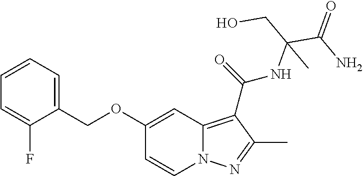 [SMILES: Cc1nn2ccc(OCc3ccccc3F)cc2c1C(=O)NC(C)(CO)C(N)=O]

Cpd 020

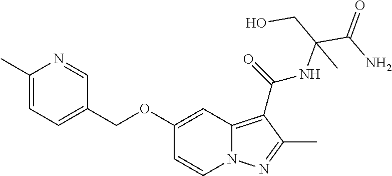 [SMILES: Cc1ccc(COc2ccn3nc(C)c(C(=O)NC(C)(CO)C(N)=O)c3c2)cn1]

Cpd 021

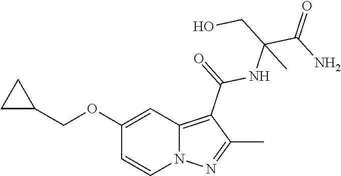 [SMILES: Cc1nn2ccc(OCC3CC3)cc2c1C(=O)NC(C)(CO)C(N)=O]

Cpd 022

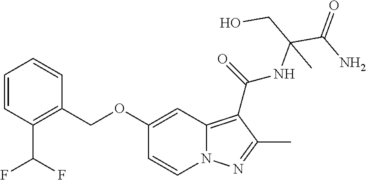 [SMILES: Cc1nn2ccc(OCc3ccccc3C(F)F)cc2c1C(=O)NC(C)(CO)C(N)=O]

Cpd 023

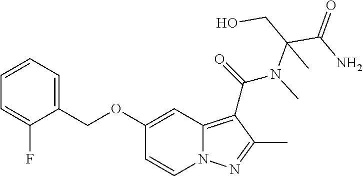 [SMILES: Cc1nn2ccc(OCc3ccccc3F)cc2c1C(=O)N(C)C(C)(CO)C(N)=O]

Cpd 024

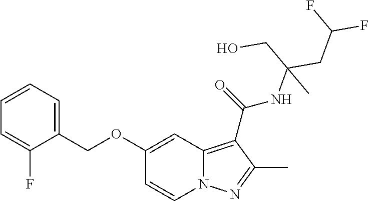 [SMILES: Cc1nn2ccc(OCc3ccccc3F)cc2c1C(=O)NC(C)(CO)CC(F)F]

Cpd 025

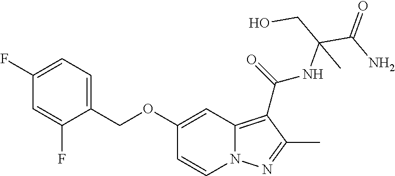 [SMILES: Cc1nn2ccc(OCc3ccc(F)cc3F)cc2c1C(=O)NC(C)(CO)C(N)=O]

Cpd 026

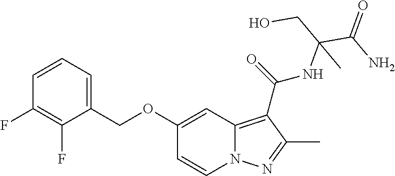 [SMILES: Cc1nn2ccc(OCc3cccc(F)c3F)cc2c1C(=O)NC(C)(CO)C(N)=O]

Cpd 027

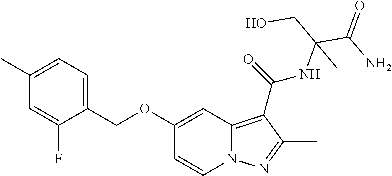 [SMILES: Cc1ccc(COc2ccn3nc(C)c(C(=O)NC(C)(CO)C(N)=O)c3c2)c(F)c1]

Cpd 028

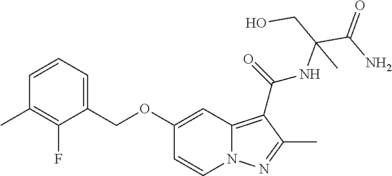 [SMILES: Cc1cccc(COc2ccn3nc(C)c(C(=O)NC(C)(CO)C(N)=O)c3c2)c1F]

Cpd 029

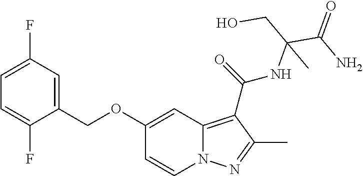 [SMILES: Cc1nn2ccc(OCc3cc(F)ccc3F)cc2c1C(=O)NC(C)(CO)C(N)=O]

Cpd 030

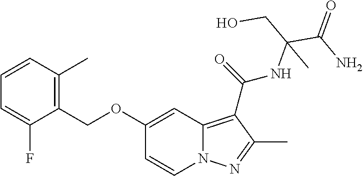 [SMILES: Cc1cccc(F)c1COc1ccn2nc(C)c(C(=O)NC(C)(CO)C(N)=O)c2c1]

Cpd 031

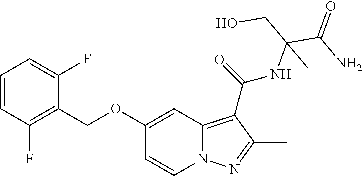 [SMILES: Cc1nn2ccc(OCc3c(F)cccc3F)cc2c1C(=O)NC(C)(CO)C(N)=O]

Cpd 032

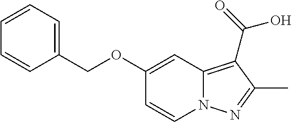 [SMILES: Cc1nn2ccc(OCc3ccccc3)cc2c1C(=O)O]

Cpd 033

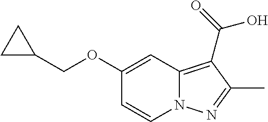 [SMILES: Cc1nn2ccc(OCC3CC3)cc2c1C(=O)O]

Cpd 034

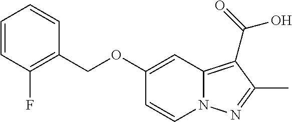 [SMILES: Cc1nn2ccc(OCc3ccccc3F)cc2c1C(=O)O]

Cpd 035

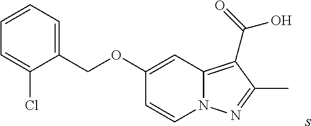

Cpd 036

63. The compound of any one of the preceding items, which is selected from the group consisting of compounds 037-091 as shown in the table below:

Structure and Compound CODE

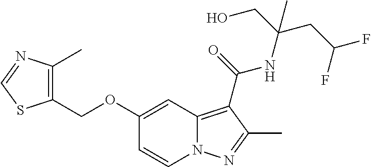 [SMILES: Cc1ncsc1COc1ccn2nc(C)c(C(=O)NC(C)(CO)CC(F)F)c2c1]

Cpd 037

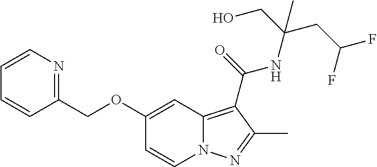 [SMILES: Cc1nn2ccc(OCc3ccccn3)cc2c1C(=O)NC(C)(CO)CC(F)F]

Cpd 038

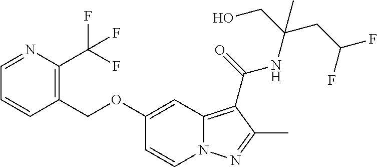 [SMILES: Cc1nn2ccc(OCc3cccnc3C(F)(F)F)cc2c1C(=O)NC(C)(CO)CC(F)F]

Cpd 039

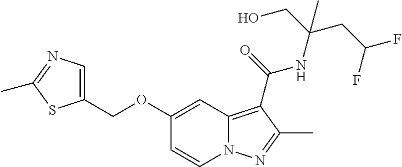 [SMILES: Cc1ncc(COc2ccn3nc(C)c(C(=O)NC(C)(CO)CC(F)F)c3c2)s1]

Cpd 040

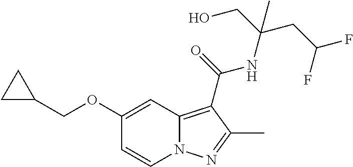 [SMILES: Cc1nn2ccc(OCC3CC3)cc2c1C(=O)NC(C)(CO)CC(F)F]

Cpd 041

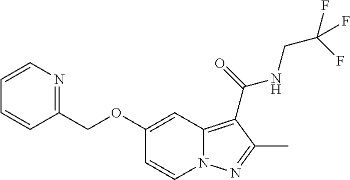 [SMILES: Cc1nn2ccc(OCc3ccccn3)cc2c1C(=O)NCC(F)(F)F]

Cpd 042

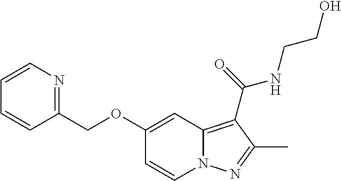 [SMILES: Cc1nn2ccc(OCc3ccccn3)cc2c1C(=O)NCCO]

Cpd 043

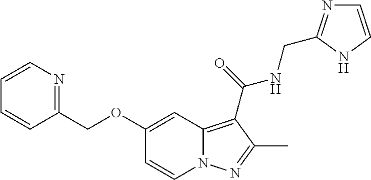 [SMILES: Cc1nn2ccc(OCc3ccccn3)cc2c1C(=O)NCc1ncc[nH]1]

Cpd 044

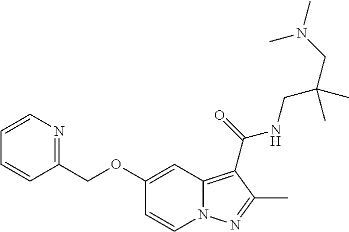 [SMILES: Cc1nn2ccc(OCc3ccccn3)cc2c1C(=O)NCC(C)(C)CN(C)C]

Cpd 045

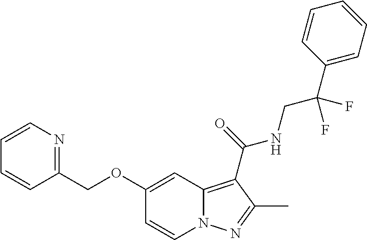 [SMILES: Cc1nn2ccc(OCc3ccccn3)cc2c1C(=O)NCC(F)(F)c1ccccc1]

Cpd 046

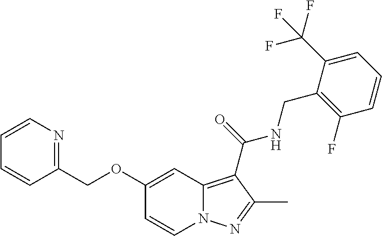 [SMILES: Cc1nn2ccc(OCc3ccccn3)cc2c1C(=O)NCc1c(F)cccc1C(F)(F)F]

Cpd 047

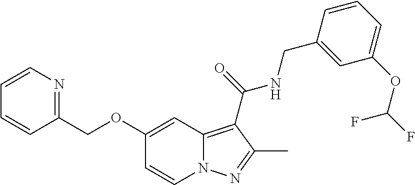 [SMILES: Cc1nn2ccc(OCc3ccccn3)cc2c1C(=O)NCc1cccc(OC(F)F)c1]

Cpd 048

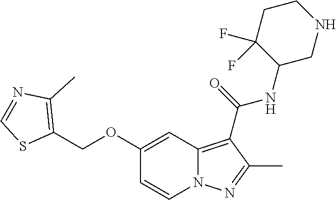 [SMILES: Cc1ncsc1COc1ccn2nc(C)c(C(=O)NC3CNCCC3(F)F)c2c1]

Cpd 049

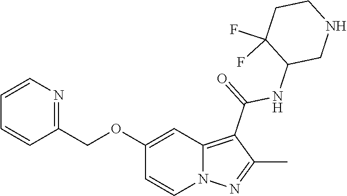 [SMILES: Cc1nn2ccc(OCc3ccccn3)cc2c1C(=O)NC1CNCCC1(F)F]

Cpd 050

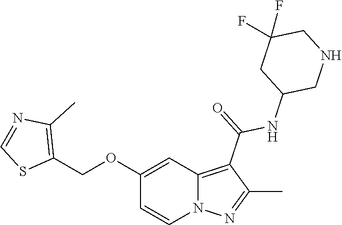 [SMILES: Cc1ncsc1COc1ccn2nc(C)c(C(=O)NC3CNCC(F)(F)C3)c2c1]

Cpd 051

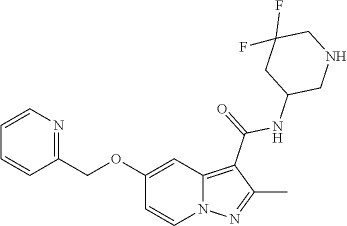 [SMILES: Cc1nn2ccc(OCc3ccccn3)cc2c1C(=O)NC1CNCC(F)(F)C1]

Cpd 052

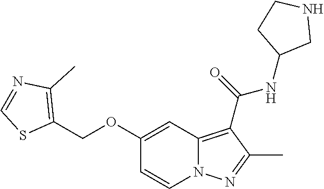 [SMILES: Cc1ncsc1COc1ccn2nc(C)c(C(=O)NC3CCNC3)c2c1]

Cpd 053

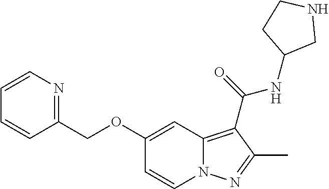 [SMILES: Cc1nn2ccc(OCc3ccccn3)cc2c1C(=O)NC1CCNC1]

Cpd 054

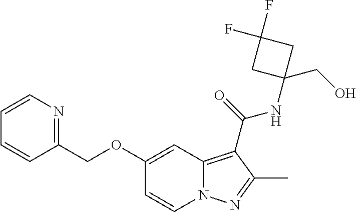 [SMILES: Cc1nn2ccc(OCc3ccccn3)cc2c1C(=O)NC1(CO)CC(F)(F)C1]

Cpd 055

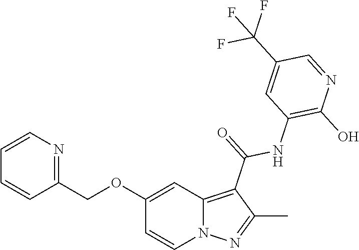 [SMILES: Cc1nn2ccc(OCc3ccccn3)cc2c1C(=O)Nc1cc(C(F)(F)F)cnc1O]

Cpd 056

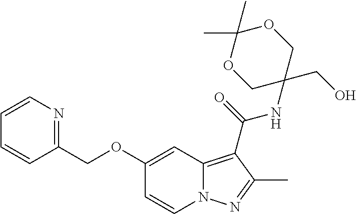 [SMILES: Cc1nn2ccc(OCc3ccccn3)cc2c1C(=O)NC1(CO)COC(C)(C)OC1]

Cpd 057

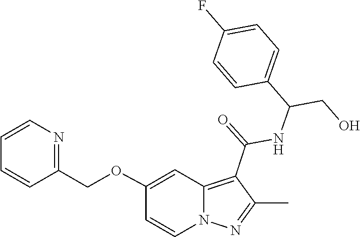 [SMILES: Cc1nn2ccc(OCc3ccccn3)cc2c1C(=O)NC(CO)c1ccc(F)cc1]

Cpd 058

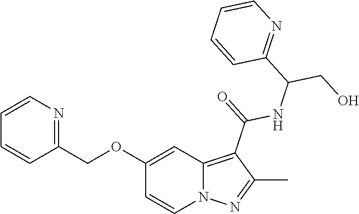 [SMILES: Cc1nn2ccc(OCc3ccccn3)cc2c1C(=O)NC(CO)c1ccccn1]

Cpd 059

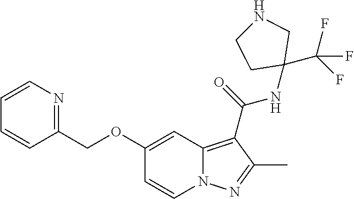 [SMILES: Cc1nn2ccc(OCc3ccccn3)cc2c1C(=O)NC1(C(F)(F)F)CCNC1]

Cpd 060

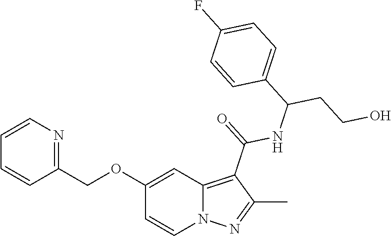 [SMILES: Cc1nn2ccc(OCc3ccccn3)cc2c1C(=O)NC(CCO)c1ccc(F)cc1]

Cpd 061

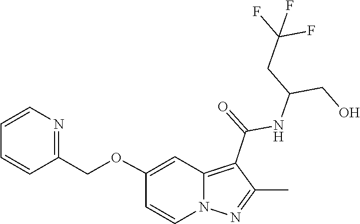 [SMILES: Cc1nn2ccc(OCc3ccccn3)cc2c1C(=O)NC(CO)CC(F)(F)F]

Cpd 062

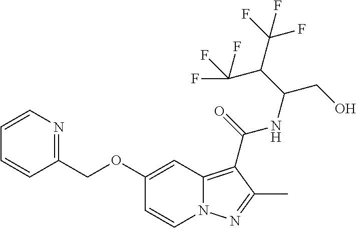 [SMILES: Cc1nn2ccc(OCc3ccccn3)cc2c1C(=O)NC(CO)C(C(F)(F)F)C(F)(F)F]

Cpd 063

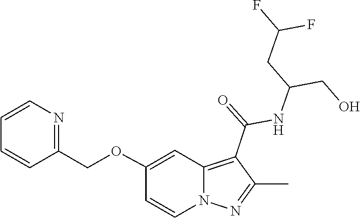 [SMILES: Cc1nn2ccc(OCc3ccccn3)cc2c1C(=O)NC(CO)CC(F)F]

Cpd 064

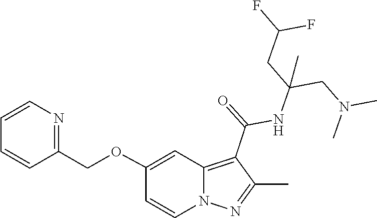 [SMILES: Cc1nn2ccc(OCc3ccccn3)cc2c1C(=O)NC(C)(CC(F)F)CN(C)C]

Cpd 065

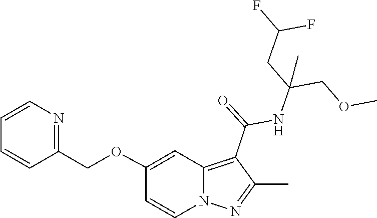 [SMILES: COCC(C)(CC(F)F)NC(=O)c1c(C)nn2ccc(OCc3ccccn3)cc12]

Cpd 066

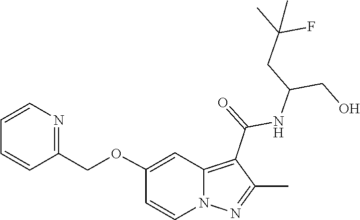 [SMILES: Cc1nn2ccc(OCc3ccccn3)cc2c1C(=O)NC(CO)CC(C)(C)F]

Cpd 067

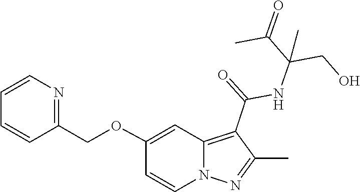 [SMILES: CC(=O)C(C)(CO)NC(=O)c1c(C)nn2ccc(OCc3ccccn3)cc12]

Cpd 068

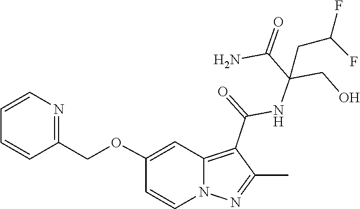 [SMILES: Cc1nn2ccc(OCc3ccccn3)cc2c1C(=O)NC(CO)(CC(F)F)C(N)=O]

Cpd 069

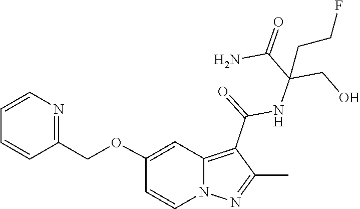 [SMILES: Cc1nn2ccc(OCc3ccccn3)cc2c1C(=O)NC(CO)(CCF)C(N)=O]

Cpd 070

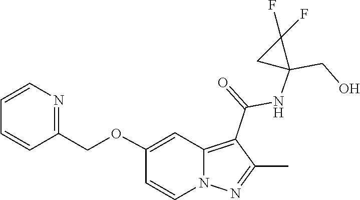 [SMILES: Cc1nn2ccc(OCc3ccccn3)cc2c1C(=O)NC1(CO)CC1(F)F]

Cpd 071

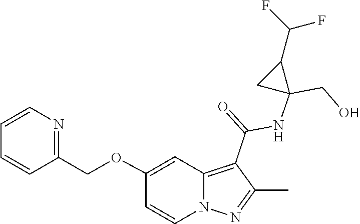 [SMILES: Cc1nn2ccc(OCc3ccccn3)cc2c1C(=O)NC1(CO)CC1C(F)F]

Cpd 072

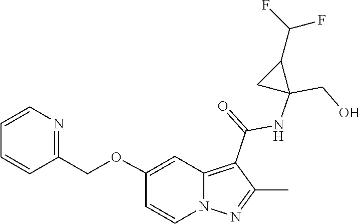 [SMILES: Cc1nn2ccc(OCc3ccccn3)cc2c1C(=O)NC1(CO)CC1C(F)F]

Cpd 073

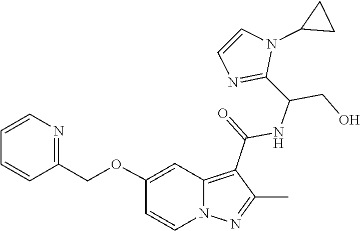 [SMILES: Cc1nn2ccc(OCc3ccccn3)cc2c1C(=O)NC(CO)c1nccn1C1CC1]

Cpd 074

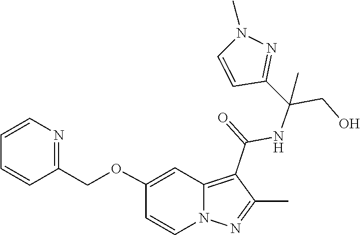 [SMILES: Cc1nn2ccc(OCc3ccccn3)cc2c1C(=O)NC(C)(CO)c1ccn(C)n1]

Cpd 075

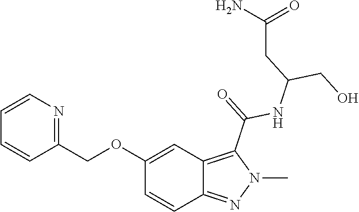 [SMILES: Cn1nc2ccc(OCc3ccccn3)cc2c1C(=O)NC(CO)CC(N)=O]

Cpd 076

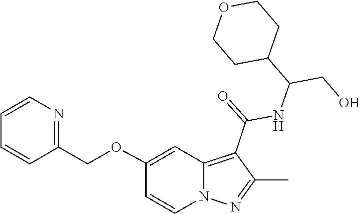 [SMILES: Cc1nn2ccc(OCc3ccccn3)cc2c1C(=O)NC(CO)C1CCOCC1]

Cpd 077

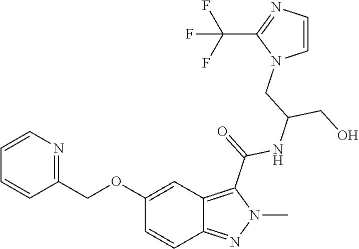 [SMILES: Cn1nc2ccc(OCc3ccccn3)cc2c1C(=O)NC(CO)Cn1ccnc1C(F)(F)F]

Cpd 078

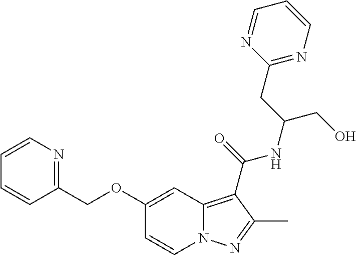 [SMILES: Cc1nn2ccc(OCc3ccccn3)cc2c1C(=O)NC(CO)Cc1ncccn1]

Cpd 079

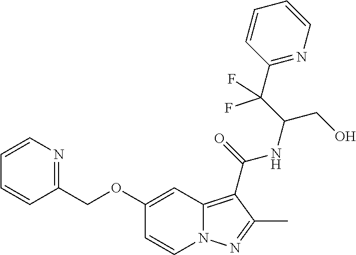 [SMILES: Cc1nn2ccc(OCc3ccccn3)cc2c1C(=O)NC(CO)C(F)(F)c1ccccn1]

Cpd 080

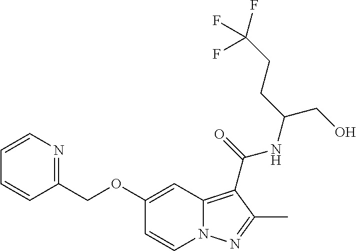 [SMILES: Cc1nn2ccc(OCc3ccccn3)cc2c1C(=O)NC(CO)CCC(F)(F)F]

Cpd 081

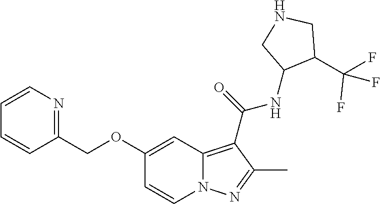 [SMILES: Cc1nn2ccc(OCc3ccccn3)cc2c1C(=O)NC1CNCC1C(F)(F)F]

Cpd 082

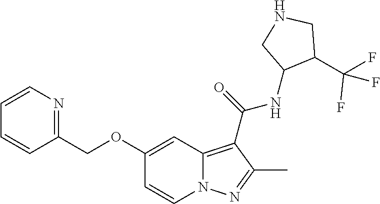 [SMILES: Cc1nn2ccc(OCc3ccccn3)cc2c1C(=O)NC1CNCC1C(F)(F)F]

Cpd 083

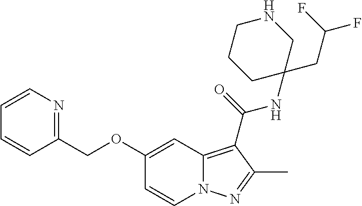 [SMILES: Cc1nn2ccc(OCc3ccccn3)cc2c1C(=O)NC1(CC(F)F)CCCNC1]

Cpd 084

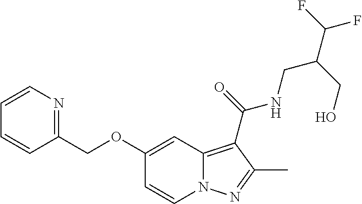 [SMILES: Cc1nn2ccc(OCc3ccccn3)cc2c1C(=O)NCC(CO)C(F)F]

Cpd 085

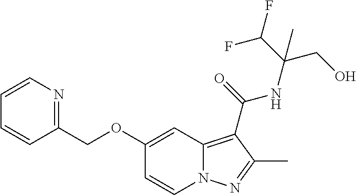 [SMILES: Cc1nn2ccc(OCc3ccccn3)cc2c1C(=O)NC(C)(CO)C(F)F]

Cpd 086

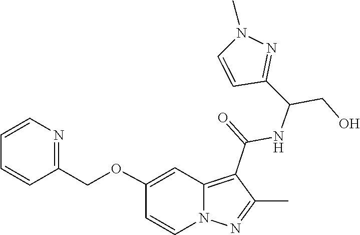 [SMILES: Cc1nn2ccc(OCc3ccccn3)cc2c1C(=O)NC(CO)c1ccn(C)n1]

Cpd 087

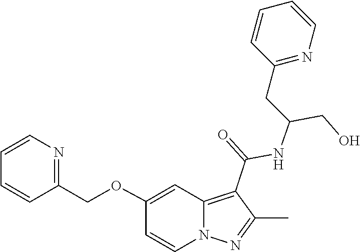 [SMILES: Cc1nn2ccc(OCc3ccccn3)cc2c1C(=O)NC(CO)Cc1ccccn1]

Cpd 088

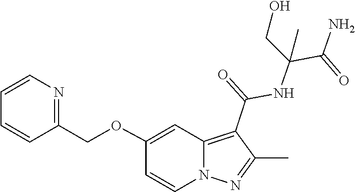 [SMILES: Cc1nn2ccc(OCc3ccccn3)cc2c1C(=O)NC(C)(CO)C(N)=O]

Cpd 089

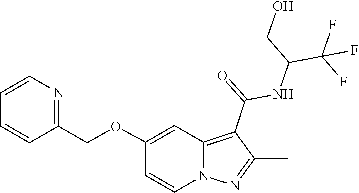 [SMILES: Cc1nn2ccc(OCc3ccccn3)cc2c1C(=O)NC(CO)C(F)(F)F]

Cpd 090

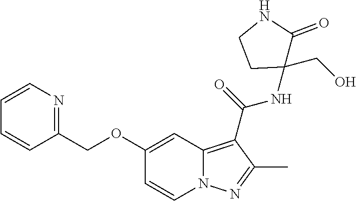 [SMILES: Cc1nn2ccc(OCc3ccccn3)cc2c1C(=O)NC1(CO)CCNC1=O]

Cpd 091

64. A pharmaceutical composition comprising a compound according to any one of the preceding items.
65. The compound according to any one of items 1 to 63 or the pharmaceutical composition according to item 64, for use in the treatment of pain.
66. The compound or the pharmaceutical composition for use in the treatment of pain according to item 65, wherein the pain is selected from nociceptive pain, inflammatory pain, and neuropathic pain; preferably post-operative pain.
67. A method of treating of pain comprising administering a compound according to any one of items 1 to 63, or a pharmaceutical composition according to any one of items 64 to 66, to a subject in need thereof.
68. The method of item 67 wherein the pain is selected from nociceptive pain, inflammatory pain, and neuropathic pain; preferably post-operative pain.
69. The compound according to any one of items 1 to 63 or the pharmaceutical composition according to claim 64, for use in the treatment of epilepsy.
70. A method of treating of epilepsy comprising administering a compound according to any one of items 1-63, or a pharmaceutical composition according to any of items 64 or 69, to a subject in need thereof.

### Examples

The following examples are provided for the purpose of illustrating the invention and by no means should be interpreted to limit the scope of the invention.

Representative compounds of the present invention can be synthesized in accordance with the general synthetic methods described below and illustrated in the schemes that follow. Since the schemes are an illustration, the invention should not be construed as being limited by the specific chemical reaction and specific conditions described in the schemes and examples. The various starting material used in the schemes are commercially available or may be prepared by methods well within the skill of persons versed in the art. The variables are as defined herein an within the skill of persons versed in the art.

Abbreviations used in the instant specification, particularly in the schemes and examples, are as follows: ABC—Aqueous solution of Ammonium Bicarbonate, ACN—acetonitrile, AcOH—Acetic acid, ADDP—1,1′-(Azodicarbonyl)dipiperidide, aq.—Aqueous, AlBN—Azobisisobutyronitrile, CAN—Ceric ammonium nitrate, COMU—(1-Cyano-2-ethoxy-2-oxoethylidenaminooxy)dimethylamino-morpholino-carbenium, hexafluorophosphate, DABCO—1,4-diazabicyclo[2.2.2]octane, DAST—Diethylaminosulfur trifluoride, DBU—1,8-Diazabicyclo[5.4.0]undec-7-ene, DCC—N,N′-dicyclohexylcarbodiimide, DCM -Dichloromethane, DEAD—Diethyl azodicarboxylate, DIA—Diastereomer, DIAD—Diisopropyl azodicarboxylate, DEA—Diethylamine, DIPEA—Diisopropyl-ethyl amine, DME—1,2-Dimethoxyethane, DMF—N,N-Dimethylformamide, DMSO—Dimethylsulfoxide, DPPA—Diphenylphosphoryl azide, 2,4-DNPH—2,4-Dinitrophenylhydrazine, DTBAD—tert-Butylazodicarboxylate, EDCl or EDC -1-Ethyl-3-(3-dimethylaminopropyl)-carbodiimide, En—Enantiomer, Et2O—Diethyl ether, EtOH—Ethanol, EtOAc—Ethyl acetate, Eq.—Equivalent, FA—Formic acid, FCC—Flash column chromatography, h—Hour, HATU—O-(7-Azabenzotriazol-1-yl)-N,N,N′,N′-tetramethyluronium hexafluorophosphate, HPLC—High performance liquid chromatography, IPA—isopropyl alcohol, LAH—Lithiumaluminiumhydrid, LG—Leaving group, MeOH—methanol, MgSO4—Magnesium sulfate, min.—Minute, Ms—Methanesulfonyl, Na₂SO₄—Sodium sulfate, NBS—N-Bromosuccinimide, nBuLi—n-Butyl lithium, NMP—1-Methyl-2-pyrrolidinone, Pd(PPh3)4—Tetrakis-(triphenylphosphine)-palladium(0), Pd2(dba)3—Tris(dibenzylideneacetone)dipalladium, Pet ether—Petroleum ether, PPh3—Triphenylphospine, PS-DIEA—Diisoprpropyl-ethyl amine supported on PolyStyrene, PS—PPh3—Triphenylphospine supported on PolyStyrene, PyBop—Benzotriazol-1-yl-oxytripyrrolidinophosphonium hexafluorophosphate, PTSA—p-Toluenesulfonic acid, RF: ratio of frontiers, RM—Reaction mixture, RP—Reverse phase, RT—Room temperature, sat.—Saturated, SEM—[2-(Trimethylsilyl)ethoxy]methyl acetal, SFC—Supercritical fluid chromatography, SPE—Solid Phase Extraction, TBDMS—Tert-Butyldimethylsilyl ether, TBAF—Tetrabutylammonium fluoride hydrate, TBAI—Tetrabutylammonium iodide, TEA—Triethylamine, THE—Tetrahydrofurane, TFA—Trifluoroacetic acid, TLC—thin layer chromatography, TPP—triphenyl phosphine, IPA—isopropyl alcohol, TMS—trimethyl silyl, T3P—Propylphosphonic anhydride.

The compounds of interest having a structure according to the general formula (A) and all other formulas described herein and embodiments thereof can be prepared as outlined in the general chemical scheme 1.

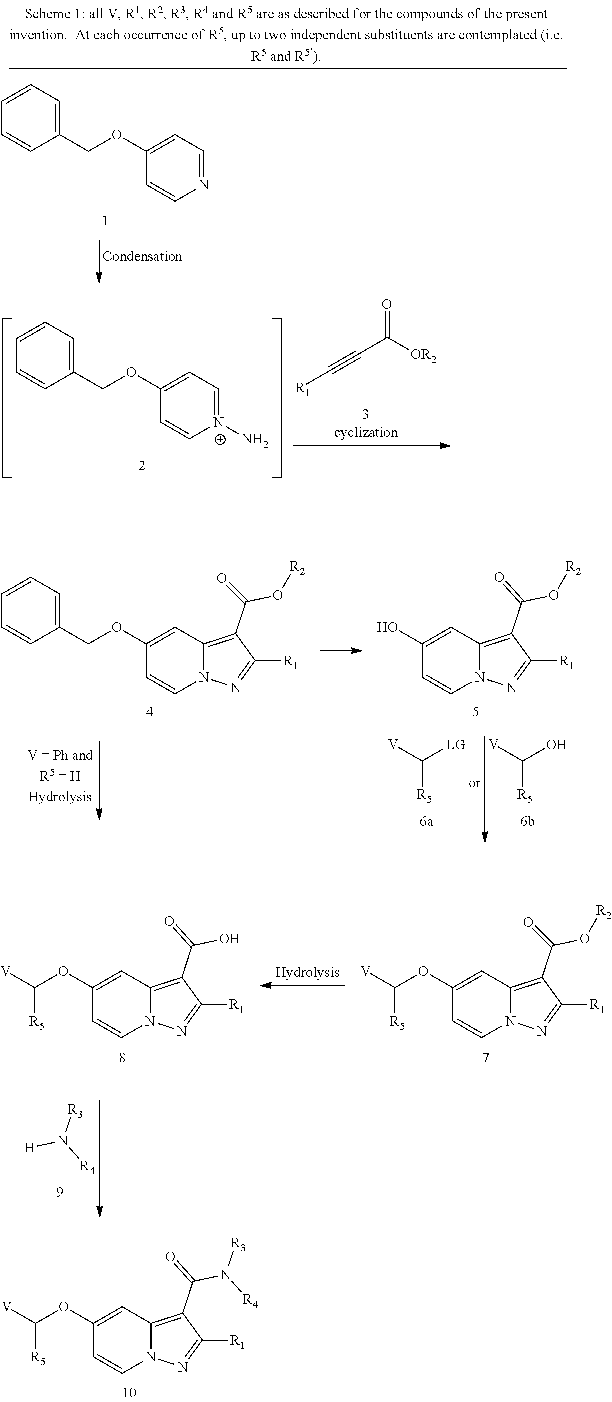

4-(Benzyloxy)pyridine of formula 1, may be reacted with a reagent bearing an electrophilic amine (e.g. 2,4-DNPH, 0-(mesitylsulfonyl)hydroxylamine, and the like) in a suitable solvent (e.g., MeCN, DME, NMP, and the like) to provide N-aminopyridinium salts of formula 2. Intermediate of formula 2 may be condensed with an alkyne ester derivatives 3 (commercially available or synthesized by procedures known to those skilled in the art), wherein R2 is an ester protecting group (e.g. methyl, ethyl, t-Bu and the like), in the presence of a base (e.g., potassium carbonate, DBU and the like) in a polar solvent (e.g., DMF, DCM, MeCN, and the like) at a temperature ranging from 0 to 100° C. to provide pyrazolo[1,5-a]pyridines of formula 4. Ester derivatives 4 may then be converted into the intermediate compounds of formula 5 via a hydrogenation reactions with a reducing agent (e.g., hydrogen gas, ammonium formate, cyclohexadiene and the like) using a catalyst (more preferably Pd or Pt) in a solvent (e.g., THF, EtOH, and the like). Intermediates of formula 5 may then be converted into the desired compounds of formula 7 via nucleophilic substitution using intermediates of formula 6a (commercially available or synthesized), wherein LG is a leaving group, in the presence of a base (e.g., DIPEA, DBU, triethylamine, Cs₂CO₃, and the like) in a polar solvent (e.g., MeCN, DMF, NMP, and the like), with or without a chelating agent (e.g., 18-crown-6, cis-anti-cis-dicyclohexano-18-crown-6, and the like) at a temperature ranging from 0 to 100° C. Alternatively, intermediates of formula 5 may instead be reacted with intermediates of formula 6b (commercially available or synthesized) in the presence of an azodicarboxylate reagent (e.g., DEAD, DIAD, ADDP, and the like) and a phosphine (e.g., tributylphosphine, triphenylphosphine and the like) in a solvent (e.g., THF, toluene, and the like) at a temperature ranging from 0 to 100° C., to provide the desired compounds of formula 7. Ester derivatives 7 may then be converted into the desired compounds of formula 8 via standard saponification reactions. Alternatively, benzyl derivatives 4 may then be converted into the desired compounds of formula 8 via standard saponification reactions The desired compounds of formula 10 may be obtained from acid derivatives of formula 8 by reaction with amine derivatives of formula 9 (commercially available or synthesized by procedures known in the art or as set forth in the examples below) under standard peptide coupling conditions (e.g. DCC, EDCl, HATU, PyBop and the like) in a polar aprotic solvent (e.g. DCM, DMF and the like).

Alternatively, carboxylic acid derivatives of formula 8, may be converted into acid chloride derivatives by procedures known to those skilled in the art or as set forth in the examples below, and then reacted with amines of formula 9 to obtain the desired pyrazolo[1,5-a]pyridines of formula 10 by procedures known to those skilled in the art or as set forth in the examples below

The compounds of interest having a structure according to the general formula (A) and all other formulas described herein and embodiments thereof can be prepared as outlined in the general chemical scheme 2.

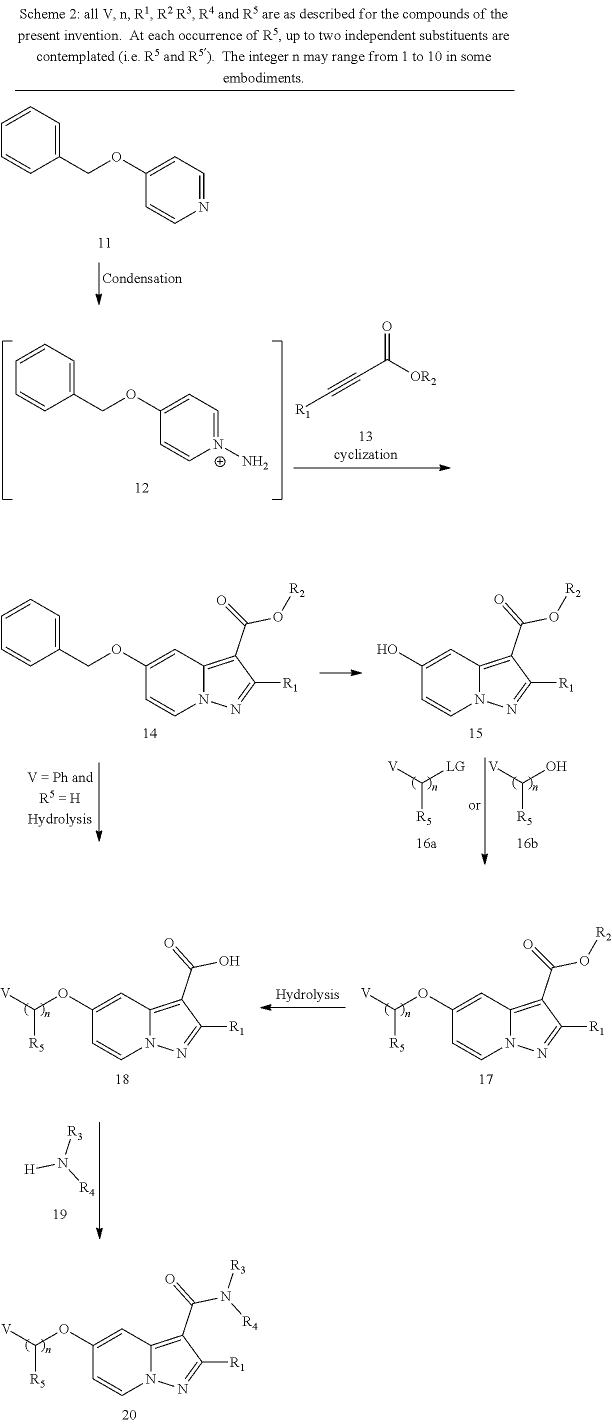

4-(Benzyloxy)pyridine of formula 11, may be reacted with a reagent bearing an electrophilic amine (e.g. 2,4-DNPH, 0-(mesitylsulfonyl)hydroxylamine, and the like) in a suitable solvent (e.g., MeCN, DMF, NMP, and the like) to provide N-aminopyridinium salts of formula 12. Intermediate of formula 12 may be condensed with an alkyne ester derivatives 13 (commercially available or synthesized by procedures known to those skilled in the art), wherein R2 is an ester protecting group (e.g. methyl, ethyl, t-Bu and the like), in the presence of a base (e.g., potassium carbonate, DBU and the like) in a polar solvent (e.g., DMF, DCM, MeCN, and the like) at a temperature ranging from 0 to 100° C. to provide pyrazolo[1,5-a]pyridines of formula 14. Ester derivatives 14 may then be converted into the intermediate compounds of formula 15 via a hydrogenation reactions with a reducing agent (e.g., hydrogen gas, ammonium formate, cyclohexadiene and the like) using a catalyst (more preferably Pd or Pt) in a solvent (e.g., THF, EtOH, and the like). Intermediates of formula 15 may then be converted into the desired compounds of formula 17 via nucleophilic substitution using intermediates of formula 16a (commercially available or synthesized), wherein LG is a leaving group, in the presence of a base (e.g., DIPEA, DBU, triethylamine, Cs₂CO₃, and the like) in a polar solvent (e.g., MeCN, DMF, NMP, and the like), with or without a chelating agent (e.g., 18-crown-6, cis-anti-cis-dicyclohexano-18-crown-6, and the like) at a temperature ranging from 0 to 100° C. Alternatively, intermediates of formula 15 may instead be reacted with intermediates of formula 16b (commercially available or synthesized) in the presence of an azodicarboxylate reagent (e.g., DEAD, DIAD, ADDP, and the like) and a phosphine (e.g., tributylphosphine, triphenylphosphine and the like) in a solvent (e.g., THF, toluene, and the like) at a temperature ranging from 0 to 10000, to provide the desired compounds of formula 17. Ester derivatives 17 may then be converted into the desired compounds of formula 18 via standard saponification reactions. Alternatively, benzyl derivatives 14 may then be converted into the desired compounds of formula 18 via standard saponification reactions The desired compounds of formula 20 may be obtained from acid derivatives of formula 18 by reaction with amine derivatives of formula 19 (commercially available or synthesized by procedures known in the art or as set forth in the examples below) under standard peptide coupling conditions (e.g. DCC, EDCl, HATU, PyBop and the like) in a polar aprotic solvent (e.g. DCM, DMF and the like). Alternatively, carboxylic acid derivatives of formula 18, may be converted into acid chloride derivatives by procedures known to those skilled in the art or as set forth in the examples below, and then reacted with amines of formula 9 to obtain the desired pyrazolo[1,5-a]pyridines of formula 20 by procedures known to those skilled in the art or as set forth in the examples below

Table 3
Exemplary Compounds
Structure and Compound CODE

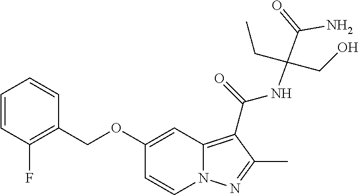 [SMILES: CCC(CO)(NC(=O)c1c(C)nn2ccc(OCc3ccccc3F)cc12)C(N)=O]

Cpd 001

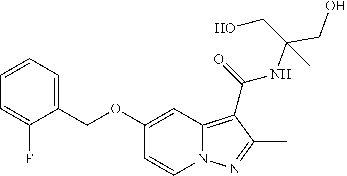 [SMILES: Cc1nn2ccc(OCc3ccccc3F)cc2c1C(=O)NC(C)(CO)CO]

Cpd 002

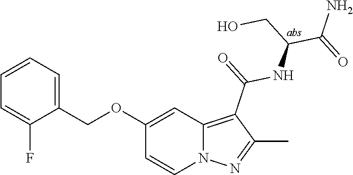 [SMILES: Cc1nn2ccc(OCc3ccccc3F)cc2c1C(=O)N[C@@H](CO)C(N)=O]

Cpd 003

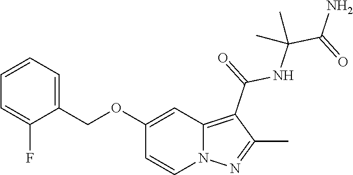 [SMILES: Cc1nn2ccc(OCc3ccccc3F)cc2c1C(=O)NC(C)(C)C(N)=O]

Cpd 004

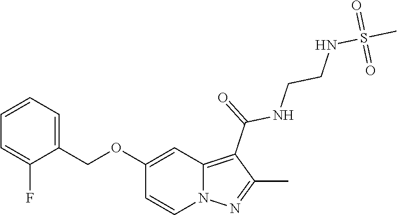 [SMILES: Cc1nn2ccc(OCc3ccccc3F)cc2c1C(=O)NCCNS(C)(=O)=O]

Cpd 005

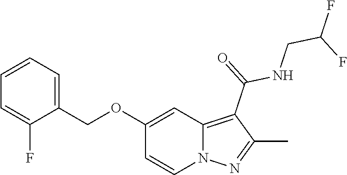 [SMILES: Cc1nn2ccc(OCc3ccccc3F)cc2c1C(=O)NCC(F)F]

Cpd 006

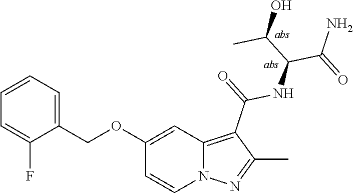 [SMILES: Cc1nn2ccc(OCc3ccccc3F)cc2c1C(=O)N[C@H](C(N)=O)[C@@H](C)O]

Cpd 007

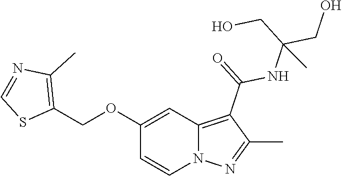 [SMILES: Cc1ncsc1COc1ccn2nc(C)c(C(=O)NC(C)(CO)CO)c2c1]

Cpd 008

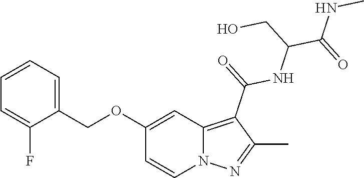 [SMILES: CNC(=O)C(CO)NC(=O)c1c(C)nn2ccc(OCc3ccccc3F)cc12]

Cpd 009

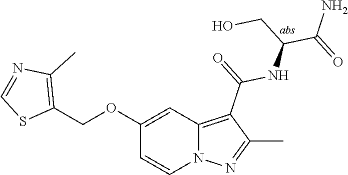 [SMILES: Cc1ncsc1COc1ccn2nc(C)c(C(=O)N[C@@H](CO)C(N)=O)c2c1]

Cpd 010

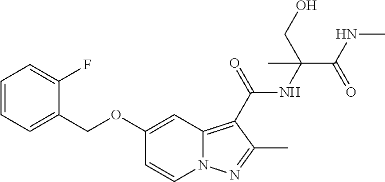 [SMILES: CNC(=O)C(C)(CO)NC(=O)c1c(C)nn2ccc(OCc3ccccc3F)cc12]

Cpd 011

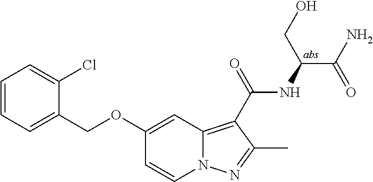 [SMILES: Cc1nn2ccc(OCc3ccccc3Cl)cc2c1C(=O)N[C@@H](CO)C(N)=O]

Cpd 012

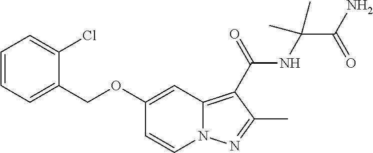 [SMILES: Cc1nn2ccc(OCc3ccccc3Cl)cc2c1C(=O)NC(C)(C)C(N)=O]

Cpd 013

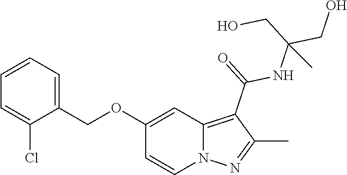 [SMILES: Cc1nn2ccc(OCc3ccccc3Cl)cc2c1C(=O)NC(C)(CO)CO]

Cpd 014

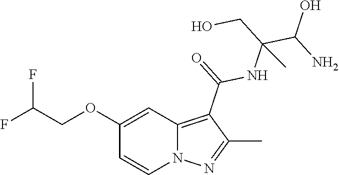 [SMILES: Cc1nn2ccc(OCC(F)F)cc2c1C(=O)NC(C)(CO)C(N)O]

Cpd 015

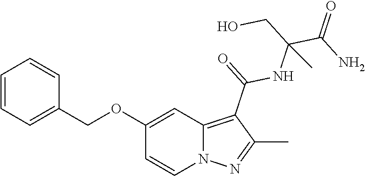 [SMILES: Cc1nn2ccc(OCc3ccccc3)cc2c1C(=O)NC(C)(CO)C(N)=O]

Cpd 016

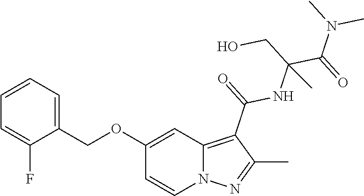 [SMILES: Cc1nn2ccc(OCc3ccccc3F)cc2c1C(=O)NC(C)(CO)C(=O)N(C)C]

Cpd 017

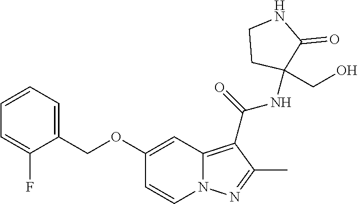 [SMILES: Cc1nn2ccc(OCc3ccccc3F)cc2c1C(=O)NC1(CO)CCNC1=O]

Cpd 018

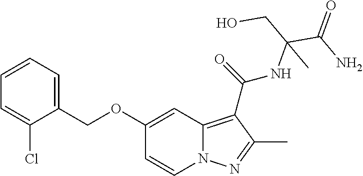 [SMILES: Cc1nn2ccc(OCc3ccccc3Cl)cc2c1C(=O)NC(C)(CO)C(N)=O]

Cpd 019

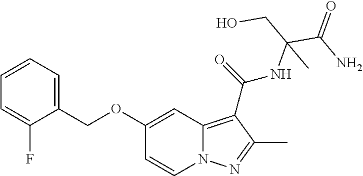 [SMILES: Cc1nn2ccc(OCc3ccccc3F)cc2c1C(=O)NC(C)(CO)C(N)=O]

Cpd 020

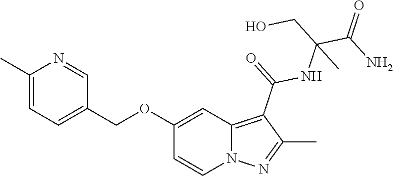 [SMILES: Cc1ccc(COc2ccn3nc(C)c(C(=O)NC(C)(CO)C(N)=O)c3c2)cn1]

Cpd 021

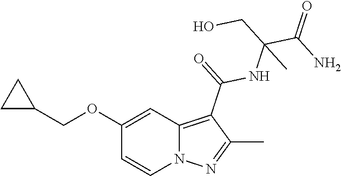 [SMILES: Cc1nn2ccc(OCC3CC3)cc2c1C(=O)NC(C)(CO)C(N)=O]

Cpd 022

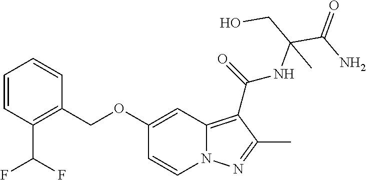 [SMILES: Cc1nn2ccc(OCc3ccccc3C(F)F)cc2c1C(=O)NC(C)(CO)C(N)=O]

Cpd 023

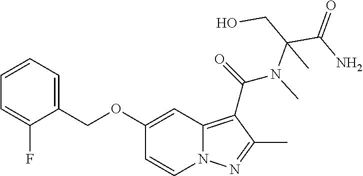 [SMILES: Cc1nn2ccc(OCc3ccccc3F)cc2c1C(=O)N(C)C(C)(CO)C(N)=O]

Cpd 024

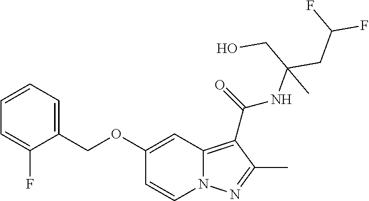 [SMILES: Cc1nn2ccc(OCc3ccccc3F)cc2c1C(=O)NC(C)(CO)CC(F)F]

Cpd 025

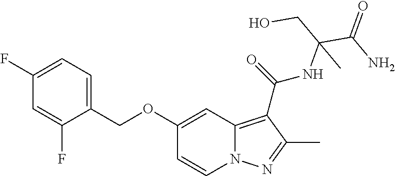 [SMILES: Cc1nn2ccc(OCc3ccc(F)cc3F)cc2c1C(=O)NC(C)(CO)C(N)=O]

Cpd 026

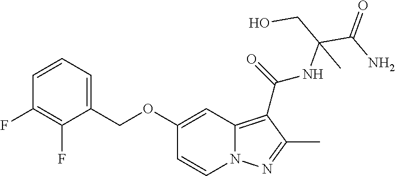 [SMILES: Cc1nn2ccc(OCc3cccc(F)c3F)cc2c1C(=O)NC(C)(CO)C(N)=O]

Cpd 027

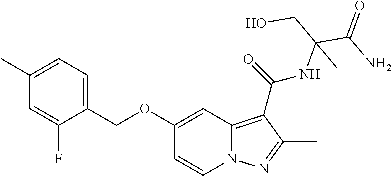 [SMILES: Cc1ccc(COc2ccn3nc(C)c(C(=O)NC(C)(CO)C(N)=O)c3c2)c(F)c1]

Cpd 028

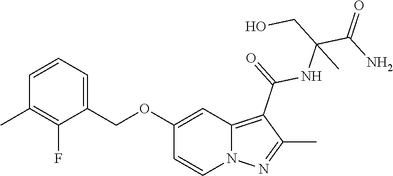 [SMILES: Cc1cccc(COc2ccn3nc(C)c(C(=O)NC(C)(CO)C(N)=O)c3c2)c1F]

Cpd 029

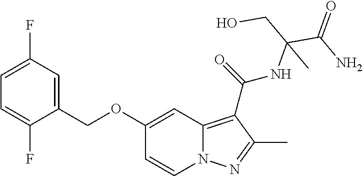 [SMILES: Cc1nn2ccc(OCc3cc(F)ccc3F)cc2c1C(=O)NC(C)(CO)C(N)=O]

Cpd 030

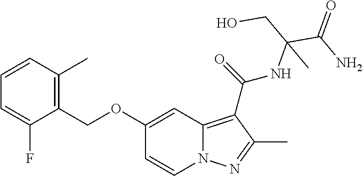 [SMILES: Cc1cccc(F)c1COc1ccn2nc(C)c(C(=O)NC(C)(CO)C(N)=O)c2c1]

Cpd 031

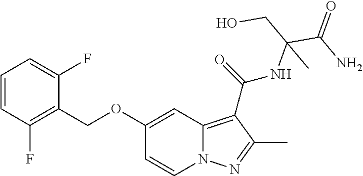 [SMILES: Cc1nn2ccc(OCc3c(F)cccc3F)cc2c1C(=O)NC(C)(CO)C(N)=O]

Cpd 032

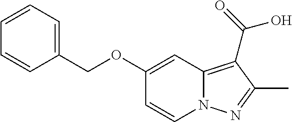 [SMILES: Cc1nn2ccc(OCc3ccccc3)cc2c1C(=O)O]

Cpd 033

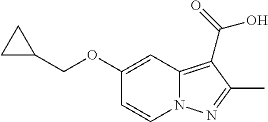 [SMILES: Cc1nn2ccc(OCC3CC3)cc2c1C(=O)O]

Cpd 034

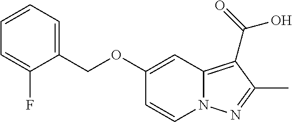 [SMILES: Cc1nn2ccc(OCc3ccccc3F)cc2c1C(=O)O]

Cpd 035

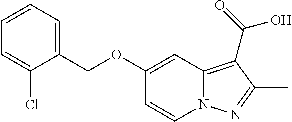 [SMILES: Cc1nn2ccc(OCc3ccccc3Cl)cc2c1C(=O)O]

Cpd 036

TABLE 4
Exemplary Compounds
Structure and Compound CODE

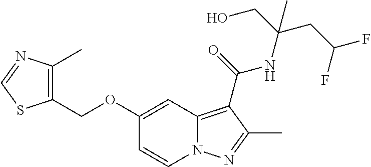 [SMILES: Cc1ncsc1COc1ccn2nc(C)c(C(=O)NC(C)(CO)CC(F)F)c2c1]

Cpd 037

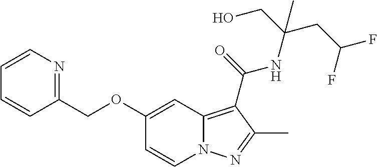 [SMILES: Cc1nn2ccc(OCc3ccccn3)cc2c1C(=O)NC(C)(CO)CC(F)F]

Cpd 038

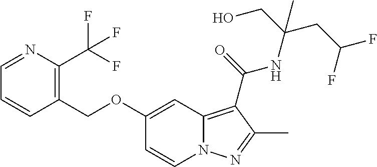 [SMILES: Cc1nn2ccc(OCc3cccnc3C(F)(F)F)cc2c1C(=O)NC(C)(CO)CC(F)F]

Cpd 039

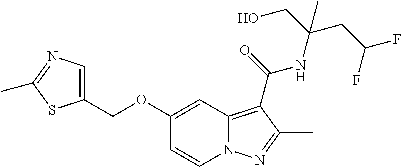 [SMILES: Cc1ncc(COc2ccn3nc(C)c(C(=O)NC(C)(CO)CC(F)F)c3c2)s1]

Cpd 040

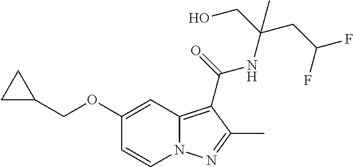 [SMILES: Cc1nn2ccc(OCC3CC3)cc2c1C(=O)NC(C)(CO)CC(F)F]

Cpd 041

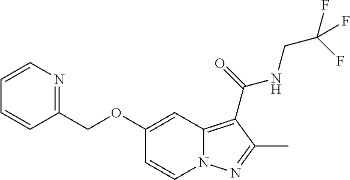 [SMILES: Cc1nn2ccc(OCc3ccccn3)cc2c1C(=O)NCC(F)(F)F]

Cpd 042

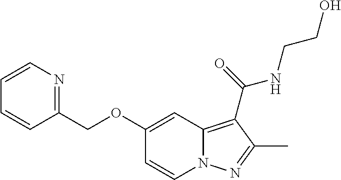 [SMILES: Cc1nn2ccc(OCc3ccccn3)cc2c1C(=O)NCCO]

Cpd 043

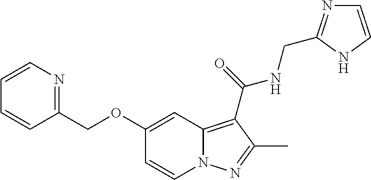 [SMILES: Cc1nn2ccc(OCc3ccccn3)cc2c1C(=O)NCc1ncc[nH]1]

Cpd 044

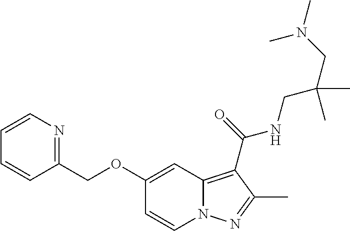 [SMILES: Cc1nn2ccc(OCc3ccccn3)cc2c1C(=O)NCC(C)(C)CN(C)C]

Cpd 045

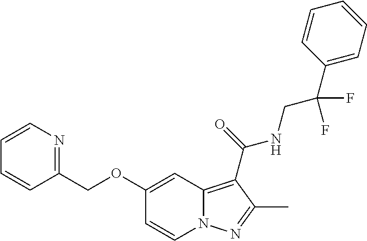 [SMILES: Cc1nn2ccc(OCc3ccccn3)cc2c1C(=O)NCC(F)(F)c1ccccc1]

Cpd 046

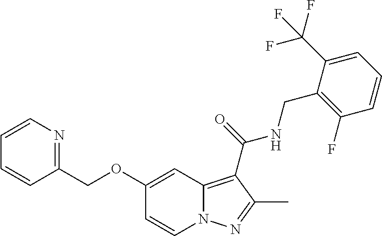 [SMILES: Cc1nn2ccc(OCc3ccccn3)cc2c1C(=O)NCc1c(F)cccc1C(F)(F)F]

Cpd 047

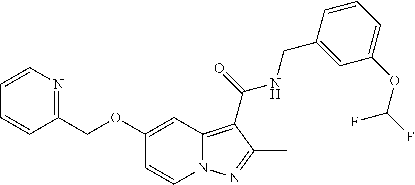 [SMILES: Cc1nn2ccc(OCc3ccccn3)cc2c1C(=O)NCc1cccc(OC(F)F)c1]

Cpd 048

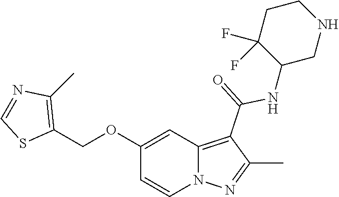 [SMILES: Cc1ncsc1COc1ccn2nc(C)c(C(=O)NC3CNCCC3(F)F)c2c1]

Cpd 049

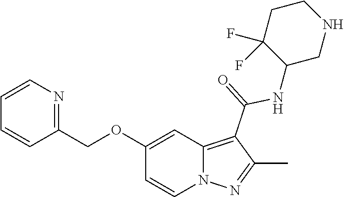 [SMILES: Cc1nn2ccc(OCc3ccccn3)cc2c1C(=O)NC1CNCCC1(F)F]

Cpd 050

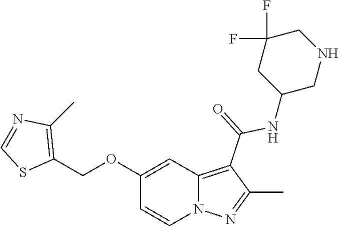 [SMILES: Cc1ncsc1COc1ccn2nc(C)c(C(=O)NC3CNCC(F)(F)C3)c2c1]

Cpd 051

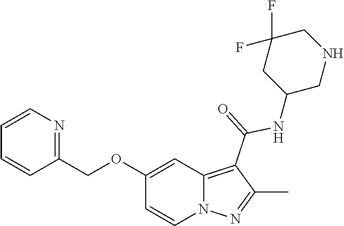 [SMILES: Cc1nn2ccc(OCc3ccccn3)cc2c1C(=O)NC1CNCC(F)(F)C1]

Cpd 052

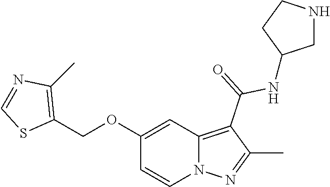 [SMILES: Cc1ncsc1COc1ccn2nc(C)c(C(=O)NC3CCNC3)c2c1]

Cpd 053

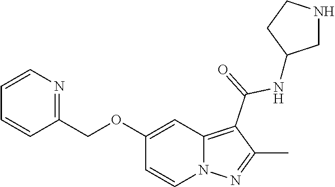 [SMILES: Cc1nn2ccc(OCc3ccccn3)cc2c1C(=O)NC1CCNC1]

Cpd 054

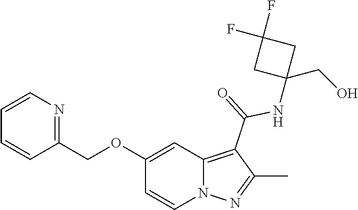 [SMILES: Cc1nn2ccc(OCc3ccccn3)cc2c1C(=O)NC1(CO)CC(F)(F)C1]

Cpd 055

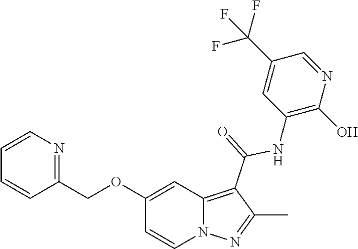 [SMILES: Cc1nn2ccc(OCc3ccccn3)cc2c1C(=O)Nc1cc(C(F)(F)F)cnc1O]

Cpd 056

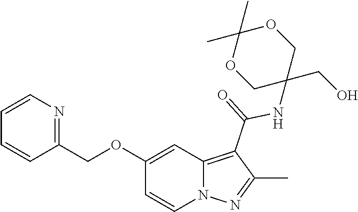 [SMILES: Cc1nn2ccc(OCc3ccccn3)cc2c1C(=O)NC1(CO)COC(C)(C)OC1]

Cpd 057

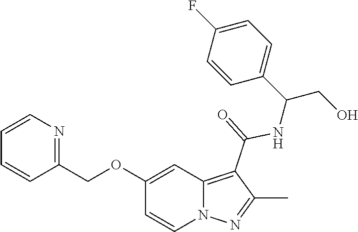 [SMILES: Cc1nn2ccc(OCc3ccccn3)cc2c1C(=O)NC(CO)c1ccc(F)cc1]

Cpd 058

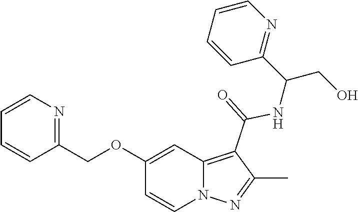 [SMILES: Cc1nn2ccc(OCc3ccccn3)cc2c1C(=O)NC(CO)c1ccccn1]

Cpd 059

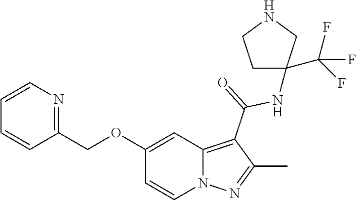 [SMILES: Cc1nn2ccc(OCc3ccccn3)cc2c1C(=O)NC1(C(F)(F)F)CCNC1]

Cpd 060

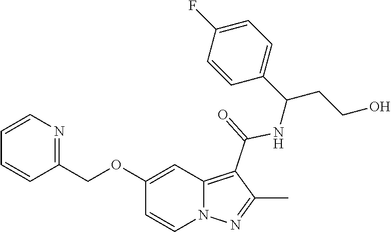 [SMILES: Cc1nn2ccc(OCc3ccccn3)cc2c1C(=O)NC(CCO)c1ccc(F)cc1]

Cpd 061

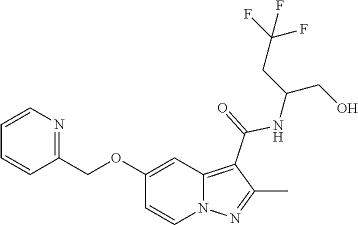 [SMILES: Cc1nn2ccc(OCc3ccccn3)cc2c1C(=O)NC(CO)CC(F)(F)F]

Cpd 062

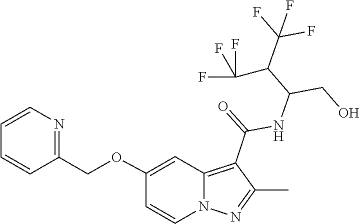 [SMILES: Cc1nn2ccc(OCc3ccccn3)cc2c1C(=O)NC(CO)C(C(F)(F)F)C(F)(F)F]

Cpd 063

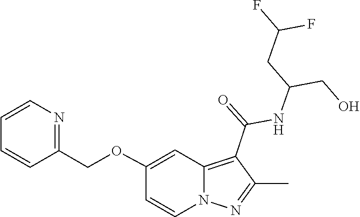 [SMILES: Cc1nn2ccc(OCc3ccccn3)cc2c1C(=O)NC(CO)CC(F)F]

Cpd 064

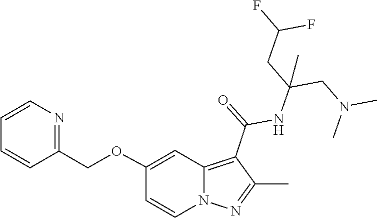 [SMILES: Cc1nn2ccc(OCc3ccccn3)cc2c1C(=O)NC(C)(CC(F)F)CN(C)C]

Cpd 065

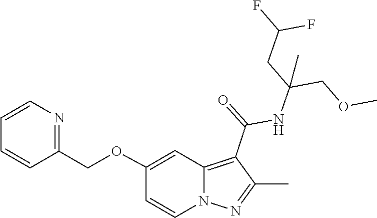 [SMILES: COCC(C)(CC(F)F)NC(=O)c1c(C)nn2ccc(OCc3ccccn3)cc12]

Cpd 066

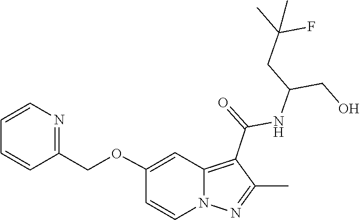 [SMILES: Cc1nn2ccc(OCc3ccccn3)cc2c1C(=O)NC(CO)CC(C)(C)F]

Cpd 067

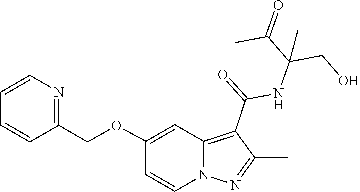 [SMILES: CC(=O)C(C)(CO)NC(=O)c1c(C)nn2ccc(OCc3ccccn3)cc12]

Cpd 068

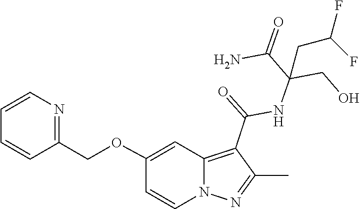 [SMILES: Cc1nn2ccc(OCc3ccccn3)cc2c1C(=O)NC(CO)(CC(F)F)C(N)=O]

Cpd 069

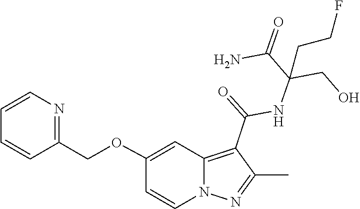 [SMILES: Cc1nn2ccc(OCc3ccccn3)cc2c1C(=O)NC(CO)(CCF)C(N)=O]

Cpd 070

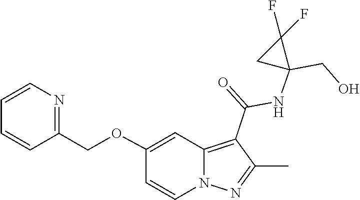 [SMILES: Cc1nn2ccc(OCc3ccccn3)cc2c1C(=O)NC1(CO)CC1(F)F]

Cpd 071

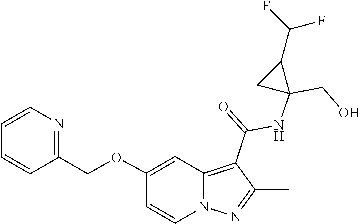 [SMILES: Cc1nn2ccc(OCc3ccccn3)cc2c1C(=O)NC1(CO)CC1C(F)F]

Cpd 072

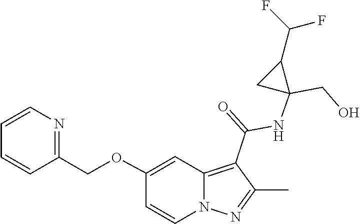 [SMILES: Cc1nn2ccc(OCc3ccccn3)cc2c1C(=O)NC1(CO)CC1C(F)F]

Cpd 073

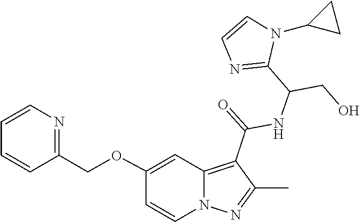 [SMILES: Cc1nn2ccc(OCc3ccccn3)cc2c1C(=O)NC(CO)c1nccn1C1CC1]

Cpd 074

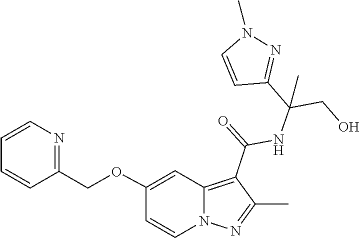 [SMILES: Cc1nn2ccc(OCc3ccccn3)cc2c1C(=O)NC(C)(CO)c1ccn(C)n1]

Cpd 075

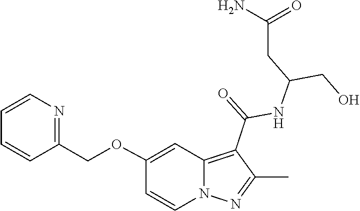 [SMILES: Cc1nn2ccc(OCc3ccccn3)cc2c1C(=O)NC(CO)CC(N)=O]

Cpd 076

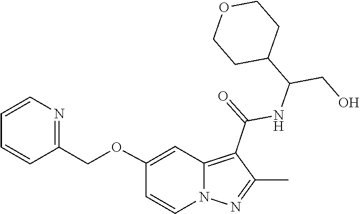 [SMILES: Cc1nn2ccc(OCc3ccccn3)cc2c1C(=O)NC(CO)C1CCOCC1]

Cpd 077

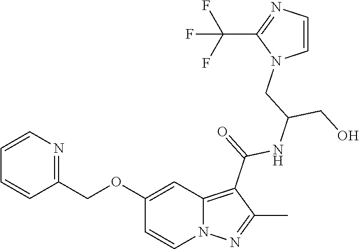 [SMILES: Cc1nn2ccc(OCc3ccccn3)cc2c1C(=O)NC(CO)Cn1ccnc1C(F)(F)F]

Cpd 078

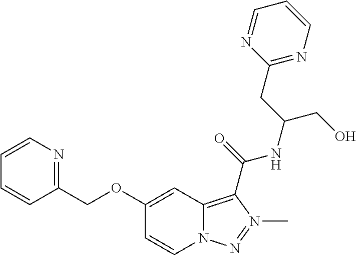

Cpd 079

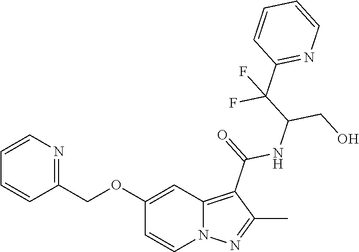 [SMILES: Cc1nn2ccc(OCc3ccccn3)cc2c1C(=O)NC(CO)C(F)(F)c1ccccn1]

Cpd 080

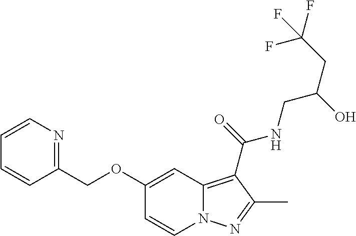 [SMILES: Cc1nn2ccc(OCc3ccccn3)cc2c1C(=O)NCC(O)CC(F)(F)F]

Cpd 081

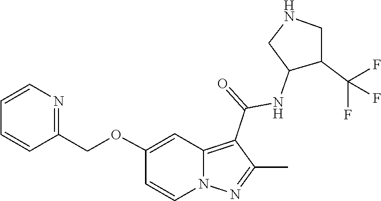 [SMILES: Cc1nn2ccc(OCc3ccccn3)cc2c1C(=O)NC1CNCC1C(F)(F)F]

Cpd 082

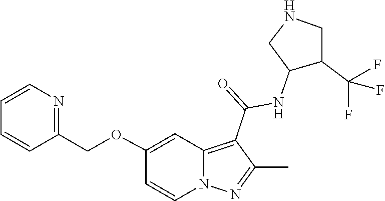 [SMILES: Cc1nn2ccc(OCc3ccccn3)cc2c1C(=O)NC1CNCC1C(F)(F)F]

Cpd 083

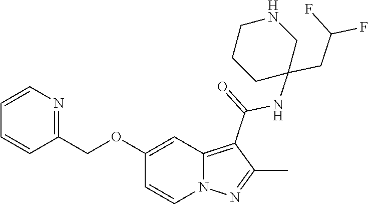 [SMILES: Cc1nn2ccc(OCc3ccccn3)cc2c1C(=O)NC1(CC(F)F)CCCNC1]

Cpd 084

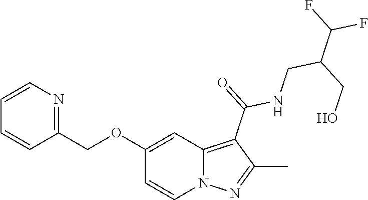 [SMILES: Cc1nn2ccc(OCc3ccccn3)cc2c1C(=O)NCC(CO)C(F)F]

Cpd 085

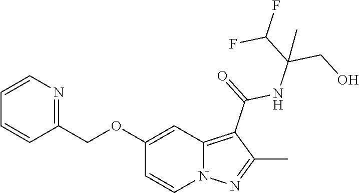 [SMILES: Cc1nn2ccc(OCc3ccccn3)cc2c1C(=O)NC(C)(CO)C(F)F]

Cpd 086

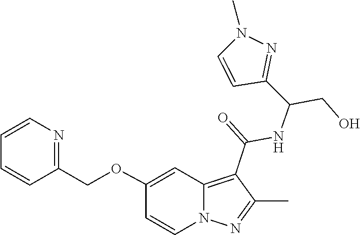 [SMILES: Cc1nn2ccc(OCc3ccccn3)cc2c1C(=O)NC(CO)c1ccn(C)n1]

Cpd 087

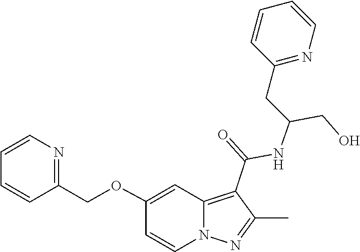 [SMILES: Cc1nn2ccc(OCc3ccccn3)cc2c1C(=O)NC(CO)Cc1ccccn1]

Cpd 088

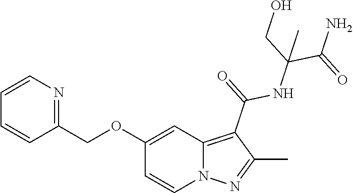 [SMILES: Cc1nn2ccc(OCc3ccccn3)cc2c1C(=O)NC(C)(CO)C(N)=O]

Cpd 089

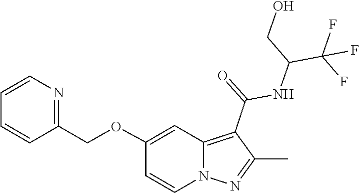 [SMILES: Cc1nn2ccc(OCc3ccccn3)cc2c1C(=O)NC(CO)C(F)(F)F]

Cpd 090

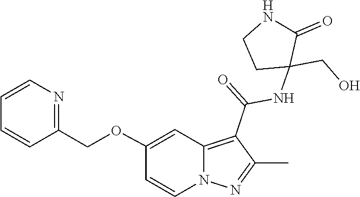 [SMILES: Cc1nn2ccc(OCc3ccccn3)cc2c1C(=O)NC1(CO)CCNC1=O]

Cpd 091

The following examples are provided for the purpose of illustrating the present invention and by no means should be interpreted to limit the scope of the present invention.

Part A represent the preparation of the compounds whereas Part B represents the pharmacological examples.

### Part A

All starting materials which are not explicitly described were either commercially available (the details of suppliers such as for example ABCR, Apollo Scientific Combi-Blocks, Enamine, FluoroChem, MatrixScientific, Maybridge, Merck, TCI etc. can be found in the SciFinder® Database for example) or the synthesis thereof has already been described precisely in the specialist literature (experimental guidelines can be found in the Reaxys® Database or the SciFinder® Database respectively, for example) or can be prepared using the conventional methods known to the person skilled in the art.

The reactions were, if necessary, carried out under an inert atmosphere (mostly argon and N₂). The number of equivalents of reagents and the amounts of solvents employed as well as the reaction temperatures and times can vary slightly between different reactions carried out by analogous methods. The work-up and purification methods were adapted according to the characteristic properties of each compound and can vary slightly for analogous methods.

The yields of the compounds prepared are not optimized.

The indication “equivalents” (“eq.” or “eq” or “equiv.”) means molar equivalents, “RT” or “rt” means room temperature T (23±7° C.), “M” are indications of concentration in mol/1, “sol.” means solution, “conc.” means concentrated. The mixing ratios of solvents are usually stated in the volume/volume ratio.

Key analytical characterization was carried out by means of ¹H-NMR spectroscopy and/or mass spectrometry (MS, m/z for [M+H]⁺ and/or [M−H]⁻) for all the exemplary compounds and selected intermediate products. In certain cases, where e.g. regioisomers and/or diatereomers could be/were formed during the reaction, additional analytics, such as, e.g. ¹³C NMR and NOE (nuclear overhauser effect) NMR experiments were in some cases performed.

Analytical instruments employed were e.g. for NMR analysis a BRUKER 400 MHz or a BRUKER 500 MHz machine (Software Topspin), alternatively a BRUKER AVANCE 300 MHz and 400 Mhz was employed. For LC/MS analysis e.g. an Agilent 1290 infinity, Mass:6150 SQD(ESI/APCI) or an Agilent 1200 SERIES, Mass:6130 SQD(ESI/APCI) (Software Chemistation) was employed. Analytical HPLCs were measured e.g. on Waters (Software Empower), an Agilent-1200-ELSD (Software Chemistation) or an Agilent-1260 (Software OpenLAB). Analytical SFC were performed e.g. on a PIC solution (Software: SFC PICLAB ONLINE), a WATERS-X5 (Software MASSLYNX) or a WATERS-UPC2 (Empower).

Preparative HPLC were performed e.g. on a Waters 2998 (Software Empower) or a YMC (Software K-Prep). Preparative SFC were performed e.g. on a Waters,SFC—200 (Software Chromscope or Super chrome), a Waters,SFC-80 (Super chrome) or a PIC,PIC-175 (Software S10-100).

Structures of example compounds that contain stereocenters are drawn and named with absolute stereochemistry, if known. In case of unknown absolute stereochemistry the compounds can be either racemic, a mixture of diastereomers, a pure diastereomer of unknown stereochemistry, or a pure enantiomer of unknown stereochemistry. Dia 1 and Dia 2 means that diastereoisomers were separated but the stereochemistry is unknown. En 1 and En 2 means that both enantiomers were separated but the absolute configuration is unknown. No suffix given after the compound code means that a compound containing stereocenters was obtained as a racemic mixture or a mixture of diastereomers, respectively, unless the chemical name of the compound specifies the exact stereochemistry.

### Synthesis of ethyl 5-(benzyloxy)-2-methylpyrazolo[1,5-a]pyridine-3-carboxylate (Int-01)

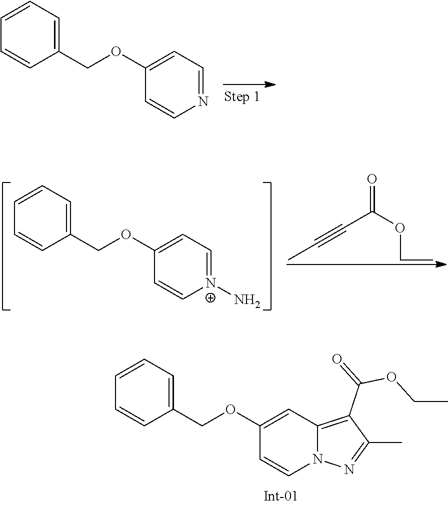

Step 1: To a stirred solution of 4-Benzyloxy-pyridine (60 g, 324.3.961 mmol) in MeCN (600 mL) was added 2,4-DNPH (64.5 g, 324.3 mmol) at RT. The reaction mixture was heated to 40° C. and stirred for 16 h at 40° C. The reaction mixture was concentrated under reduce pressure keeping water bath temperature below 40° C. to afford 1-Amino-4-benzyloxy-pyridinium. To a solution of 1-amino-4-benzyloxy-pyridinium in DMF (600 mL) was added potassium carbonate (447.5 g, 3243.2 mmol) and followed by but-2-ynoic acid methyl ester (56.6 mL, 486.4 mmol) at room temperature. The reaction mixture was stirred for 16 h at room temperature. The RM was quenched with ice cold water and extracted with EtOAc: MTBE (1:3, 3 L). Combined organic layers were washed with brine (900 mL×2), dried over anhydrous Na₂SO₄ and concentrated under reduced pressure. The crude was purified by column chromatography over silica gel (100-200 mesh) using 20 to 25% EtOAc in Pet ether as an eluent to afford ethyl 5-(benzyloxy)-2-methylpyrazolo[1,5-a]pyridine-3-carboxylate (Int-01) (20 g, 19.8% over two step) as an off-white solid. ¹H-NMR (400 MHz, CDCl3) d [ppm]: 8.21-8.23 (m, 1H), 7.26-7.47 (m, 7H), 6.60-6.62 (m, 1H), 5.15 (s, 2H), 4.36 (q, 3H), 2.62 (s, 3H), 1.40 (t, 3H).

### Synthesis of 2-amino-3-((tert-butyldimethylsilyl)oxy)-2-methylpropanamide (Int-02)

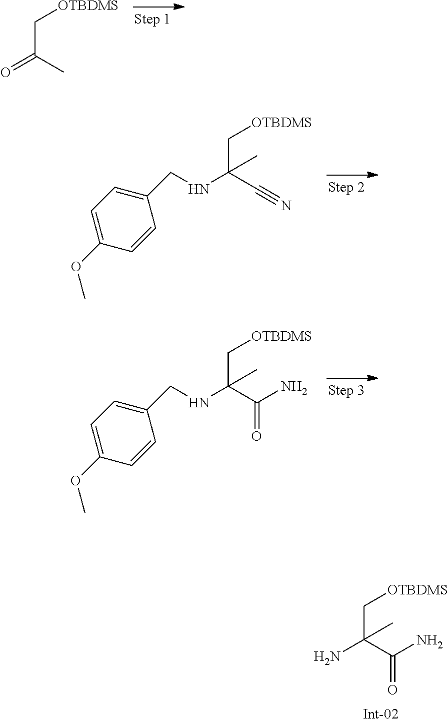 [SMILES: CC(=O)CO[Si](C)(C)C(C)(C)C ; COc1ccc(CNC(C)(C#N)CO[Si](C)(C)C(C)(C)C)cc1 ; COc1ccc(CNC(C)(CO[Si](C)(C)C(C)(C)C)C(N)=O)cc1 ; CC(N)(CO[Si](C)(C)C(C)(C)C)C(N)=O]

Step 1: To a stirred solution of 1-(tert-Butyl-dimethyl-silanyloxy)-propan-2-one (25 g, 132.7 mmol) in EtOH (250 ml) was added 4-methoxy benzylamine (19.08 ml, 146 mmol) at room temperature. Trimethylsillylcyanide (19.93 ml, 159.286 mmol) followed by ammonium chloride (2.13 g, 39.8 mmol) were added to the RM at RT. The RM was stirred at 80° C. for 16 h. The RM was concentrated. The crude was partitioned between EtOAc and saturated sodium bicarbonate solution. Organic layer was washed with brine solution, dried over sodium sulphate and concentrated. The crude was purified on FCC (using 100-200 silica gel, eluted with 10% EtOAc-Hexane) to afford 3-(tert-Butyl-dimethyl-silanyloxy)-2-(4-methoxy-benzylamino)-2-methyl-propionitrile (25 g, 57%) as yellow liquid. ¹H-NMR (400 MHz, DMSO) δ [ppm]: 7.25-7.23 (d, 2H), 6.68-6.86 (d, 2H), 3.72-3.68 (m, 6H), 3.51-3.48 (m, 1H), 1.36 (s, 3H), 0.87 (s, 9H), 0.06 (s, 6H).

Step 2: To a stirred solution of 3-(tert-Butyl-dimethyl-silanyloxy)-2-(4-methoxy-benzylamino)-2-methyl-propionitrile (10 g, 29.9 mmol) in DMSO (100 ml) was added potassium carbonate (28.92 g, 209.243 mmol) at RT. Hydrogen peroxide (14.03 ml, 298.92 mmol) was added drop wise to the reaction at 0° C. The RM was stirred for 16 h at RT. The RM was quenched with ice cold water and extracted with MTBE. Organic part was dried over Na₂SO₄ and concentrated. Resultant crude material was purified on FCC (using 100-200 silica gel, eluted with 50% EtOAc-Hexane) to afford 3-(tert-Butyl-dimethyl-silanyloxy)-2-(4-methoxy-benzylamino)-2-methyl-propionamide (3.4 g, 33%) as oily liquid. ¹H-NMR (400 MHz, DMSO) δ [ppm]: 7.26-7.24 (d, 3H), 7.06 (s, 1H), 6.87-6.85 (d, 2H), 3.72 (s, 3H), 3.70-3.68 (m, 1H), 3.58-3.56 (m, 1H), 3.51-3.50 (d, 2H), 2.04 (m, 1H), 1.15 (s, 3H), 0.85 (s, 9H), 0.03 (s, 6H).

Step 3:To a stirred solution of -(tert-Butyl-dimethyl-silanyloxy)-2-(4-methoxy-benzylamino)-2-methyl-propionamide (3 g, 8.509 mmol) in MeOH (60 ml) was added palladium hydroxide (1.5 g) was added at RT. The RM was stirred for 4 h under H₂ gas balloon pressure at RT. The RM was filtered through celite bed, washed with 10% MeOH-DCM. Combine filtrate was concentrated to afford 2-amino-3-((tert-butyldimethylsilyl)oxy)-2-methylpropanamide (Int-02) (1.5 g, 76%) as off white solid. ¹H-NMR (400 MHz, DMSO) δ [ppm]: 7.25 (s, 1H), 6.95 (s, 1H), 3.77-3.74 (d, 1H), 3.70-3.68 (m, 1H), 3.27-3.24 (d, 1H), 1.82 (s, 2H), 1.04 (s, 3H), 0.85 (s, 9H), 0.02 (s, 6H).

### Synthesis of ethyl 5-hydroxy-2-methylpyrazolo[1,5-a]pyridine-3-carboxylate (Int-03)

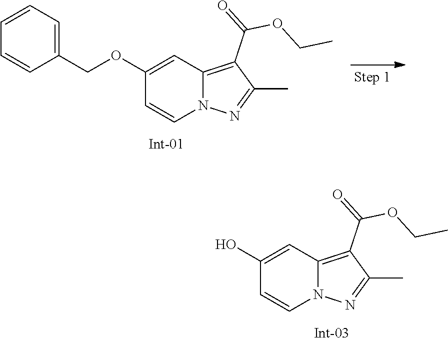 [SMILES: CCOC(=O)c1c(C)nn2ccc(OCc3ccccc3)cc12 ; CCOC(=O)c1c(C)nn2ccc(O)cc12]

Step 1 To a stirred solution of 5-benzyloxy-2-methyl-pyrazolo[1,5-a]pyridine-3-carboxylic acid ethyl ester (int-01) (10.0 g) in MeOH (650 mL), 10% Pd/C (2.5 g) was added at RT. The RM was stirred for 4 h in H₂ gas under balloon pressure. The RM was filtered through celite bed and celite bed was washed with 10% MeOH in dichloromethane (50 mL). Combined filtrate was concentrated under reduced pressure. The crude was triturated with MTBE and n-pentane (1:9, 100 mL) to afford ethyl 5-hydroxy-2-methylpyrazolo[1,5-a]pyridine-3-carboxylate (Int-03) (5.0 g, 70%) as an off-white solid. ¹H-NMR (400 MHz, DMSO-d6) d [ppm]: 10.89 (s, 1H), 8.49-8.51 (m, 1H), 7.20 (s, 1H), 6.59-6.62 (m, 1H), 4.24 (q, 2H), 2.47 (s, 3H), 1.32 (t, 3H).

### Synthesis of 2-amino-2-(hydroxymethyl)butanamide (Int-4)

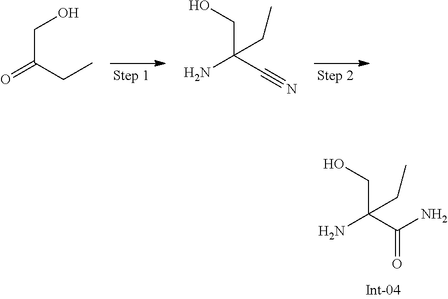

Step 1: A mixture of sodium cyanide (4.45 g, 90.90 mmol), ammonium chloride (4.9 g, 90.90 mmol), and ammonia solution (7M in methanol, 120 mL) in methanol (65 ml) was stirred at RT and stirred for 10 min, then 1-hydroxybutan-2-one (4.0 g, 45.45 mmol) was added. The reaction mixture was stirred for 24 h at RT and filter through celite bed, celite bed was washed with methanol (50 mL). Combined filtrate was concentrated under vacuum. To the residue was added ethyl acetate (80 mL) and the mixture was filtered. The filtrate was concentrated under vacuum. The residue was co-distilled with dichloromethane (2×50 mL) to afford 2-amino-2-(hydroxymethyl)butanenitrile (4.2 g) as a pale yellow residue. TLC system: 100% Ethyl acetate; RF: 0.3.

Step 2: To a stirred solution of 2-amino-2-(hydroxymethyl)butanenitrile (2.0 g, 17.54 mmol) in ethanol (20 mL) cooled to 0° C. was added KOH (1.47 g, 26.31 mmol) followed by 30% H₂O₂(2 mL) drop wise at 0° C. Reaction mixture was stirred for 1 h at RT. The reaction progress was monitored by TLC and reaction mixture was concentrated under vacuum. The crude was diluted with 10% methanol in dichloromethane (2×50 ml), stirred for 20 minutes and filtered. Filtrate was concentrated under reduced pressure to afford 2-amino-2-(hydroxymethyl)butanamide (Int-04) (1.8 g) as a thicky residue. TLC system: 10% Methanol in dichloromethane; RF: 0.1.

### Synthesis of Synthesis of 2-amino-4,4-difluorobutan-1-ol (Int-05)

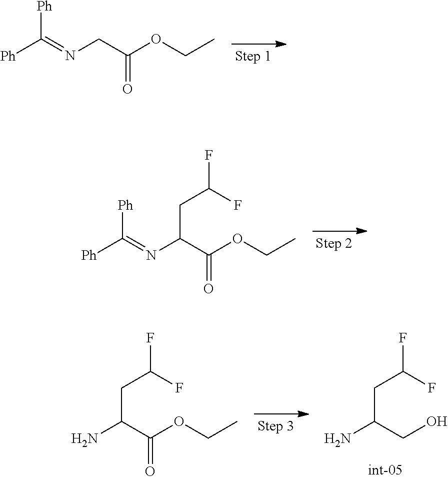 [SMILES: CCOC(=O)CN=C(c1ccccc1)c1ccccc1 ; CCOC(=O)C(CC(F)F)N=C(c1ccccc1)c1ccccc1 ; CCOC(=O)C(N)CC(F)F ; NC(CO)CC(F)F]

Step 1: To a solution of t-BuOK (13.2 g, 117.836 mmol) in DMF (150 mL) was added ethyl 2-((diphenylmethylene)amino) acetate (30 g, 112.225 mmol) at 0° C. After 30 min, 1,1-difluoro-2-iodoethane (24.9 g, 130.181 mmol) was added over 10 min maintaining the temperature at −5° C. to 5° C. The RM was stirred at 5° C. for 1 h. After completion, the RM was diluted with saturated NH₄Cl solution (100 mL), extracted with EtOAc (3×100 mL). Combined organic layer was washed with brine (100 mL), dried over Na₂SO₄, filtered and concentrated under reduced pressure. The residue was purified by FCC on silica gel using 5% EtOAc in pet ether as an eluent to afford ethyl 2-((diphenylmethylene)amino)-4,4-difluorobutanoate as a pale yellow liquid (30 g, 80.67%). ¹H NMR (400 MHz, CDCl₃) δ ppm: 7.64 (d, 2H), 7.42-7.48 (m, 4H), 7.31-7.39 (m, 2H), 7.18-7.25 (m, 2H), 5.75-5.91 (tt, 1H), 4.27-4.30 (m, 1H), 4.12-4.21 (m, 2H), 2.45-2.53 (m, 2H), 1.25 (t, 3H)

Step 2: To a solution of ethyl 2-((diphenylmethylene)amino)-4,4-difluorobutanoate (8.0 g, 24.143 mmol) in Hexane (40 ml) was added 1N HCL (80 ml) at RT. The RM was stirred at RT for 4 h. After completion, the RM was washed with Hexane (2×40 mL). The aqueous solution was basified with NaHCO₃ (pH -8) and extracted with EtOAc (2×100 mL). The combined organic layer was dried over Na₂SO₄ and concentrated under reduced pressure to afford the desired product as a brown liquid (2.5 g). ¹H NMR (400 MHz, CDCl₃) δ ppm: 5.91-6.22 (m, 1H), 4.20 (q, 2H), 3.61-3.65 (m, 1H), 2.24-2.36 (m, 1H), 1.98-2.00 (m, 1H), 1.27 (t, 3H).

Step 3: To a solution of ethyl 2-amino-4,4-difluorobutanoate (2.5 g, 14.956 mmol) in EtOH (30 mL) was added Sodium borohydride (1.697 g, 44.868 mmol) at 0° C. The RM was stirred at RT for 6 h. The RM was diluted with saturated NH₄Cl (20.0 mL), extracted with EtOAc (3×50 mL). Combined organic layer was washed with brine (20.0 mL), dried over anhydrous Na₂SO₄, filtered and concentrated under reduced pressure to afford 2-amino-4,4-difluorobutan-1-ol (Int-05) as a yellow liquid (1.2 g, 64.13%).

### Synthesis of Synthesis of 4,4-difluoro-1-methoxy-2-methylbutan-2-amine hydrochloride (Int-06)

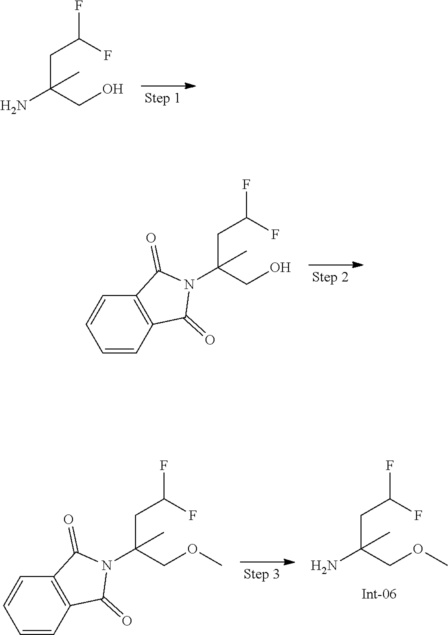

Step 1: To a solution of 2-amino-4,4-difluoro-2-methylbutan-1-ol (2.5 g, 17.96 mmol) in toluene (25 mL) was added phthalic anhydride (2.66 g, 17.96 mmol). The RM was stirred at 120° C. for 24 h in a sealed tube. After consumption, the RM was concentrated under reduced pressure. The residue was purified by FCC on silica gel using 25% EtOAc in pet ether as an eluent to afford 2-(4,4-difluoro-1-hydroxy-2-methylbutan-2-yl)isoindoline-1,3-dione as a pale yellow gummy (2.0 g, yield: 41.34%). ¹H NMR (400 MHz, CDCl₃) δ ppm: 7.79-7.84 (m, 2H), 7.71-7.75 (m, 2H), 6.06-6.37 (dt, 1H), 4.03-4.12 (m, 2H), 3.62 (t, 1H), 2.87-3.00 (m, 1H), 2.29-2.43 (m, 1H), 1.66 (s, 3H).

Step 2: To a solution of 2-(4,4-difluoro-1-hydroxy-2-methylbutan-2-yl)isoindoline-1,3-dione (2.6 g, 9.657 mmol) in DMF (26 mL) were added NaH (60% in mineral oil) (463.51 mg, 19.313 mmol), Iodomethane (1.194 mL, 19.31 mmol) at 0° C. The RM was stirred at RT for 2 h. After consumption, the RM was quenched with ice cold water (20 mL) and extracted with EtOAc (2×40 mL). Organic layer was dried over Na₂SO₄, filtered and concentrated under reduced pressure. The residue was purified by FCC on silica gel using 25% EtOAc in pet ether as an eluent to afford 2-(4,4-difluoro-1-methoxy-2-methylbutan-2-yl)isoindoline-1,3-dione as a pale yellow gummy (1.1 g, yield: 40.21%). ¹H NMR (400 MHz, CDCl₃) δ ppm: 7.76-7.81 (m, 2H), 7.67-7.72 (m, 2H), 5.87-6.17 (dt, 1H), 3.99 (d, 1H), 3.79 (d, 2H), 3.36 (d, 3H), 2.77-2.90 (m, 1H), 2.40-2.53 (m, 1H), 1.82 (s, 3H).

Step 3: To a solution of 2-(4,4-difluoro-1-methoxy-2-methylbutan-2-yl)isoindoline-1,3-dione (1.1 g, 3.883 mmol) in IPA (22 mL) and water (2.2 mL) was added NaBH₄ (733.9 mg, 19.41 mmol) at 0° C. The RM was stirred at RT for 16 h. After completion, the RM was concentrated and the resulting residue was quenched with cold water (30 mL), extracted with EtOAc (2×30 mL). Organic layer was washed with brine (20 mL), dried over anhydrous Na₂SO₄, filtered and concentrated under reduced pressure. The residue was treated with 6N HCl (22 mL) and heated to 80° C. for 2 h. The RM was concentrated under reduced pressure and the obtained residue was triturated with diethyl ether (2×30 mL). The solid was filtered and dried under vacuum to afford 4,4-difluoro-1-methoxy-2-methylbutan-2-amine hydrochloride (Int-06) as an off white solid (650 mg, yield: 88.27%). ¹H NMR (400 MHz, DMSO-de) 5 ppm: 8.40 (s, 3H), 6.20-6.50 (dt, 1H), 3.43 (s, 2H), 3.33 (s, 3H), 2.19-2.30 (m, 2H), 1.29 (s, 3H).

### Synthesis of methyl 1-amino-2,2-difluorocyclopropane-1-carboxylate hydrobromide (Int-07)

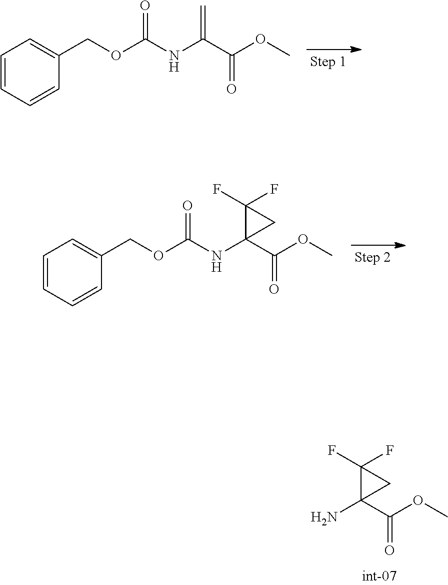

Step 1: To z solution of methyl 2-(((benzyloxy)carbonyl)amino)acrylate (2.5 g, 10.627 mmol) in toluene (50 mL) was added NaF (44.61 mg, 1.063 mmol) at RT and heated to 110° C. After 10 min, a solution of trimethylsilyl 2,2-difluoro-2-(fluorosulfonyl)acetate (5.236 mL, 26.56 mmol) in toluene (50 mL) was added dropwise over 4 h to the RM. The RM was stirred at 110° C. for 18 h. After completion, the RM was cooled to 0° C. and quenched with sat Na₂CO₃ solution (70 mL), extracted with EtOAc (2×70 mL). The combined organic layers were washed with brine solution (50 mL), dried over Na₂SO₄, filtered and concentrated under reduced pressure. The residue was purified by FCC on silica gel using 15% EtOAc in pet ether as an eluent to afford methyl 1-(((benzyloxy)carbonyl)amino)-2,2-difluorocyclopropane-1-carboxylate as a pale yellow liquid (1.4 g, yield: 46.18%). ¹H NMR (400 MHz, CDCl₃) 5 ppm: 7.32-7.38 (m, 5H), 5.57 (s, 1H), 5.14 (s, 2H), 3.77 (s, 3H), 2.66-2.71 (m, 1H), 1.75-1.95 (m, 1H).

Step 2: Methyl 1-(((benzyloxy)carbonyl)amino)-2,2-difluorocyclopropane-1-carboxylate (1.4 g, 4.908 mmol) and HBr in acetic acid (33%) (7.5 mL) was stirred at RT for 2 h. After consumption, the RM was concentrated under reduced pressure and the obtained residue was triturated with diethyl ether (2×30 mL). The obtained solid was filtered and dried under vacuum to afford methyl 1-amino-2,2-difluorocyclopropane-1-carboxylate hydrobromide (Int-07) as a pale brown solid (1.0 g, Yield: 87.81%). ¹H NMR (400 MHz, DMSO-de) 6 ppm: 9.15 (s, 2H), 3.82 (s, 3H), 2.69-2.78 (m, 1H), 2.43-2.51 (m, 1H).

### Synthesis of (cis-1-amino-2-(difluoromethyl)cyclopropyl)methanol hydrochloride (Int-08)

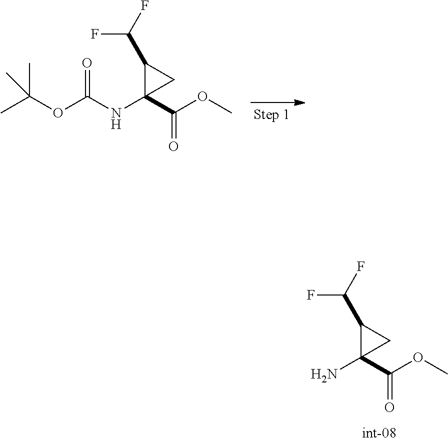 [SMILES: COC(=O)[C@]1(NC(=O)OC(C)(C)C)C[C@@H]1C(F)F ; COC(=O)[C@]1(N)C[C@@H]1C(F)F]

Step 1: To a solution of tert-butyl (cis-2-(difluoromethyl)-1-(hydroxymethyl)cyclopropyl)carbamate (880 mg, 3.318 mmol) in DCM (10 mL) was added 4 M HCl in Dioxane (5.0 mL) at RT. The RM was stirred at RT for 1 h. After consumption, the RM was concentrated under reduced pressure and the obtained residue was triturated with diethyl ether (2×20 mL). The residue was dried under vacuum to afford cis-1-amino-2-(difluoromethyl)cyclopropyl)methanol hydrochloride (Int-08) as pale yellow gummy (700 mg). ¹H NMR (400 MHz, CDCl₃) δ ppm: 9.19 (s, 2H), 6.00-5.70 (dt, 1H), 3.70 (s, 3H), 2.80-2.70 (m, 1H), 2.23-2.18 (m, 1H), 1.90-1.86 (m, 1H).

### Synthesis of 2-amino-2-(1-cyclopropyl-1H-imidazol-2-yl)ethan-1-ol hydrochloride (Int-09)

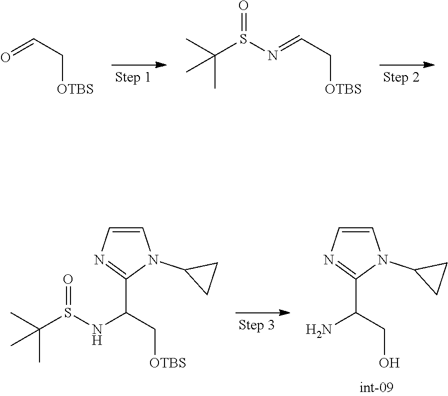 [SMILES: CC(C)(C)[Si](C)(C)OCC=O ; CC(C)(C)S(=O)/N=C/CO[Si](C)(C)C(C)(C)C ; CC(C)(C)S(=O)NC(CO[Si](C)(C)C(C)(C)C)c1nccn1C1CC1 ; NC(CO)c1nccn1C1CC1]

Step 1: To a solution of 2-((tert-butyldimethylsilyl)oxy)acetaldehyde (2 g, 11.47 mmol) in DCM (30 mL) were added 2-methylpropane-2-sulfinamide (1.53 g, 12.62 mmol) and copper(II)sulphate (3.66 g, 22.94 mmol) at RT under N₂ atmosphere. The RM was stirred at RT for 12 h. After completion, the RM was filtered and rinsed with DCM (50 mL). The filtrate was concentrated under reduced pressure. The residue was purified by FCC on silica gel using 10% EtOAc in Pet. Ether as an eluent to afford (E/Z)—N-(2-((tert-butyldimethylsilyl)oxy)ethylidene)-2-methylpropane-2-sulfinamide as a pale yellow gum (1.5 g, yield: 47.11%). ¹H NMR (400 MHz, CDCl₃) 5 ppm: 7.96 (s, 1H), 4.44 (s, 2H), 1.10 (s, 9H). 0.82 (s, 9H).

Step 2: To a solution of 1-cyclopropyl-1H-imidazole (500 mg, 4.62 mmol) in THE (22 mL) was treated with nBuLi (5 mL, 2.5 M) drop wise at −78° C. The RM was stirred for 2 h at -78° C. After 2 h, a solution of (E)-N-(2-((tert-butyldimethylsilyl)oxy)ethylidene)-2-methylpropane-2-sulfinamide (1.54 g, 5.54 mmol) in THE (2 mL) was added drop wise to the RM. The RM was stirred for 1.5 h. After completion, the RM was quenched with sat. NH₄Cl (25 mL) at 0° C., extracted with EtOAc (3×30 mL). The combined organic layers were washed with brine (30 mL) and concentrated to afford compound (1.5 g, 84%).

Step 3:To q solution of N-(2-((tert-butyldimethylsilyl)oxy)-1-(1-cyclopropyl-1H-imidazol-2-yl)ethyl)-2-methylpropane-2-sulfinamide (1.3 g, 3.37 mmol) in 1,4-dioxane (10 mL) was added 4M HCl in 1,4-dioxane (5 mL) at 0° C. The RM was stirred at RT for 16 h. After consumption, the RM was concentrated under reduced pressure. The residue was triturated with DCM (2×5 mL) and dried under vacuum to afford 2-amino-2-(1-cyclopropyl-1H-imidazol-2-yl)ethan-1-ol hydrochloride (Int-09) as a brown gummy (800 mg, 88%).

### Synthesis of ethyl 2-amino-2-(1-methyl-1H-pyrazol-3-yl)propanoate (Int-10)

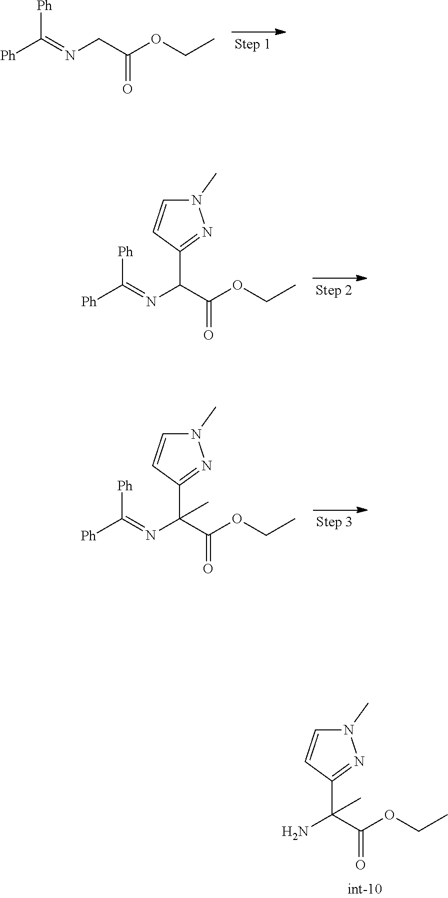 [SMILES: CCOC(=O)CN=C(c1ccccc1)c1ccccc1 ; CCOC(=O)C(N=C(c1ccccc1)c1ccccc1)c1ccn(C)n1 ; CCOC(=O)C(C)(N=C(c1ccccc1)c1ccccc1)c1ccn(C)n1 ; CCOC(=O)C(C)(N)c1ccn(C)n1]

Step 1:To a solution of ethyl 2-((diphenylmethylene)amino)acetate (5.0 g, 18.704 mmol), 3-bromo-1-methyl-1H-pyrazole (2.850 mL, 28.056 mmol) in toluene (50 mL, taken in 100 mL sealed tube) was added K₃PO₄ (11.907 g, 56.113 mmol). The RM was degassed with argon for 20 min. Pd(t-Bu3P)2 (955.87 mg, 1.870 mmol) was added at RT. The resulted RM was stirred at 100° C. for 16 h. After completion, the RM was diluted with cold water (100 mL), extracted with EtOAc (2×100 mL). Combined organic layers were washed with brine (100 mL), dried over Na₂SO₄, filtered and concentrated under reduced pressure. The residue was purified by FCC on silica gel using 12% EtOAc in pet ether as an eluent to afford ethyl 2-((diphenylmethylene)amino)-2-(1-methyl-1H-pyrazol-3-yl) acetate as a pale yellow gummy (2.5 g, yield: 38.47%). ¹H NMR (400 MHz, CDCl₃) 5 ppm: 7.68-7.71 (q, 2H), 7.42-7.44 (m, 3H), 7.36-7.38 (m, 1H), 7.30-7.33 (m, 3H), 7.17-7.20 (m, 2H), 6.45 (d, 1H), 5.34 (s, 1H), 4.15-4.19 (m, 2H), 3.84 (s, 3H), 1.22 (t, 3H).

Step 2: To a solution of ethyl 2-((diphenylmethylene)amino)-2-(1-methyl-1H-pyrazol-3-yl)acetate (1.8 g, 5.181 mmol,) in DMF (20 mL, taken in a sealed tube) was added NaH (60%) (621 mg, 15.543 mmol) portion wise at 0° C. The RM was stirred for 30 min at 0° C. After 30 min, Iodomethane (1.613 mL, 25.90 mmol) was added. The RM was stirred at RT for 16 h. After completion, the RM was quenched with cold water (50 mL), extracted with EtOAc (2×50 mL). Combined organic layers were washed with brine (50 mL), dried over anhydrous Na₂SO₄, filtered and concentrated under reduced pressure. The residue was purified by FCC on silica gel using 20% EtOAc in pet ether as an eluent to afford ethyl 2-((diphenylmethylene)amino)-2-(1-methyl-1H-pyrazol-3-yl)propanoate as a pale brown gummy (1.0 g, yield: 53.40%). ¹H NMR (400 MHz, DMSO-de) 5 ppm: 7.48-7.52 (m, 3H), 7.34-7.42 (m, 6H), 6.34 (d, 2H), 5.75 (s, 1H), 3.63-3.75 (m, 5H), 1.62 (s, 3H), 1.00 (t, 3H).

Step 3: To a solution of ethyl 2-((diphenylmethylene)amino)-2-(1-methyl-1H-pyrazol-3-yl)propanoate (1.6 g, 4.427 mmol) in Hexane (15 mL) was added 1N HCl (aq) (30 mL) at RT. The RM was stirred for 16 h. After completion, the RM was extracted with EtOAc (3×20 mL). Aqueous layer was basified with sat. NaHCO₃ solution (pH˜0.8), extracted with 10% MeOH in DCM (3×30 mL). Combined organic layer was dried over Na₂SO₄, filtered and concentrated under reduced pressure to afford ethyl 2-amino-2-(1-methyl-1H-pyrazol-3-yl)propanoate (Int-10) as a brown liquid (630 mg, 72.24%). ¹H NMR (400 MHz, DMSO-de) 5 ppm: 7.54 (s, 1H), 6.18 (s, 1H), 4.00-4.07 (m, 2H), 3.99 (s, 3H), 2.21 (s, 2H), 1.50 (s, 3H), 1.12 (t, 3H).

### Synthesis of tert-butyl trans-3-amino-4-(trifluoromethyl)pyrrolidine-1-carboxylate (Int-11)

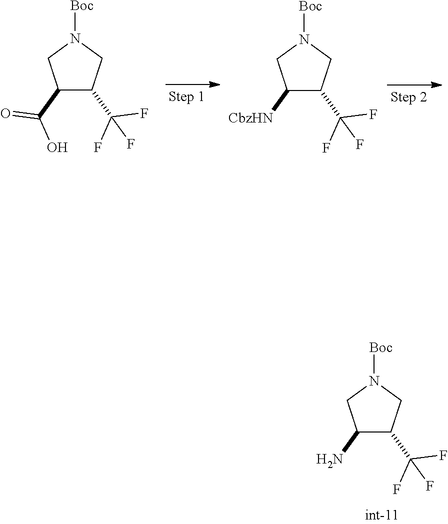 [SMILES: CC(C)(C)OC(=O)N1C[C@H](C(=O)O)[C@@H](C(F)(F)F)C1 ; CC(C)(C)OC(=O)N1C[C@H](NC(=O)OCc2ccccc2)[C@@H](C(F)(F)F)C1 ; CC(C)(C)OC(=O)N1C[C@H](N)[C@@H](C(F)(F)F)C1]

Step 1: To a solution of Trans-1-(tert-butoxycarbonyl)-4-(trifluoromethyl)pyrrolidine-3-carboxylic acid (3 g, 10.591 mmol) in xylene (25 mL) were added TEA (1.771 mL, 12.710 mmol) and DPPA (2.505 mL, 11.651 mmol) dropwise at RT. The RM was heated to 130° C. and stirred for 1 h. After 1 h, phenyl methanol (1.211 mL, 11.651 mmol) was added into the RM and stirred at 130° C. for 3 h. After cooling to RT, the RM was quenched with 1N NaOH solution (80.0 mL), washed with EtOAc (2×50 mL). The aqueous layer was washed acidified with aqueous citric acid solution (pH˜5), extracted with DCM (2×80 mL). Organic layers were washed with brine (50 mL), dried over Na₂SO₄, filtered and concentrated under reduced pressure. The residue was purified by FCC on silica gel using 15% EtOAc in pet ether as an eluent to afford tert-butyl trans-3-(((benzyloxy)carbonyl)amino)-4-(trifluoromethyl)pyrrolidine-1-carboxylate as a yellow solid (2.2 g, 53.48%). ¹H NMR (400 MHz, DMSO-de) 5 ppm: 7.32-7.38 (m, 7H), 5.10 (s, 3H), 4.69 (d, 1H), 4.35-4.40 (m, 1H), 3.77-3.82 (m, 1H), 3.49 (s, 2H), 2.94 (s, 2H), 1.45 (s, 9H).

Step 2: To a solution of tert-butyl trans-3-(((benzyloxy)carbonyl)amino)-4-(trifluoromethyl)pyrrolidine-1-carboxylate (2.4 g, 6.179 mmol) in EtOAc (20 mL) was added 10% Pd/C (1.315 g, 12.359 mmol). The RM was stirred at RT for 16 h under hydrogen balloon pressure. After completion, the RM was filtered through a celite pad, washed with EtOAc (2×100 mL) and the filtrate was concentrated under reduce pressure. The residue was purified by FCC on silica gel using 45% EtOAc in pet ether as an eluent to afford tert-butyl trans-3-amino-4-(trifluoromethyl)pyrrolidine-1-carboxylate (Int-11) as a colorless liquid (1.3 g, 82.74%). ¹H NMR (400 MHz, DMSO-de) 5 ppm: 3.49-3.62 (m, 3H), 3.26-3.31 (m, 2H), 2.96 (s, 1H), 2.85 (s, 1H), 1.82-1.98 (m, 2H), 1.39 (s, 9H).

### Synthesis of benzyl 3-amino-3-(2,2-difluoroethyl)piperidine-1-carboxylate (Int-12)

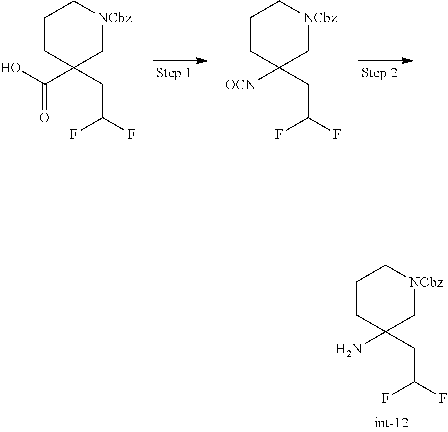

Step 1: To a solution of 1-((benzyloxy)carbonyl)-3-(2,2-difluoroethyl)piperidine-3-carboxylic acid (2.0 g, 6.114 mmol) in toluene (20 mL) was added TEA (1.704 mL, 12.228 mmol) and DPPA (1.446 mL, 6.725 mmol) dropwise at RT. The RM was heated at 100° C. for 2 h. After cooling to RT, the RM was diluted with water, extracted with EtOAc (2×100 mL). The combined organic layers were washed with brine (50 mL), dried over Na₂SO₄, filtered and concentrated under reduced pressure to afford benzyl 3-(2,2-difluoroethyl)-3-isocyanatopiperidine-1-carboxylate as a pale yellow liquid (1.8 g, 90.78%).

Step 2: To a solution of benzyl 3-(2,2-difluoroethyl)-3-isocyanatopiperidine-1-carboxylate (1.8 g, 5.550 mmol) in 1,4-dioxane (18 mL) was added an aqueous solution 6M HCl (18 mL). The RM was heated to 45° C. for 16 h. After completion, the RM was concentrated. The residue was diluted with water (50 mL), washed with EtOAc (100 mL). Aqueous layer was basified with 1N NaOH solution (50 mL), extracted with DCM (2×100 mL). The combined organic layers were washed with brine (10 mL), dried over Na₂SO₄, filtered and concentrated under reduced pressure to afford benzyl 3-amino-3-(2,2-difluoroethyl)piperidine-1-carboxylate (Int-12) as a pale yellow liquid (900 mg, 54.36%). ¹H NMR (400 MHz, DMSO de) 6 ppm: 7.37-7.30 (m, 5H), 6.37-6.07 (m, 1H), 5.06 (s, 2H), 3.56 (s, 4H), 3.38-3.16 (m, 3H), 1.90-1.79 (m, 2H), 1.65-1.55 (m, 4H), 1.43-1.38 (m, 2H).

### Synthesis of ethyl 2-amino-2-(1-methyl-1H-pyrazol-3-yl)acetate (Int-13)

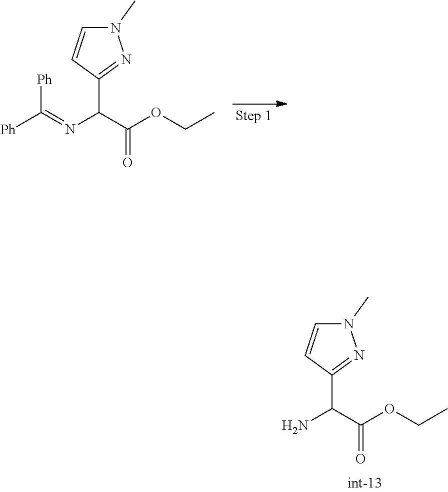 [SMILES: CCOC(=O)C(N=C(c1ccccc1)c1ccccc1)c1ccn(C)n1 ; CCOC(=O)C(N)c1ccn(C)n1]

Step 1: To a solution of ethyl 2-((diphenylmethylene)amino)-2-(1-methyl-1H-pyrazol-3-yl)acetate (2.4 g, 6.908 mmol, 1.0 eq) in Hexane (10 mL) was added 1N HCl (aq) (25 mL) at RT. The RM was stirred for 16 h. After completion, the RM was extracted with EtOAc (2×30 mL). Aqueous layer was basified with sat. NaHCO₃ solution (pH˜0.8), extracted with 10% MeOH in DCM (3×50 mL). The combined organic layer was dried over Na₂SO₄, filtered and concentrated under reduced pressure to afford ethyl 2-amino-2-(1-methyl-1H-pyrazol-3-yl)acetate (Int-13) (900 mg, 71.11%). ¹H NMR (400 MHz, DMSO-de) 5 ppm: 7.57 (d, 1H), 6.15 (d, 1H), 4.43 (s, 1H), 4.11-4.01 (m, 2H), 3.70 (s, 3H), 2.07 (s, 2H), 1.14 (t, 3H).

### Synthesis of 2-amino-3-hydroxy-2-methylpropanamide (Int-14)

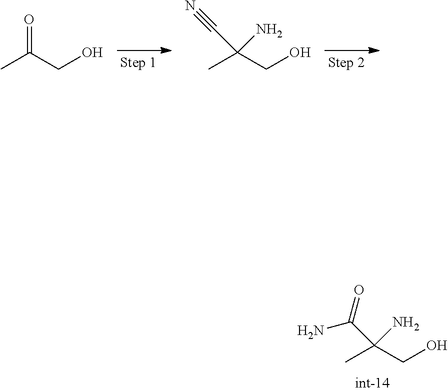

Step 1: To a mixture of sodium cyanide (3.97 g, 81.08 mmol), ammonium chloride (4.29 g, 81.08 mmol), and ammonia solution (7M in methanol, 30 mL) was added, after 10 min, 1-hydroxyacetone (3.0 g, 40.54 mmol,). The RM was stirred for RT, under nitrogen, for 23 h and then concentrated under vacuum. To the residue was added EtOAc (200 mL) and the solid was filtered. The filtrate was concentrated under vacuum and to the residue was added DCM (1×50 mL). The solution was cooled to −20° C. and allowed to stand for 60 h at RT. The precipitate was collected by filtration, washed with cold DCM and dried under vacuum to afford 2-amino-3-hydroxy-2-methylpropanenitrile (2.0 g, 50%).

Step 2: To a stirred solution of amino-3-hydroxy-2-methylpropanenitrile (2.0 g, 20 mmol), in ethanol (30 mL), at 0° C., was added KOH (1.62 g, 30 mmol, 1.5 eq), then H₂O₂(2 mL) was added drop wise at 0° C. The RM was stirred at RT for 18 h. The RM was concentrated under reduced pressure. The residue was diluted with 10% MeOH in DCM (2×100 ml), stirred for 20 minutes, then filtered and the filtrate was concentrated under reduced pressure to afford 2-amino-3-hydroxy-2-methylpropanamide (Int-14) (0.6 g, 26%).

### Synthesis of Ethyl 3-amino-2-oxopyrrolidine-3-carboxylate hydrochloride (Int-15)

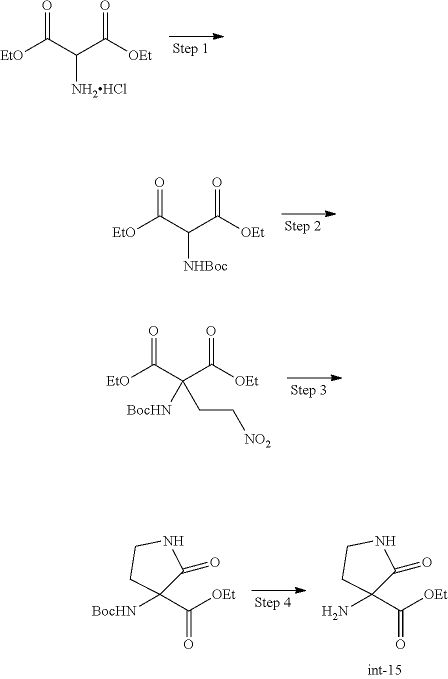

Step 1: Boc anhydride (35.8 mL, 155.9 mmol) was added to a solution of diethyl 2-aminomalonate hydrochloride (30 g, 141.7 mmol) and TEA (60.18 mL, 425.25 mmol) in DCM (400 mL) at 0° C. The RM was stirred at RT for 16 h. The RM was diluted with ice water (500 mL) and the organic layer was separated, organic layer was washed with brine (200 mL), dried over Na₂SO₄, filtered and concentrated under reduced pressure to afford diethyl 2-((tert-butoxycarbonyl)amino)malonate (35.8 g) as a colorless.

Step 2: To a solution of diethyl 2-((tert-butoxycarbonyl)amino)malonate (10 g, 36.36 mmol) and sodium ethoxide (1.97 g, 29.08 mmol) in ethanol (300 mL) cooled to 0° C., was added a solution of nitroethene (6.64 g, 90.90 mmol) dissolved in diethyl ether (15 mL). The RM was slowly allowed to reach RT. Then, the RM was stirred for 2 h. The RM was diluted with ice water (500 mL) and the organic layer was separated, washed with brine (100 mL), dried over Na₂SO₄, filtered and concentrated under reduced pressure to give residue. Residue was purified by reverse phase GRACE flash chromatography using 0.1% FA in water and ACN as an eluent. The fractions were collected and concentrated under vacuum to afford diethyl 2-((tert-butoxycarbonyl)amino)-2-(2-nitroethyl)malonate (4.1 g).

Step 3: Raney-Ni (5.3 g) was added to a solution of diethyl 2-((tert-butoxycarbonyl)amino)-2-(2-nitroethyl)malonate (4.0 g, 11.49 mmol) in ethanol (50 mL) in Parr hydrogenation vessel under inert atmosphere. The RM was subjected to hydrogenation under 70 psi at RT for 16 h. The RM was filtered through celite pad, and the celite pad was washed with ethanol. The combined filtrate was concentrated under reduced pressure to afford ethyl 3-((tert-butoxycarbonyl)amino)-2-oxopyrrolidine-3-carboxylate (1.6 g) as a colorless oil.

Step 4: HCl (4M in dioxane, 3.6 mL) was added to a stirred solution of ethyl 3-((tert-butoxycarbonyl)amino)-2-oxopyrrolidine-3-carboxylate (1.6 g, 5.9 mmol) in DCM (30 mL) at 0° C. The RM was stirred at RT for 2 h. The RM was concentrated under vacuum. The residue was triturated with diethyl ether (10 mL), filtered and dried under vacuum to afford ethyl 3-amino-2-oxopyrrolidine-3-carboxylate hydrochloride (1.03 g) as an off-white solid.

### Synthesis of 3-Amino-3-(hydroxymethyl)pyrrolidin-2-one (Int-16—En1) and (Int-16—En2)

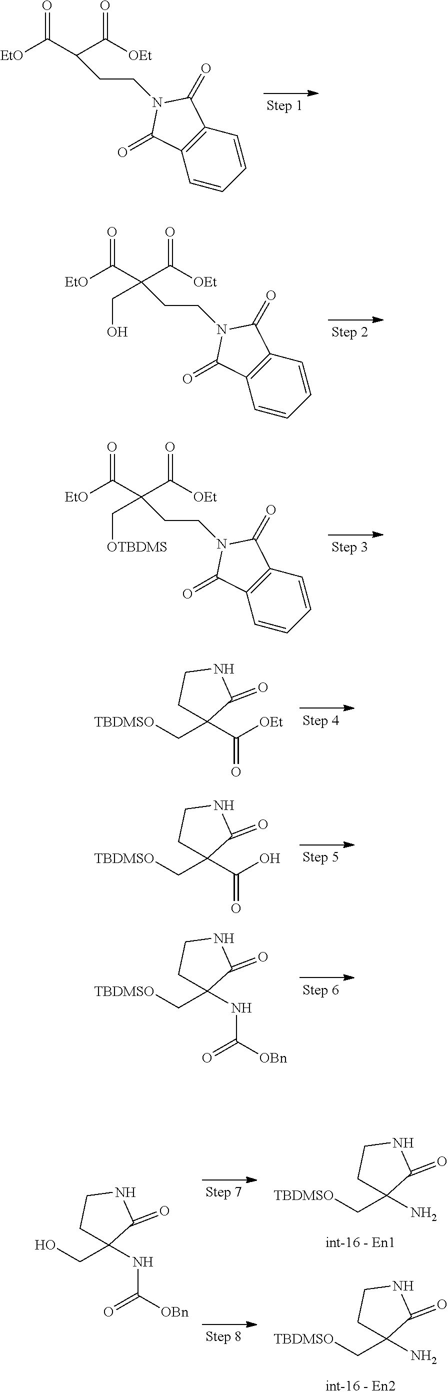

Step 1: To a stirred solution of diethyl 2-(2-(1,3-dioxoisoindolin-2-yl)ethyl)malonate (60 g, 179.99 mmol) in 1,4-Dioxane (600 mL) was added TEA (49.90 mL, 359.99 mmol) at 0° C. After 15 minutes, formaldehyde (29.18 g, 359.99 mmol) was added at 0° C. The RM was warmed to RT and stirred at 80° C. for 16 h. After completion, the RM was diluted with ice-cold water (200 mL) and extracted with EtOAc (2×300 mL). The combined organic layers were washed with brine (150 mL), dried over Na₂SO₄, filtered, and concentrated under reduced pressure. The residue was purified by FCC on silica gel using a gradient of 0 to 40% EtOAc in pet ether to afford diethyl 2-(2-(1,3-dioxoisoindolin-2-yl)ethyl)-2-(hydroxymethyl)malonate (59 g, 90%) as pale yellow gummy. ¹H NMR (400 MHz, CDCl₃) δ ppm: 7.86-7.82 (m, 2H), 7.74-7.69 (m, 2H), 4.25-4.18 (m, 4H), 4.07 (d, 2H), 3.83-3.79 (m, 2H), 2.79 (t, 1H), 2.33-2.30 (m, 2H), 1.28 (t, 6H).

Step 2: To a stirred solution of diethyl 2-(2-(1,3-dioxoisoindolin-2-yl)ethyl)-2-(hydroxymethyl)malonate (68 g, 187.13 mmol) in DCM (700 mL) was added Imidazole (25.48 g, 374.27 mmol) at 0° C. and stirred for 15 min. Then, tert-Butyldimethylsilyl chloride (33.84 g, 224.56 mmol) at 0° C. as added. The RM was stirred for 16 h at RT. After completion, the RM was diluted with ice cold water (300 mL) and extracted with DCM (2×500 mL). The combined organic layers were washed with brine (200 mL), dried over Na₂SO₄, filtered, and concentrated under reduced pressure. The residue was purified by FCC on silica gel using a gradient of 10% EtOAc in pet ether to afford diethyl 2-(((tert-butyldimethylsilyl)oxy)methyl)-2-(2-(1,3-dioxoisoindolin-2-yl)ethyl)malonate (68 g, 77%) as an off-white solid. ¹H NMR (400 MHz, CDCl₃) δ ppm: 7.84-7.82 (m, 2H), 7.71-7.69 (m, 2H), 4.18-4.11 (m, 6H), 3.76-3.72 (m, 2H), 2.41-2.37 (m, 2H), 1.26 (t, 6H), 0.89 (s, 9H), 0.08 (s, 6H). Step 3: To a stirred solution of diethyl 2-(((tert-butyldimethylsilyl)oxy)methyl)-2-(2-(1,3-dioxoisoindolin-2-yl)ethyl)malonate (68 g, 142.37 mmol) in Ethanol (700 mL) was added Hydrazine hydrate (10.69 g, 213.55 mmol) at 0° C. The RM was stirred for 16 h at RT. After completion, the RM was diluted with ice cold water (300 mL) and extracted with EtOAc (2×500 mL). The combined organic layers were washed with brine solution (200 mL), dried over Na₂SO₄, filtered, and concentrated under reduced pressure. The residue was purified by FCC on silica gel using a gradient of 0-30% EtOAc in Pet. ether to afford ethyl 3-(((tert-butyldimethylsilyl)oxy)methyl)-2-oxopyrrolidine-3-carboxylate (30.7 g, 71%) as an off white solid. ¹H NMR (400 MHz, CDCl₃) 5 ppm: 5.76 (s, 1H), 4.22-4.17 (m, 2H), 4.06 (d, 1H), 3.94 (d, 1H), 3.46-3.42 (m, 1H), 3.36-3.35 (m, 1H), 2.59-2.55 (m, 1H), 2.49-2.43 (m, 1H), 1.27 (t, 3H), 0.87 (d, 9H), 0.06 (d, 6H).

Step 4: To a solution of ethyl 3-(((tert-butyldimethylsilyl)oxy)methyl)-2-oxopyrrolidine-3-carboxylate (29 g, 96.19 mmol) in EtOH (120 mL), THE (60 mL) and H₂O (30 mL) was added LiOH·H2O (20.18 g, 480.99 mmol) portion wise at 0° C. The RM was stirred for 16 h at RT. After completion, the volatiles were removed under reduced pressure. The residue was diluted with cold water (20 mL) and then acidified with saturated aqueous Citric acid solution (pH˜4). The solid was filtered, washed with water (10 mL) followed by water (10 mL), dried under vacuum to afford 3-(((tert-butyldimethylsilyl)oxy)methyl)-2-oxopyrrolidine-3-carboxylic acid (16.7 g, 63%) as a white solid. ¹H NMR (400 MHz, DMSO-de) 5 ppm: 7.82 (s, 1H), 3.88 (d, 1H), 3.70 (d, 1H), 3.27-3.21 (m, 1H), 3.17-3.12 (m, 1H), 2.34-2.32 (m, 1H), 2.24-2.22 (m, 1H), 0.84 (s, 9H), 0.01 (d, 6H).

Step 5: To a stirred solution of 3-(((tert-butyldimethylsilyl)oxy)methyl)-2-oxopyrrolidine-3-carboxylic acid (9.5 g, 34.74 mmol) in THE (40 mL) and benzene (120 mL) were added TEA (14.65 mL, 104.24 mmol) followed by Diphenylphosphoryl azide (19.12 g, 69.49 mmol) at RT. The RM was stirred at RT for 2 h. After completion, the RM was quenched with ice cold water (100 mL) and extracted with EtOAc (2×200 mL). The combined organic layers were washed with saturated aqueous sodium bicarbonate (2×50 mL), brine solution (50 mL), then dried over Na₂SO₄, filtered, and concentrated under reduced. The residue was dissolved in THE (40 mL) and benzene (120 mL). Then, Benzyl alcohol (7.51 g, 69.49 mmol) was added at RT. The RM was stirred at 55° C. for 16 h. After completion, the RM was diluted with ice-cold water (100 mL) and extracted with EtOAc (2×200 mL). The combined organic layers were washed with brine solution (100 mL), dried over Na₂SO₄, filtered, and concentrated under reduced pressure. The residue was purified by FCC on silica gel using a gradient of 0-40% EtOAc in Pet. ether to afford benzyl (3-(((tert-butyldimethylsilyl)oxy)methyl)-2-oxopyrrolidin-3-yl)carbamate (9 g, 68%). ¹H NMR (400 MHz, CDCl₃) δ ppm: 7.37-7.29 (m, 5H), 5.84 (brs, 1H), 5.42 (brs, 1H), 5.07 (s, 2H), 3.85 (d, 1H), 3.67 (d, 1H), 3.39-3.33 (m, 2H), 2.63-2.62 (m, 1H), 2.53-2.51 (m, 1H), 0.88 (s, 9H), 0.05 (d, 6H).

Step 6: To a stirred solution of benzyl (3-(((tert-butyldimethylsilyl)oxy)methyl)-2-oxopyrrolidin-3-yl)carbamate (11 g, 29.05 mmol) in MeOH (150 mL) was added PTSA monohydrate (2.21 g, 11.62 mmol) in MeOH (50 mL) over 2 h at 0° C. The RM was stirred for 16 h at RT. After completion, the RM was concentrated under reduced pressure and the residue was quenched with ice-cold water (50 mL) and extracted with 10% MeOH in DCM (3×50 mL). The combined organic layer was dried over Na₂SO₄, filtered, and concentrated under reduced pressure. The residue was triturated with n-pentane (3×15 mL) followed by diethyl ether (15 mL), filtered and dried under vacuum to afford benzyl (3-(hydroxymethyl)-2-oxopyrrolidin-3-yl)carbamate (7 g, 91%) as an off white solid. ¹H NMR (400 MHz, DMSO-de) 6 ppm: 7.66 (s, 1H), 7.38-7.29 (m, 5H), 7.08 (s, 1H), 4.98-4.95 (m, 3H), 3.46-3.38 (m, 2H), 3.18-3.15 (m, 1H), 3.13-3.07 (m, 1H), 2.28-2.25 (m, 2H). 3.5 g of benzyl (3-(hydroxymethyl)-2-oxopyrrolidin-3-yl)carbamate was purified by chiral SFC prep. [Preparative SFC Conditions: Column: Chiral peak IF (250×30×5 μm), % CO2: 65%, % Co-solvent: 35% (100% Methanol), Total Flow: 90 g/min, Back Pressure: 100.0 bar, Temperature: 30° C., Wavelength: 215 nm, Stack time: 7.2 min, Solubility: 100 ml of MeOH.] The collected pure fractions were concentrated under reduced pressure to afford two isomers En1 (the first eluting) and En2 (the second eluting).

Step 7: To a stirred solution of benzyl (3-(hydroxymethyl)-2-oxopyrrolidin-3-yl)carbamate En1 (1.4 g, 5.297 mmol) in Ethanol (30 mL) was added Pd/C (450 mg) at RT.

The RM was stirred under hydrogen gas pressure (70 psi, Parr shaker) at RT for 16 h. After completion, the RM was filtered through a small pad of Celite, and washed with MeOH (50 mL). The filtrate concentrated under reduced pressure. The residue was triturated with diethyl ether (2×5 mL) and dried under high vacuum to afford 3-amino-3-(hydroxymethyl)pyrrolidin-2-one (Int-16—En1) (610 mg, 88%) as an off-white solid. 1H NMR (400 MHz, DMSO-d6) δ ppm: 7.54 (s, 1H), 4.73 (t, 1H), 3.36-3.34 (m, 1H), 3.18-3.12 (m, 2H), 3.10-3.04 (m, 1H), 2.22-2.15 (m, 1H), 1.76-1.72 (m, 1H), 1.56 (s, 2H). UPLC: Rt=2.29 min (98%).

Step 8: To a stirred solution of benzyl (3-(hydroxymethyl)-2-oxopyrrolidin-3-yl)carbamate En2 (1.2 g, 4.54 mmol) in Ethanol (30 mL) was added Pd/C (350 mg) at RT. The RM was stirred under hydrogen gas pressure (70 psi, Parr shaker) at RT for 16 h. After completion, the RM was filtered through a small pad of Celite, and washed with MeOH (50 mL). The filtrate was concentrated under reduced pressure. The residue was triturated with diethyl ether (2×5 mL) and dried under high vacuum to afford 3-amino-3-(hydroxymethyl)pyrrolidin-2-one (Int-16—En2) (550 mg, Yield: 93%) as an off-white solid. ¹H NMR (400 MHz, DMSO-de) 5 ppm: 7.54 (s, 1H), 4.73 (brs, 1H), 3.36-3.32 (m, 1H), 3.18-3.12 (m, 2H), 3.10-3.04 (m, 1H), 2.22-2.15 (m, 1H), 1.76-1.71 (m, 1H), 1.57 (s, 2H).

### Synthesis of 5-(benzyloxy)-2-methylpyrazolo[1,5-a]pyridine-3-carboxylic acid (Cpd 033)

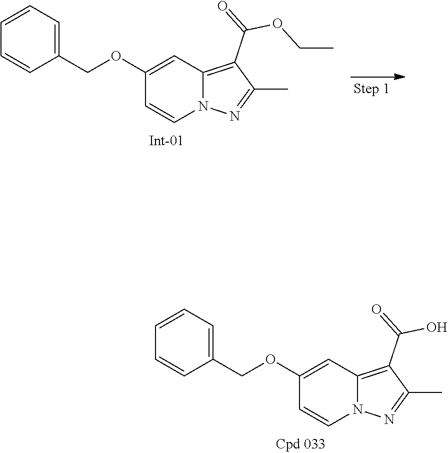 [SMILES: CCOC(=O)c1c(C)nn2ccc(OCc3ccccc3)cc12 ; Cc1nn2ccc(OCc3ccccc3)cc2c1C(=O)O]

Step 1: A sealed tube was charged with ethyl 5-(benzyloxy)-2-methylpyrazolo[1,5-a]pyridine-3-carboxylate (Int-01) (0.14 g, 0.45 mmol, 1 eq) and a mixture of EtOH/MeOH/THF/H2O (1/1/0.5/1.5 mL). Then potassium hydroxide (0.126 g; 2.26 mmol; 5 eq) was added. The tube was sealed and irradiated in a microwave oven at 160° C. for 30 min. The mixture was cooled to ambient temperature and the solvent was evaporated. The remaining was diluted with water and acidified with 2N hydrochloric acid. The precipitate was collected by filtration, washed with water, acetone then hot ethyl acetate and dried to afford 0.117 g (92%) of 5-(benzyloxy)-2-methylpyrazolo[1,5-a]pyridine-3-carboxylic acid (Cpd 033) as a white solid.

### Synthesis of 5-[(2-fluorophenyl)methoxy]-2-methylpyrazolo[1,5-a]pyridine-3-carboxylic acid (Cpd 035)

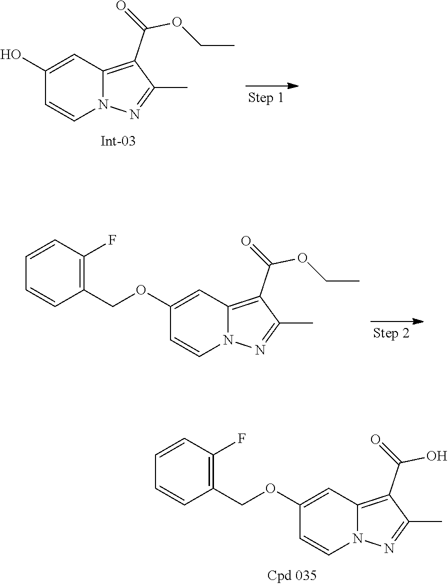 [SMILES: CCOC(=O)c1c(C)nn2ccc(O)cc12 ; CCOC(=O)c1c(C)nn2ccc(OCc3ccccc3F)cc12 ; Cc1nn2ccc(OCc3ccccc3F)cc2c1C(=O)O]

Step 1: To a stirred solution of ethyl 5-hydroxy-2-methylpyrazolo[1,5-a]pyridine-3-carboxylate (Int-03) (2.0 g, 9.09 mmol) in MeCN (25 mL) was added Cs₂CO₃ (5.9 g, 18.18 mmol) and followed by addition of 2-fluorobenzyl methanesulfonate (2.78 g, 13.6 mmol) at RT. The reaction mixture was heated to 80° C., stirred for 6 h at 80° C. The reaction mixture was filtered over celite bed, and celite bed was washed with ethyl acetate (2×30 mL). The filtrate was concentrated under reduced pressure to get crude. Crude was purified by column chromatography over silica gel (100-200 mesh) using 15% ethyl acetate in pet ether as an eluent to afford ethyl 5-((2-fluorobenzyl)oxy)-2-methylpyrazolo[1,5-a]pyridine-3-carboxylate (2.5 g, 83%) as an off-white solid. TLC system: 40% Ethyl acetate in Pet ether; RF: 0.6.

Step 2: To a stirred solution of ethyl 5-((2-fluorobenzyl)oxy)-2-methylpyrazolo[1,5-a]pyridine-3-carboxylate (2.0 g, 6.09 mmol) in EtOH:THF:H2O (1:1:1, 30 mL) was added NaOH (1.46 g, 36.5 mmol) at RT. The reaction mixture was stirred for 6 h at 90° C. The reaction mixture was concentrated, diluted with water (50 mL), acidified to pH -2 with 1N aq. HCl solution, and stirred for 30 minutes. The precipitated solid was filtered, washed with water (10 mL) and dried under vacuum to afford 5-[(2-fluorophenyl)methoxy]-2-methylpyrazolo[1,5-a]pyridine-3-carboxylic acid (Cpd 035) (1.5 g, 82%) as an off-white solid. TLC system: 50% Ethyl acetate in Pet ether; RF: 0.2.

The following compound was prepared in a similar manner (use of appropriate reagents (chiral or racemic) and purification methods (including chiral HPLC or chiral SFC) known to the person skilled in the art) as described for Cpd 035: Cpd 034.

### Synthesis of 2-({5-[(2-fluorophenyl)methoxy]-2-methylpyrazolo[1,5-a]pyridin-3-yl}formamido)-3-hydroxy-2-methylpropanamide (Cpd 020)

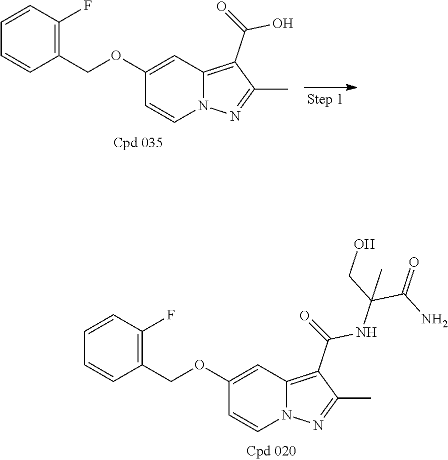 [SMILES: Cc1nn2ccc(OCc3ccccc3F)cc2c1C(=O)O ; Cc1nn2ccc(OCc3ccccc3F)cc2c1C(=O)NC(C)(CO)C(N)=O]

Step 1: To a stirred solution of 5-((2-fluorobenzyl)oxy)-2-methylpyrazolo[1,5-a]pyridine-3-carboxylic acid (Cpd 035) (500 mg, 1.6 mmol) in DMF (10 mL) was added HATU (1.26 g, 3.3 mmol), DIPEA (0.6 mL, 3.3 mmol) at 0° C. and followed by addition of 2-amino-3-hydroxy-2-methylpropanamide (600 mg, crude). The reaction mixture was stirred for 16 h at RT. The reaction mixture was diluted with water (25 mL) and extracted with ethyl acetate (3×20 mL). Combined organic layers were washed with water (2×25 mL), dried over anhydrous Na₂SO₄ and concentrated under reduced pressure to get crude. Crude was purified by grace column chromatography using 0.1% formic acid in water and acetonitrile as an eluent to afford 2-({5-[(2-fluorophenyl)methoxy]-2-methylpyrazolo[1,5-a]pyridin-3-yl}formamido)-3-hydroxy-2-methylpropanamide (Cpd 020) (90 mg, 14%) as an off-white solid. TLC system: 70% Ethyl acetate in Pet ether; RF: 0.1.

A preparative chiral SFC was performed on the racemic mixture of Cpd 020 to afford Cpd 020—En1 and Cpd 020—En2.

The following compounds were prepared in a similar manner (use of appropriate reagents (chiral or racemic) and purification methods (including chiral HPLC or chiral SFC) known to the person skilled in the art) as described for Cpd 020—En1 and Cpd 020—En2: Cpd 001, Cpd 002, Cpd 003, Cpd 004, Cpd 005, Cpd 006, Cpd 007, Cpd 008, Cpd 010, Cpd 012, Cpd 013, Cpd 014, Cpd 025—En1, Cpd 025—En2, Cpd 031—En1, Cpd 031—En2, Cpd 055, Cpd 056, Cpd 057, Cpd 058—En1, Cpd 058—En2, Cpd 059—En1, Cpd 059—En2, Cpd 061—En1, Cpd 061—En2, Cpd 062—En1, Cpd 062—En2, Cpd 063—En1, Cpd 063—En2, Cpd 064—En1, Cpd 064—En2, Cpd 066—En1, Cpd 066—En2, Cpd 068—En1, Cpd 068—En2, Cpd 074—En1, Cpd 074—En2, Cpd 081—En1, Cpd 081—En2, Cpd 090—En1, Cpd 090—En2, Cpd 091—En1, Cpd 091—En2.

### Synthesis of 5-[(2-fluorophenyl)methoxy]-N-[3-(hydroxymethyl)-2-oxopyrrolidin-3-yl]-2-methylpyrazolo[1,5-a]pyridine-3-carboxamide (Cpd 018)

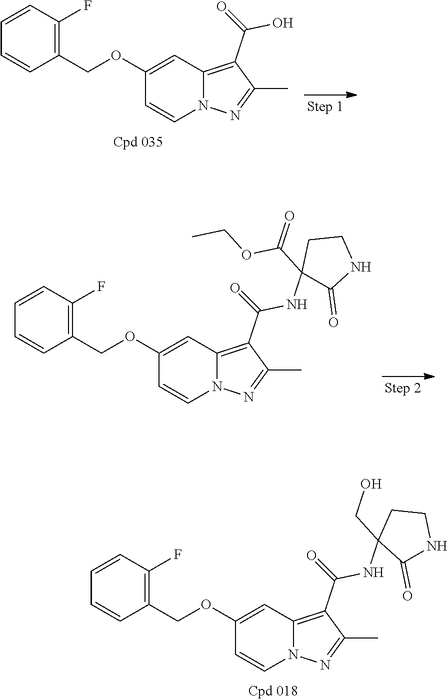 [SMILES: Cc1nn2ccc(OCc3ccccc3F)cc2c1C(=O)O ; CCOC(=O)C1(NC(=O)c2c(C)nn3ccc(OCc4ccccc4F)cc23)CCNC1=O ; Cc1nn2ccc(OCc3ccccc3F)cc2c1C(=O)NC1(CO)CCNC1=O]

Step 1: To a stirred solution of 5-((2-fluorobenzyl)oxy)-2-methylpyrazolo[1,5-a]pyridine-3-carboxylic acid (Cpd 035) (500 mg, 1.60 mmol) in DMF (10 mL) was added HATU (1.26 g, 3.3 mmol), DIPEA (0.6 mL, 3.3 mmol) at 0° C. and followed by addition of ethyl 3-amino-2-oxopyrrolidine-3-carboxylate (429 mg, 2.4 mmol). The reaction mixture was stirred for 16 h at RT and reaction progress was monitored by TLC. The reaction mixture was diluted with water (25 mL) and extracted with ethyl acetate (2×25 mL). Combined organic layers were washed with water (2×25 mL), dried over anhydrous Na₂SO₄ and concentrated under reduced pressure to get crude. Crude was purified by grace column chromatography using 0.1% formic acid in water and acetonitrile as an eluent to afford ethyl 3-(5-((2-fluorobenzyl)oxy)-2-methylpyrazolo[1,5-a]pyridine-3-carboxamido)-2-oxopyrrolidine-3-carboxylate (300 mg, 40%) as a white solid. TLC system: 50% Ethyl acetate in Pet ether; RF: 0.15.

Step 2: To a stirred solution of ethyl 3-(5-((2-fluorobenzyl)oxy)-2-methylpyrazolo[1,5-a]pyridine-3-carboxamido)-2-oxopyrrolidine-3-carboxylate (300 mg, 0.66 mmol) in methanol (20 mL) was added NaBH₄ (100 mg, 2.64 mmol) at 0° C. The reaction mixture was stirred for 1 h at RT and the reaction progress was monitored by TLC. The reaction mixture was concentrated, diluted with ice water (20 mL), and stirred for 15 min. The precipitated solid was filtered and dried under vacuum to afford 5-[(2-fluorophenyl)methoxy]-N-[3-(hydroxymethyl)-2-oxopyrrolidin-3-yl]-2-methylpyrazolo[1,5-a]pyridine-3-carboxamide (Cpd 018) (210 mg, 77%) as a white solid. TLC system: 70% Ethyl acetate in pet ether; RF: 0.15.

A preparative chiral SFC was performed on the racemic mixture of Cpd 018 to afford Cpd 018—En1 and Cpd 018—En2.

### Synthesis of 2-({5-[(2-fluorophenyl)methoxy]-2-methylpyrazolo[1,5-a]pyridin-3-yl}formamido)-3-hydroxy-N,2-dimethylpropanamide (Cpd 011)

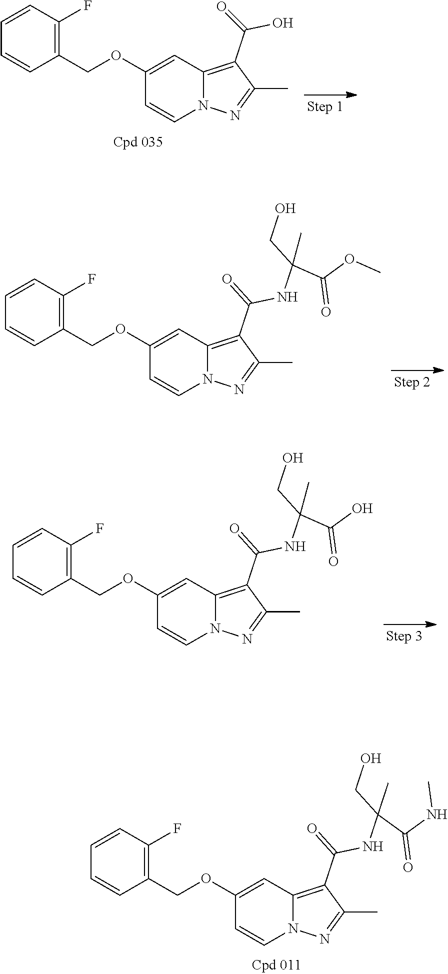 [SMILES: Cc1nn2ccc(OCc3ccccc3F)cc2c1C(=O)O ; COC(=O)C(C)(CO)NC(=O)c1c(C)nn2ccc(OCc3ccccc3F)cc12 ; Cc1nn2ccc(OCc3ccccc3F)cc2c1C(=O)NC(C)(CO)C(=O)O ; CNC(=O)C(C)(CO)NC(=O)c1c(C)nn2ccc(OCc3ccccc3F)cc12]

Step 1: To a solution of 5-((2-fluorobenzyl)oxy)-2-methylpyrazolo [1,5-a]pyridine-3-carboxylic acid (Cpd 035) (1.2 g, 4.00 mmol), HATU (3.0 g, 8.00 mmol) and DIPEA (2.6 mL, 16.00 mmol) in DMF (20 mL) was added methyl 2-amino-3-hydroxy-2-methylpropanoate (798 mg, 6.00 mmol) at 0° C. under argon atmosphere. The resulting reaction mixture was stirred at room temperature for 16 h and reaction progress was monitored by TLC. The reaction mixture was diluted with ice water (50 mL) and the precipitated solid was collected by filtration. The crude was purified by GRACE flash chromatography using 0.1% formic acid in water and acetonitrile as an eluent to afford methyl 2-(5-((2-fluorobenzyl)oxy)-2-methylpyrazolo[1,5-a]pyridine-3-carboxamido)-3-hydroxy-2-methylpropanoate (1.0 g, 62%) as an off-white solid. TLC system: 50% Ethyl acetate in pet-ether; RF: 0.2.

Step 2: A solution of NaOH (192.7 mg, 4.81 mmol) in water (15 mL) was added to a stirred solution of methyl 2-(5-((2-fluorobenzyl)oxy)-2-methylpyrazolo[1,5-a]pyridine-3-carboxamido)-3-hydroxy-2-methylpropanoate (1.0 g, 2.40 mmol) in MeOH (20 mL) and THE (20 mL) at RT. The RM was stirred for 16 h at 70° C. The reaction mixture was concentrated under reduced pressure, diluted with water (20 mL), pH was adjusted to -3 with 1 N aq. HCl solution and extracted with ethyl acetate (3×50 mL). Combined organic layers were dried over anhydrous Na₂SO₄ and concentrated under reduced pressure to afford 2-(5-((2-fluorobenzyl)oxy)-2-methylpyrazolo[1,5-a]pyridine-3-carboxamido)-3-hydroxy-2-methylpropanoic acid (700 mg, 72%) as an off-white solid. Proceeded to next step without purification. TLC system: 10% MeOH in dichloromethane; RF: 0.2.

Step 3: To a solution of 2-(5-((2-fluorobenzyl)oxy)-2-methylpyrazolo[1,5-a]pyridine-3-carboxamido)-3-hydroxy-2-methylpropanoic acid (300 mg, 074 mmol), HATU (568 mg, 1.49 mmol) and DIPEA (0.49 mL, 2.99 mmol) in DMF (10 mL) was added methenamine hydrochloride (151 mg, 2.24 mmol) at 0° C. under argon atmosphere. The resulting reaction mixture was stirred at room temperature for 16 h and reaction progress was monitored by TLC. The reaction mixture was diluted with ice water (50 mL) and the precipitated solid was collected by filtration to give crude. Crude was purified by GRACE flash chromatography using 0.1% formic acid in water and acetonitrile as an eluent to afford 2-({5-[(2-fluorophenyl)methoxy]-2-methylpyrazolo[1,5-a]pyridin-3-yl}formamido)-3-hydroxy-N,2-dimethylpropanamide (Cpd 011) (200 mg; 64%) as an off-white solid. TLC system: 50% Ethyl acetate in pet-ether; RF: 0.3.

A preparative chiral SFC was performed on the racemic mixture of Cpd 011 to afford Cpd 011—En1 and Cpd 011—En2.

The following compounds were prepared in a similar manner (use of appropriate reagents (chiral or racemic) and purification methods (including chiral HPLC or chiral SFC) known to the person skilled in the art) as described for Cpd 011—En1 and Cpd 011—En2 Cpd 009—En1, Cpd 009—En2, Cpd 017—En1, Cpd 017—En2,

### Synthesis of 5-[(2-chlorophenyl)methoxy]-2-methylpyrazolo[1,5-a]pyridine-3-carboxylic acid (Cpd 036)

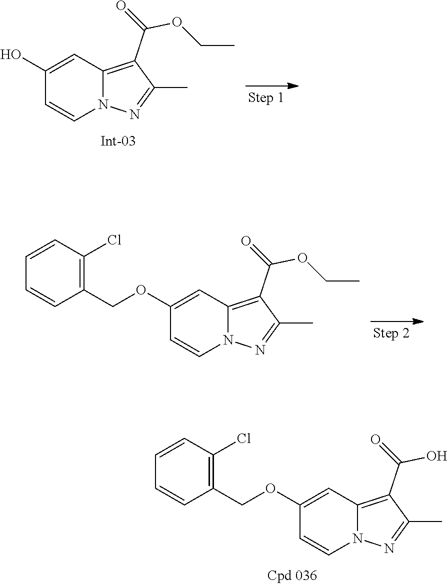 [SMILES: CCOC(=O)c1c(C)nn2ccc(O)cc12 ; CCOC(=O)c1c(C)nn2ccc(OCc3ccccc3Cl)cc12 ; Cc1nn2ccc(OCc3ccccc3Cl)cc2c1C(=O)O]

Step 1: To a pre-stirred solution of ethyl 5-hydroxy-2-methylpyrazolo[1,5-a]pyridine-3-carboxylate (Int-03) (500 mg, 2.272 mmol) and (2-chlorophenyl)methanol (645.4 mg, 4.545 mmol) in THE (30 mL) was added ADDP (1.1 g, 4.545 mmol) and n-Bu3P (0.918 g, 4.545 mmol) at 0° C. The reaction mixture was stirred for 16 h at RT and reaction progress was monitored by TLC. The reaction mixture was diluted with water (100 mL) and extracted with ethyl acetate (2×100 mL). Combined organic layers were washed with brine (100 mL), dried over anhydrous Na₂SO₄ and concentrated under reduced pressure to get crude. Crude was purified by column chromatography using 5% ethyl acetate in pet ether as an eluent to afford ethyl 5-((2-chlorobenzyl)oxy)-2-methylpyrazolo[1,5-a]pyridine-3-carboxylate (550 mg, 70%) as an off-white solid. TLC system: 30% Ethyl acetate in pet ether; RF: 0.5.

Step 2: To a pre-stirred solution of ethyl 5-((2-chlorobenzyl)oxy)-2-methylpyrazolo[1,5-a]pyridine-3-carboxylate (500 mg, 1.92 mmol) in Ethanol (15 mL) was added 2N NaOH solution (10 mL) at RT and the resulting reaction mixture was stirred for 16 h at 80° C. The reaction progress was monitored by TLC. Reaction mixture was concentrated under reduced pressure and diluted with water (40 mL), pH was adjusted to -3 with 1N aq. HCl solution. Solid precipitated was collected by filtration, dried under vacuum to afford 5-[(2-chlorophenyl)methoxy]-2-methylpyrazolo[1,5-a]pyridine-3-carboxylic acid (Cpd 036) (450 mg, 97%) as a pale yellow solid. TLC system: 50% Ethyl acetate in pet ether; RF: 0.2

### Synthesis of 2-({5-[(2-chlorophenyl)methoxy]-2-methylpyrazolo[1,5-a]pyridin-3-yl}formamido)-3-hydroxy-2-methylpropanamide (Cpd 019)

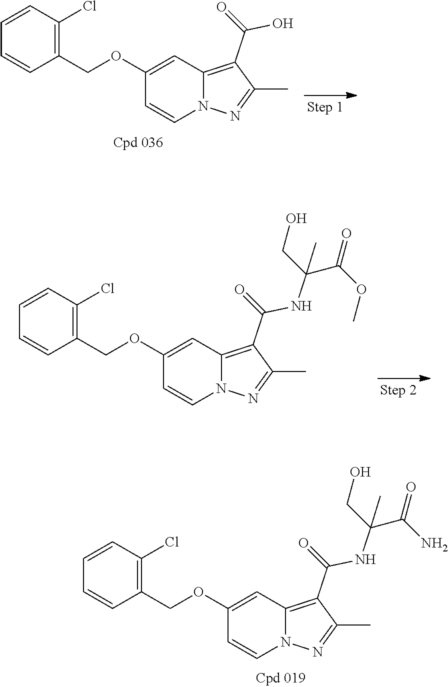 [SMILES: Cc1nn2ccc(OCc3ccccc3Cl)cc2c1C(=O)O ; COC(=O)C(C)(CO)NC(=O)c1c(C)nn2ccc(OCc3ccccc3Cl)cc12 ; Cc1nn2ccc(OCc3ccccc3Cl)cc2c1C(=O)NC(C)(CO)C(N)=O]

Step 1: Methyl 2-amino-3-hydroxy-2-methylpropanoate (420 mg, 3.164 mmol) was added to a stirred solution of 5-(benzyloxy)-2-methylpyrazolo[1,5-a]pyridine-3-carboxylic acid (Cpd 036) (500 mg, 1.582 mmol), HATU (1.2 g, 3.164 mmol) and DIPEA (1.4 mL, 7.911 mmol) in DMF (5 mL) at 0° C. and the resulting reaction mixture was stirred at room temperature for 16 h. The reaction mixture was diluted with water (60 mL) and extracted with 10% methanol in dichloromethane (2×20 mL). Combined organic layers were dried over anhydrous Na₂SO₄ and concentrated under reduced pressure to afford methyl 2-(5-((2-chlorobenzyl)oxy)-2-methylpyrazolo[1,5-a]pyridine-3-carboxamido)-3-hydroxy-2-methylpropanoate (450 mg, crude) as a white solid. TLC system: 5% Methanol in dichloromethane; RF: 0.01.

Step 2: Ammonia in methanol (7M) (25 mL) was added to a solution of methyl (5-((2-chlorobenzyl)oxy)-2-methylpyrazolo[1,5-a]pyridine-3-carbonyl)serinate (450 mg, crude) in a sealed tube. the reaction mixture was stirred at 90° C. for 16 h. The reaction was monitored by TLC. The reaction mixture was cooled and concentrated under reduced pressure to get crude. Crude was purified by GRACE flash chromatography using 45 to 50% acetonitrile and 0.1% formic acid in water as an eluent to afford 2-({5-[(2-chlorophenyl)methoxy]-2-methylpyrazolo[1,5-a]pyridin-3-yl}formamido)-3-hydroxy-2-methylpropanamide (Cpd 019) (120 mg, 23%) as an off-white solid. TLC system: 10% Methanol in dichloromethane; RF: 0.01

A preparative chiral SFC was performed on the racemic mixture of Cpd 019 to afford Cpd 019—En1 and Cpd 019—En2.

The following compounds were prepared in a similar manner (use of appropriate reagents (chiral or racemic) and purification methods (including chiral HPLC or chiral SFC) known to the person skilled in the art) as described for Cpd 019—En1 and Cpd 019—En2: Cpd 015—En1, Cpd 015—En2, Cpd 016—En1, Cpd 016—En2, Cpd 021—En1, Cpd 021—En2, Cpd 022—En1, Cpd 022—En2, Cpd 023—En1, Cpd 023—En2, Cpd 026—En1, Cpd 026—En2, Cpd 027—En1, Cpd 027—En2, Cpd 028—En1, Cpd 028—En2, Cpd 029—En1, Cpd 029—En2, Cpd 030—En1, Cpd 030—En2, Cpd 032—En1, Cpd 032—En2.

### Synthesis of2-(1-{5-[(2-fluorophenyl)methoxy]-2-methylpyrazolo[1,5-a]pyridin-3-yl}-N-methylformamido)-3-hydroxy-2-methylpropanamide (Cpd 024)

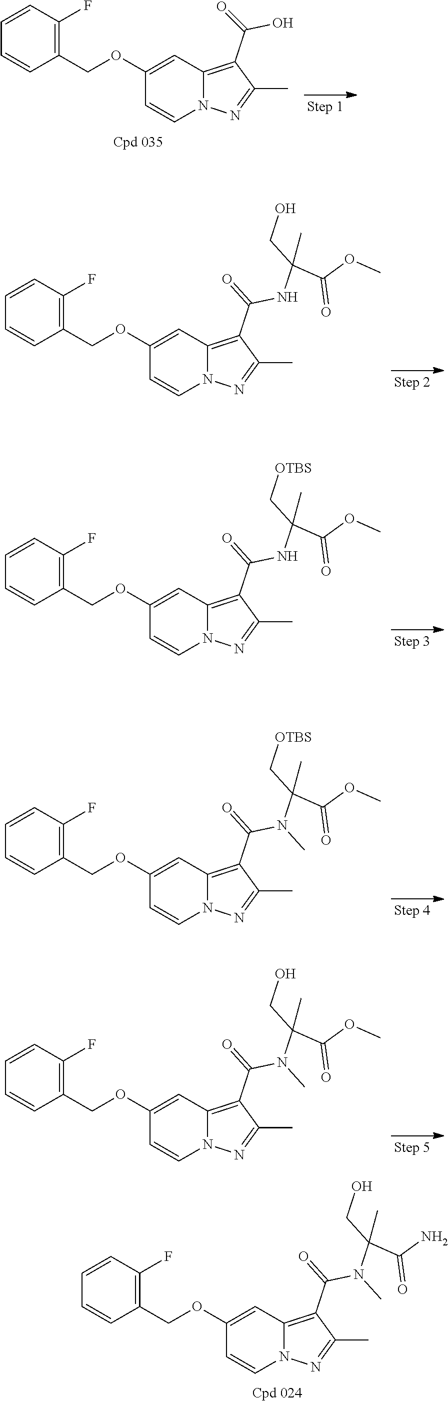 [SMILES: Cc1nn2ccc(OCc3ccccc3F)cc2c1C(=O)O ; COC(=O)C(C)(CO)NC(=O)c1c(C)nn2ccc(OCc3ccccc3F)cc12 ; COC(=O)C(C)(CO[Si](C)(C)C(C)(C)C)NC(=O)c1c(C)nn2ccc(OCc3ccccc3F)cc12 ; COC(=O)C(C)(CO[Si](C)(C)C(C)(C)C)N(C)C(=O)c1c(C)nn2ccc(OCc3ccccc3F)cc12 ; COC(=O)C(C)(CO)N(C)C(=O)c1c(C)nn2ccc(OCc3ccccc3F)cc12 ; Cc1nn2ccc(OCc3ccccc3F)cc2c1C(=O)N(C)C(C)(CO)C(N)=O]

Step 1: To a solution of 5-((2-fluorobenzyl)oxy)-2-methylpyrazolo [1,5-a]pyridine-3-carboxylic acid (Cpd 035) (1.2 g, 4.00 mmol), HATU (3.0 g, 8.00 mmol) and DIPEA (2.6 mL, 16.00 mmol) in DMF (20 mL) was added methyl 2-amino-3-hydroxy-2-methylpropanoate (798 mg, 6.00 mmol) at 0° C. under argon atmosphere. The resulting reaction mixture was stirred at room temperature for 16 h and reaction progress was monitored by TLC. The reaction mixture was diluted with ice water (50 mL) and the precipitated solid was collected by filtration. The crude was purified by GRACE flash chromatography using 0.1% formic acid in water and acetonitrile as an eluent to afford methyl 2-(5-((2-fluorobenzyl)oxy)-2-methylpyrazolo[1,5-a]pyridine-3-carboxamido)-3-hydroxy-2-methylpropanoate (1.0 g, 62%) as an off-white solid. TLC system: 50% Ethyl acetate in pet-ether; RF: 0.2.

Step 2: To a solution of methyl 2-(5-(2-fluorobenzyloxy)-2-methylpyrazolo[1,5-a]pyridine-3-carboxamido)-3-hydroxy-2-methylpropanoate (1.0 g, 2.409 mmol), in dichloromethane (50 mL) was added Imidazole (0.490 g, 7.228 mmol) and TBSCl (0.542 g, 3.614 mmol) at RT. The resulting reaction mixture was stirred at room temperature for 3 h and reaction progress was monitored by TLC. The reaction mixture was poured into ice water (150 mL) and extracted with dichloromethane (2×150 mL). Combined organic layers were washed with water (2×150 mL), brine (150 mL), dried over anhydrous Na₂SO₄ and concentrated under reduced pressure to afford methyl 3-(tert-butyldimethylsilyloxy)-2-(5-(2-fluorobenzyloxy)-2-methylpyrazolo[1,5-a]pyridine-3-carboxamido)-2-methylpropanoate (1.5 g, crude) as a pale brown gummy solid. TLC system: 50% ethyl acetate in n-Hexane RF: 0.6.

Step 3: To a solution of methyl 3-(tert-butyldimethylsilyloxy)-2-(5-(2-fluorobenzyloxy)-2-methylpyrazolo[1,5-a]pyridine-3-carboxamido)-2-methylpropanoate (1.5 g, 2.835 mmol) in THE (100 mL) was added NaH (216 mg, 5.671 mmol) and Methyl iodide (805 mg, 5.671 mmol) at 0° C. The RM was stirred at RT for 3 h and reaction progress was monitored by TLC. The reaction mixture was poured into ice water (150 mL) and extracted with ethyl acetate (2×150 mL). Combined organic layers were washed with water (2×150 mL), brine (150 mL), dried over anhydrous Na₂SO₄ and concentrated under reduced pressure to afford methyl 3-(tert-butyldimethylsilyloxy)-2-(5-(2-fluorobenzyloxy)-N,2-dimethylpyrazolo[1,5-a]pyridine-3-carboxamido)-2-methylpropanoate (1.5 g, Crude) as a pale brown gummy solid. TLC system: 30% ethyl acetate in n-Hexane RF: 0.2.

Step 4: To a solution of methyl 3-(tert-butyldimethylsilyloxy)-2-(5-(2-fluorobenzyloxy)-N,2-dimethylpyrazolo[1,5-a]pyridine-3-carboxamido)-2-methylpropanoate (1.5 g, 2.762 mmol) in THE (50 mL) was added TBAF in THE (1M, 8.2 mL, 8.287 mmol) at 0° C. The resulting reaction mixture was stirred at room temperature for 1 h and reaction progress was monitored by TLC. The reaction mixture was poured into ice water (150 mL) and extracted with ethyl acetate (2×100 mL). Combined organic layers were washed with water (2×100 mL), brine (100 mL), dried over anhydrous Na₂SO₄ and concentrated under reduced pressure to give crude. Crude product was purified by GRACE flash chromatography using 30 to 50% acetonitrile and 0.1% formic acid in water to afford methyl 2-(5-(2-fluorobenzyloxy)-N,2-dimethylpyrazolo[1,5-a]pyridine-3-carboxamido)-3-hydroxy-2-methylpropanoate as an off -white solid (500 mg, 39%). TLC system: 70% ethyl acetate in n-Hexane RF: 0.2.

Step 5: Ammonia in methanol (7M, 25 mL) was added to a solution of methyl 2-(5-(2-fluorobenzyloxy)-N,2-dimethylpyrazolo[1,5-a]pyridine-3-carboxamido)-3-hydroxy-2-methylpropanoate (450 mg) in a sealed tube. The reaction mixture was heated to 100° C. and stirred at 100° C. for 16 h. The reaction was monitored by TLC. The reaction mixture was cooled and concentrated under reduced pressure to get crude. Crude was purified by GRACE flash chromatography using 20 to 30% acetonitrile in 0.1% formic acid—water as an eluent to afford 2-(1-{5-[(2-fluorophenyl)methoxy]-2-methylpyrazolo[1,5-a]pyridin-3-yl}-N-methylformamido)-3-hydroxy-2-methylpropanamide (Cpd 024) (125 mg, 18%) as an off-white solid. TLC system: 10% MeOH in dichloromethane; RF: 0.01

A preparative chiral SFC was performed on the racemic mixture of Cpd 024 to afford Cpd 024—En1 and Cpd 024—En2.

### Synthesis of N-(4,4-difluoro-1-hydroxy-2-methylbutan-2-yl)-2-methyl-5-[(pyridin-2-yl)methoxy]pyrazolo[1,5-a]pyridine-3-carboxamide (Cpd 038)

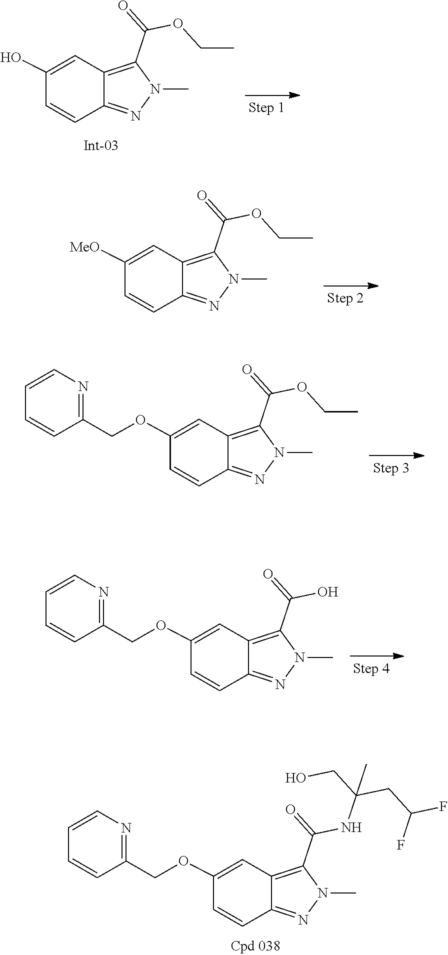 [SMILES: CCOC(=O)c1c2cc(O)ccc2nn1C ; CCOC(=O)c1c2cc(OC)ccc2nn1C ; CCOC(=O)c1c2cc(OCc3ccccn3)ccc2nn1C ; Cn1nc2ccc(OCc3ccccn3)cc2c1C(=O)O ; Cn1nc2ccc(OCc3ccccn3)cc2c1C(=O)NC(C)(CO)CC(F)F]

Step 1: To a solution of ethyl 5-hydroxy-2-methylpyrazolo[1,5-a]pyridine-3-carboxylate (Int-03) (10.0 g, 45.40 mmol) in DCM (100 mL) was added TEA (13.9 mL, 99.89 mmol) at RT. The RM was cooled to 0° C., MsCl (3.86 mL, 49.94 mmol) was added. The RM was stirred for 30 min at the 0° C. The RM was stirred for 4 h at RT. The RM was quenched with saturated NaHCO₃ (50 mL), extracted with DCM (2×100 mL). Combined organic layer was washed with brine (100 mL) and dried over Na₂SO₄, filtered and concentrated under reduced pressure to afford ethyl 2-methyl-5-((methylsulfonyl)oxy)pyrazolo[1,5-a]pyridine-3-carboxylate as a pale brown solid (12.4 g, 91.54%). ¹H NMR (400 MHz, CDCl₃) 5 ppm: 8.43 (d, 1H), 7.97 (d, 1H), 6.88 (dd, 1H), 4.38 (q, 2H), 3.26 (s, 3H), 2.67 (s, 3H), 1.43 (t, 3H). Step 2: To a solution of ethyl 2-methyl-5-((methylsulfonyl)oxy)pyrazolo[1,5-a]pyridine-3-carboxylate (6 g, 20.113 mmol) in DMF (60.0 ml) were added pyridin-2-ylmethanol (2.195 g, 20.113 mmol), Cs2CO₃ (9.830 g, 30.170 mmol) at RT. The RM was stirred for 16 h. The RM was quenched with water (100 mL), extracted with EtOAc (2×100 mL). The combined organic layers were washed with brine (100 mL), dried over Na₂SO₄, filtered and concentrated under reduced pressure. The residue was purified by FCC on silica gel using 30% EtOAc in pet ether as an eluent to afford ethyl 2-methyl-5-(pyridin-2-ylmethoxy)pyrazolo[1,5-a]pyridine-3-carboxylate as a white solid (4.5 g, 94%). ¹H NMR (400 MHz, CDCl₃) 5 ppm: 8.64 (d, 1H), 8.25 (d, 1H), 7.73-7.77 (m, 1H), 7.50 (d, 1H), 7.45 (d, 1H), 7.25-7.29 (m, 1H), 6.66 (dd, 1H), 5.29 (s, 2H), 4.35 (q, 2H), 2.62 (s, 3H), 1.39 (t, 3H).

Step 3: To a solution of ethyl 2-methyl-5-(pyridin-2-ylmethoxy)pyrazolo[1,5-a]pyridine-3-carboxylate (4.5 g, 14.454 mmol) in MeOH:THF:H₂O (1:1:0.5) (50.0 mL) was added NaOH (5.781 g, 144.536 mmol) at RT. The RM was stirred at 50° C. for 16 h. After completion, the volatiles were removed under reduced pressure. The residue was acidified with AcOH (pH˜4), precipitated solid was filtered, washed with water (50.0 mL) and dried to afford 2-methyl-5-(pyridin-2-ylmethoxy)pyrazolo[1,5-a]pyridine-3-carboxylic acid as an off-white solid (3.2 g). ¹H NMR (400 MHz, DMSO-de) 5 ppm: 12.16 (br, s, 1H), 8.59 (d, 2H), 7.86 (dt, 1H), 7.56 (d, 1H), 7.36-7.39 (m, 2H), 6.83 (dd, 1H), 5.29 (s, 2H), 2.50 (s, 3H).

Step 4: To a solution of 2-methyl-5-(pyridin-2-ylmethoxy)pyrazolo[1,5-a]pyridine-3-carboxylic acid (600 mg, 2.119 mmol) and HATU (1.208 g, 3.179 mmol) in DMF (10.0 mL) were added DIPEA (1.10 mL, 6.358 mmol) and 2-amino-4,4-difluoro-2-methylbutan-1-ol (442 mg, 3.179 mmol) at 0° C. The RM was stirred at RT for 2 h. After completion, the RM was diluted with cold water (50 mL), extracted with EtOAc (2×50 mL). The combined organic layers were washed with a saturated solution of NaHCO₃ (50 mL), brine (50 mL), dried over Na₂SO₄, filtered and concentrated under reduced pressure. The compound was purified by Prep. HPLC. [Prep. HPLC conditions: Mobile phase: 10 mM ABC in Water, Mobile phase B: ACN, Column: YMC TRIART C18 (25×150 mm) 5 μm, FLOW: 19 ml/min, Method: (T in min./% of B): 0/25, 2/25, 10/40, 15.50/40, 15.51/98, 18/98, 18.10/25, 21/25, Solubility: ACN+Water+THF, Temperature: RT.] The desired fractions were evaporated and lyophilized to afford N-(4,4-difluoro-1-hydroxy-2-methylbutan-2-yl)-2-methyl-5-[(pyridin-2-yl)methoxy]pyrazolo[1,5-a]pyridine-3-carboxamide (Cpd 038) as an off-white solid (400 mg, 46.67%).

A preparative chiral SFC was performed on the racemic mixture of Cpd 038 to afford Cpd 038—En1 and Cpd 038—En2.

The following compounds were prepared in a similar manner (use of appropriate reagents (chiral or racemic) and purification methods (including chiral HPLC or chiral SFC) known to the person skilled in the art) as described for Cpd 038—En1 and Cpd 038—En2: Cpd 037—En1, Cpd 037—En2, Cpd 042, Cpd 043, Cpd 045, Cpd 046, Cpd 047, Cpd 048,

### Synthesis of N-[(1H-imidazol-2-yl)methyl]-2-methyl-5-[(pyridin-2-yl)methoxy]pyrazolo[1,5-a]pyridine-3-carboxamide (Cpd 044)

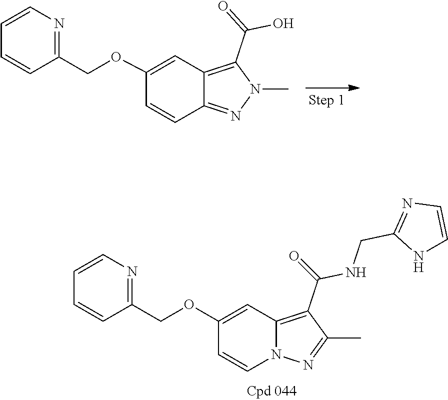 [SMILES: Cn1nc2ccc(OCc3ccccn3)cc2c1C(=O)O ; Cc1nn2ccc(OCc3ccccn3)cc2c1C(=O)NCc1ncc[nH]1]

Step 1: To a solution of 2-methyl-5-(pyridin-2-ylmethoxy)pyrazolo[1,5-a]pyridine-3-carboxylic acid (200 mg, 0.706 mmol), EDCl (202 mg, 1.059 mmol) and 1-Hydroxybenzotriazole hydrate (143 mg, 1.059 mmol) in DMF (5.0 mL) were added DIPEA (0.65 mL, 3.53 mmol) and (1H-imidazol-2-yl)methanamine dihydrochloride (238 mg, 1.412 mmol) at 0° C. The RM stirred at RT for 16 h. After completion, the RM was diluted with cold water (30 mL), extracted with EtOAc (2×100 mL). The combined organic layers were washed with saturated NaHCO₃ solution (30 mL), brine (30 mL), dried over Na₂SO₄, filtered and concentrated under reduced pressure. The compound was purified by Prep. HPLC. [Prep. HPLC conditions: Mobile phase: 10 mM ABC in Water, Mobile phase B: ACN, Column: XB, C18 (250×19) mm, 5 μm, FLOW: 13 ml/min, Method: (T in min./% of B): 0/20, 2/20, 12/45, Solubility: ACN+Water+DMSO, Temperature: RT.] The desired fractions were evaporated and lyophilized to afford N-[(1H-imidazol-2-yl)methyl]-2-methyl-5-[(pyridin-2-yl)methoxy]pyrazolo[1,5-a]pyridine-3-carboxamide (Cpd 044) as an off-white solid (58 mg, 22%).

### Synthesis of N-(4,4-difluoropiperidin-3-yl)-2-methyl-5-[(pyridin-2-yl)methoxy]pyrazolo[1,5-a]pyridine-3-carboxamide (Cpd 050)

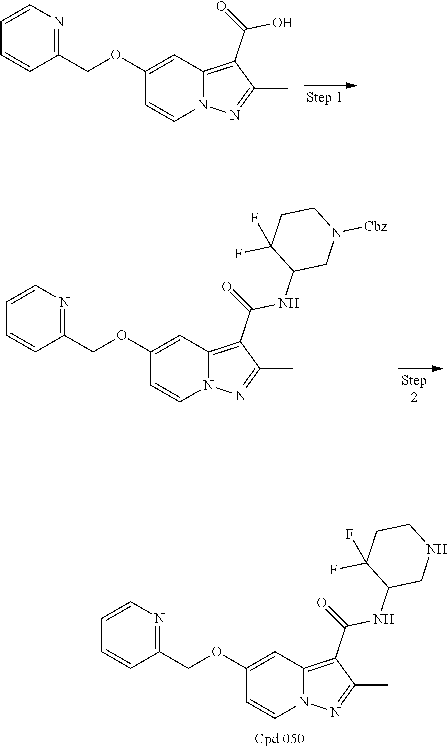 [SMILES: Cc1nn2ccc(OCc3ccccn3)cc2c1C(=O)O ; Cc1nn2ccc(OCc3ccccn3)cc2c1C(=O)NC1CN(C(=O)OCc2ccccc2)CCC1(F)F ; Cc1nn2ccc(OCc3ccccn3)cc2c1C(=O)NC1CNCCC1(F)F]

Step 1: To a solution of 2-methyl-5-(pyridin-2-ylmethoxy)pyrazolo[1,5-a]pyridine-3-carboxylic acid (250 mg, 0.882 mmol) in DMF (10 mL) were added HATU (500 mg, 1.32 mmol), DIPEA (0.5 mL, 2.65 mmol) and tert-butyl 3-amino-4,4-difluoropiperidine-1-carboxylate (310 mg, 1.32 mmol) at 0° C. The RM was stirred at RT for 16 h. After completion, the RM was diluted with cold water (30 mL), extracted with EtOAc (2×30 mL). The combined organic layers were washed with sat. bicarbonate solution (30 mL), brine (30 mL), dried Na₂SO₄, filtered and concentrated under reduced pressure to afford tert-butyl 4,4-difluoro-3-(2-methyl-5-(pyridin-2-ylmethoxy) pyrazolo[1,5-a]pyridine-3-carboxamido) piperidine-1-carboxylate as off-white solid. (325 mg, 73%). ¹H NMR (400 MHz, DMSO-de) 5 ppm: 8.60-8.55 (m, 2H), 7.88-7.83 (m, 1H), 7.57-7.53 (m, 2H), 7.39-7.36 (m, 1H), 7.23 (s, 1H), 6.79-6.76 (m, 1H), 5.26 (s, 2H), 4.50-4.32 (m, 1H), 3.95-3.72 (m, 2H), 3.15 (t, 2H), 2.4-2.21 (m, 1H), 2.15-1.95 (m, 1H), 1.46 (s, 9H) (CH3 protons merged in DMSO peak).

Step 2: To a solution of tert-butyl 4,4-difluoro-3-(2-methyl-5-(pyridin-2-ylmethoxy) pyrazolo[1,5-a]pyridine-3-carboxamido) piperidine-1-carboxylate (300 mg, 0.598 mmol) in DCM (10 mL) was added 4N HCl in dioxane (0.8 mL) at 0° C. The RM was stirred at RT for 2 h. After completion, the RM was concentrated under reduced pressure. The residue was triturated with n-pentane (20 mL) and dried to afford crude compound as an off white solid (HCl salt). The solid was dissolved in water (25 mL), basified with sat sodium bicarbonate solution (pH˜10), extracted with EtOAc (2×25 mL). Combined organic layers were washed with brine (30 mL), dried over sodium sulphate, concentrated under reduced pressure to afford N-(4,4-difluoropiperidin-3-yl)-2-methyl-5-[(pyridin-2-yl)methoxy]pyrazolo[1,5-a]pyridine-3-carboxamide (Cpd 050).as off white solid (170 mg, 71%).

A preparative chiral SFC was performed on the racemic mixture of Cpd 050 to afford Cpd 050—En1 and Cpd 050—En2.

The following compounds were prepared in a similar manner (use of appropriate reagents (chiral or racemic) and purification methods (including chiral HPLC or chiral SFC) known to the person skilled in the art) as described for Cpd 050—En1 and Cpd 050—En2 Cpd 049—En1, Cpd 049—En2, Cpd 084—En1, Cpd 084—En2.

### Synthesis of N-(5,5-difluoropiperidin-3-yl)-2-methyl-5-[(pyridin-2-yl)methoxy]pyrazolo[1,5-a]pyridine-3-carboxamide (Cpd 052)

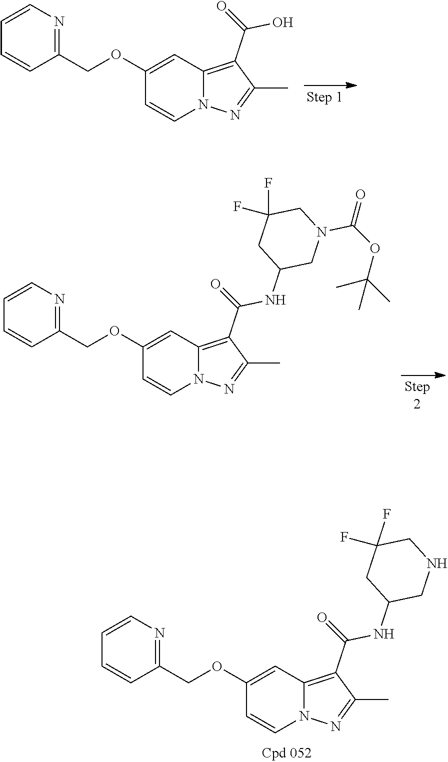 [SMILES: Cc1nn2ccc(OCc3ccccn3)cc2c1C(=O)O ; Cc1nn2ccc(OCc3ccccn3)cc2c1C(=O)NC1CN(C(=O)OC(C)(C)C)CC(F)(F)C1 ; Cc1nn2ccc(OCc3ccccn3)cc2c1C(=O)NC1CNCC(F)(F)C1]

Step 1: To a solution of 2-methyl-5-(pyridin-2-ylmethoxy)-2H-indazole-3-carboxylic acid (300 mg, 1.059 mmol) in DMF (20 mL) were added HATU (805.3 mg, 2.118 mmol), DIPEA (0.92 mL, 5.295 mmol) and tert-butyl 5-amino-3,3-difluoropiperidine-1-carboxylate (300.2 mg, 1.271 mmol) at 0° C. The RM was stirred at RT for 16 h. After completion, the RM was treated with ice-cold water (30 mL), extracted with EtOAc (2×40 mL). Combined organic layers were washed with saturated NaHCO₃ (20 mL), brine (20 mL), dried over Na₂SO₄, filtered and concentrated under reduced pressure. The residue was purified by FCC on silica gel using 2% MeOH in DCM as an eluent to afford tert-butyl,3,3-difluoro-5-(2-methyl-5-(pyridin-2-ylmethoxy)pyrazolo[1,5-a]pyridine-3-carboxamido) piperidine-1-carboxylate as a pale yellow liquid (500 mg).

Step 2: To a solution of N-(5,5-difluoropiperidin-3-yl)-2-methyl-5-(pyridin-2-ylmethoxy)pyrazolo[1,5-a]pyridine-3-carboxamide (500 mg, 0.998 mmol) in DCM (30 mL) was added 4N HCl in 1,4-Dioxane (2.0 mL) at 0° C. The RM was stirred at RT for 3 h. After completion, the RM was concentrated under reduced pressure. The residue was basified with a saturated solution of NaHCO₃ (20 mL), extracted with DCM (2×30 mL). Combined organic layer was dried over Na₂SO₄, filtered and concentrated under reduced pressure. The compound was purified by Prep. HPLC. [Prep. HPLC conditions: Mobile phase: 10 mM ABC in Water, Mobile phase B: ACN, Column: X-SELECT CSH C18(250×19 mm) 5 μm, FLOW: 17 ml/min, Method: (T in min./% of B): 0/15, 2/15, 10/35, 15/35, 15.1/100, 20/100, 20.10/15, 23/15, Solubility: ACN+Water+THF, Temperature: RT.] The desired fractions were evaporated and lyophilized to afford N-(5,5-difluoropiperidin-3-yl)-2-methyl-5-[(pyridin-2-yl)methoxy]pyrazolo[1,5-a]pyridine-3-carboxamide (Cpd 052) (110 mg, 27.49%).

A preparative chiral SFC was performed on the racemic mixture of Cpd 052 to afford Cpd 052—En1 and Cpd 052—En2.

The following compounds were prepared in a similar manner (use of appropriate reagents (chiral or racemic) and purification methods (including chiral HPLC or chiral SFC) known to the person skilled in the art) as described for Cpd 052—En1 and Cpd 052—En2: Cpd 051—En1, Cpd 051—En2, Cpd 053—En1, Cpd 053—En2, Cpd 054—En1, Cpd 054—En2, Cpd 082—En1, Cpd 082—En2

### Synthesis of N-[2,2-difluoro-1-(hydroxymethyl)cyclopropyl]-2-methyl-5-[(pyridin-2-yl)methoxy]pyrazolo[1,5-a]pyridine-3-carboxamide (Cpd 071)

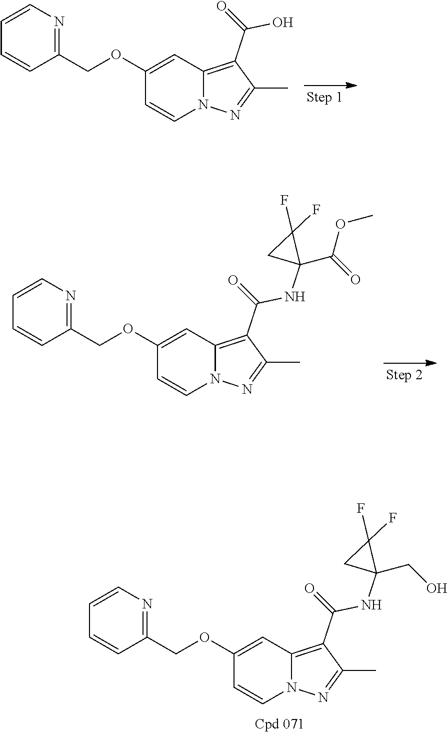 [SMILES: Cc1nn2ccc(OCc3ccccn3)cc2c1C(=O)O ; COC(=O)C1(NC(=O)c2c(C)nn3ccc(OCc4ccccn4)cc23)CC1(F)F ; Cc1nn2ccc(OCc3ccccn3)cc2c1C(=O)NC1(CO)CC1(F)F]

Step 1: To a solution of 2-methyl-5-(pyridin-2-ylmethoxy)pyrazolo[1,5-a]pyridine-3-carboxylic acid (400 mg, 1.412 mmol) in THE (10 mL) were added PyBroP (987.36 mg, 2.118 mmol), DIPEA (1.231 mL, 7.060 mmol) at RT. The RM stirred for 10 min. After 10 min, methyl 1-amino-2,2-difluorocyclopropane-1-carboxylate hydro bromide (655.23 mg, 2.824 mmol) was added. The RM was stirred at RT for 6 h. After completion, the RM was diluted with ice-cold water (20 mL), extracted with EtOAc (2×30 mL). Combined organic layers were washed with saturated solution of NaHCO₃ (20 mL), brine (20 mL), dried over Na₂SO₄, filtered and concentrated under reduced pressure. The residue was purified by FCC on silica gel using 5% MeOH in DCM as an eluent to afford methyl 2,2-difluoro-1-(2-methyl-5-(pyridin-2-ylmethoxy)pyrazolo[1,5-a]pyridine-3-carboxamido) cyclopropane-1-carboxylate as an off white solid (190 mg, 32.32%). ¹H NMR (400 MHz, DMSO-de) 6 ppm: 8.57-8.60 (m, 2H), 8.40 (d, 1H), 7.84-7.88 (dt, 1H), 7.50 (d, 1H), 7.36-7.39 (dt, 1H), 7.31 (d, 1H), 6.78-6.81 (dd, 1H), 5.28 (s, 2H), 3.70 (s, 3H), 2.60-2.67 (m, 1H), 2.10-2.13 (m, 1H).

Step 2: To a solution of methyl 2,2-difluoro-1-(2-methyl-5-(pyridin-2-ylmethoxy)pyrazolo[1,5-a]pyridine-3-carboxamido) cyclopropane-1-carboxylate (180 mg, 0.432 mmol) in ethanol (5.0 mL) was added NaBH₄ (49.06 mg, 1.297 mmol) portion wise at 0° C. The RM was stirred at RT for 16 h. After completion, the RM was concentrated under reduced pressure and the obtained residue was diluted with water (30 mL) and extracted with DCM (2×30 mL). Organic layer was dried over Na₂SO₄, filtered and concentrated under reduced pressure. The compound was purified by Prep. HPLC. [Prep. HPLC conditions: Mobile phase: 0.1% FA in water, Mobile phase B: ACN, Column: LUNA C 18 (21.2×250) 5 μm, FLOW: 16 ml/min, Method: (T in min./% of B): 0/10, 2/10, 10/30, 14/30, 14.1/98, 17/98, 17.1/10, 20/10. Solubility: ACN+Water+THF, Temperature: RT.] The desired fractions were evaporated and lyophilized to afford N-[2,2-difluoro-1-(hydroxymethyl)cyclopropyl]-2-methyl-5-[(pyridin-2-yl)methoxy]pyrazolo[1,5-a]pyridine-3-carboxamide (Cpd 071) as an off-white solid (13 mg, 7.74%).

### Synthesis of N-[cis-2-(difluoromethyl)-1-(hydroxymethyl)cyclopropyl]-2-methyl-5-[(pyridin-2-yl)methoxy]pyrazolo[1,5-a]pyridine-3-carboxamide (Cpd 072)

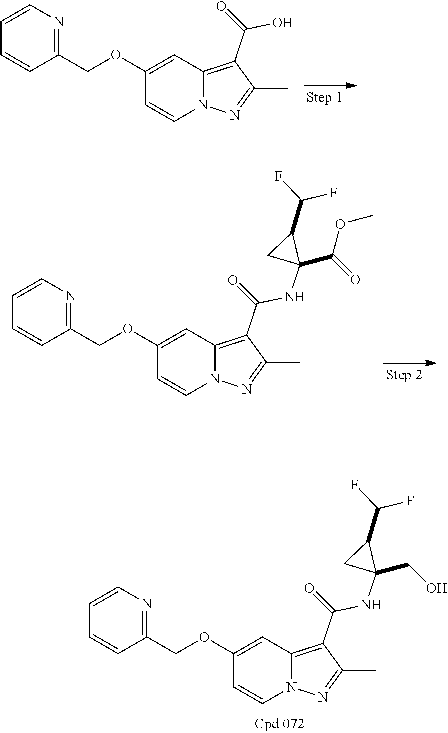 [SMILES: Cc1nn2ccc(OCc3ccccn3)cc2c1C(=O)O ; COC(=O)[C@@]1(NC(=O)c2c(C)nn3ccc(OCc4ccccn4)cc23)C[C@H]1C(F)F ; Cc1nn2ccc(OCc3ccccn3)cc2c1C(=O)N[C@]1(CO)C[C@H]1C(F)F]

Step 1: To a solution of 2-methyl-5-(pyridin-2-ylmethoxy)pyrazolo[1,5-a]pyridine-3-carboxylic acid (300 mg, 1.059 mmol) in DMF (5.0 mL) were added HATU (603.99 mg, 1.588 mmol), DIPEA (0.923 mL, 5.295 mmol) at RT and stirred for 10 min. After 10 min, methyl 1-amino-2-(difluoromethyl)cyclopropane-1-carboxylate hydrochloride (320.23 mg, 1.588 mmol) was added. The RM was stirred at 50° C. for 48 h. After completion, the RM was diluted with ice-cold water (20 mL), extracted with DCM (2×20 mL). Combined organic layers were washed with saturated NaHCO₃ (20 mL), brine (20 mL), dried over Na₂SO₄, filtered and concentrated under reduced pressure to afford methyl 2-(difluoromethyl)-1-(2-methyl-5-(pyridin-2-ylmethoxy)pyrazolo[1,5-a]pyridine-3-carboxamido) cyclopropane-1-carboxylate as a pale yellow solid (240 mg, yield: 52.65%). ¹H NMR (400 MHz, DMSO-de) 6 ppm: 8.60 (d, 1H), 8.55 (d, 1H), 8.27 (s, 1H), 7.84-7.88 (dt, 1H), 7.56 (d, 1H), 7.36-7.39 (dt, 1H), 7.28 (d, 1H), 6.75-6.78 (dd, 1H), 5.82-6.12 (dt, 1H), 5.30 (s, 2H), 3.65 (s, 3H), 2.49 (s, 3H), 2.20-2.23 (m, 1H), 1.87 (t, 1H), 1.52-1.56 (m, 1H).

Step 2: To a solution of methyl 2-(difluoromethyl)-1-(2-methyl-5-(pyridin-2-ylmethoxy)pyrazolo[1,5-a]pyridine-3-carboxamido)cyclopropane-1-carboxylate (240 mg, 0.558 mmol) in ethanol (10 mL) was added NaBH₄ (105.38 mg, 2.788 mmol) portion wise at 0° C. The RM was stirred at RT for 16 h. After completion, the RM was concentrated under reduced pressure. The residue was diluted with water (30 mL), extracted with DCM (2×30 mL). Combined organic layer was dried over Na₂SO₄, filtered and concentrated under reduced pressure. The compound was purified by Prep. HPLC. [Prep. HPLC conditions: Mobile phase: 10 mM ABC in water, Mobile phase B: ACN, Column: YMC C18 (25×150) mm, 5 μm, FLOW: 22 ml/min, Method: (T in min./% of B): 0/25, 2/25, 10/55, 13/55, 13.2/100, 17/100, 17.1/25, 20/25, Solubility: ACN+Water+THF, Temperature: RT.] The desired fractions were evaporated and lyophilized to afford N-[cis-2-(difluoromethyl)-1-(hydroxymethyl)cyclopropyl]-2-methyl-5-[(pyridin-2-yl)methoxy]pyrazolo[1,5-a]pyridine-3-carboxamide (Cpd 072) as an off-white solid (130 mg, 57.94%).

A preparative chiral SFC was performed on the racemic mixture of Cpd 072 to afford Cpd 072—En1 and Cpd 072—En2.

The following compounds were prepared in a similar manner (use of appropriate reagents (chiral or racemic) and purification methods (including chiral HPLC or chiral SFC) known to the person skilled in the art) as described for Cpd 072—En1 and Cpd 072—En2: Cpd 075—En1, Cpd 075—En2, Cpd 077—En1, Cpd 077—En2, Cpd 079—En1, Cpd 079—En2, Cpd 087—En1, Cpd 087—En2, Cpd 088—En1, Cpd 088—En2

### Synthesis of (S)-4-hydroxy-3-({2-methyl-5-[(pyridin-2-yl)methoxy]pyrazolo[1,5-a]pyridin-3-yl}formamido)butanamide (Cpd 076—En1)

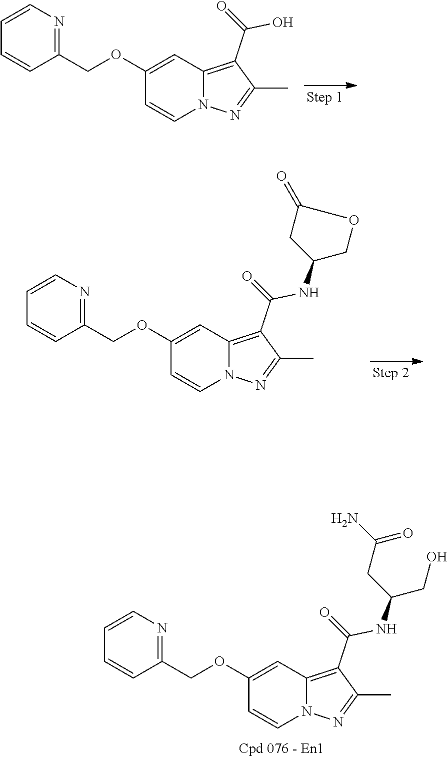 [SMILES: Cc1nn2ccc(OCc3ccccn3)cc2c1C(=O)O ; Cc1nn2ccc(OCc3ccccn3)cc2c1C(=O)N[C@@H]1COC(=O)C1 ; Cc1nn2ccc(OCc3ccccn3)cc2c1C(=O)N[C@H](CO)CC(N)=O]

Step 1: To a solution of 2-methyl-5-(pyridin-2-ylmethoxy)pyrazolo[1,5-a]pyridine-3-carboxylic acid (200 mg, 0.706 mmol) in DMF (10 mL) were added HATU (805 mg, 2.11 mmol), DIPEA (2.1 mL, 3.53 mmol) and (S)-3-Amino-g-butyrolactone hydrochloride (155 mg, 1.13 mmol) at 0° C. The RM was stirred at RT for 16 h under nitrogen atmosphere. After completion, the RM was diluted with cold water (100 mL), extracted with EtOAc (2×100 mL). The combined organic layers were washed sodium bicarbonate solution (2×20 mL), dried over Na₂SO₄, filtered and concentrated under reduced pressure. The residue was purified by FCC on silica gel using 10% MeOH in DCM as an eluent to afford (S)-2-methyl-N-(5-oxotetrahydrofuran-3-yl)-5-(pyridin-2-ylmethoxy)pyrazolo [1,5-a]pyridine-3-carboxamide as an off white solid (160 mg, 90%).

Step 2: To a solution of (S)-2-methyl-N-(5-oxotetrahydrofuran-3-yl)-5-(pyridin-2-ylmethoxy)pyrazolo[1,5-a]pyridine-3-carboxamide (250 mg, 0.682 mmol) in aqueous ammonia (15 mL) was stirred at RT for 16 h. After completion, the RM was concentrated under reduced pressure. The compound was purified by Prep. HPLC. [Prep. HPLC conditions: Mobile phase: 0.1% FA in water, Mobile phase B: ACN, Column: Sunfire (19×250) 5 μm, FLOW: 18 ml/min, Method: (T in min./% of B): 0/10, 2/10, 10/50, 12/50, 12.4/100, 19.02/100, 19.1/10, 20/4.10, Solubility: ACN+THF, Temperature: RT.] The desired fractions were evaporated and lyophilized to afford (S)-4-hydroxy-3-({2-methyl-5-[(pyridin-2-yl)methoxy]pyrazolo[1,5-a]pyridin-3-yl}formamido)butanamide (Cpd 076—En1) as an off-white solid (21.6 mg, 8%).

The following compounds was prepared in a similar manner (use of appropriate reagents (chiral or racemic) and purification methods (including chiral HPLC or chiral SFC) known to the person skilled in the art) as described for Cpd 076—En1: Cpd 076—En2.

### Synthesis of 3-hydroxy-2-methyl-2-({2-methyl-5-[(pyridin-2-yl)methoxy]pyrazolo[1,5-a]pyridin-3-yl}formamido)propanamide (Cpd 089)

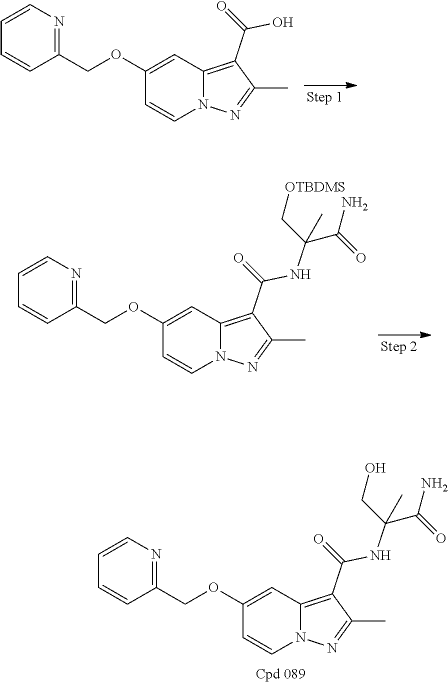 [SMILES: Cc1nn2ccc(OCc3ccccn3)cc2c1C(=O)O ; Cc1nn2ccc(OCc3ccccn3)cc2c1C(=O)NC(C)(CO[Si](C)(C)C(C)(C)C)C(N)=O ; Cc1nn2ccc(OCc3ccccn3)cc2c1C(=O)NC(C)(CO)C(N)=O]

Step 1: To a solution of 2-methyl-5-(pyridin-2-ylmethoxy)pyrazolo[1,5-a]pyridine-3-carboxylic acid (400 mg, 1.413 mmol) in DMF (5.0 mL) were added HATU (805 mg, 2.119 mmol), DIPEA (0.739 ml, 4.239 mmol) and 2-amino-3-((tert-butyldimethylsilyl)oxy)-2-methylpropanamide (393.359 mg, 1.696 mmol) at 0° C. The RM was stirred at RT for 16 h. After completion, the RM was quenched with water (50 mL), extracted with EtOAc (2×100 mL). The combined organic layers were washed with sat. NaHCO₃ (50 mL), brine (50 mL), dried over Na₂SO₄, filtered and concentrated under reduced pressure to afford N-(1-amino-3-((tert-butyldimethylsilyl)oxy)-2-methyl-1-oxopropan-2-yl)-2-methyl-5-(pyridin-2-ylmethoxy)pyrazolo[1,5-a]pyridine-3-carboxamide as an off white solid (650 mg, 92.44%). ¹H NMR (400 MHz, DMSO-de) 5 ppm: 8.62 (d, 1H), 8.59 (d, 1H), 7.88 (t, 1H), 7.58 (d, 1H), 7.50 (m, 2H), 7.40-7.36 (m, 2H), 7.34 (s, 1H), 7.28 (s, 1H), 7.09 (d, 1H), 6.81 (m, 2H), 5.77 (s, 1H), 5.27 (s, 2H), 4.20 (d, 1H), 4.06 (d, 1H), 3.96 (d, 1H), 3.72 (s, 1H), 3.40-3.19 (m, 2H), 2.91 (s, 1H), 2.75 (s, 1H), 2.71 (s, 3H), 2.57 (s, 3H), 2.24 (s, 1H), 1.55 (s, 3H), 1.25-1.03 (m, 6H), 0.90-0.784 (m, 15H).Step 2: To a solution of N-(1-amino-3-((tert-butyldimethylsilyl)oxy)-2-methyl-1-oxopropan-2-yl)-2-methyl-5-(pyridin-2-ylmethoxy)pyrazolo[1,5-a]pyridine-3-carboxamide (750 mg, 1.509 mmol) in THE (40 mL) was added TBAF (1M in THF) (4.527 ml, 4.527 mmol) at RT. The RM was stirred for 3 h. After completion, the RM was diluted with water (100 mL), extracted with EtOAc (3×100 mL). The combined organic phases were washed with sat. NH₄Cl solution (100 mL), brine solution (100 mL), dried over Na₂SO₄, filtered and concentrated under reduced pressure. The compound was purified by Prep. HPLC. [Prep. HPLC conditions: Mobile phase: 10 mM ABC in water, Mobile phase B: ACN, Column: LUNA C18 (21.2×250) 5 μm, FLOW: 18 ml/min, Method: (T in min./% of B): 00/10, 2/10, 10/40, 11/40, 11.10/98, 16/98, 16.10/10, 20/10, Solubility: ACN+Water+DMSO, Temperature: RT.] The desired fractions were evaporated and lyophilized to afford 3-hydroxy-2-methyl-2-({2-methyl-5-[(pyridin-2-yl)methoxy]pyrazolo[1,5-a]pyridin-3-yl}formamido)propanamide (Cpd 089) (100 mg, 17.28%).

A preparative chiral SFC was performed on the racemic mixture of Cpd 089 to afford Cpd 089—En1 and Cpd 089—En2.

### Analytical Data

| Cpd | [M + H]+ | ¹H NMR |
|---|---|---|
| Code | (m/z): | (δ ppm) |
| Cpd 001 | 415.4 | (400 MHz, DMSO-d6): δ 8.55 (d, 1H), 7.63 (t, 1H), 7.62-7.54 (m, |
| | | 2H), 7.50-7.43(m, 1H), 7.41 (s, 1H), 7.34 (s, 1H), 7.30-7.24 (m, |
| | | 2H), 6.71 (dd, 1H), 5.25-5.19 (m, 2H), 4.97 (t, 1H), 4.16-4.12 (m, |
| | | 1H), 3.79-3.75 (m, 1H), 2.57 (s, 3H), 2.45-2.37 (m, 1H), 1.75- |
| | | 1.70 (m, 1H), 0.72 (t, 3H). |
| Cpd 002 | | (600 MHz, DMSO-d₆) δ 8.515 (dd, 1H), 7.638-7.579 (m, 1H), |
| | | 7.500-7.422 (m, 2H), 7.313 - 7.232 (m, 2H), 6.696 (ddd, 1H), |
| | | 6.657 (s, 1H), 5.220 (s, 2H), 5.055 (td, 2H), 3.632 (dd, 2H), 3.515 |
| | | (dd, 2H), 1.314 (d, 3H). |
| Cpd 003 | | (600 MHz, DMSO-d₆) δ 8.536 (d, 1H), 7.614 (td, 1H), 7.544 (d, 1H), |
| | | 7.496-7.421 (m, 2H), 7.311-7.232 (m, 2H), 7.155-7.085 (m, |
| | | 2H), 6.719 (dd, 1H), 5.236 (s, 2H), 5.027-4.969 (m, 1H), 4.447 (dt, |
| | | 1H), 3.776 (ddd, 1H), 3.719 (dt, 1H), 2.555 (s, 3H). |
| Cpd 004 | | (600 MHz, DMSO-d₆) δ 8.522 (d, 1H), 7.609 (td, 1H), 7.494 (d, 1H), |
| | | 7.489-7.433 (m, 1H), 7.328 (s, 1H), 7.315 (s, 1H), 7.304-7.233 |
| | | (m, 2H), 7.063 (s, 1H), 6.707 (dd, 1H), 5.246 (s, 2H), 2.551 (s, 3H), |
| | | 1.558 (s, 6H). |
| Cpd 008 | | (600 MHz, DMSO-d₆) δ 9.011 (s, 1H), 8.504 (d, 1H), 7.435 (d, 1H), |
| | | 6.670 (dd, 1H), 6.637 (s, 1H), 5.389 (s, 2H), 5.101-5.041 (m, 2H), |
| | | 3.630 (dd, 2H), 3.509 (dd, 2H), 2.496 (s, 3H), 2.456 (s, 3H), 1.313 |
| | | (s, 3H). |
| Cpd 009 - | | (600 MHz, DMSO-d₆) δ 8.541 (d, 1H), 7.886 (q, 1H), 7.613 (td, 1H), |
| En1 | | 7.524 (d, 1H), 7.460 (tdd, 1H), 7.311-7.231 (m, 2H), 7.103 (d, 1H), |
| | | 6.726 (dd, 1H), 5.241 (s, 2H), 5.042-4.984 (m, 1H), 4.454 (dt, 1H), |
| | | 3.755 (dt, 1H), 3.692 (dt, 1H), 2.630 (d, 3H), 2.560 (s, 3H). |
| Cpd 009 - | | (600 MHz, DMSO-d₆) δ 8.541 (d, 1H), 7.886 (d, 1H), 7.613 (td, 1H), |
| En2 | | 7.524 (d, 1H), 7.460 (tdd, 1H), 7.312-7.231 (m, 2H), 7.103 (d, 1H), |
| | | 6.726 (dd, 1H), 5.241 (s, 2H), 5.014 (tt, 1H), 4.454 (dt, 1H), 3.754 |
| | | (dt, 1H), 3.692 (dt, 1H), 2.630 (d, 3H), 2.560 (s, 3H). |
| Cpd 010 | | (600 MHz, DMSO-d₆) δ 9.015 (s, 1H), 8.535 (d, 1H), 7.535 (d, 1H), |
| | | 7.436 (s, 1H), 7.138 (s, 1H), 7.082 (d, 1H), 6.700 (dd, 1H), 5.409 (s, |
| | | 2H), 4.998 (td, 1H), 4.446 (dt, 1H), 3.776 (dt, 1H), 3.716 (dt, 1H), |
| | | 2.559 (s, 3H), 2.459 (s, 3H). |
| Cpd 011 - | 415.2 | (600 MHz, DMSO-d₆) δ 8.527 (dd, 1H), 7.751 (q, 1H), 7.608 (td, |
| En1 | | 1H), 7.554-7.517 (m, 1H), 7.505-7.392 (m, 1H), 7.305-7.230 |
| | | (m, 2H), 7.198 (s, 1H), 6.716 (dd, 1H), 5.232 (s, 2H), 5.166 (t, 1H), |
| | | 3.830 (dd, 1H), 3.722 (dd, 1H), 2.637 (d, 3H), 2.566 (s, 3H), 1.509 |
| | | (s, 3H). |
| Cpd 011 - | 415.2 | (600 MHz, DMSO-d₆) δ 8.526 (d, 1H), 7.751 (q, 1H), 7.607 (td, 1H), |
| En2 | | 7.535 (d, 1H), 7.457 (tdd, 1H), 7.303-7.230 (m, 2H), 7.197 (s, 1H), |
| | | 6.716 (dd, 1H), 5.231 (s, 2H), 3.829 (dd, 1H), 3.721 (dd, 1H), 2.637 |
| | | (d, 3H), 2.565 (s, 3H), 1.509 (s, 3H). |
| Cpd 012 | | (600 MHz, DMSO-d₆) δ 8.550 (d, 1H), 7.663 (dd, 1H), 7.568-7.517 |
| | | (m, 2H), 7.466-7.382 (m, 3H), 7.135 (s, 1H), 7.101 (d, 1H), 6.745 |
| | | (dd, 1H), 5.257 (s, 2H), 4.991 (ddd, 1H), 4.441 (dt, 1H), 3.774 (ddd, |
| | | 1H), 3.717 (dt, 1H), 2.562 (s, 3H). |
| Cpd 013 | | (600 MHz, DMSO-d₆) δ 8.536 (d, 1H), 7.685-7.636 (m, 1H), 7.543 |
| | | (dd, 1H), 7.489 (d, 1H), 7.466-7.383 (m, 2H), 7.320 (d, 2H), 7.060 |
| | | (s, 1H), 6.759-6.709 (m, 1H), 5.265 (s, 2H), 2.557 (s, 3H), 1.555 |
| | | (s, 6H). |
| Cpd 014 | | (600 MHz, DMSO-d₆) δ 8.528 (d, 1H), 7.660 (dd, 1H), 7.567-7.519 |
| | | (m, 1H), 7.467-7.384 (m, 3H), 6.723 (dd, 1H), 6.654 (s, 1H), 5.241 |
| | | (s, 2H), 5.044 (t, 2H), 3.630 (dd, 2H), 3.514 (dd, 2H), 2.504 (s, 3H), |
| | | 1.312 (s, 3H). |
| Cpd 015 - | 357.2 | (400 MHz, DMSO-d₆): δ 8.56 (d, 1H), 7.45 (d, 1H), 7.39 (s, 1H), |
| En1 | | 7.28 (s, 1H), 7.20 (s, 1H), 6.73 (dd, 1H), 6.44 (tt, 1H), 5.13 (s, 1H), |
| | | 4.48-4.38 (m, 2H), 3.91 (d, 1H), 3.74 (m, 1H), 2.56 (s, 3H), 1.51 (s, |
| | | 3H) |
| Cpd 015 - | 357.2 | (400 MHz, DMSO-d₆): δ 8.56 (d, 1H), 7.45 (d, 1H), 7.39 (s, 1H), |
| En2 | | 7.27 (s, 1H), 7.20 (s, 1H), 6.74 (dd, 1H), 6.44 (tt, 1H), 5.11 (t, 1H), |
| | | 4.48-4.38 (m, 2H), 3.94-3.88 (m, 1H), 3.77-3.71 (m, 1H), 2.56 |
| | | (s, 3H), 1.51 (s, 3H). |
| Cpd 016 - | 383.2 | (400 MHz, DMSO-d₆): δ 8.53 (d, 1H), 7.53-7.49 (m, 3H), 7.44- |
| En1 | | 7.33 (m, 4H), 7.25 (s, 1H), 7.21 (s, 1H), 6.72 (dd, 1H), 5.20-5.14 |
| | | (m, 3H), 3.92 (d, 1H), 3.74 (d, 1H), 2.55 (s, 3H), 1.51 (s, 3H). |
| Cpd 016 - | 383.2 | (400 MHz, DMSO-d₆): δ 8.53 (d, 1H), 7.53-7.49 (m, 3H), 7.43- |
| En2 | | 7.34 (m, 4H), 7.25 (s, 1H), 7.21 (s, 1H), 6.72 (dd, 1H), 5.19 (s, 2H), |
| | | 5.12 (s, 1H), 3.94-3.89 (m, 1H), 3.78-3.72 (m, 1H), 2.55 (s, 3H), |
| | | 1.51 (s, 3H). |
| Cpd 017 - | 429.2 | 400 MHz, DMSO-d6): δ 8.53 (d, 1H), 7.61-7.58 (m, 1H), 7.49- |
| En1 | | 7.40 (m, 2H), 7.36 (d, 1H), 7.31-7.23 (m, 2H), 6.71 (dd, 1H), 5.23 |
| | | (s, 2H), 4.92 (t, 1H), 3.80-3.68 (m, 2H), 2.93 (bs, 6H), 2.50 (s, 3H), |
| | | 1.46 (s, 3H). |
| Cpd 017 - | 429.2 | (400 MHz, DMSO-d₆): δ 8.53 (d, 1H), 7.59 (t, 1H), 7.49-7.43 (m, |
| En2 | | 2H), 7.36 (d, 1H), 7.30-7.24 (m, 2H), 6.71 (dd, 1H), 5.23 (s, 2H), |
| | | 4.96 (brs, 1H), 3.79-3.69 (m, 2H), 2.93 (brs, 6H), 2.50 (s, 3H), 1.46 |
| | | (s, 3H). |
| Cpd 018 - | 413.2 | (400 MHz, DMSO-d₆): δ 8.54 (d, 1H), 7.76 (s, 1H), 7.62 (t, 1H), 7.50- |
| En1 | | 7.41 (m, 2H), 7.31-7.23 (m, 2H), 6.90 (s, 1H), 6.71 (dd, 1H), 5.31 |
| | | (s, 1H), 5.22 (s, 2H), 3.65-3.55 (m, 2H), 3.27-3.15 (m, 2H), 2.52- |
| | | 2.48 (m, 3H), 2.45-2.39 (m, 2H |
| Cpd 018 - | 413.2 | (400 MHz, DMSO-d₆): δ 8.54 (d, 1H), 7.76 (s, 1H), 7.62 (t, 1H), |
| En2 | | 7.50-7.41 (m, 2H), 7.31-7.23 (m, 2H), 6.92 (s, 1H), 6.71 (dd, 1H), |
| | | 5.31 (s, 1H), 5.23 (s, 2H), 3.65-3.55 (m, 2H), 3.28-3.15 (m, 2H), |
| | | 2.52-2.48 (m, 3H), 2.45-2.39 (m, 2H). |
| Cpd 019 - | 417.2 | (400 MHz, DMSO-d₆): δ 8.55 (d, 1H), 7.67 (d, 1H), 7.56-7.52 (m, |
| En1 | | 2H), 7.45-7.38 (m, 3H), 7.28 (s, 1H), 7.20 (s, 1H), 6.74 (dd, 1H), |
| | | 5.24 (s, 2H), 5.13 (s, 1H), 3.94-3.89 (m, 1H), 3.77-3.72 (m, 1H), |
| | | 2.55 (s, 3H), 1.52 (s, 3H) |
| Cpd 019 - | 417.2 | (400 MHz, DMSO-d₆): δ 8.55 (d, 1H), 7.68-7.65 (m, 1H), 7.56- |
| En2 | | 7.52 (m, 2H), 7.46-7.38 (m, 3H), 7.29 (s, 1H), 7.20 (s, 1H), 6.74 |
| | | (dd, 1H), 5.24 (s, 2H), 5.14 (s, 1H), 3.92 (d, 1H), 3.74 (d, 1H), 2.56 |
| | | (s, 3H), 1.51 (s, 3H). |
| Cpd 020 - | 401.2 | (400 MHz, DMSO-d6): δ 8.54 (d, 1H), 7.62 (t, 1H), 7.56 (d, 1H), |
| En1 | | 7.50-7.42 (m, 1H), 7.39 (brs, 1H), 7.31-7.22 (m, 3H), 7.21 (brs, |
| | | 1H), 6.70 (dd, 1H), 5.22 (s, 2H), 5.12 (t, 1H), 3.95-3.88 (m, 1H), |
| | | 3.80-3.71 (m, 1H), 2.56 (s, 3H), 1.52 (s, 3H). |
| Cpd 020 - | 401.2 | (400 MHz, DMSO-d6): δ 8.54 (d, 1H), 7.63 (t, 1H), 7.56 (d, 1H), |
| En2 | | 7.50-7.42 (m, 1H), 7.39 (brs, 1H), 7.31-7.22 (m, 3H), 7.21 (brs, |
| | | 1H), 6.70 (dd, 1H), 5.22 (s, 2H), 5.12 (t, 1H), 3.95-3.88 (m, 1H), |
| | | 3.80-3.71 (m, 1H), 2.56 (s, 3H), 1.52 (s, 3H). |
| Cpd 021 - | 398.2 | (400 MHz, DMSO-d6): δ 8.57 (s, 1H), 8.54 (d, 1H), 7.81 (dd, 1H), |
| En1 | | 7.55 (d, 1H), 7.40 (s, 1H), 7.31 (d, 1H), 7.26 (s, 1H), 7.21 (s, 1H), |
| | | 6.71 (dd, 1H), 5.19 (s, 3H), 3.94-3.89 (m, 1H), 3.77-3.73 (m, 1H), |
| | | 2.56 (s, 3H), 2.50 (s, 3H), 1.52 (s, 3H). |
| Cpd 021 - | 398.2 | 400 MHz, DMSO-d₆): δ 8.57 (d, 1H), 8.54 (d, 1H), 7.81 (d, 1H), 7.55 |
| En2 | | (d, 1H), 7.40 (s, 1H), 7.30 (m, 1H), 7.26 (s, 1H), 7.21 (s, 1H), 6.71 |
| | | (dd, 1H), 5.19 (s, 2H), 5.12 (t, 1H), 3.95-3.89 (m, 1H), 3.78-3.72 |
| | | (m, 1H), 2.56 (s, 3H), 2.50 (s, 3H), 1.52 (s, 3H). |
| Cpd 022 - | 347.2 | (400 MHz, DMSO-d6): δ 8.50 (d, 1H), 7.40-7.34 (m, 2H), 7.25- |
| En1 | | 7.18 (d, 2H), 6.66 (dd, 1H), 5.11 (s, 1H), 3.91-3.90 (m, 3H), 3.76- |
| | | 3.70 (m, 1H), 2.54 (s, 3H), 1.50 (s, 3H), 1.30-1.23 (m, 1H), 0.63- |
| | | 0.57 (m, 2H), 0.41-0.36 (m, 2H), |
| Cpd 022 - | 347.2 | (400 MHz, DMSO-d₆): δ 8.50 (d, 1H), 7.39-7.33 (m, 2H), 7.23- |
| En2 | | 7.18 (m, 2H), 6.66 (dd, 1H), 5.11 (s, 1H), 3.91-3.88 (m, 3H), 3.75- |
| | | 3.71 (m, 1H), 2.54 (s, 3H), 1.50 (s, 3H), 1.31-1.22 (m, 1H), 0.63- |
| | | 0.58 (m, 2H), 0.40-0.36 (m, 2H). |
| Cpd 023 - | 433.2 | (400 MHz, DMSO-d₆): δ 8.54 (d, 1H), 7.70-7.65 (m, 2H), 7.63- |
| En1 | | 7.52 (m, 3H), 7.42-7.14 (m, 4H), 6.71 (dd, 1H), 5.34 (s, 2H), 5.14 |
| | | (bs, 1H), 3.92 (d, 1H), 3.75 (d, 1H), 2.57 (s, 3H), 1.52 (s, 3H). |
| Cpd 023 - | 433.2 | (400 MHz, DMSO-d₆): δ 8.54 (d, 1H), 7.70-7.66 (m, 2H), 7.63- |
| En2 | | 7.53 (m, 3H), 7.42-7.14 (m, 4H), 6.71 (dd, 1H), 5.34 (s, 2H), 5.14 |
| | | (bs, 1H), 3.92 (d, 1H), 3.75 (d, 1H), 2.57 (s, 3H), 1.52 (s, 3H). |
| Cpd 024 - | 415.2 | 400 MHz, DMSO-d₆): δ 8.60 (d, 1H), 7.66-7.62 (m, 2H), 7.51- |
| En1 | | 7.43 (m, 1H), 7.39 (s, 1H), 7.31-7.25 (m, 2H), 7.16 (s, 1H), 6.78 |
| | | (dd, 1H), 5.34-5.27 (m, 2H), 4.32-4.23 (m, 2H), 2.50 (s, 3H), 2.30- |
| | | 2.21 (s, 4H), 1.25 (s, 3H). |
| Cpd 024 - | 415.2 | (400 MHz, DMSO-d₆): δ 8.60 (d, 1H), 7.66-7.62 (m, 2H), 7.50- |
| En2 | | 7.44 (m, 1H), 7.39 (s, 1H), 7.32-7.25 (m, 2H), 7.16 (s, 1H ), 6.77 |
| | | (dd, 1H), 5.34-5.27 (m, 2H), 4.32-4.23 (m, 2H), 2.50 (s, 3H), 2.32- |
| | | 2.22 (s, 4H), 1.25 (s, 3H). |
| Cpd 025 - | 422.2 | (400 MHz, DMSO-d₆): δ 8.52 (d, 1H), 7.60 (t, 1H), 7.50-7.43 (m, |
| En1 | | 1H), 7.36 (d, 1H), 7.31-7.23 (m, 2H), 6.83 (s, 1H), 6.70 (dd, 1H), |
| | | 6.37-6.07 (tt, 1H), 5.25 (s, 1H), 5.22 (s, 2H), 3.63-3.53 (m, 2H), |
| | | 2.51-2.32 (m, 5H), 1.42 (s, 3H). |
| Cpd 025 - | 422.2 | (400 MHz, DMSO-d₆): δ 8.52 (d, 1H), 7.64-7.57 (m, 1H), 7.50- |
| En2 | | 7.42 (m, 1H), 7.36 (d, 1H), 7.31-7.23 (m, 2H), 6.83 (s, 1H), 6.70 |
| | | (dd, 1H), 6.22 (tt, 1H), 5.26 (s, 1H), 5.22 (s, 2H), 3.58 (q, 2H), 2.48 |
| | | (s, 3H), 2.47-2.35 (m, 2H), 1.39 (s, 3H). |
| Cpd 026 - | 419.2 | (500 MHz, DMSO-d₆): δ 8.54 (d, 1H), 7.71-7.66 (m, 1H), 7.55 (d, |
| En1 | | 1H), 7.40 (s, 1H), 7.36-7.32 (m, 1H), 7.28 (s, 1H), 7.22 (s, 1H), |
| | | 7.18-7.14 (m, 1H), 6.71 (dd, 1H), 5.20 (s, 2H), 5.12 (t, 1H), 3.94- |
| | | 3.91 (m, 1H), 3.76-3.73 (m, 1H), 2.56 (s, 3H), 1.52 (s, 3H). |
| Cpd 026 - | 419.2 | (500 MHz, DMSO-d₆): δ 8.54 (d, 1H), 7.71-7.66 (m, 1H), 7.55 (d, |
| En2 | | 1H), 7.40 (s, 1H), 7.36-7.32 (m, 1H), 7.28 (s, 1H), 7.22 (s, 1H), |
| | | 7.17-7.14 (m, 1H), 6.71 (dd, 1H), 5.20 (s, 2H), 5.12 (t, 1H), 3.94- |
| | | 3.91 (m, 1H), 3.76-3.73 (m, 1H), 2.56 (s, 3H), 1.52 (s, 3H). |
| Cpd 027 - | 419.2 | (400 MHz, DMSO-d₆): δ 8.55 (d, 1H), 7.56 (d, 1H), 7.55-7.44 (m, |
| En1 | | 2H), 7.43 (s, 1H), 7.31-7.24 (m, 2H), 7.21 (s, 1H), 6.73 (dd, 1H), |
| | | 5.28 (s, 2H), 5.11 (t, 1H), 3.94-3.90 (m, 1H), 3.77-3.73 (m, 1H), |
| | | 2.56 (s, 3H), 1.52 (s, 3H). |
| Cpd 027 - | 419.3 | (400 MHz, DMSO-d₆): δ 8.55 (d, 1H), 7.56 (d, 1H), 7.50-7.40 (m, |
| En2 | | 3H), 7.30-7.21 (m, 3H), 6.73 (dd, 1H), 5.28 (s, 2H), 5.11 (t, 1H), |
| | | 3.94-3.90 (m, 1H), 3.77-3.73 (m, 1H), 2.56 (s, 3H), 1.52 (s, 3H). |
| Cpd 028 - | 415.2 | (500 MHz, DMSO-d₆): δ 8.53 (d, 1H), 7.53 (d, 1H), 7.47 (t, 1H), |
| En1 | | 7.39 (brs, 1H), 7.28 (s, 1H), 7.21 (brs, 1H), 7.12-7.06 (m, 2H), 6.69 |
| | | (dd, 1H), 5.17 (s, 2H), 5.14 (s, 1H), 3.92 (d, 1H), 3.75 (d, 1H), 2.55 |
| | | (s, 3H), 2.34 (s, 3H), 1.52 (s, 3H). |
| Cpd 028 - | 415.2 | (500 MHz, DMSO-d₆): δ 8.53 (d, 1H), 7.53 (d, 1H), 7.47 (t, 1H), |
| En2 | | 7.39 (brs, 1H), 7.28 (s, 1H), 7.21 (brs, 1H), 7.12-7.06 (m, 2H), 6.69 |
| | | (dd, 1H), 5.17 (s, 2H), 5.14 (s, 1H), 3.92 (d, 1H), 3.75 (d, 1H), 2.55 |
| | | (s, 3H), 2.34 (s, 3H), 1.52 (s, 3H). |
| Cpd 029 - | 415.2 | (400 MHz, DMSO-d₆): δ 8.53 (d, 1H), 7.54 (d, 1H), 7.43-7.40 (m, |
| En1 | | 2H), 7.34-7.28 (m, 2H), 7.21 (brs, 1H), 7.14 (t, 1H), 6.71 (dd, 1H), |
| | | 5.20 (s, 2H), 5.13 (s, 1H), 3.92 (d, 1H), 3.75 (d, 1H), 2.56 (s, 3H), |
| | | 2.27 (s, 3H), 1.52 (s, 3H). |
| Cpd 029 - | 415.2 | (400 MHz, DMSO-d₆): δ 8.53 (d, 1H), 7.55 (d, 1H), 7.43-7.39 (m, |
| En2 | | 2H), 7.34-7.28 (m, 2H), 7.21 (brs, 1H), 7.14 (t, 1H), 6.71 (dd, 1H), |
| | | 5.20 (s, 2H), 5.13 (s, 1H), 3.92 (d, 1H), 3.75 (d, 1H), 2.56 (s, 3H), |
| | | 2.27 (s, 3H), 1.52 (s, 3H), |
| Cpd 030 - | 419.3 | (400 MHz, DMSO-d₆): δ 8.55 (d, 1H), 7.55 (d, 1H), 7.52-7.48 (m, |
| En1 | | 1H), 7.40 (s, 1H), 7.38-7.29 (m, 2H), 7.28 (s, 1H), 7.21 (s, 1H), |
| | | 6.75 (dd, 1H), 5.38-5.21 (m, 3H), 3.94-3.90 (m, 1H), 3.76-3.73 |
| | | (m, 1H), 2.56 (s, 3H), 1.52 (s, 3H). |
| Cpd 030 - | 419.3 | (400 MHz, DMSO-d₆): δ 8.55 (d, 1H), 7.55 (d, 1H), 7.52-7.48 (m, |
| En2 | | 1H), 7.42 (s, 1H), 7.38-7.29 (m, 2H), 7.27 (s, 1H), 7.21 (s, 1H), |
| | | 6.75 (dd, 1H), 5.22 (s, 2H), 5.11 (t, 1H), 3.94-3.90 (m, 1H), 3.77- |
| | | 3.73 (m, 1H), 2.56 (s, 3H), 1.52 (s, 3H). |
| Cpd 031 - | 415.2 | (400 MHz, DMSO-d₆): δ 8.53 (d, 1H), 7.61 (d, 1H), 7.42-7.33 (m, |
| En1 | | 2H), 7.29 (s, 1H), 7.26 (s, 1H), 7.16-7.07 (m, 2H), 6.67 (dd, 1H), |
| | | 5.19 (s, 2H), 5.13 (s, 1H), 3.95-3.89 (m, 1H), 3.80-3.72 (m, 1H), |
| | | 2.56 (s, 3H), 2.39 (s, 3H), 1.53 (s, 3H). |
| Cpd 031 - | 415.2 | (400 MHz, DMSO-d₆): δ 8.53 (d, 1H), 7.61 (d, 1H), 7.42-7.33 (m, |
| En2 | | 2H), 7.29 (s, 1H), 7.21 (s, 1H), 7.16-7.07 (m, 2H), 6.67 (dd, 1H), |
| | | 5.19 (s, 2H), 5.13 (s, 1H), 3.95-3.89 (m, 1H), 3.80-3.72 (m, 1H), |
| | | 2.56 (s, 3H), 2.39 (s, 3H), 1.53 (s, 3H). |
| Cpd 032 - | 419.2 | (400 MHz, DMSO-d₆): δ 8.54 (d, 1H), 7.60-7.53 (m, 2H), 7.39 (brs, |
| En1 | | 1H), 7.30 (s, 1H), 7.23-7.19 (m, 3H), 6.68 (dd, 1H), 5.20 (s, 2H), |
| | | 5.16 (bs, 1H), 3.91 (d, 1H), 3.75 (d, 1H), 2.56 (s, 3H), 1.52 (s, 3H). |
| Cpd 032 - | 419.2 | 400 MHz, DMSO-d₆): δ 8.54 (d, 1H), 7.60-7.55 (m, 2H), 7.39 (brs, |
| En2 | | 1H), 7.30 (s, 1H), 7.23-7.19 (m, 3H), 6.68 (dd, 1H), 5.20 (s, 2H), |
| | | 5.14 (bs, 1H), 3.91 (d, 1H), 3.75 (d, 1H), 2.56 (s, 3H), 1.52 (s, 3H). |
| Cpd 033 | 283 | (300 MHz, DMSO-d₆) d ppm 12.14 (br. s., 1 H), 8.52 (d, 1 H), 7.41- |
| | | 7.52 (m, 2 H), 7.24-7.40 (m, 4 H), 6.73 (d, 1 H), 5.16 (s, 2 H), 2.45 |
| | | (s, 3H). |
| Cpd 034 | 247.2 | (400 MHz, DMSO-d6): δ 12.15 (s, 1H), 8.54 (d, 1H), 7.23 (d, 1H), |
| | | 6.72 (dd, 1H), 3.93 (d, 2H), 2.49 (s, 3H), 1.28-1.21 (m, 1H), 0.63- |
| | | 0.56 (m, 2H), 0.42-0.37 (m, 2H). |
| Cpd 035 | 301.1 | (300 MHz, DMSO-d₆) d ppm 12.20 (br. s., 1 H), 8.58 (d, 1 H), 7.61 |
| | | (t, 1H), 7.48-7.43 (m, 2H), 7.26 (q, 2H), 6.77 (d, 1H)5.27 (s, 2H). |
| Cpd 036 | 317.1 | (300 MHz, DMSO-d₆) d ppm: 8.58 (d, 1 H), 7.66 (d, 1H), 7.56-7.40 |
| | | (m, 4H), 6.81 (d, 1H), 5.29 (s, 2H), 2.53 (s, 3H) |
| Cpd 037 - | 425.19 | (400 MHz, DMSO-D₆) δ ppm: 9.02 (s, 1 H), 8.52 (d, 1 H), 7.37 (d, |
| En1 | | 1 H), 6.78 (s, 1 H), 6.67 (dd, 1 H), 6.07-6.38 (m, 1 H), 5.40 (s, 2 H), |
| | | 5.25 (t, 2 H), 3.54-3.62 (m, 2 H), 2.50-2.67 (m, 1 H), 2.49 (s, |
| | | 3 H), 2.46 (s, 3 H), 2.29-2.43 (m, 1 H), 1.39 (s, 3 H). |
| Cpd 037 - | 425.19 | (400 MHz, DMSO-D₆) δ ppm: 9.02 (s, 1 H), 8.52 (d, 1 H), 7.37 (d, |
| En2 | | 1 H), 6.78 (s, 1 H), 6.67 (dd, 1 H), 6.07-6.38 (m, 1 H), 5.40 (s, 2 H), |
| | | 5.25 (t, 2 H), 3.54-3.62 (m, 2 H), 2.50-2.67 (m, 1 H), 2.49 (s, |
| | | 3 H), 2.46 (s, 3 H), 2.29-2.43 (m, 1 H), 1.39 (s, 3 H). |
| Cpd 038 - | 405.25 | (400 MHz, DMSO-D₆) δ ppm: 8.59-8.61 (m, 1 H), 8.53 (d, 1 H), |
| En1 | | 7.84-7.88 (m, 1 H), 7.56 (d, 1 H), 7.36-7.39 (m, 1 H), 7.29 (d, |
| | | 1 H), 6.81 (s, 1 H), 6.74 (dd, 1 H), 6.05-6.35 (m, 1 H), 5.26 (s, 2 H), |
| | | 5.21-5.25 (m, 1 H), 3.53-3.61 (m, 2 H), 2.51-2.58 (m, 1 H), |
| | | 2.49 (s, 3 H), 2.27-2.41 (m, 1 H), 1.37 (s, 3 H). |
| Cpd 038 - | 405.25 | (400 MHz, DMSO-D₆) δ ppm: 8.59-8.61 (m, 1 H), 8.53 (d, 1 H), |
| En2 | | 7.84-7.88 (m, 1 H), 7.56 (d, 1 H), 7.36-7.39 (m, 1 H), 7.29 (d, |
| | | 1 H), 6.81 (s, 1 H), 6.74 (dd, 1 H), 6.05-6.35 (m, 1 H), 5.26 (s, 2 H), |
| | | 5.21-5.25 (m, 1 H), 3.53-3.61 (m, 2 H), 2.51-2.58 (m, 1 H), |
| | | 2.49 (s, 3 H), 2.27-2.41 (m, 1 H), 1.37 (s, 3 H). |
| Cpd 042 | 365.15 | (400 MHz, DMSO-D₆) δ ppm: 8.57-8.60 (m, 2 H), 7.93 (t, 1 H), |
| | | 7.84-7.92 (m, 1 H), 7.55-7.57 (d, 1 H), 7.36-7.39 (m, 1 H), 7.30- |
| | | 7.31 (d, 1 H), 6.79-6.81 (m, 1 H), 5.29 (s, 2 H), 4.02-4.11 (m, |
| | | 2 H), 2.49-2.50 (m, 3 H). |
| Cpd 043 | 327.25 | (400 MHz, DMSO-D₆) δ ppm: 8.59-8.61 (m, 1 H), 8.50 (d, 1 H), |
| | | 7.86-7.90 (m, 1 H), 7.58 (d, 1 H), 7.35-7.41 (m, 2 H), 7.27 (t, |
| | | 1 H), 6.77 (dd, 1 H), 5.27 (s, 2 H), 4.74 (t, 1 H), 3.54 (q, 2 H), 3.35 |
| | | (m, 2 H), 2.50 (s, 3 H). |
| Cpd 044 | 363.22 | (400 MHz, DMSO-D₆) δ ppm: 11.74 (s, 1 H), 8.60-8.61 (m, 1 H), |
| | | 8.54 (d, 1 H), 7.86 (dt, 1 H), 7.79 (t, 1 H), 7.58 (d, 1 H), 7.53 (d, |
| | | 1 H), 7.36-7.39 (m, 1 H), 7.01 (brs, 1 H), 6.83 (brs, 1 H), 6.76 (dd, |
| | | 1 H), 5.29 (s, 2 H), 4.49 (d, 2 H), 2.53 (s, 3 H). |
| Cpd 045 | 396.42 | (400 MHz, DMSO-D₆) δ ppm: 8.59-8.61 (m, 1 H), 8.54 (d, 1 H), |
| | | 7.83-7.87 (m, 1 H), 7.64 (t, 1 H), 7.54 (d, 1 H), 7.36-7.39 (m, |
| | | 2 H), 6.76 (dd, 1 H), 5.26 (s, 2 H), 3.19 (d, 2 H), 2.49-2.51 (m, 3 H), |
| | | 2.24 (s, 6 H), 2.19 (s, 2 H), 0.89 (s, 6 H). |
| Cpd 046 | 423.34 | (400 MHz, DMSO-D₆) δ ppm: 8.60-8.62 (m, 1 H), 8.54 (d, 1 H), 7.85- |
| | | 7.89 (dt, 1 H), 7.77 (t, 1 H), 7.56 (t, 3 H), 7.49 (t, 3 H), 7.37-7.40 |
| | | (m, 1 H), 7.19 (d, 1 H), 6.74-6.77 (m, 1 H), 5.22 (s, 2 H), 4.00- |
| | | 4.08 (m, 2 H), 2.41 (s, 3 H). |
| Cpd 047 | 459.15 | (400 MHz, DMSO-D₆) δ ppm: 8.59 (d, 1 H), 8.52 (d, 1 H), 7.83- |
| | | 7.88 (m, 1 H), 7.76 (t, 1 H), 7.53-7.63 (m, 4 H), 7.36-7.39 (m, |
| | | 1 H), 7.22 (d, 1 H), 6.73 (dd, 1 H), 5.22 (s, 2 H), 4.64 (d, 2 H), 2.43 |
| | | (s, 3 H) |
| Cpd 048 | 439.2 | (400 MHz, DMSO-D₆) δ ppm: 8.58-8.59 (dt, 1 H), 8.55 (dd, 1 H), |
| | | 7.95 (t, 1 H), 7.83-7.87 (td, 1 H), 7.55-7.56 (dd, 1 H), 7.02-7.40 |
| | | (m, 7 H), 6.75-6.78 (dd, 1 H), 5.27 (s, 2 H), 4.50 (d, 2 H), 2.49- |
| | | 2.51 (m, 3 H). |
| Cpd 049 - | 422.2 | (400 MHz, DMSO-D₆) δ ppm: 9.02 (s, 1 H), 8.54 (d, 1 H), 7.34- |
| En1 | | 7.30 (m, 2 H), 6.70-6.68 (m, 1 H), 5.40 (s, 2 H), 4.37-4.32 (m, |
| | | 1 H), 2.96-2.82 (m, 2 H), 2.74-2.63 (m, 2 H), 2.45 (s, 3 H), 2.46 (s, |
| | | 3 H), 1.94-1.92 (m, 1 H), 1.90-1.83 (m, 1 H); |
| Cpd 049 - | 422.2 | (400 MHz, DMSO-D₆) δ ppm: 9.02 (S, 1 H), 8.55-8.49 (m, 1 H), |
| En2 | | 7.35-7.30 (m, 2 H), 6.70-6.67 (m, 1 H), 5.40 (s, 2 H), 4.38-4.32 |
| | | (m, 1 H), 2.99-2.81 (m, 2 H), 2.70-2.59 (m, 2 H), 2.49 (s, 3 H), |
| | | 2.45 (s, 3 H), 2.18-2.03 (m, 1 H), 1.93-1.85 (m, 1 H); |
| Cpd 050 - | 402.4 | (400 MHz, DMSO-D₆) δ ppm: 8.60 (d, 1 H), 8.55 (d, 1 H), 7.88-7.83 |
| En1 | | (m, 1 H), 7.56 (d, 1 H), 7.39-7.36 (m, 2 H), 7.24 (s, 1 H), 6.78- |
| | | 6.75 (m, 1 H), 5.26 (s, 2 H), 4.36-4.29 (m, 1 H), 2.91 (t, 2 H), 2.69- |
| | | 2.65 (m, 2 H), 2.49 (s, 4 H), 2.12-2.03 (m, 1 H), 1.95-1.84 (m, 1 H). |
| Cpd 050 - | 402.3 | (400 MHz, DMSO-D₆) δ ppm: 8.60 (d, 1 H), 8.55 (d, 1 H), 7.88- |
| En2 | | 7.83 (m, 1 H), 7.56 (d, 1 H), 7.38 (t, 2 H), 7.23 (d, 1 H), 6.78-6.75 |
| | | (m, 1 H), 5.26 (s, 2 H), 4.35-4.29 (m, 1 H), 2.91 (m, 2 H), 2.65 (t, |
| | | 2 H), 2.49 (s, 4 H), 2.12-2.03 (m, 1 H), 1.92-1.84 (m, 1 H). |
| Cpd 051 - | 422.3 | (400 MHz, DMSO-D₆) δ ppm: 9.02 (s, 1 H), 8.52 (d, 1 H), 7.33 (d, |
| En1 | | 1 H), 7.22 (d, 1 H), 6.67 (dd, 1 H), 5.41 (s, 2 H), 4.12-4.13 (m, 1 H), |
| | | 3.16-3.17 (m, 2 H), 2.62-2.98 (m, 2 H), 2.50 (s, 3 H), 2.46 (s, |
| | | 3 H), 2.29-2.35 (m, 1 H), 2.00-2.12 (m, 1 H) |
| Cpd 051 - | 422.3 | (400 MHz, DMSO-D₆) δ ppm: 1H NMR (400 MHz, DMSO-D6) δ |
| En2 | | ppm: 9.02 (s, 1H), 8.52 (d, 1 H), 7.34 (dd, 1 H), 7.22 (d, 1 H), 6.67 |
| | | (dd, 1 H), 5.41 (s, 2 H), 4.11-4.12 (m, 1 H), 2.95-2.98 (m, 2 H), |
| | | 2.67-2.84 (m, 2 H), 2.49 (s, 3 H), 2.46 (s, 3 H), 2.29-2.38 (m, |
| | | 1 H), 2.00-2.12 (m, 1 H) |
| Cpd 052 - | 402.3 | (400 MHz, DMSO-D₆) δ ppm: 8.60 (d, 1 H), 8.54 (d, 1 H), 7.85- |
| En1 | | 7.88 (m, 1 H), 7.56 (d, 1 H), 7.37 (t, 1 H), 7.27 (s, 1 H), 7.23 (d, |
| | | 1 H), 6.74-6.76 (m, 1 H), 5.28 (s, 2 H), 4.10 (bs, 1 H), 2.93-2.99 (m, |
| | | 2 H), 2.50-2.73 (m, 2 H), 2.49 (s, 1 H), 2.48 (s, 3 H), 2.30-2.33 |
| | | (m, 1 H), 2.08-2.12 (m, 1 H), |
| Cpd 052 - | 402.3 | (400 MHz, DMSO-D₆) δ ppm: 8.60 (d, 1 H), 8.54 (d, 1 H), 7.85-7.88 |
| En2 | | (m, 1 H), 7.56 (d, 1 H), 7.37 (t, 1 H), 7.27 (s, 1 H), 7.23 (d, 1 H), |
| | | 6.74-6.76 (m, 1 H), 5.28 (s, 2 H), 4.10 (bs, 1 H), 2.93-2.99 (m, 2 H), |
| | | 2.50-2.73 (m, 2 H), 2.49 (s, 1 H), 2.48 (s, 3 H), 2.30-2.33 (m, 1 H), |
| | | 2.0-2.1 (m, 1 H). |
| Cpd 053 - | 372.3 | (400 MHz, DMSO-D₆) δ ppm: 9.02 (S, 1H), 8.50 (d, 1H), 7.27-7.31 |
| En 1 | | (M, 2H), 6.65 (dd, 1H), 5.41 (s, 2H), 4.30-4.33 (m, 1H), 2.88-2.98 |
| | | (m, 1H), 2.65-2.76 (m, 1H), 2.49 (s, 3H), 2.46 (s, 3H), 1.94-2.01 (m, |
| | | 1H), 1.65-1.66 (m, 1H); |
| Cpd 053 - | 327.3 | (400 MHz, DMSO-D₆) δ ppm: 9.02 (S, 1H), 8.50 (d, 1H), 7.29-7.31 |
| En 2 | | (M, 2H), 6.65 (dd, 1H), 5.41 (s, 2H), 4.30-4.32 (m, 1H), 2.88-2.98 |
| | | (m, 1H), 2.65-2.76 (m, 1H), 2.49 (s, 3H), 2.45 (s, 3H), 1.94-2.07 (m, |
| | | 1H), 1.65-1.76 (m, 1H) |
| Cpd 054 - | 352.3 | (400 MHz, DMSO-D₆) δ ppm: 8.60 (d, 1 H), 8.52 (d, 1 H), 7.86 (td, |
| En1 | | 1 H), 7.55 (d, 1 H), 7.38 (m, 1 H), 7.31 (d, 1 H), 7.22-7.23 (m, 1 H), |
| | | 6.73 (dd, 1 H), 5.28 (s, 2H), 4.28-4.42 (m, 1 H), 2.88-2.96 (m, |
| | | 2 H), 2.64-2.76 (m, 2 H), 1.93-1.99 (m, 1 H), 1.59-1.70 (m, 1 H) |
| Cpd 054 - | 352.3 | (400 MHz, DMSO-D₆) δ ppm: 8.60 (d, 1 H), 8.52 (d, 1 H), 7.86 (td, |
| En2 | | 1 H), 7.55 (d, 1 H), 7.38 (m, 1 H), 7.31 (d, 1 H), 7.22-7.23 (m, 1 H), |
| | | 6.73 (dd, 1 H), 5.28 (s, 2H), 4.28-4.42 (m, 1 H), 2.88-2.96 (m, |
| | | 2 H), 2.64-2.76 (m, 2 H), 1.93-1.99 (m, 1 H), 1.59-1.70 (m, 1 H). |
| Cpd 055 | 403.23 | (400 MHz, DMSO-D₆) δ ppm: 8.59-8.61 (m, 1 H), 8.54 (d, 1 H), |
| | | 7.84-7.88 (m, 1 H), 7.58 (t, 2 H), 7.36-7.39 (m, 1 H), 7.27 (d, |
| | | 1 H), 6.75 (dd, 1 H), 5.26 (s, 3 H), 3.62 (s, 2 H), 2.84 (s, 4 H), 2.50 |
| | | (s, 3 H). |
| Cpd 056 | 444.2 | (400 MHz, DMSO-D₆) δ ppm: 12.71 (s, 1 H), 8.91 (s, 1 H), 8.65 (d, |
| | | 1 H), 8.61 (d, 1 H), 8.48 (d, 1 H), 7.87 (dt, 1 H), 7.70 (s, 1 H), 7.64 |
| | | (d, 1 H), 7.60 (d, 1 H), 7.37-7.40 (m, 1 H), 6.88 (dd, 1 H), 5.32 (s, |
| | | 2 H), 2.67 (s, 3 H). |
| Cpd 057 | 427.37 | (400 MHz, DMSO-D₆) δ ppm: 8.60 (d, 1 H), 8.57 (d, 1 H), 8.55 (d, |
| | | 1 H), 7.86 (dt, 1 H), 7.57 (d, 1 H), 7.36-7.39 (m, 2 H), 6.75-6.77 |
| | | (m, 2 H), 5.26 (s, 2 H), 5.10 (t, 1 H), 4.21 (d, 2 H), 3.85 (d, 2 H), |
| | | 3.76 (d, 2 H), 2.50 (s, 3 H), 1.42 (s, 3 H), 1.35 (s, 3 H). |
| Cpd 058 - | 421.3 | (400 MHz, DMSO-D₆) δ ppm: 8.54-8.59 (dd, 2 H), 7.83-7.87 (dt, |
| En1 | | 1 H), 7.61 (d, 1 H), 7.55 (d, 1 H), 7.39-7.46 (m, 2 H), 7.37 (t, 1 H), |
| | | 7.33 (d, 1 H), 7.14 (t, 2 H), 6.74-6.76 (dd, 1H), 5.25 (s, 2 H), 5.01- |
| | | 5.06 (m, 2 H), 3.66-3.70 (m, 2 H), 2.49-2.52 (m, 3 H). |
| Cpd 058 - | 421.3 | (400 MHz, DMSO-D₆) δ ppm: 8.54-8.59 (dd, 2 H), 7.83-7.87 (dt, |
| En2 | | 1 H), 7.61 (d, 1 H), 7.55 (d, 1 H), 7.39-7.46 (m, 2 H), 7.37 (t, 1 H), |
| | | 7.33 (d, 1 H), 7.14 (t, 2 H), 6.74-6.76 (dd, 1H), 5.25 (s, 2 H), 5.01- |
| | | 5.06 (m, 2 H), 3.66-3.70 (m, 2 H), 2.49-2.52 (m, 3 H). |
| Cpd 059 - | 404.3 | (400 MHz, DMSO-D₆) δ ppm: 8.60-8.55 (m, 3 H), 7.88-7.83 (m, |
| En1 | | 1 H), 7.79-7.76 (m, 1 H), 7.62 (d, 1 H), 7.57 (d, 1 H), 7.46-7.44 (m, |
| | | 2 H), 7.39-7.36 (m, 1 H), 7.30-7.27 (m, 1 H), 6.78-6.76 (dd, 1 H), |
| | | 5.27 (s, 2 H), 5.16-5.11 (q, 1 H), 4.99 (m, 1 H), 3.79 (m, 2 H), 2.57 |
| | | (s, 3 H). |
| Cpd 059 - | 404.3 | (400 MHz, DMSO-D₆) δ ppm: 8.60-8.55 (m, 3 H), 7.88-7.83 (m, |
| En2 | | 1 H), 7.79-7.76 (m, 1 H), 7.62 (d, 1 H), 7.57 (d, 1 H), 7.46-7.44 (m, |
| | | 2 H), 7.39-7.36 (m, 1 H), 7.30-7.27 (m, 1 H), 6.78-6.76 (dd, 1 H), |
| | | 5.27 (s, 2 H), 5.16-5.11 (q, 1 H), 4.99 (m, 1 H), 3.79 (m, 2 H), 2.57 |
| | | (s, 3 H). |
| Cpd 061 - | 435.36 | (400 MHz, DMSO-D₆) δ ppm: 8.58 (d, 1 H), 8.54 (d, 1 H), 7.83- |
| En1 | | 7.88 (m, 2 H), 7.54 (d, 1 H), 7.36-7.43 (m, 3 H), 7.25 (d, 1 H), 7.14 |
| | | (t, 2 H), 6.74 (dd, 1 H), 5.25 (s, 2 H), 5.13-5.18 (m, 1 H), 4.77 (s, |
| | | 1 H), 3.45 (brs, 2 H), 2.50 (s, 3 H), 1.87-2.03 (m, 2 H). |
| Cpd 061 - | 435.32 | (400 MHz, DMSO-D₆) δ ppm: 8.58 (d, 1 H), 8.54 (d, 1 H), 7.83- |
| En2 | | 7.88 (m, 2 H), 7.54 (d, 1 H), 7.36-7.43 (m, 3 H), 7.25 (d, 1 H), 7.14 |
| | | (t, 2 H), 6.74 (dd, 1 H), 5.25 (s, 2 H), 5.13-5.18 (m, 1 H), 4.77 (s, |
| | | 1 H), 3.45 (brs, 2 H), 2.50 (s, 3 H), 1.87-2.03 (m, 2 H). |
| Cpd 062 - | 409.2 | (400 MHz, DMSO-D₆) δ ppm: 8.60 (dd, 1 H), 8.54 (d, 1 H), 7.86 (dt, |
| En1 | | 1 H), 7.56 (d, 1 H), 7.36-7.39 (m, 1 H), 7.27-7.29 (m, 2 H), 6.75 |
| | | (dd, 1 H), 5.26 (s, 2 H), 5.07 (t, 1 H), 4.28-4.33 (m, 1 H), 3.51- |
| | | 3.56 (m, 1 H), 3.35-3.41 (m, 1 H), 2.55-2.67 (m, 2 H), 2.49 (s, 3 H). |
| Cpd 062 - | 409.2 | (400 MHz, DMSO-D₆) δ ppm: 8.60 (dd, 1 H), 8.54 (d, 1 H), 7.86 (dt, |
| En2 | | 1 H), 7.56 (d, 1 H), 7.36-7.39 (m, 1 H), 7.27-7.29 (m, 2 H), 6.75 |
| | | (dd, 1 H), 5.26 (s, 2 H), 5.07 (t, 1 H), 4.28-4.33 (m, 1 H), 3.51- |
| | | 3.56 (m, 1 H), 3.35-3.41 (m, 1 H), 2.55-2.67 (m, 2 H), 2.49 (s, 3 H). |
| Cpd 063 - | 477.3 | (400 MHz, DMSO-D₆) δ ppm: 8.59 (d, 1 H), 8.56 (d, 1 H), 7.84- |
| En1 | | 7.88 (m, 1 H), 7.56 (d, 1 H), 7.36-7.39 (m, 1 H), 7.32 (d, 1 H), 7.28 |
| | | (d, 1 H), 6.77 (dd, 1 H), 5.20-5.27 (m, 3 H), 4.91-4.93 (m, 1 H), |
| | | 4.13-4.18 (m, 1 H), 3.58-3.64 (m, 2 H), 2.50 (s, 3 H). |
| Cpd 063 - | 477.3 | (400 MHz, DMSO-D₆) δ ppm: 8.59 (d, 1 H), 8.56 (d, 1 H), 7.84- |
| En2 | | 7.88 (m, 1 H), 7.56 (d, 1 H), 7.36-7.39 (m, 1 H), 7.32 (d, 1 H), 7.28 |
| | | (d, 1 H), 6.77 (dd, 1H), 5.20-5.27 (m, 3 H), 4.91-4.93 (m, 1 H), |
| | | 4.13-4.18 (m, 1 H), 3.58-3.64 (m, 2 H), 2.50 (s, 3 H) |
| Cpd 064 - | 391.3 | (400 MHz, DMSO-D₆) δ ppm: 8.60 (d, 1 H), 8.53 (d, 1 H), 7.86 (dt, |
| En1 | | 1 H), 7.56 (d, 1 H), 7.36-7.39 (m, 1 H), 7.31 (d, 1 H), 7.20 (d, 1 H), |
| | | 6.74 (dd, 1 H), 6.00-6.30 (tt, 1 H), 5.27 (s, 2 H), 4.97 (brs, 1 H), |
| | | 4.11-4.21 (m, 1 H), 3.52-3.54 (m, 1 H), 3.42-3.44 (m, 1 H), |
| | | 2.50 (s, 3 H), 2.08-2.23 (m, 2 H). |
| Cpd 064 - | 391.2 | (400 MHz, DMSO-D₆) δ ppm: 8.60 (d, 1 H), 8.53 (d, 1 H), 7.86 (dt, |
| En2 | | 1 H), 7.56 (d, 1 H), 7.36-7.39 (m, 1 H), 7.31 (d, 1 H), 7.20 (d, 1 H), |
| | | 6.74 (dd, 1 H), 6.00-6.30 (tt, 1 H), 5.27 (s, 2 H), 4.97 (brs, 1 H), |
| | | 4.11-4.21 (m, 1 H), 3.52-3.54 (m, 1 H), 3.42-3.44 (m, 1 H), |
| | | 2.50 (s, 3 H), 2.08-2.23 (m, 2 H). LCMS: Rt = 1.72 min (99.85%), |
| Cpd 066 - | 419.3 | (400 MHz, DMSO-D₆) δ ppm: 8.60-8.59 (m, 1 H), 8.54 (d, 1 H), |
| En1 | | 7.84-7.88 (dt, 1 H), 7.56 (d, 1 H), 7.36-7.39 (m, 3 H), 7.25 (d, |
| | | 1 H), 6.92 (S, 1 H), 6.73-6.76 (dd, 1 H), 6.03-6.34 (dt, 1 H), 5.26 |
| | | (s, 2 H), 3.52-3.58 (q, 2 H), 3.33 (s, 3 H), 2.49-2.55 (m, 1 H), |
| | | 2.46 (s, 3 H), 2.29-2.38 (m, 1 H), 1.41 (s, 3 H) |
| Cpd 066 - | 419.3 | (400 MHz, DMSO-D₆) δ ppm: 8.60-8.59 (m, 1 H), 8.54 (d, 1 H), |
| En2 | | 7.84-7.88 (dt, 1 H), 7.56 (d, 1 H), 7.36-7.39 (m, 3 H), 7.25 (d, |
| | | 1 H), 6.91 (S, 1 H), 6.73-6.76 (dd, 1 H), 6.03-6.34 (dt, 1 H), 5.26 |
| | | (s, 2 H), 3.52-3.58 (q, 2 H), 3.33 (s, 3 H), 2.49-2.55 (m, 1 H), |
| | | 2.46 (s, 3 H), 2.29-2.39 (m, 1 H), 1.41 (s, 3 H) |
| Cpd 068 - | 383.3 | (400 MHz, DMSO-D₆) δ ppm: 8.60-8.55 (m, 2 H), 7.88-7.83 (m, |
| En1 | | 1 H), 7.57 (d, 1 H), 7.44 (s, 1 H), 7.39-7.33 (m, 2 H), 6.78-6.76 |
| | | (m, 1 H), 5.28 (s, 2 H), 5.13 (s, 1 H), 3.70 (s, 2 H), 2.52-2.49 (m, |
| | | 3 H), 2.10 (s, 3 H), 1.37 (s, 3 H); |
| Cpd 068 - | 383.3 | (400 MHz, DMSO-D₆) δ ppm: 8.60-8.55 (m, 2 H), 7.88-7.83 (m, |
| En2 | | 1 H), 7.57 (d, 2 H), 7.44 (s, 1 H), 7.39-7.33 (m, 2 H), 6.78-6.76 |
| | | (m, 1 H), 5.28 (s, 2 H), 5.12 (s, 1 H), 3.69 (s, 2 H), 2.52-2.49 (m, |
| | | 3 H), 2.10 (s, 3 H), 1.37 (s, 3 H); |
| Cpd 071 | 389.2 | (400 MHz, DMSO-D₆) δ ppm: 8.54-8.61 (m, 2 H), 7.85-7.89 (m, |
| | | 2 H), 7.57 (d, 1 H), 7.37-7.40 (m, 1 H), 7.31 (d, 1 H), 6.76-6.78 |
| | | (dd, 1 H), 5.29 (s, 2 H), 5.04 (t, 1 H), 3.62-3.83 (m, 2 H), 2.49 (s, |
| | | 3 H), 1.66-1.88 (m, 1 H). |
| Cpd 072 - | 403.3 | (400 MHz, DMSO-D₆) δ ppm: 8.60 (d, 1 H), 8.52 (d, 1 H), 7.84- |
| En1 | | 7.88 (dt, 1 H), 7.76 (s, 1 H), 7.57 (d, 1 H), 7.36-7.40 (dt, 1 H), 7.29 |
| | | (d, 1 H), 6.73-6.75 (dd, 1H), 5.83-6.13 (dt, 1 H), 5.29 (s, 2 H), |
| | | 5.05 (s, 1 H), 3.81 (d, 1 H), 3.68 (d, 1 H), 2.50 (s, 3 H), 1.67-1.74 |
| | | (m, 1 H), 1.20 (d, 2 H). |
| Cpd 072 - | 403.3 | (400 MHz, DMSO-D₆) δ ppm: 8.60 (d, 1 H), 8.52 (d, 1 H), 7.84- |
| En2 | | 7.88 (dt, 1 H), 7.76 (s, 1 H), 7.57 (d, 1 H), 7.36-7.40 (dt, 1 H), 7.29 |
| | | (d, 1 H), 6.73-6.75 (dd, 1H), 5.83-6.13 (dt, 1 H), 5.29 (s, 2 H), |
| | | 5.05 (s, 1 H), 3.81 (d, 1 H), 3.68 (d, 1 H), 2.50 (s, 3 H), 1.67-1.74 |
| | | (m, 1 H), 1.20 (d, 2 H). |
| Cpd 074 - | 433.3 | (400 MHz, DMSO-D₆) δ ppm: 8.60 (d, 1 H), 8.54 (d, 1 H), 7.86 (dt, |
| En1 | | 1 H), 7.58 (d, 1 H), 7.52 (d, 1 H), 7.49 (d, 1 H), 7.39-7.36 (m, 1 H), |
| | | 7.05 (d, 1 H), 6.80 (d, 1 H), 6.75 (dd, 1 H), 5.49 (q, 1 H), 5.28 (q, |
| | | 2 H), 4.99 (t, 1 H), 3.84-3.79 (m, 2 H), 3.48-3.43 (m, 1 H), 2.49 (s, |
| | | 3 H), 1.02-0.95 (m, 4 H) |
| Cpd 074 - | 433.3 | (400 MHz, DMSO-D₆) δ ppm: 8.61 (d, 1 H), 8.54 (d, 1 H), 7.86 (dt, |
| En2 | | 1 H), 7.58 (d, 1 H), 7.52 (d, 1 H), 7.49 (d, 1 H), 7.39-7.36 (m, 1 H), |
| | | 7.05 (d, 1 H), 6.80 (d, 1 H), 6.75 (dd, 1 H), 5.49 (q, 1 H), 5.28 (q, |
| | | 2 H), 4.98 (t, 1 H), 3.86-3.77 (m, 2 H), 3.48-3.44 (m, 1 H), 2.49 (s, |
| | | 3 H), 1.02-0.96 (m, 4 H). |
| Cpd 075 - | 421.3 | (400 MHz, DMSO-D₆) δ ppm: 8.55-8.59 (m, 2 H), 7.84-7.88 (m, |
| En1 | | 1 H), 7.57 (t, 2 H), 7.46 (d, 1 H), 7.36-7.39 (q, 1 H), 7.26 (s, 1 H), |
| | | 6.77 (dd, 1 H), 6.15 (d, 1 H), 5.26 (s, 2 H), 5.12 (t, 1 H), 3.80 (s, |
| | | 3 H), 3.74 (d, 2 H), 2.56 (s, 3 H), 1.68 (s, 3 H). |
| Cpd 075 - | 421.3 | (400 MHz, DMSO-D₆) δ ppm: 8.55-8.59 (m, 2 H), 7.84-7.85 (m, |
| En2 | | 1 H), 7.57 (t, 2 H), 7.46 (d, 1 H), 7.37 (q, 1 H), 7.26 (s, 1 H), 6.77 (dd, |
| | | 1 H), 6.15 (d, 1 H), 5.26 (s, 2 H), 5.12 (t, 1 H), 3.80 (s, 3 H), 3.74 (d, |
| | | 2 H), 2.56 (s, 3 H), 1.68 (s, 3 H). LCMS: Rt = 1.70 min (99.73%) |
| Cpd 076 - | 384.2 | (400 MHz, DMSO-D₆) δ ppm: 8.60 (d, 1 H), 8.53 (d, 1 H), 7.86 (t, |
| En1 | | 1 H), 7.57 (d, 1 H), 7.43 (br s, 2 H), 7.39-7.36 (m, 1 H), 7.32 (d, 1 H), |
| | | 6.91 (br s, 1 H), 6.74 (dd, 1 H), 5.27 (s, 2 H), 4.85-4.83 (m, 1 H), |
| | | 4.27-4.22 (m, 1 H), 3.54-3.41 (m, 2 H), 2.45 (s, 3 H), 2.43 (t, 2 H) |
| Cpd 076 - | 384.2 | (400 MHz, DMSO-D₆) δ ppm: 8.60 (d, 1 H), 8.53 (d, 1 H), 7.86 (t, |
| En2 | | 1 H), 7.57 (d, 1 H), 7.43 (br s, 2 H), 7.39-7.36 (m, 1 H), 7.32 (d, 1 H), |
| | | 6.91 (br s, 1 H), 6.74 (dd, 1 H), 5.27 (s, 2 H), 4.85-4.83 (m, 1 H), |
| | | 4.27-4.22 (m, 1 H), 3.54-3.41 (m, 2 H), 2.45 (s, 3 H), 2.43 (t, 2 H). |
| Cpd 077 - | 411.4 | (400 MHz, DMSO-D₆) δ ppm: 8.60 (dd, 1 H), 8.53 (d, 1 H), 7.86 (td, |
| En1 | | 1 H), 7.56 (d, 1 H), 7.38 (dt, 1 H), 7.32 (d, 1 H), 6.95 (d, 1 H), 6.74 |
| | | (dd, 1 H), 5.26 (s, 2H), 4.72 (t, 1 H), 3.83-3.88 (m, 3 H), 3.49- |
| | | 3.59 (m, 2 H), 3.21-3.29 (m, 2 H), 2.49 (s, 3 H), 1.82-1.90 (m, |
| | | 1 H), 1.61 (d, 2 H), 1.26-1.37 (m, 2 H). |
| Cpd 077 - | 411.4 | (400 MHz, DMSO-D₆) δ ppm: 8.60 (dd, 1 H), 8.53 (d, 1 H), 7.86 (td, |
| En2 | | 1 H), 7.56 (d, 1 H), 7.38 (dt, 1 H), 7.32 (d, 1 H), 6.95 (d, 1 H), 6.74 |
| | | (dd, 1 H), 5.26 (s, 2H), 4.72 (t, 1 H), 3.83-3.87 (m, 3 H), 3.50- |
| | | 3.57 (m, 2 H), 3.21-3.31 (m, 2 H), 2.49 (s, 3 H), 1.82-1.90 (m, |
| | | 1 H)), 1.60 (d, 2 H), 1.29-1.37 (m, 2 H). |
| Cpd 079 - | 419.3 | (400 MHz, DMSO-D₆) δ ppm: 8.72 (d, 2 H), 8.60-8.62 (m, 1 H), |
| En 1 | | 8.51 (d, 1 H), 7.85-7.89 (m, 1 H), 7.58 (d, 1 H), 7.37-7.40 (m, |
| | | 1 H), 7.31 (t, 2 H), 7.25 (d, 1 H), 6.72-6.74 (dd, 1 H), 5.29 (s, 2 H), |
| | | 4.88 (t, 1 H), 4.50 (m, 1 H), 3.56-3.60 (m, 1 H), 3.42-3.48 (m, |
| | | 1 H), 3.25-3.30 (m, 1 H), 3.07-3.12 (m, 1 H), 2.41 (s, 3 H). |
| Cpd 079 - | 419.3 | (400 MHz, DMSO-D₆) δ ppm: 8.72 (d, 2 H), 8.60-8.62 (m, 1 H), |
| En 2 | | 8.51 (d, 1 H), 7.85-7.89 (m, 1 H), 7.58 (d, 1 H), 7.37-7.40 (m, |
| | | 1 H), 7.31 (t, 2 H), 7.25 (d, 1 H), 6.72-6.74 (dd, 1 H), 5.29 (s, 2 H), |
| | | 4.88 (t, 1 H), 4.50 (m, 1 H), 3.57-3.60 (m, 1 H), 3.45-3.46 (m, |
| | | 1 H), 3.25-3.30 (m, 1 H), 3.07-3.12 (m, 1 H), 2.41 (s, 3 H). |
| Cpd 081 - | 409.3 | (400 MHz, DMSO-D₆) δ ppm: 8.60 (dd, 1 H), 8.54 (d, 1 H), 7.86 (dt, |
| En1 | | 1 H), 7.57 (d, 1 H), 7.36-7.40 (m, 3 H), 6.76 (dd, 1 H), 5.31-5.32 |
| | | (m, 1 H), 5.27 (s, 2 H), 3.97-3.99 (m, 1 H), 3.29-3.35 (m, 2 H), |
| | | 2.50 (m, 3 H), 2.45-2.50 (m, 1 H), 2.30-2.36 (m, 1 H). |
| Cpd 081 - | 409.3 | (400 MHz, DMSO-D₆) δ ppm: 8.60 (dd, 1 H), 8.54 (d, 1 H), 7.86 (dt, |
| En2 | | 1 H), 7.57 (d, 1 H), 7.36-7.40 (m, 3 H), 6.76 (dd, 1 H), 5.31-5.32 |
| | | (m, 1 H), 5.27 (s, 2 H), 3.97-3.99 (m, 1 H), 3.29-3.35 (m, 2 H), |
| | | 2.50 (m, 3 H), 2.45-2.50 (m, 1 H), 2.30-2.36 (m, 1 H). |
| Cpd 082 - | 420.2 | (400 MHz, DMSO-D₆) δ ppm: 8.60 (dd, 1H), 8.54 (dd, 1 H), 7.83- |
| En1 | | 7.88 (m, 1 H), 7.60 (d, 1 H), 7.56 (d, 1 H), 7.36-7.39 (m, 1 H), 7.25 |
| | | (d, 1 H), 6.75-6.77 (m, 1 H), 5.29 (s, 2 H), 4.46-4.53 (m, 1 H), |
| | | 3.20-3.25 (m, 1 H), 3.01-3.10 (m, 2 H), 2.84-2.88 (m, 1 H), |
| | | 2.67-2.71 (M, 2 H), 2.50 (s, 3 H) |
| Cpd 082 - | 420.2 | (400 MHz, DMSO-D₆) δ ppm: 8.60 (dd, 1H), 8.54 (dd, 1 H), 7.83- |
| En2 | | 7.88 (m, 1 H), 7.60 (d, 1 H), 7.56 (d, 1 H), 7.36-7.39 (m, 1 H), 7.25 |
| | | (d, 1 H), 6.75-6.77 (m, 1 H), 5.29 (s, 2 H), 4.46-4.53 (m, 1 H), |
| | | 3.20-3.25 (m, 1 H), 3.01-3.10 (m, 2 H), 2.84-2.88 (m, 1 H), |
| | | 2.67-2.71 (M, 2 H), 2.50 (s, 3 H) |
| Cpd 084 - | 430.3 | (400 MHz, DMSO-D₆) δ ppm: 8.54-8.60 (m, 2 H), 7.83-8.87 (dt, |
| En1 | | 1 H), 7.56 (d, 1 H), 7.33-7.48 (m, 2 H), 6.84 (s, 1 H), 6.74-6.76 (dd, |
| | | 1 H), 6.00-6.24 (m, 1 H), 5.23-5.30 (m, 2 H), 3.04 (d, 1H), 2.83 |
| | | (d, 1H), 2.63-2.71 (m, 2 H), 2.47-2.54 (m, 4 H), 2.36 (d, 1 H), |
| | | 2.19-2.23 (m, 1 H), 1.35-1.52 (m, 3 H), 123 (s, 1 H), |
| Cpd 084 - | 430.3 | (400 MHz, DMSO-D₆) δ ppm: 8.54-8.60 (m, 2 H), 7.82-8.87 (m, |
| En2 | | 1 H), 7.62 (d, 1 H), 7.44 (d, 1H), 7.35-7.38 (m, 1 H), 6.84 (s, 1 H), |
| | | 6.74-6.76 (dd, 1 H), 6.00-6.23 (m, 1 H), 5.21-5.30 (m, 2 H), |
| | | 3.04 (d, 1H), 2.83 (d, 1H), 2.63-2.71 (m, 2 H), 2.47-2.54 (m, |
| | | 4 H), 2.36 (d, 1 H), 2.19-2.23 (m, 1 H), 1.35-1.51 (m, 3 H), 1.23 (s, |
| | | 1 H), |
| Cpd 087 - | 407.3 | (400 MHz, DMSO-D₆) δ ppm: 8.60 (d, 1 H), 8.54 (d, 1 H), 7.88- |
| En1 | | 7.84 (m, 1 H), 7.58-7.56 (m, 2 H), 7.40-7.36 (m, 3 H), 6.76-6.74 |
| | | (dd, 1 H), 6.20-6.19 (d, 1 H), 5.27 (s, 2 H), 5.15-5.10 (q, 1 H), |
| | | 4.85 (s, 1 H), 3.79 (s, 3 H), 3.71 (d, 2 H), 2.52 (s, 3 H). |
| Cpd 087 - | 407.3 | (400 MHz, DMSO-D₆) δ ppm: 8.61-8.59 (m, 1 H), 8.54 (d, 1 H), |
| En2 | | 7.88-7.84 (m, 1 H), 7.58-7.56 (m, 2 H), 7.40-7.36 (m, 3 H), 6.76- |
| | | 6.74 (dd, 1 H), 6.20-6.19 (d, 1 H), 5.27 (s, 2 H), 5.15-5.10 (q, |
| | | 1 H), 4.86 (s, 1 H), 3.79 (s, 3 H), 3.71 (d, 2 H), 2.52 (s, 3 H). |
| Cpd 088 - | 418.3 | (400 MHz, DMSO-D₆) δ ppm: 8.61 (d, 1 H), 8.48-8.52 (m, 2 H), |
| En1 | | 7.87 (t, 1 H), 7.69 (t, 1 H), 7.57 (d, 1 H), 7.38 (t, 1 H), 7.26-7.32 |
| | | (m, 3 H), 7.18-7.21 (m, 1 H), 6.72-6.75 (m, 1 H), 5.25 (t, 2 H), |
| | | 4.88 (t, 1 H), 4.36-4.38 (m, 1 H), 3.53-3.58 (m, 1 H), 3.31-3.43 |
| | | (m, 1 H), 3.09-3.13 (m, 1 H), 2.97-3.03 (m, 1 H), 2.32 (s, 3 H). |
| Cpd 088 - | 418.3 | (400 MHz, DMSO-D₆) δ ppm: 8.61 (d, 1 H), 8.48-8.52 (m, 2 H), |
| En2 | | 7.87 (t, 1 H), 7.69 (t, 1 H), 7.57 (d, 1 H), 7.38 (t, 1 H), 7.26-7.32 |
| | | (m, 3 H), 7.18-7.21 (m, 1 H), 6.72-6.75 (m, 1 H), 5.25 (t, 2 H), |
| | | 4.88 (t, 1 H), 4.36-4.38 (m, 1 H), 3.53-3.58 (m, 1 H), 3.31-3.43 |
| | | (m, 1 H), 3.09-3.13 (m, 1 H), 2.97-3.03 (m, 1 H), 2.32 (s, 3 H). |
| Cpd 089 - | 384.3 | (400 MHz, DMSO-D₆) δ ppm: 8.60 (d, 1 H), 8.54 (d, 1 H), 7.86 (t, |
| En1 | | 1 H), 7.57 (d, 1 H), 7.50 (d, 1 H), 7.38 (t, 2 H), 7.26 (s, 1 H), 7.21 (s, |
| | | 1 H), 6.78-6.75 (m, 1 H), 5.25 (s, 2 H), 5.11 (t, 1 H), 3.93-3.89 (m, |
| | | 1 H), 3.76-3.72 (m, 1 H), 2.55 (s, 3 H), 1.51 (s, 3 H). |
| Cpd 089 - | 384.3 | (400 MHz, DMSO-D₆) δ ppm: 8.60 (d, 1 H), 8.54 (d, 1 H), 7.86 (t, |
| En2 | | 1 H), 7.57 (d, 1 H), 7.50 (d, 1 H), 7.38 (t, 2 H), 7.26 (s, 1 H), 7.21 (s, |
| | | 1 H), 6.78-6.75 (m, 1 H), 5.26 (s, 2 H), 5.11 (t, 1 H), 3.93-3.89 (m, |
| | | 1 H), 3.76-3.72 (m, 1 H), 2.55 (s, 3 H), 1.51 (s, 3 H). |
| Cpd 090 - | 395.3 | (400 MHz, DMSO-D₆) δ ppm: 8.59-8.60 (m, 1 H), 8.57 (s, 1 H), |
| En1 | | 7.86 (t, 1 H), 7.67 (d, 1 H), 7.56 (d, 1 H), 7.36-7.39 (m, 1 H), 7.26 |
| | | (d, 1 H), 6.79 (dd, 1 H), 5.27 (s, 2 H), 5.23 (s, 1 H), 4.77-4.81 (m, |
| | | 1 H), 3.73-3.80 (m, 2 H), 2.50 (s, 3 H). |
| Cpd 090 - | 395.2 | (400 MHz, DMSO-D₆) δ ppm: 8.59-8.60 (m, 1 H), 8.57 (s, 1 H), |
| En2 | | 7.86 (t, 1 H), 7.67 (d, 1 H), 7.56 (d, 1 H), 7.36-7.39 (m, 1 H), 7.26 |
| | | (d, 1 H), 6.79 (dd, 1 H), 5.27 (s, 2 H), 5.23 (s, 1 H), 4.77-4.81 (m, |
| | | 1 H), 3.73-3.80 (m, 2 H), 2.50 (s, 3 H) |
| Cpd 091 - | 396.3 | (400 MHz, DMSO-D₆) δ ppm: 8.61 (d, 1 H), 8.55 (d, 1 H) 7.88-7.86 |
| En1 | | (m, 1 H), 7.75 (s, 1 H), 7.56 (d, 1 H), 7.39-7.37 (m, 2 H), 6.87 (s, |
| | | 1 H), 6.77-6.75 (m, 1 H), 5.26 (s, 3 H), 3.64-3.56 (m, 2 H), 3.28- |
| | | 317 (m, 2 H), 2.63-2.43 (m, 3 H), 2.38-2.36 (m, 2 H); |
| Cpd 091 - | 396.2 | (400 MHz, DMSO-D₆) δ ppm: 8.61 (d, 1 H), 8.55 (d, 1 H) 7.88-7.86 |
| En2 | | (m, 1 H), 7.75 (s, 1 H), 7.56 (d, 1 H), 7.39-7.37 (m, 2 H), 6.87 (s, |
| | | 1 H), 6.77-6.75 (m, 1 H), 5.26-5.23 (m, 3 H), 3.64-3.56 (m, 2 H), |
| | | 3.28-317 (m, 2 H), 2.50-2.49 (m, 3 H), 2.49-2.43 (m, 2 H) |

### Chiral Analytical Data

Chiral SFC

| | | | co- | Flow | | |
|---|---|---|---|---|---|---|
| Cpd | Column | | solvent | [g/ | RT | Purity |
| Code | Name | co-solvent | % | min] | [min] | [%] |
| Cpd 011 - | (1) | MeOH | 30 | 3 | 1.52 | 99.9 |
| En1 | | | | | | |
| Cpd 011 - | (1) | MeOH | 30 | 3 | 2.26 | 99.9 |
| En2 | | | | | | |
| Cpd 017 - | (7) | MeOH | 40 | 3 | 2.48 | 99.9 |
| En1 | | | | | | |
| Cpd 017 - | (7) | MeOH | 40 | 3 | 3.23 | 98.8 |
| En2 | | | | | | |
| Cpd 018 - | (7) | 0.5% DEA in | 40 | 4 | 3.49 | 99.9 |
| En1 | | EtOH | | | | |
| Cpd 018 - | (7) | 0.5% DEA in | 40 | 4 | 10.02 | 99.9 |
| En2 | | EtOH | | | | |
| Cpd 024 - | (3) | 0.5% DEA in | 40 | 3 | 4.51 | 99.9 |
| En1 | | MeOH | | | | |
| Cpd 024 - | (3) | 0.5% DEA in | 40 | 3 | 6.51 | 99.9 |
| En2 | | MeOH | | | | |
| Cpd 025 - | (5) | MeOH | 30 | 3 | 1.52 | 99.8 |
| En1 | | | | | | |
| Cpd 025 - | (5) | MeOH | 30 | 3 | 1.88 | 99.1 |
| En2 | | | | | | |
| Cpd 026 - | (2) | 30 nM | 20 | 3 | 7.13 | 99.7 |
| En1 | | MeOHNH3 in | | | | |
| | | IPA | | | | |
| Cpd 026 - | (2) | 30 nM | 20 | 3 | 9.06 | 96.4 |
| En2 | | MeOHNH3 in | | | | |
| | | IPA | | | | |
| Cpd 028 - | (1) | 0.5% IPAmine in | 20 | 3 | 4.66 | 98.7 |
| En1 | | IPA | | | | |
| Cpd 028 - | (1) | 0.5% IPAmine in | 20 | 3 | 5.66 | 96.3 |
| En2 | | IPA | | | | |
| Cpd 029 - | (1) | 0.5% IPAmine in | 20 | 3 | 4.56 | 99.4 |
| En1 | | IPA | | | | |
| Cpd 029 - | (1) | 0.5% IPAmine in | 20 | 3 | 5.55 | 96.8 |
| En2 | | IPA | | | | |
| Cpd 031 - | (1) | IPA | 20 | 3 | 3.45 | 98.7 |
| En1 | | | | | | |
| Cpd 031 - | (1) | IPA | 20 | 3 | 4.54 | 92.5 |
| En2 | | | | | | |
| Cpd 032 - | (1) | IPA | 25 | 3 | 2.17 | 99 |
| En1 | | | | | | |
| Cpd 032 - | (1) | IPA | 25 | 3 | 2.89 | 97.4 |
| En2 | | | | | | |
| Cpd 037 - | (6) | MeOH | 30 | 3 | 3.43 | 99.7 |
| En1 | | | | | | |
| Cpd 037 - | (6) | MeOH | 30 | 3 | 4.03 | 95.9 |
| En2 | | | | | | |
| Cpd 038 - | (2) | MeOH | 20 | 3 | 4.34 | 99.8 |
| En1 | | | | | | |
| Cpd 038 - | (2) | MeOH | 20 | 3 | 5.11 | 99.7 |
| En2 | | | | | | |
| Cpd 050 - | (15) | MeOH | 40 | 4 | 3.28 | 99.6 |
| En1 | | | | | | |
| Cpd 050 - | (15) | MeOH | 40 | 4 | 6.45 | 99.3 |
| En2 | | | | | | |
| Cpd 051 - | (16) | MeOH | 40 | 3 | 4.01 | 99.3 |
| En1 | | | | | | |
| Cpd 051 - | (16) | MeOH | 40 | 3 | 7.92 | 99.6 |
| En2 | | | | | | |
| Cpd 052 - | (2) | 0.5% DEA in | 40 | 3 | 2.56 | 99.4 |
| En1 | | MeOH | | | | |
| Cpd 052 - | (2) | 0.5% DEA in | 40 | 3 | 3.94 | 99.1 |
| En2 | | MeOH | | | | |
| Cpd 053 - | (3) | 0.5% DEA in | 30 | 3 | 5.07 | 99.2 |
| En 1 | | MeOH | | | | |
| Cpd 053 - | (3) | 0.5% DEA in | 30 | 3 | 6.67 | 98.5 |
| En 2 | | MeOH | | | | |
| Cpd 054 - | (15) | 0.5% DEA in | 40 | 4 | 4.02 | 99.9 |
| En1 | | MeOH | | | | |
| Cpd 054 - | (15) | 0.5% DEA in | 40 | 4 | 9.91 | 99.8 |
| En2 | | MeOH | | | | |
| Cpd 058 - | (14) | MeOH | 30 | 3 | 8.86 | 99.96 |
| En1 | | | | | | |
| Cpd 058 - | (14) | MeOH | 30 | 3 | 11.91 | 99.92 |
| En2 | | | | | | |
| Cpd 059 - | (3) | MeOH | 40 | 3 | 9.82 | 99.97 |
| En1 | | | | | | |
| Cpd 059 - | (3) | MeOH | 40 | 3 | 12.23 | 99.5 |
| En2 | | | | | | |
| Cpd 061 - | (7) | MeOH | 40 | 3 | 8.18 | 99.9 |
| En1 | | | | | | |
| Cpd 061 - | (7) | MeOH | 40 | 3 | 10.67 | 99.9 |
| En2 | | | | | | |
| Cpd 062 - | (7) | MeOH | 30 | 3 | 3.82 | 99.8 |
| En1 | | | | | | |
| Cpd 062 - | (7) | MeOH | 30 | 3 | 6.21 | 99.5 |
| En2 | | | | | | |
| Cpd 063 - | (16) | MeOH | 30 | 3 | 2.04 | 99.9 |
| En1 | | | | | | |
| Cpd 063 - | (16) | MeOH | 30 | 3 | 7.06 | 99.9 |
| En2 | | | | | | |
| Cpd 064 - | (6) | MeOH | 40 | 4 | 2.09 | 99.9 |
| En1 | | | | | | |
| Cpd 064 - | (6) | MeOH | 40 | 4 | 5.07 | 99.9 |
| En2 | | | | | | |
| Cpd 066 - | (16) | MeOH | 40 | 3 | 2.77 | 99.9 |
| En1 | | | | | | |
| Cpd 066 - | (16) | MeOH | 40 | 3 | 3.59 | 99.7 |
| En2 | | | | | | |
| Cpd 068 - | (15) | 0.5% IPAmine in | 30 | 3 | 5.31 | 99.6 |
| En1 | | IPA | | | | |
| Cpd 068 - | (15) | 0.5% IPAmine in | 30 | 3 | 7.87 | 99.6 |
| En2 | | IPA | | | | |
| Cpd 072 - | (16) | MeOH | 30 | 3 | 3.64 | 99.7 |
| En1 | | | | | | |
| Cpd 072 - | (16) | MeOH | 30 | 3 | 4.64 | 99.1 |
| En2 | | | | | | |
| Cpd 074 - | (2) | 0.5% DEA in | 25 | 3 | 5.99 | 99.2 |
| En1 | | MeOH | | | | |
| Cpd 074 - | (2) | 0.5% DEA in | 25 | 3 | 3.28 | 99.9 |
| En2 | | MeOH | | | | |
| Cpd 075 - | (15) | MeOH | 35 | 3 | 5.85 | 99.8 |
| En1 | | | | | | |
| Cpd 075 - | (15) | MeOH | 35 | 3 | 6.72 | 98.8 |
| En2 | | | | | | |
| Cpd 076 - | (12) | 0.5% DEA in | 40 | 4 | 1.37 | 99.7 |
| En1 | | MeOH | | | | |
| Cpd 076 - | (12) | 0.5% DEA in | 30 | 3 | 5.06 | 98.8 |
| En2 | | MeOH | | | | |
| Cpd 077 - | (2) | MeOH | 30 | 3 | 2.47 | 98.2 |
| En1 | | | | | | |
| Cpd 077 - | (2) | MeOH | 30 | 3 | 4.42 | 96.2 |
| En2 | | | | | | |
| Cpd 079 - | (2) | MeOH | 50 | 3 | 4.57 | 99.9 |
| En 1 | | | | | | |
| Cpd 079 - | (2) | MeOH | 50 | 3 | 7.38 | 99.8 |
| En 2 | | | | | | |
| Cpd 081 - | (16) | 0.5% IPAmine in | 30 | 3 | 8.71 | 99.9 |
| En1 | | IPA | | | | |
| Cpd 081 - | (16) | 0.5% IPAmine in | 30 | 3 | 10.37 | 97.8 |
| En2 | | IPA | | | | |
| Cpd 082 - | (15) | 0.5% DEA in | 35 | 3 | 1.91 | 99.2 |
| En1 | | MeOH | | | | |
| Cpd 082 - | (15) | 0.5% DEA in | 35 | 3 | 5.13 | 99.2 |
| En2 | | MeOH | | | | |
| Cpd 084 - | (7) | 0.5% DEA in | 30 | 3 | 5.88 | 97.9 |
| En1 | | EtOH | | | | |
| Cpd 084 - | (7) | 0.5% DEA in | 30 | 3 | 7.1 | 97.3 |
| En2 | | EtOH | | | | |
| Cpd 087 - | (3) | MeOH | 40 | 3 | 7.14 | 99.8 |
| En1 | | | | | | |
| Cpd 087 - | (3) | MeOH | 40 | 3 | 10.77 | 99.2 |
| En2 | | | | | | |
| Cpd 088 - | (6) | MeOH | 50 | 4 | 4.05 | 99.7 |
| En1 | | | | | | |
| Cpd 088 - | (6) | MeOH | 50 | 4 | 6.32 | 98.9 |
| En2 | | | | | | |
| Cpd 089 - | (13) | 0.5% IPAmine in | 40 | 3 | 5.7 | 99.6 |
| En1 | | IPA | | | | |
| Cpd 089 - | (13) | 0.5% IPAmine in | 40 | 3 | 6.82 | 99.2 |
| En2 | | IPA | | | | |
| Cpd 090 - | (6) | MeOH | 25 | 3 | 1.25 | 99.9 |
| En1 | | | | | | |
| Cpd 090 - | (6) | MeOH | 25 | 3 | 2.16 | 99.7 |
| En2 | | | | | | |
| Cpd 091 - | (15) | MeOH | 40 | 3 | 4.59 | 99.8 |
| En1 | | | | | | |
| Cpd 091 - | (15) | MeOH | 40 | 3 | 5.87 | 99.3 |
| En2 | | | | | | |

ChiralpakAD-3(4.6*150 mm)3.5 μm=(1); ChiralpakAD-H(4.6*250 mm)5 μm=(2); Chiralpak IC (4.6*250 mm)5 μm=(3); Chiralpak IE-3(4.6*150 mm)3 μm=(4); Chiralpak IF-3(4.6*150 mm)3 μm=(5); Chiralpak IF (4.6*250 mm)5 μm=(6); Chiralpak IG-3(4.6*150 mm)3 μm=(7); ChiralCel OD-3(4.6*150 mm)3 μm=(8); ChiralCel OH-3(4.6*150 mm)3 μm=(9); ChiralCel OZ-3(4.6*150 mm)3 μm=(10); C-AMYLOSE A=(11); (R,R) WHELK-01 (4.6*150 mm)3.5 μm=(12); Chiralpak IK (4.6*250 mm)5 μm=(13), CHIRALART Amylose C—NeO (4.6*250 mm)5 μm=(14); CHIRALCEL OX-3 (4.6*150 mm) 3 μm=(15); LUX CELLULOSE-4 (4.6*150 mm)3 μm=(16).

Chiral HPLC

| | | | | Flow | | |
|---|---|---|---|---|---|---|
| Cpd | | | Ratio | [mL/ | RT | Purity |
| Code | Column | Phase A/B | A/B | min] | [min] | [%] |
| Cpd 015 - | (3) | nHex/EtOH | 70/30 | 1 | 10.03 | 99.9 |
| En1 | | | | | | |
| Cpd 015 - | (3) | nHex/EtOH | 70/30 | 1 | 12.32 | 98.8 |
| En2 | | | | | | |
| Cpd 016 - | (3) | nHex/EtOH | 50/50 | 1 | 8.8 | 99.9 |
| En1 | | | | | | |
| Cpd 016 - | (3) | nHex/EtOH | 50/50 | 1 | 10.33 | 99.9 |
| En2 | | | | | | |
| Cpd 019 - | (3) | nHex/EtOH | 50/50 | 1 | 9.27 | 99.4 |
| En1 | | | | | | |
| Cpd 019 - | (3) | nHex/EtOH | 50/50 | 1 | 10.36 | 97.7 |
| En2 | | | | | | |
| Cpd 020 - | (3) | nHex/EtOH | 60/40 | 1 | 13.6 | 99.7 |
| En1 | | | | | | |
| Cpd 020 - | (3) | nHex/EtOH | 60/40 | 1 | 16.52 | 97.6 |
| En2 | | | | | | |
| Cpd 021 - | (3) | nHex/EtOH | 50/50 | 1 | 22.61 | 99.9 |
| En1 | | | | | | |
| Cpd 021 - | (3) | nHex/EtOH | 50/50 | 1 | 34.91 | 99.7 |
| En2 | | | | | | |
| Cpd 022 - | (1) | nHex/EtOH | 70/30 | 1 | 14.15 | 99.8 |
| En1 | | | | | | |
| Cpd 022 - | (1) | nHex/EtOH | 70/30 | 1 | 15.32 | 98.4 |
| En2 | | | | | | |
| Cpd 023 - | (1) | nHex/IPA | 80/20 | 1 | 12.41 | 99.2 |
| En1 | | | | | | |
| Cpd 023 - | (1) | nHex/IPA | 80/20 | 1 | 15.93 | 95.6 |
| En2 | | | | | | |
| Cpd 027 - | (5) | nHex/EtOH | 40/60 | 3 | 19.17 | 99.8 |
| En1 | | | | | | |
| Cpd 027 - | (5) | nHex/EtOH | 40/60 | 3 | 23.29 | 98.4 |
| En2 | | | | | | |
| Cpd 030 - | (1) | nHex/EtOH | 70/30 | 1 | 7.13 | 99.7 |
| En1 | | | | | | |
| Cpd 030 - | (1) | nHex/EtOH | 70/30 | 1 | 12.61 | 95.6 |
| En2 | | | | | | |
| Cpd 049 - | (3) | DIPA/MeOH | 0.2/99.8 | 1 | 12.22 | 99.6 |
| En1 | | | | | | |
| Cpd 049 - | (3) | DIPA/MeOH | 0.2/99.8 | 1 | 18.03 | 99.4 |
| En2 | | | | | | |

Chiralpak AD-H (250 × 4.6 mm) 5 μm = (1); Chiralpak IA (4.6 × 250 mm) 5 μm = (2); Chiralpak IG (250 × 4.6 mm) 5 μm = (3); Lux Amylose-2 (250 × 4.6 mm) 5 μm = (4); Lux Cellulose-2 (250 × 4.6 mm) 5 μm = (5).

### Part B

### Monitoring the TRPM3 Ion Channel Driven Ca2+ Uptake.

In order to monitor the inhibition of the mouse TRPM3α2 (mTRPM3) ion channel by the compounds of the invention, a cellular system making use of an mTRPM3alpha2 or hTRPM3 overexpressing cell line (flip-in HEK293) was used. The TRPM3 channel was stimulated/opened with Pregnenolone sulfate (PS) (50 μM) which results in Ca²⁺ influx.

For mTRPM3, the intracellular Ca²⁺ was measured with a Calcium responsive dye, Fluor-4 AM ester (Invitrogen). Cells were cultured until a confluence of 80-90%, washed with Versene (Invitrogen) and detached from the surface by a short incubation with 0.05% Trypsin (Invitrogen). The tyrosination process was stopped by the addition of complete cell culture medium (DMEM, glutamax, 10% FCS, NEAA, Pen-Strep). Cells were collected and resuspended in Krebs buffer without Calcium at RT.

Prior the cell seeding (±2000 cells/well into a black, 384 well plate (Greiner)) the diluted compound was added in the assay plate, together with the PS dissolved in Krebs buffer containing Calcium. This resulted in a 2.4 mM Ca²⁺ assay solution. Directly after cell addition the plates were read on an Envision fluorescence reader (Perkin Elmer) by an Excitation of 485 nM and emission at 535 nM.

Channel inhibition was calculated compared to a non-PS stimulated control versus a condition stimulated with PS (50 μM) with vehicle. The ability of the compounds of the invention to inhibit this activity was determined as: Percentage inhibition=[1−((RFU determined for sample with test compound present−RFU determined for sample with positive control inhibitor) divided by (RFU determined in the presence of vehicle−RFU determined for sample with positive control inhibitor))]*100.

The activities of the Example compounds cpd 001-091 tested are depicted in the table below. All tested compounds show an activity in the Fluo-4 AM assay. The activity ranges A, B and C refer to IC50 values in the Fluo-4 AM assay as follows: “A”: IC₅₀<1 μM; “B”: 1 μM≤IC₅₀≤20 μM and “C”: IC₅₀>20 μM.

| Cpd Code | IC50 |
|---|---|
| Cpd 001 | A |
| Cpd 002 | B |
| Cpd 003 | A |
| Cpd 004 | B |
| Cpd 005 | B |
| Cpd 006 | B |
| Cpd 007 | A |
| Cpd 008 | B |
| Cpd 009 - En1 | B |
| Cpd 009 - En2 | C |
| Cpd 010 | B |
| Cpd 011 - En1 | A |
| Cpd 011 - En2 | B |
| Cpd 012 | A |
| Cpd 013 | B |
| Cpd 014 | A |
| Cpd 015 - En1 | B |
| Cpd 015 - En2 | C |
| Cpd 016 - En1 | A |
| Cpd 016 - En2 | B |
| Cpd 017 - En1 | B |
| Cpd 017 - En2 | C |
| Cpd 018 - En1 | B |
| Cpd 018 - En2 | A |
| Cpd 019 - En1 | A |
| Cpd 019 - En2 | B |
| Cpd 020 - En1 | A |
| Cpd 020 - En2 | B |
| Cpd 021 - En1 | B |
| Cpd 021 - En2 | C |
| Cpd 022 - En1 | A |
| Cpd 022 - En2 | C |
| Cpd 023 - En1 | A |
| Cpd 023 - En2 | B |
| Cpd 024 - En1 | B |
| Cpd 024 - En2 | B |
| Cpd 025 - En1 | B |
| Cpd 025 - En2 | A |
| Cpd 026 - En1 | A |
| Cpd 026 - En2 | B |
| Cpd 027 - En1 | B |
| Cpd 027 - En2 | B |
| Cpd 028 - En1 | A |
| Cpd 028 - En2 | B |
| Cpd 029 - En1 | A |
| Cpd 029 - En2 | A |
| Cpd 030 - En1 | A |
| Cpd 030 - En2 | B |
| Cpd 031 - En1 | A |
| Cpd 031 - En2 | A |
| Cpd 032 - En1 | A |
| Cpd 032 - En2 | B |
| Cpd 033 | C |
| Cpd 034 | C |
| Cpd 035 | C |
| Cpd 036 | C |
| Cpd 037 - En1 | B |
| Cpd 037 - En2 | A |
| Cpd 038 - En1 | B |
| Cpd 038 - En2 | A |
| Cpd 042 | C |
| Cpd 043 | C |
| Cpd 044 | B |
| Cpd 045 | B |
| Cpd 046 | C |
| Cpd 047 | B |
| Cpd 048 | C |
| Cpd 049 - En1 | B |
| Cpd 049 - En2 | C |
| Cpd 050 - En1 | C |
| Cpd 050 - En2 | B |
| Cpd 051 - En1 | C |
| Cpd 051 - En2 | B |
| Cpd 052 - En1 | B |
| Cpd 052 - En2 | C |
| Cpd 053 - En 1 | C |
| Cpd 053 - En 2 | B |
| Cpd 054 - En1 | C |
| Cpd 054 - En2 | B |
| Cpd 055 | B |
| Cpd 056 | C |
| Cpd 057 | C |
| Cpd 058 - En1 | C |
| Cpd 058 - En2 | C |
| Cpd 059 - En1 | C |
| Cpd 059 - En2 | B |
| Cpd 061 - En1 | C |
| Cpd 061 - En2 | C |
| Cpd 062 - En1 | B |
| Cpd 062 - En2 | C |
| Cpd 063 - En1 | C |
| Cpd 063 - En2 | A |
| Cpd 064 - En1 | B |
| Cpd 064 - En2 | C |
| Cpd 066 - En1 | C |
| Cpd 066 - En2 | C |
| Cpd 068 - En1 | B |
| Cpd 068 - En2 | B |
| Cpd 071 | C |
| Cpd 072 - En1 | B |
| Cpd 072 - En2 | C |
| Cpd 074 - En1 | B |
| Cpd 074 - En2 | B |
| Cpd 075 - En1 | C |
| Cpd 075 - En2 | C |
| Cpd 076 - En1 | C |
| Cpd 076 - En2 | C |
| Cpd 077 - En1 | C |
| Cpd 077 - En2 | C |
| Cpd 079 - En 1 | C |
| Cpd 079 - En 2 | C |
| Cpd 081 - En1 | C |
| Cpd 081 - En2 | B |
| Cpd 082 - En1 | B |
| Cpd 082 - En2 | A |
| Cpd 087 - En1 | C |
| Cpd 087 - En2 | B |
| Cpd 088 - En1 | B |
| Cpd 088 - En2 | C |
| Cpd 089 - En1 | C |
| Cpd 089 - En2 | A |
| Cpd 090 - En1 | B |
| Cpd 090 - En2 | B |
| Cpd 091 - En1 | A |
| Cpd 091 - En2 | C |

1. A compound of formula (I), a stereo-isomeric form, a physiologically acceptable salt, solvate and/or polymorph thereof 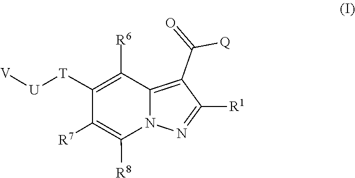
wherein
R¹ represents —F, —Cl, —Br, —I, —CN, —R^{W}, —OR^{W}, —OC(═O)R^{W}, —NR^{W}R^{X}, —NR^{W}C(═O)R^{X}, —SR^{W}, —S(═O)R^{W}, —S(═O)₂R^{W}, —C(═O)R^{W}, —C(═O)OR^{W}, or —C(═O)NR^{W}R^{X};
Q represents —OR² or —NR³R⁴;
R² represents —R^{Y};
R³ represents —OH or —R^{Y};
R⁴ represents —R^{Y} or —S(═O)₂R^{Y};
or R³ and R⁴ together form a 4, 5, 6, 7 or 8 membered heterocycle containing 1 to 3 heteroatoms selected from N, O and S, saturated or unsaturated, unsubstituted or mono- or polysubstituted;
T represents —O— and U represents —CR⁵R^{5′}—; or T represents —CR⁵R^{5′}— and U represents —O—;
R⁵ and R^{5′} independently of one another represent —R^{Y};
R⁶, R⁷ and R⁸ independently of one another represent —F, —Cl, —Br, —I, —CN, —NO₂, —SF₅, —R^{W}, —OR^{W}, —OC(═O)R^{W}, —NR^{W}R^{X}, —NR^{W}C(═O)R^{X}, —SR^{W}, —S(═O)R^{W}, —S(═O)₂R^{W}, —C(═O)R^{W}, —C(═O)OR^{W}, or —C(═O)NR^{W}R^{X};
V represents 3-14-membered heterocycloalkyl, saturated or unsaturated; 3-14-membered cycloalkyl, saturated or unsaturated; 5-14-membered aryl; C₁-C₆ alkyl or 5-14-membered heteroaryl; in each case unsubstituted, mono- or polysubstituted with substituents independently of one another selected from —F, —Cl, —Br, —I, —CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z};
wherein
R^{W} and R^{X} independently of one another and in each case independently represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted; or
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
R^{Y} and R^{Z} independently of one another and in each case independently represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted;
3-14-membered heterocycloalkyl, saturated or unsaturated, unsubstituted, mono- or polysubstituted; wherein said 3-14-membered heterocycloalkyl is optionally connected through —C₁-C₆-alkylene- or —C₁-C₆-heteroalkylene-, in each case saturated or unsaturated, unsubstituted, mono- or polysubstituted;
6-14-membered aryl, unsubstituted, mono- or polysubstituted; wherein said 6-14-membered aryl is optionally connected through —C₁-C₆-alkylene- or —C₁-C₆-heteroalkylene-, in each case saturated or unsaturated, unsubstituted, mono- or polysubstituted; or
5-14-membered heteroaryl, unsubstituted, mono- or polysubstituted; wherein said 5-14-membered heteroaryl is optionally connected through —C₁-C₆-alkylene- or —C₁-C₆-heteroalkylene-, in each case saturated or unsaturated, unsubstituted, mono- or polysubstituted;
or R^{Y} and R^{Z} together form a 4, 5, 6, 7 or 8 membered heterocycle containing 1 to 3 heteroatoms selected from N, O and S, saturated or unsaturated, unsubstituted or mono- or polysubstituted;
and wherein “mono- or polysubstituted” in each case independently means substituted with one or more substituents independently of one another selected from —F, —Cl, —Br, —I, —CN, —C₁₋₆-alkyl, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-O—CF₃, —C₁₋₆-alkylene-O—CF₂H, —C₁₋₆-alkylene-O—CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)—C₁₋₆-alkyl, —C₁₋₆-alkylene-C(═O)—C₁₋₆-alkyl, —C(═O)OH, —C₁₋₆-alkylene-C(═O)—OH, —C(═O)—OC₁₋₆-alkyl, —C₁₋₆-alkylene-C(═O)—OC₁₋₆-alkyl, —C(═O)O—C₁₋₆-alkylene-CF₃, —C(═O)—NH₂, —C₁₋₆-alkylene-C(═O)—NH₂, —C(═O)—NH(C₁₋₆-alkyl), —C₁₋₆-alkylene-C(═O)—NH(C₁₋₆-alkyl), —C(═O)—N(C₁₋₆-alkyl)₂, —C₁₋₆-alkylene-C(═O)—N(C₁₋₆-alkyl)₂, —C(═O)—NH(OH), —C₁₋₆-alkylene-C(═O)—NH(OH), —OH, —C₁₋₆-alkylene-OH, ═O, —OCF₃, —OCF₂H, —OCFH₂, —OCF₂Cl, —OCFCl₂, —O—C₁₋₆-alkyl, —C₁₋₆-alkylene-O—C₁₋₆-alkyl, —O—C₁₋₆-alkylene-O—C₁₋₆-alkyl, —O—C₁₋₆-alkylene-NH₂, —O—C₁₋₆-alkylene-NH—C₁₋₆-alkyl, —O—C₁₋₆-alkylene-N(C₁₋₆-alkyl)₂, —O—C(═O)—C₁₋₆-alkyl, —C₁₋₆-alkylene-O—C(═O)—C₁₋₆-alkyl, —O—C(═O)—O—C₁₋₆-alkyl, —C₁₋₆-alkylene-O—C(═O)—O—C₁₋₆-alkyl, —O—C(═O)—NH(C₁₋₆-alkyl), —C₁₋₆-alkylene-O—C(═O)—NH(C₁₋₆-alkyl), —O—C(═O)—N(C₁₋₆-alkyl)₂, —C₁₋₆-alkylene-O—C(═O)—N(C₁₋₆-alkyl)₂, —O—S(═O)₂—NH₂, —C₁₋₆-alkylene-O—S(═O)₂—NH₂, —O—S(═O)₂—NH(C₁₋₆-alkyl), —C₁₋₆-alkylene-O—S(═O)₂—NH(C₁₋₆-alkyl), —O—S(═O)₂—N(C₁₋₆-alkyl)₂, —C₁₋₆-alkylene-O—S(═O)₂—N(C₁₋₆-alkyl)₂, —NH₂, —NO, —NO₂, —C₁₋₆-alkylene-NH₂, —NH(C₁₋₆-alkyl), —N(3-14-membered cycloalkyl)(C₁₋₆-alkyl), —N(C₁₋₆-alkyl)-C₁₋₆-alkylene-OH, —N(H)—C₁₋₆-alkylene-OH, —C₁₋₆-alkylene-NH(C₁₋₆-alkyl), —N(C₁₋₆-alkyl)₂, —C₁₋₆-alkylene-N(C₁₋₆-alkyl)₂, —NH—C(═O)—C₁₋₆-alkyl, —C₁₋₆-alkylene-NH—C(═O)—C₁₋₆-alkyl, —NH—C(═O)—O—C₁₋₆-alkyl, —C₁₋₆-alkylene-NH—C(═O)—O—C₁₋₆-alkyl, —NH—C(═O)—NH₂, —C₁₋₆-alkylene-NH—C(═O)—NH₂, —NH—C(═O)—NH(C₁₋₆-alkyl), —C₁₋₆-alkylene-NH—C(═O)—NH(C₁₋₆-alkyl), —NH—C(═O)—N(C₁₋₆-alkyl)₂, —C₁₋₆-alkylene-NH—C(═O)—N(C₁₋₆-alkyl)₂, —N(C₁₋₆-alkyl)-C(═O)—C₁₋₆-alkyl, —C₁₋₆-alkylene-N(C₁₋₆-alkyl)-C(═O)—C₁₋₆-alkyl, —N(C₁₋₆-alkyl)-C(═O)—O—C₁₋₆-alkyl, —C₁₋₆-alkylene-N(C₁₋₆-alkyl)-C(═O)—O—C₁₋₆-alkyl, —N(C₁₋₆-alkyl)-C(═O)—NH₂, —C₁₋₆-alkylene-N(C₁₋₆-alkyl)-C(═O)—NH₂, —N(C₁₋₆-alkyl)-C(═O)—NH(C₁₋₆-alkyl), —C₁₋₆-alkylene-N(C₁₋₆-alkyl)-C(═O)—NH(C₁₋₆-alkyl), —N(C₁₋₆-alkyl)-C(═O)—N(C₁₋₆-alkyl)₂, —C₁₋₆-alkylene-N(C₁₋₆-alkyl)-C(═O)—N(C₁₋₆-alkyl)₂, —NH—S(═O)₂OH, —C₁₋₆-alkylene-NH—S(═O)₂OH, —NH—S(═O)₂—C₁₋₆-alkyl, —C₁₋₆-alkylene-NH—S(═O)₂—C₁₋₆-alkyl, —NH—S(═O)₂—O—C₁₋₆-alkyl, —C₁₋₆-alkylene-NH—S(═O)₂—O—C₁₋₆-alkyl, —NH—S(═O)₂—NH₂, —C₁₋₆-alkylene-NH—S(═O)₂—NH₂, —NH—S(═O)₂—NH(C₁₋₆-alkyl), —C₁₋₆-alkylene-NH—S(═O)₂—NH(C₁₋₆-alkyl), —NH—S(═O)₂N(C₁₋₆-alkyl)₂, —C₁₋₆-alkylene-NH—S(═O)₂N(C₁₋₆-alkyl)₂, —N(C₁₋₆-alkyl)-S(═O)₂—OH, —C₁₋₆-alkylene-N(C₁₋₆-alkyl)-S(═O)₂—OH, —N(C₁₋₆-alkyl)-S(═O)₂—C₁₋₆-alkyl, —C₁₋₆-alkylene-N(C₁₋₆-alkyl)-S(═O)₂—C₁₋₆-alkyl, —N(C₁₋₆-alkyl)-S(═O)₂—O—C₁₋₆-alkyl, —C₁₋₆-alkylene-N(C₁₋₆-alkyl)-S(═O)₂—O—C₁₋₆-alkyl, —N(C₁₋₆-alkyl)-S(═O)₂—NH₂, —C₁₋₆-alkylene-N(C₁₋₆-alkyl)-S(═O)₂—NH₂, —N(C₁₋₆-alkyl)-S(═O)₂—NH(C₁₋₆-alkyl), —C₁₋₆-alkylene-N(C₁₋₆-alkyl)-S(═O)₂—NH(C₁₋₆-alkyl), —N(C₁₋₆-alkyl)-S(═O)₂—N(C₁₋₆-alkyl)₂, —C₁₋₆-alkylene-N(C₁₋₆-alkyl)-S(═O)₂—N(C₁₋₆-alkyl)₂, —SH, ═S, —SF₅, —SCF₃, —SCF₂H, —SCFH₂, —S—C₁₋₆-alkyl, —C₁₋₆-alkylene-S—C₁₋₆-alkyl, —S(═O)—C₁₋₆-alkyl, —C₁₋₆-alkylene-S(═O)—C₁₋₆-alkyl, —S(═O)₂—C₁₋₆-alkyl, —C₁₋₆-alkylene-S(═O)₂—C₁₋₆-alkyl, —S(═O)₂—OH, —C₁₋₆-alkylene-S(═O)₂—OH, —S(═O)₂—O—C₁₋₆-alkyl, —C₁₋₆-alkylene-S(═O)₂—O—C₁₋₆-alkyl, —S(═O)₂—NH₂, —C₁₋₆-alkylene-S(═O)₂—NH₂, —S(═O)₂—NH(C₁₋₆-alkyl), —C₁₋₆-alkylene-S(═O)₂—NH(C₁₋₆-alkyl), —S(═O)₂—N(C₁₋₆-alkyl)₂, —C₁₋₆-alkylene-S(═O)₂—N(C₁₋₆-alkyl)₂, 3-14-membered cycloalkyl, —C₁₋₆-alkylene-(3-14-membered cycloalkyl), 3 to 14-membered heterocycloalkyl, —C₁₋₆-alkylene-(3 to 14-membered heterocycloalkyl), -phenyl, —C₁₋₆-alkylene-phenyl, 5 to 14-membered heteroaryl, —C₁₋₆-alkylene-(5 to 14-membered heteroaryl), —O-(3-14-membered cycloalkyl), —O-(3 to 14-membered heterocycloalkyl), —O-phenyl, —O-(5 to 14-membered heteroaryl), —C(═O)-(3-14-membered cycloalkyl), —C(═O)-(3 to 14-membered hetero-cycloalkyl), —C(═O)-phenyl, —C(═O)-(5 to 14-membered heteroaryl), —S(═O)₂-(3-14-membered cycloalkyl), —S(═O)₂-(3 to 14-membered heterocycloalkyl), —S(═O)₂-phenyl, —S(═O)₂-(5 to 14-membered heteroaryl).

2. The compound of claim 1, wherein R³ represents —H.

3. The compound of claim 2, wherein R⁴ represents a residue other than —H.

4. The compound of any one of claims 1 to 3, wherein R¹ represents -methyl or ethyl.

5. The compound of any one of claims 1 to 3, wherein T represents —O— and U represents —CR⁵R^{5′}—.

6. The compound of any one of claims 1 to 5, wherein the R^{Y} representing each of R⁵ and R^{5′} is H.

7. The compound of one of claims 1 to 6, wherein V represents (i) 5-14-membered heteroaryl selected from benzimidazole, benzisoxazole, benzoxazole, benzodioxole, benzofuran, benzothiadiazole, benzothiazole, benzothiophene, carbazole, cinnoline, dibenzofuran, furane, furazane, imidazole, imidazopyridine, indazole, indole, indolizine, isobenzofuran, isoindole, isoquinoline, isothiazole, isoxazole, naphthyridine, oxadiazole, oxazole, oxindole, phthalazine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, and [1,2,4]triazolo[4,3-a]pyrimidine; in each case unsubstituted, mono- or polysubstituted with substituents independently of one another selected from —F, —Cl, —CN, —OH, ═O, —C₁₋₆-alkyl, —CHF₂, —CF₃, —C₁₋₆-alkylene-NH₂, —C₁₋₆-alkylene-NHC(═O)O—C₁₋₆-alkyl, —C₁₋₆-alkylene-OH, —C₁₋₆-alkylene-CHF₂, —C₁₋₆-alkylene-CF₃, —C₁₋₆-alkylene-cyclopropyl, -cyclopropyl, —O-cyclopropyl, —C₁₋₆-alkylene-NHC(═O)—O—C₁₋₆-alkyl, —C(═O)O—C₁₋₆-alkyl, —N(C₁₋₆-alkyl)₂, —OC₁₋₆-alkyl, —OCF₃, —O—C₁₋₆-alkylene-N(C₁₋₆-alkyl)₂, —S(═O)₂—C₁₋₆-alkyl, -azetidine, —C₁₋₆-alkylene-O-tetrahydropyran, or -piperazine substituted with —C₁₋₆-alkyl; particularly in each case unsubstituted, mono- or polysubstituted with substituents independently of one another selected from —F, —Cl, —CN, —OH, ═O, —C₁₋₆-alkyl, —CHF₂, —CF₃, —C₁₋₆-alkylene-NH₂, —C₁₋₆-alkylene-NHC(═O)O—C₁₋₆-alkyl, —C₁₋₆-alkylene-OH, —C₁₋₆-alkylene-NHC(═O)—O—C₁₋₆-alkyl, —C(═O)O—C₁₋₆-alkyl, —N(C₁₋₆-alkyl)₂, —OC₁₋₆-alkyl, —OCF₃, —O—C₁₋₆-alkylene-N(C₁₋₆-alkyl)₂, —S(═O)₂—C₁₋₆-alkyl, -azetidine, —C₁₋₆-alkylene-O-tetrahydropyran, or -piperazine substituted with —C₁₋₆-alkyl; or represents (ii) -oxetanyl, unsubstituted, mono- or polysubstituted.

8. The compound of any one of claims 1 to 6, wherein the 3-14-membered cycloalkyl, saturated or unsaturated within the definition of V is cyclopropyl, cyclobutyl, cyclopentyl, cyclohexyl, cycloheptyl, cyclooctyl, cyclononyl, or cyclodecyl including unfused or unbridged, fused, or bridged cycloalkyls; in each case unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

9. The compound of any one of claims 1 to 6 wherein the 5-14-membered aryl within the definition of V is phenyl or another 5-14-membered aryl, unsubstituted, mono- or polysubstituted with substituents independently of one another selected from —F, —Cl, —Br, —I, CF₃, —CF₂H, C₁-C₆ alkyl, —CN, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

10. The compound of any one of claims 1 to 6 wherein the 3-14-membered heterocycloalkyl within the definition of V is selected from azepane, 1,4-oxazepane, azetane, azetidine, aziridine, azocane, diazepane, dioxane, dioxolane, dithiane, dithiolane, imidazolidine, isothiazolidine, isoxalidine, morpholine, oxazolidine, oxane, oxepane, oxetane, oxirane, piperazine, piperidine, pyrazolidine, pyrrolidine, quinuclidine, tetrahydrofurane, tetrahydropyrane, tetrahydrothiopyrane, thiazolidine, thietane, thiirane, thiolane, thiomorpholine, indoline, dihydrobenzofuran, dihydrobenzo-thiophene, 1,1-dioxothia-cyclohexane, 2-azaspiro[3.3]heptane, 2-oxaspiro[3.3]heptane, 7-azaspiro[3.5]nonane, 8-azabicyclo[3.2.1]octane, 9-azabicyclo[3.3.1]nonane, hexahydro-1H-pyrrolizine, hexa-hydro-cyclopenta[c]pyrrole, octahydro-cyclopenta[c]pyrrole, and octahydro-pyrrolo[1,2-a]pyrazine; in each case unsubstituted, mono- or polysubstituted with substituents independently of one another selected from —F, —Cl, —Br, —I, OF₃, —CF₂H, C₁-C₆ alkyl, —ON, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR, —S(═O)R^{Y}, —S(═O)₂R^{Y}, —C(═O)R^{Y}, —C(═O)OR^{Y}, or —C(═O)NR^{Y}R^{Z}.

11. The compound of any one of claims 1 to 6 wherein V represents C₁-C₆ alkyl or C₁-C₆ heteroalkyl, saturated or unsaturated, unsubstituted, mono- or polysubstituted with substituents independently of one another selected from —F, —Cl, —Br, —I, CF₃, —CF₂H, C₁-C₆ alkyl, —ON, —NO, —NO₂, ═O, ═S, —SF₅, —R^{Y}, —OR^{Y}, —OC(═O)R^{Y}, —NR^{Y}R^{Z}, —NR^{Y}C(═O)R^{Z}, —SR^{Y}, —S(═O)R^{Y}, —S(═O)₂R^{Y}, —C(═O)R^{Y}, —C(═O)OR^{Y}, or —C(═O)NR^{Y}R^{Z}.

12. The compound of any one of claims 1 to 6 wherein V is a residue selected from the group consisting of:

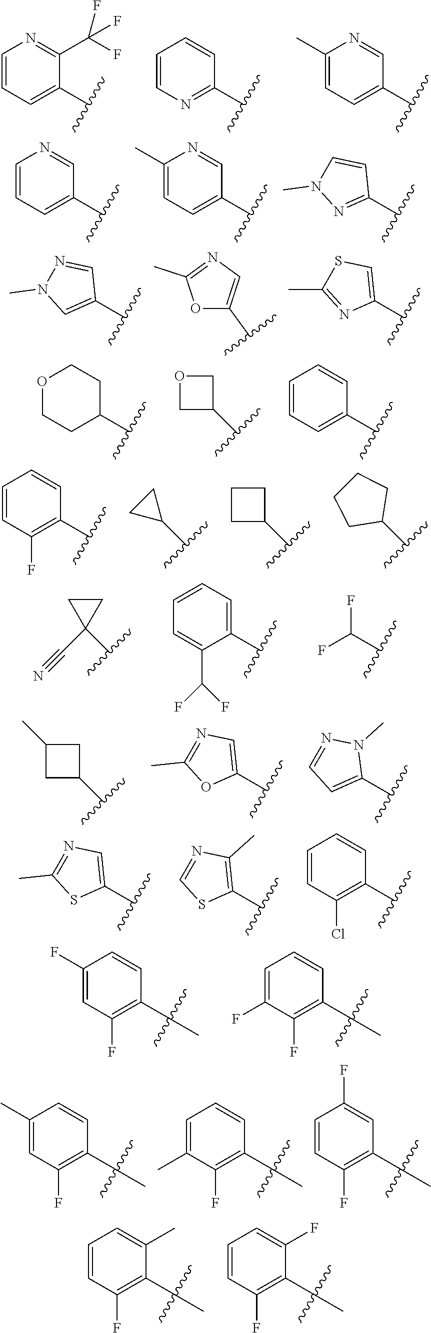

13. The compound according to claim 1, wherein R¹ represents —H, —F, —Cl, —Br, —I, —C₁₋₆-alkyl, —O—C₁₋₆-alkyl, —C₁₋₆-alkylene-O—C₁₋₆-alkyl, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, —C₁₋₆-alkylene-N(C₁₋₆-alkyl)-C₁₋₆-alkylene-CF₃, —C(═O)C₁₋₆-alkyl, —C(═O)OC₁₋₆-alkyl, —C(═O)NH₂, —C(═O)NHC₁₋₆-alkyl, —C(═O)N(C₁₋₆-alkyl)₂, —S(═O)—C₁₋₆-alkyl, —S(═O)₂—C₁₋₆-alkyl, —O—C₁₋₆-alkyl, -cyclopropyl unsubstituted, cyclobutyl unsubstituted, cyclopentyl unsubstituted or cyclohexyl unsubstituted.

14. The compound according to claim 1, wherein R³ represents —H, —OH, —C₁₋₆-alkyl, —C₁₋₆-alkylene-OH, —C₁₋₆-alkylene-O—C₁₋₆-alkyl, —C₁₋₆-alkylene-NH₂, —C₁₋₆-alkylene-NH(C₁₋₆-alkyl), —C₁₋₆-alkylene-N(C₁₋₆-alkyl)₂, —CF₃, —CF₂H, —CFH₂, —CF₂Cl, —CFCl₂, —C₁₋₆-alkylene-CF₃, —C₁₋₆-alkylene-CF₂H, —C₁₋₆-alkylene-CFH₂, —C₁₋₆-alkylene-NH—C₁₋₆-alkylene-CF₃, or —C₁₋₆-alkylene-N(C₁₋₆-alkyl)-C₁₋₆-alkylene-CF₃.

15. The compound according to any one of claims 1 to 14, wherein R⁴ represents
—H;
—S(═O)₂C₁₋₆-alkyl, saturated, unsubstituted, monosubstituted or polysubstituted with —F;
—S(═O)₂(3-14-membered cycloalkyl), saturated, unsubstituted;
—C₁₋₆-alkyl, saturated, unsubstituted mono- or polysubstituted;
3-14-membered cycloalkyl or —C₁₋₆-alkylene-(3-14-membered cycloalkyl), each unsubstituted, mono- or polysubstituted;
3-14-membered heterocycloalkyl or —C₁₋₆-alkylene-(3-14-membered heterocycloalkyl), unsubstituted, mono- or polysubstituted;
-phenyl, or —C₁₋₆-alkylene-phenyl, each unsubstituted, mono- or polysubstituted; or
5-14-membered heteroaryl or —C₁₋₆-alkylene-(5-14-membered heteroaryl), each unsubstituted, mono- or polysubstituted.

16. The compound according to any one of claims 1 to 14 wherein R⁴ represents a 3-14-membered cycloalkyl (preferably a 3, 4, 5 or 6-membered cycloalkyl), saturated or unsaturated, unsubstituted, mono- or polysubstituted; wherein said 3-14-membered cycloalkyl is connected through —C₁-C₆-alkylene-, saturated or unsaturated, unsubstituted, mono- or polysubstituted; or a 3-14-membered heterocycloalkyl (preferably a 4, 5 or 6-membered heterocycloalkyl), saturated or unsaturated, unsubstituted, mono- or polysubstituted; wherein said 3-14-membered heterocycloalkyl is connected through —C₁-C₆-alkylene-, saturated or unsaturated, unsubstituted, mono- or polysubstituted; or a 6-14-membered aryl (preferably a 6-membered aryl), unsubstituted, mono- or polysubstituted; wherein said 6-14-membered aryl is connected through —C₁-C₆-alkylene-, saturated or unsaturated, unsubstituted, mono- or polysubstituted; or a 5-14-membered heteroaryl (preferably a 5 or 6-membered heteroaryl), unsubstituted, mono- or polysubstituted; wherein said 5-14-membered heteroaryl is connected through —C₁-C₆-alkylene-, saturated or unsaturated, unsubstituted, mono- or polysubstituted.

17. The compound according to any one of claims 1 to 16 wherein R³ is H and R⁴ is a residue selected from the group consisting of:

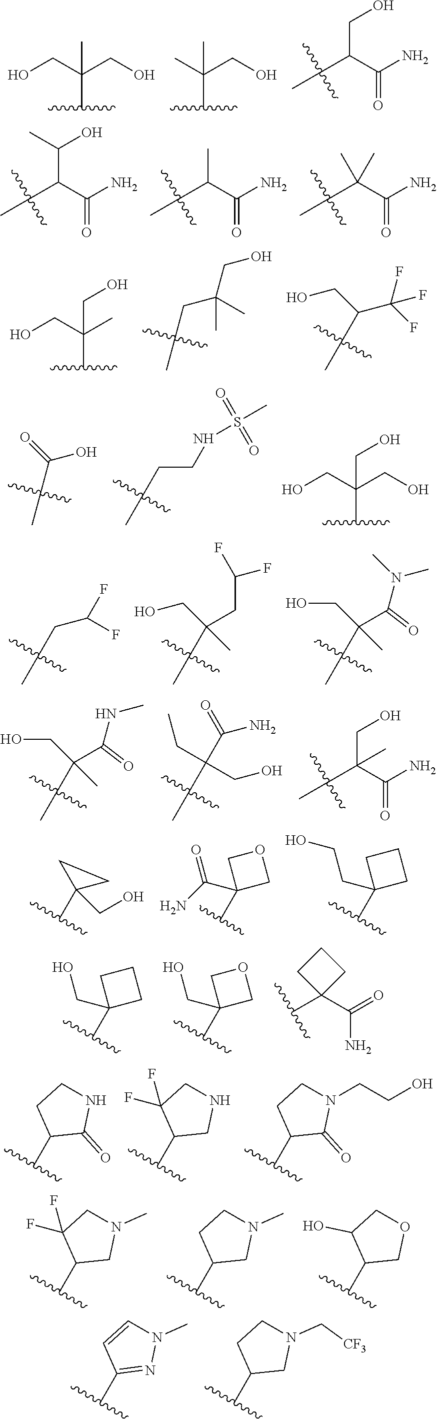

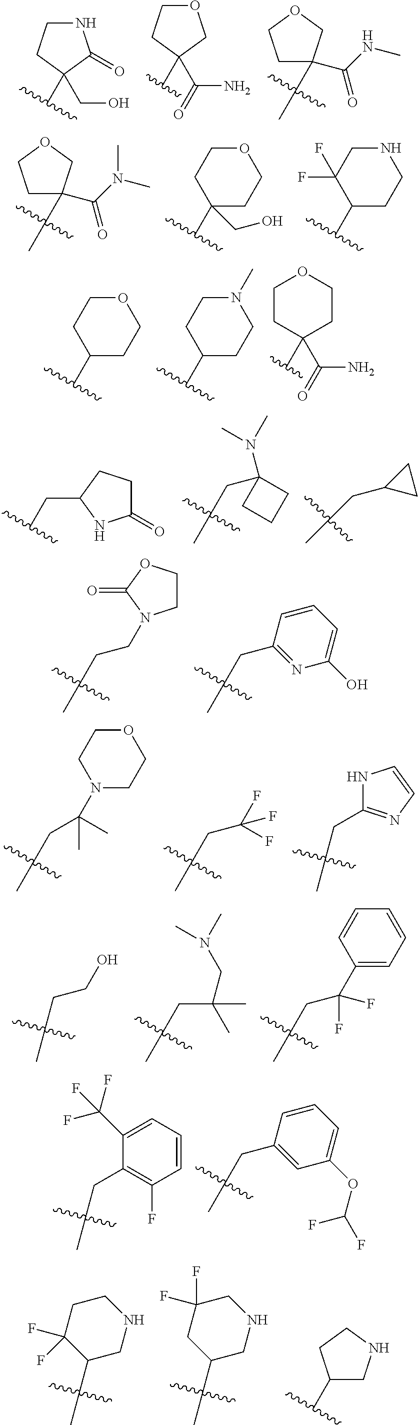

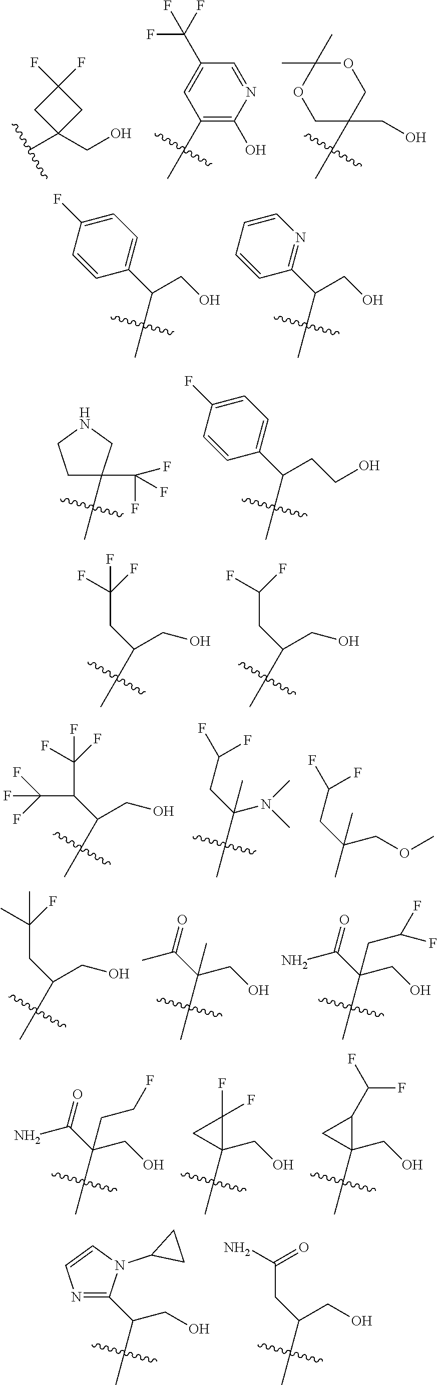

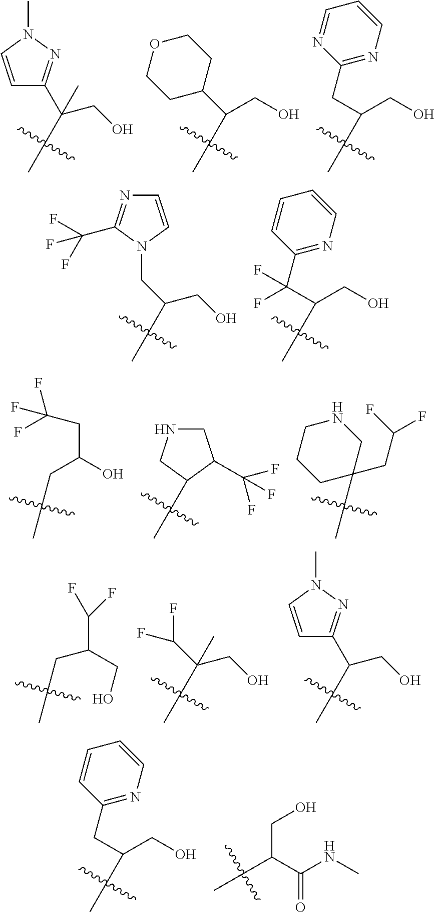

18. The compound of claim 1 wherein R³ and R⁴ together form a heterocycle selected from the group consisting of pyrrolidine, piperidine, morpholine, and piperazine, in each case unsubstituted, mono- or polysubstituted with substituents independently of one another selected from the group consisting of —F, —C₁₋₆-alkyl, —NH₂, —NHCH₃, —N(CH₃)₂, —C(═O)NH—C₁₋₆-alkyl, —C(═O)N(C₁₋₆-alkyl)₂, —C(═O)O—C₁₋₆-alkyl, —NHC(═O)O—C₁₋₆-alkyl, -pyridyl unsubstituted, and 1,2,4-oxadiazole unsubstituted or monosubstituted with —C₁₋₆-alkyl.

19. The compound of claim 1, wherein R⁵ and R^{5′} independently of one another represent
—H;
—C₁-C₆-alkyl, saturated or unsaturated, unsubstituted, mono- or polysubstituted;
—C₁-C₆-heteroalkyl, saturated or unsaturated, unsubstituted, mono- or polysubstituted;
3-14-membered cycloalkyl, saturated or unsaturated, unsubstituted, mono- or polysubstituted; wherein said 3-14-membered cycloalkyl is optionally connected through —C₁-C₆-alkylene- or —C₁-C₆-heteroalkylene-, in each case saturated or unsaturated, unsubstituted, mono- or polysubstituted.

20. The compound according to any one of claims 1 to 19, wherein R⁶, R⁷ and R⁸ independently of one another represent
—H, —F, —Cl, —Br, —I, —OH, —SH, —SF₅, —CN, —NO₂, —C(═O)OH, —NH₂,
—C₁₋₆-alkyl, —CF₃, —CHF₂, —CH₂F,
—O—C₁₋₆-alkyl, —OCF₃, —OCHF₂, —OCH₂F,
—NHC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—N(C₁₋₆-alkyl)₂ unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—C(═O)OC₁₋₆-alkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂;
—OC(═O)C₁₋₆-alkyl unsubstituted or substituted with one or more substituents independently of one another selected from —OH, ═O, —F, —C, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂; or
—C₁₋₆-heteroalkyl unsubstituted or substituted with one or more substituents independently of one another selected from —OH, ═O, —F, —Cl, —Br, —I, —SH, ═S, —CN, —CF₃, —CHF₂, —CH₂F, —OCF₃, —OCHF₂, —OCH₂F, SF₅, —NO₂, —C(═O)OH, —NH₂, and —C(═O)NH₂.

21. The compound of claim 1, which is selected from the group consisting of compounds 001-036 as shown in the table below:
Structure and Compound CODE 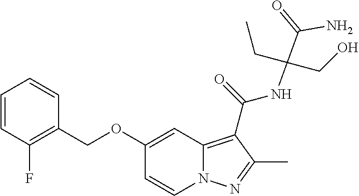 [SMILES: CCC(CO)(NC(=O)c1c(C)nn2ccc(OCc3ccccc3F)cc12)C(N)=O]
Cpd 001 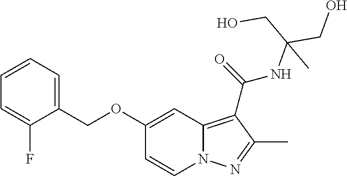 [SMILES: Cc1nn2ccc(OCc3ccccc3F)cc2c1C(=O)NC(C)(CO)CO]
Cpd 002 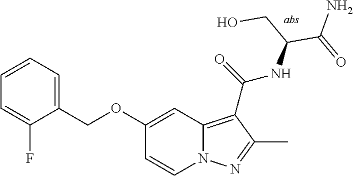 [SMILES: Cc1nn2ccc(OCc3ccccc3F)cc2c1C(=O)N[C@@H](CO)C(N)=O]
Cpd 003 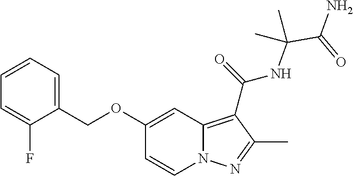 [SMILES: Cc1nn2ccc(OCc3ccccc3F)cc2c1C(=O)NC(C)(C)C(N)=O]
Cpd 004 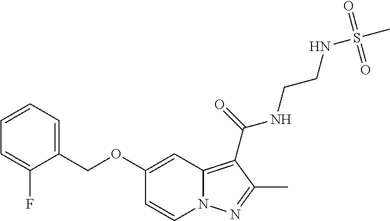 [SMILES: Cc1nn2ccc(OCc3ccccc3F)cc2c1C(=O)NCCNS(C)(=O)=O]
Cpd 005 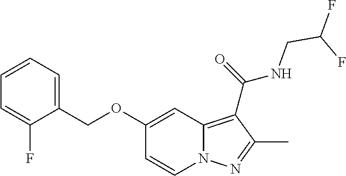 [SMILES: Cc1nn2ccc(OCc3ccccc3F)cc2c1C(=O)NCC(F)F]
Cpd 006 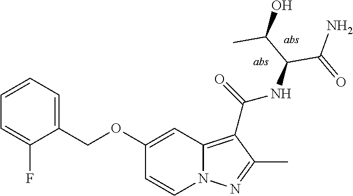 [SMILES: Cc1nn2ccc(OCc3ccccc3F)cc2c1C(=O)N[C@H](C(N)=O)[C@@H](C)O]
Cpd 007 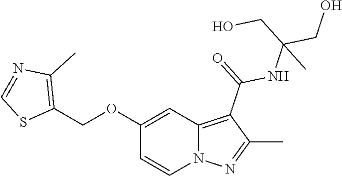 [SMILES: Cc1ncsc1COc1ccn2nc(C)c(C(=O)NC(C)(CO)CO)c2c1]
Cpd 008 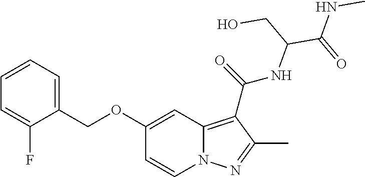 [SMILES: CNC(=O)C(CO)NC(=O)c1c(C)nn2ccc(OCc3ccccc3F)cc12]
Cpd 009 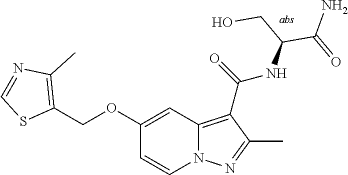 [SMILES: Cc1ncsc1COc1ccn2nc(C)c(C(=O)N[C@@H](CO)C(N)=O)c2c1]
Cpd 010 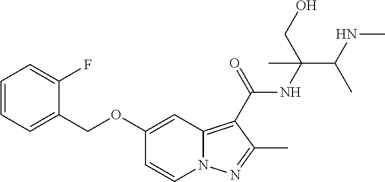 [SMILES: CNC(C)C(C)(CO)NC(=O)c1c(C)nn2ccc(OCc3ccccc3F)cc12]
Cpd 011 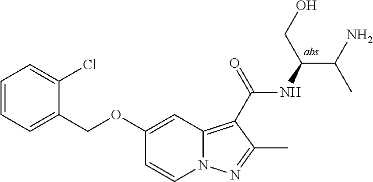 [SMILES: Cc1nn2ccc(OCc3ccccc3Cl)cc2c1C(=O)N[C@@H](CO)C(C)N]
Cpd 012 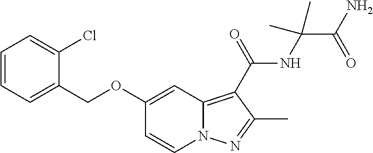 [SMILES: Cc1nn2ccc(OCc3ccccc3Cl)cc2c1C(=O)NC(C)(C)C(N)=O]
Cpd 013 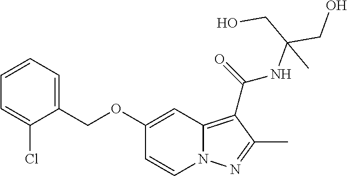 [SMILES: Cc1nn2ccc(OCc3ccccc3Cl)cc2c1C(=O)NC(C)(CO)CO]
Cpd 014 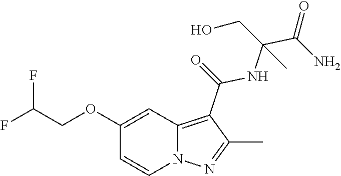 [SMILES: Cc1nn2ccc(OCC(F)F)cc2c1C(=O)NC(C)(CO)C(N)=O]
Cpd 015 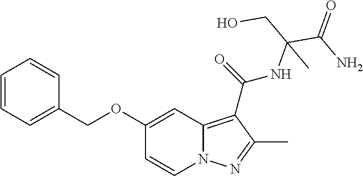 [SMILES: Cc1nn2ccc(OCc3ccccc3)cc2c1C(=O)NC(C)(CO)C(N)=O]
Cpd 016 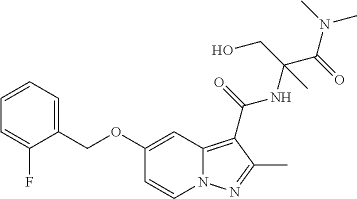 [SMILES: Cc1nn2ccc(OCc3ccccc3F)cc2c1C(=O)NC(C)(CO)C(=O)N(C)C]
Cpd 017 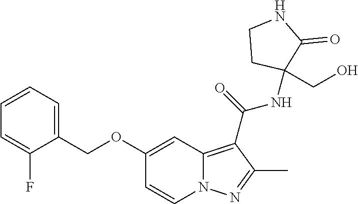 [SMILES: Cc1nn2ccc(OCc3ccccc3F)cc2c1C(=O)NC1(CO)CCNC1=O]
Cpd 018 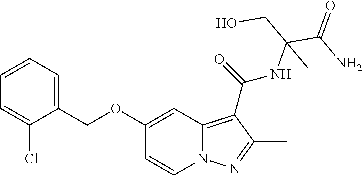 [SMILES: Cc1nn2ccc(OCc3ccccc3Cl)cc2c1C(=O)NC(C)(CO)C(N)=O]
Cpd 019 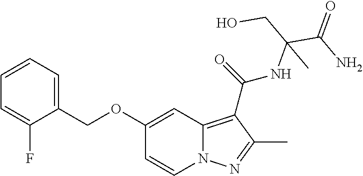 [SMILES: Cc1nn2ccc(OCc3ccccc3F)cc2c1C(=O)NC(C)(CO)C(N)=O]
Cpd 020 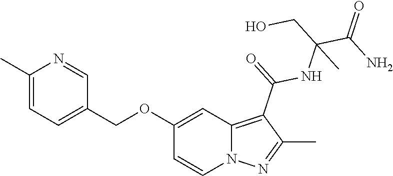 [SMILES: Cc1ccc(COc2ccn3nc(C)c(C(=O)NC(C)(CO)C(N)=O)c3c2)cn1]
Cpd 021 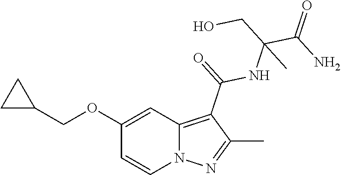 [SMILES: Cc1nn2ccc(OCC3CC3)cc2c1C(=O)NC(C)(CO)C(N)=O]
Cpd 022 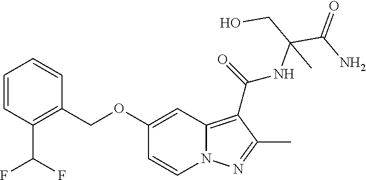 [SMILES: Cc1nn2ccc(OCc3ccccc3C(F)F)cc2c1C(=O)NC(C)(CO)C(N)=O]
Cpd 023 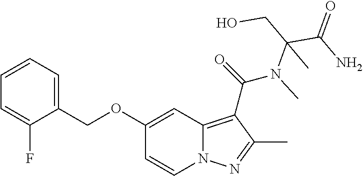 [SMILES: Cc1nn2ccc(OCc3ccccc3F)cc2c1C(=O)N(C)C(C)(CO)C(N)=O]
Cpd 024 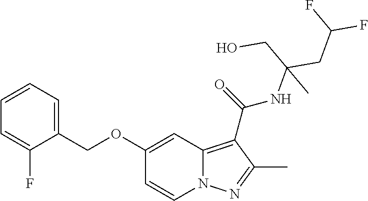 [SMILES: Cc1nn2ccc(OCc3ccccc3F)cc2c1C(=O)NC(C)(CO)CC(F)F]
Cpd 025 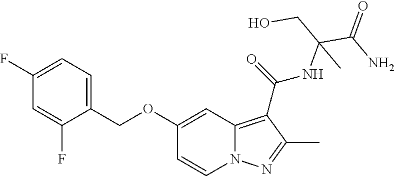 [SMILES: Cc1nn2ccc(OCc3ccc(F)cc3F)cc2c1C(=O)NC(C)(CO)C(N)=O]
Cpd 026 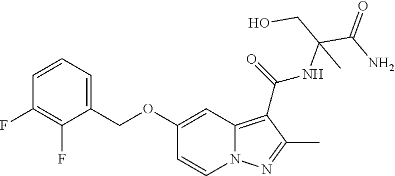 [SMILES: Cc1nn2ccc(OCc3cccc(F)c3F)cc2c1C(=O)NC(C)(CO)C(N)=O]
Cpd 027 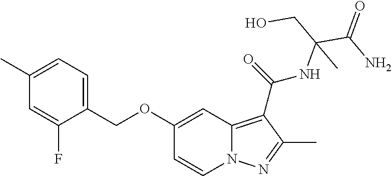 [SMILES: Cc1ccc(COc2ccn3nc(C)c(C(=O)NC(C)(CO)C(N)=O)c3c2)c(F)c1]
Cpd 028 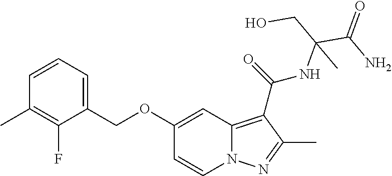 [SMILES: Cc1cccc(COc2ccn3nc(C)c(C(=O)NC(C)(CO)C(N)=O)c3c2)c1F]
Cpd 029 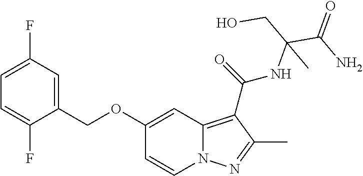 [SMILES: Cc1nn2ccc(OCc3cc(F)ccc3F)cc2c1C(=O)NC(C)(CO)C(N)=O]
Cpd 030 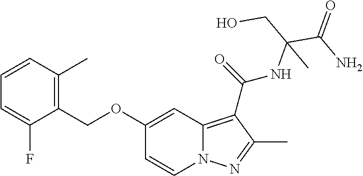 [SMILES: Cc1cccc(F)c1COc1ccn2nc(C)c(C(=O)NC(C)(CO)C(N)=O)c2c1]
Cpd 031 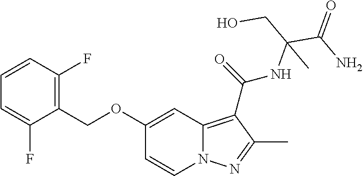 [SMILES: Cc1nn2ccc(OCc3c(F)cccc3F)cc2c1C(=O)NC(C)(CO)C(N)=O]
Cpd 032 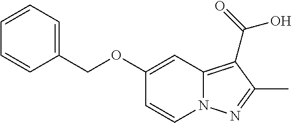 [SMILES: Cc1nn2ccc(OCc3ccccc3)cc2c1C(=O)O]
Cpd 033 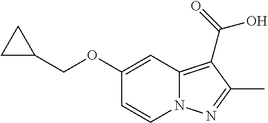 [SMILES: Cc1nn2ccc(OCC3CC3)cc2c1C(=O)O]
Cpd 034 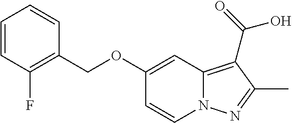 [SMILES: Cc1nn2ccc(OCc3ccccc3F)cc2c1C(=O)O]
Cpd 035 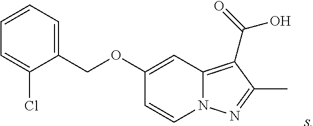
Cpd 036

22. The compound of claim 1, which is selected from the group consisting of compounds 037-091 as shown in the table below:
Structure and Compound CODE 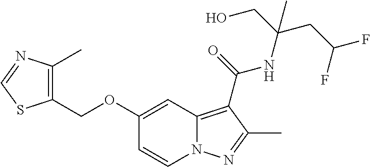 [SMILES: Cc1ncsc1COc1ccn2nc(C)c(C(=O)NC(C)(CO)CC(F)F)c2c1]
Cpd 037 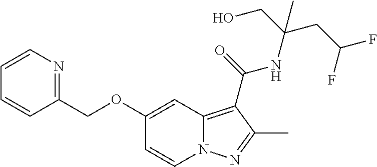 [SMILES: Cc1nn2ccc(OCc3ccccn3)cc2c1C(=O)NC(C)(CO)CC(F)F]
Cpd 038 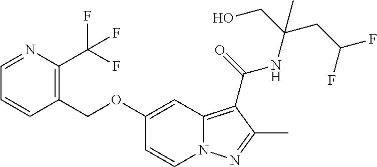 [SMILES: Cc1nn2ccc(OCc3cccnc3C(F)(F)F)cc2c1C(=O)NC(C)(CO)CC(F)F]
Cpd 039 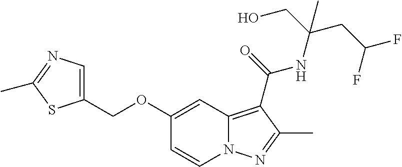 [SMILES: Cc1ncc(COc2ccn3nc(C)c(C(=O)NC(C)(CO)CC(F)F)c3c2)s1]
Cpd 040 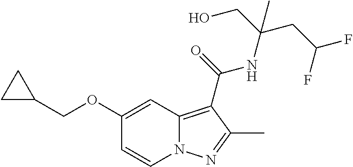 [SMILES: Cc1nn2ccc(OCC3CC3)cc2c1C(=O)NC(C)(CO)CC(F)F]
Cpd 041 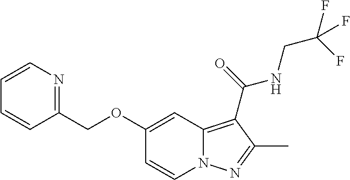 [SMILES: Cc1nn2ccc(OCc3ccccn3)cc2c1C(=O)NCC(F)(F)F]
Cpd 042 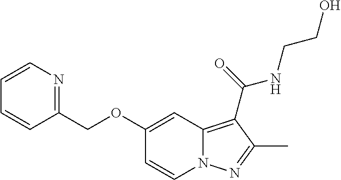 [SMILES: Cc1nn2ccc(OCc3ccccn3)cc2c1C(=O)NCCO]
Cpd 043 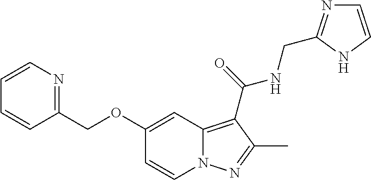 [SMILES: Cc1nn2ccc(OCc3ccccn3)cc2c1C(=O)NCc1ncc[nH]1]
Cpd 044 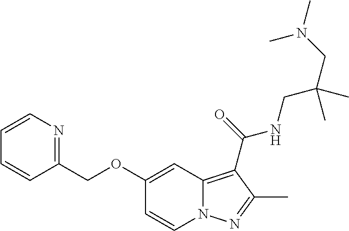 [SMILES: Cc1nn2ccc(OCc3ccccn3)cc2c1C(=O)NCC(C)(C)CN(C)C]
Cpd 045 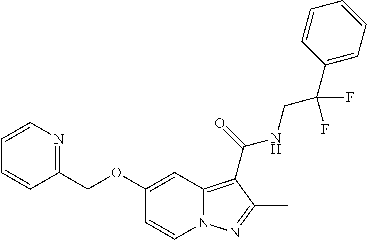 [SMILES: Cc1nn2ccc(OCc3ccccn3)cc2c1C(=O)NCC(F)(F)c1ccccc1]
Cpd 046 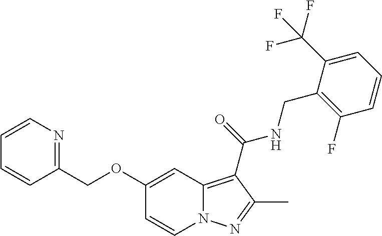 [SMILES: Cc1nn2ccc(OCc3ccccn3)cc2c1C(=O)NCc1c(F)cccc1C(F)(F)F]
Cpd 047 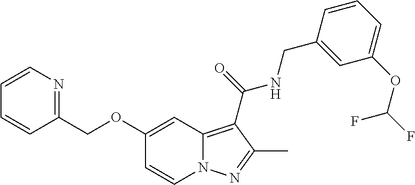 [SMILES: Cc1nn2ccc(OCc3ccccn3)cc2c1C(=O)NCc1cccc(OC(F)F)c1]
Cpd 048 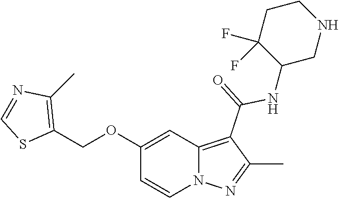 [SMILES: Cc1ncsc1COc1ccn2nc(C)c(C(=O)NC3CNCCC3(F)F)c2c1]
Cpd 049 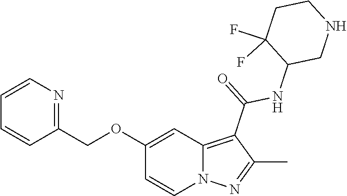 [SMILES: Cc1nn2ccc(OCc3ccccn3)cc2c1C(=O)NC1CNCCC1(F)F]
Cpd 050 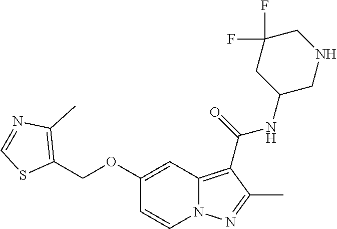 [SMILES: Cc1ncsc1COc1ccn2nc(C)c(C(=O)NC3CNCC(F)(F)C3)c2c1]
Cpd 051 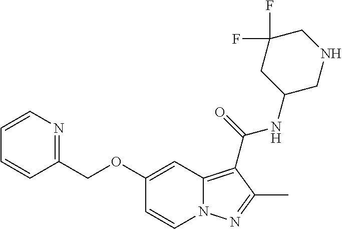 [SMILES: Cc1nn2ccc(OCc3ccccn3)cc2c1C(=O)NC1CNCC(F)(F)C1]
Cpd 052 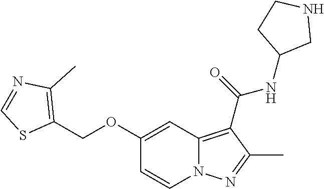 [SMILES: Cc1ncsc1COc1ccn2nc(C)c(C(=O)NC3CCNC3)c2c1]
Cpd 053 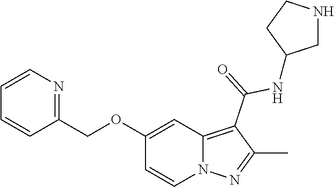 [SMILES: Cc1nn2ccc(OCc3ccccn3)cc2c1C(=O)NC1CCNC1]
Cpd 054 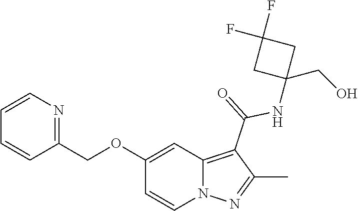 [SMILES: Cc1nn2ccc(OCc3ccccn3)cc2c1C(=O)NC1(CO)CC(F)(F)C1]
Cpd 055 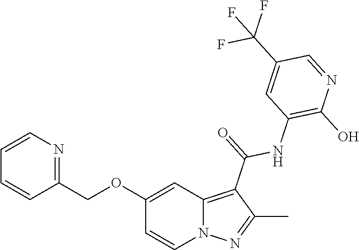 [SMILES: Cc1nn2ccc(OCc3ccccn3)cc2c1C(=O)Nc1cc(C(F)(F)F)cnc1O]
Cpd 056 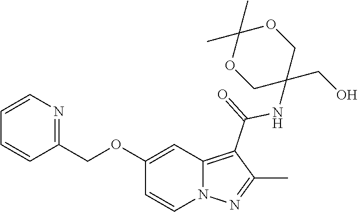 [SMILES: Cc1nn2ccc(OCc3ccccn3)cc2c1C(=O)NC1(CO)COC(C)(C)OC1]
Cpd 057 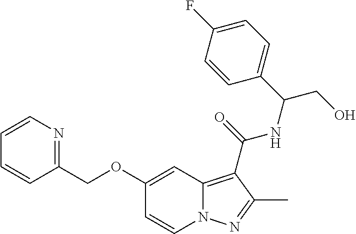 [SMILES: Cc1nn2ccc(OCc3ccccn3)cc2c1C(=O)NC(CO)c1ccc(F)cc1]
Cpd 058 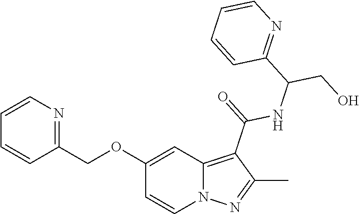 [SMILES: Cc1nn2ccc(OCc3ccccn3)cc2c1C(=O)NC(CO)c1ccccn1]
Cpd 059 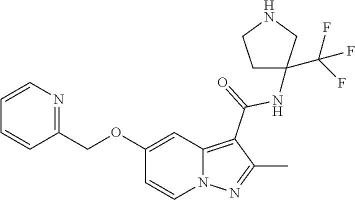 [SMILES: Cc1nn2ccc(OCc3ccccn3)cc2c1C(=O)NC1(C(F)(F)F)CCNC1]
Cpd 060 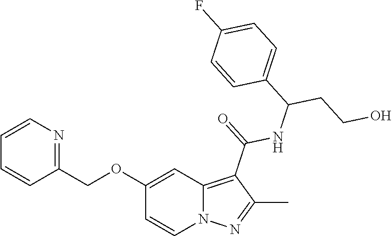 [SMILES: Cc1nn2ccc(OCc3ccccn3)cc2c1C(=O)NC(CCO)c1ccc(F)cc1]
Cpd 061 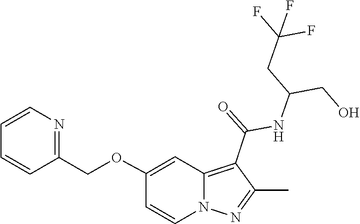 [SMILES: Cc1nn2ccc(OCc3ccccn3)cc2c1C(=O)NC(CO)CC(F)(F)F]
Cpd 062 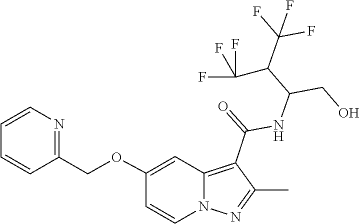 [SMILES: Cc1nn2ccc(OCc3ccccn3)cc2c1C(=O)NC(CO)C(C(F)(F)F)C(F)(F)F]
Cpd 063 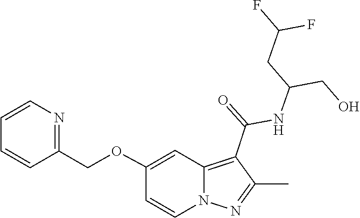 [SMILES: Cc1nn2ccc(OCc3ccccn3)cc2c1C(=O)NC(CO)CC(F)F]
Cpd 064 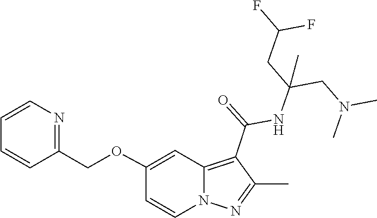 [SMILES: Cc1nn2ccc(OCc3ccccn3)cc2c1C(=O)NC(C)(CC(F)F)CN(C)C]
Cpd 065 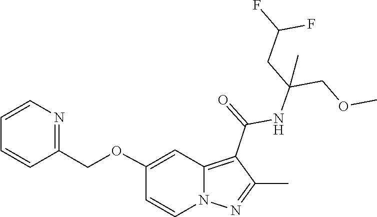 [SMILES: COCC(C)(CC(F)F)NC(=O)c1c(C)nn2ccc(OCc3ccccn3)cc12]
Cpd 066 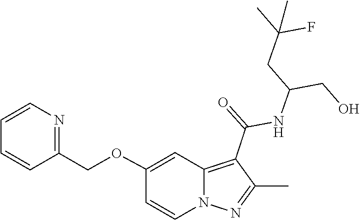 [SMILES: Cc1nn2ccc(OCc3ccccn3)cc2c1C(=O)NC(CO)CC(C)(C)F]
Cpd 067 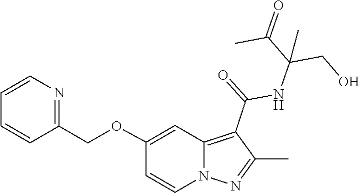 [SMILES: CC(=O)C(C)(CO)NC(=O)c1c(C)nn2ccc(OCc3ccccn3)cc12]
Cpd 068 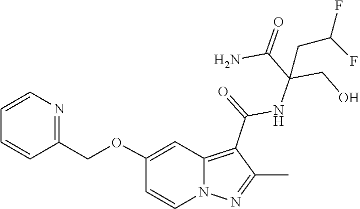 [SMILES: Cc1nn2ccc(OCc3ccccn3)cc2c1C(=O)NC(CO)(CC(F)F)C(N)=O]
Cpd 069 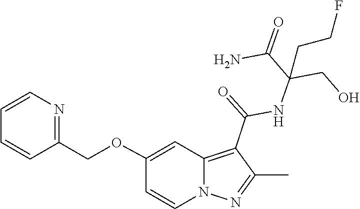 [SMILES: Cc1nn2ccc(OCc3ccccn3)cc2c1C(=O)NC(CO)(CCF)C(N)=O]
Cpd 070 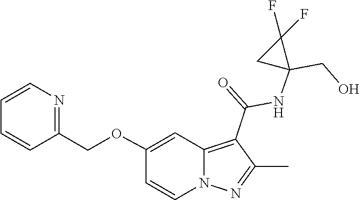 [SMILES: Cc1nn2ccc(OCc3ccccn3)cc2c1C(=O)NC1(CO)CC1(F)F]
Cpd 071 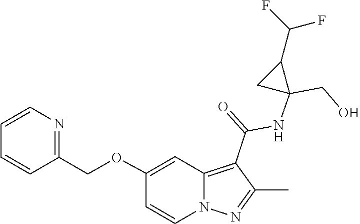 [SMILES: Cc1nn2ccc(OCc3ccccn3)cc2c1C(=O)NC1(CO)CC1C(F)F]
Cpd 072 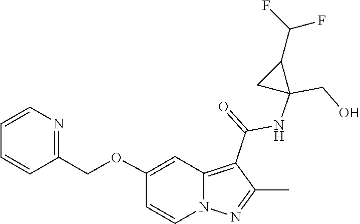 [SMILES: Cc1nn2ccc(OCc3ccccn3)cc2c1C(=O)NC1(CO)CC1C(F)F]
Cpd 073 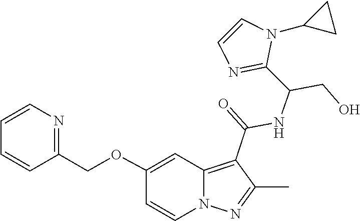 [SMILES: Cc1nn2ccc(OCc3ccccn3)cc2c1C(=O)NC(CO)c1nccn1C1CC1]
Cpd 074 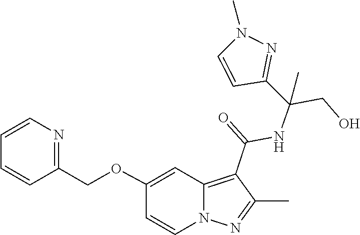 [SMILES: Cc1nn2ccc(OCc3ccccn3)cc2c1C(=O)NC(C)(CO)c1ccn(C)n1]
Cpd 075 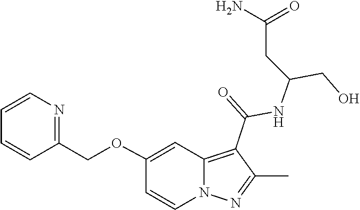 [SMILES: Cc1nn2ccc(OCc3ccccn3)cc2c1C(=O)NC(CO)CC(N)=O]
Cpd 076 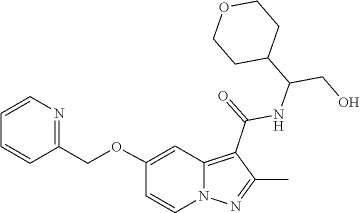 [SMILES: Cc1nn2ccc(OCc3ccccn3)cc2c1C(=O)NC(CO)C1CCOCC1]
Cpd 077 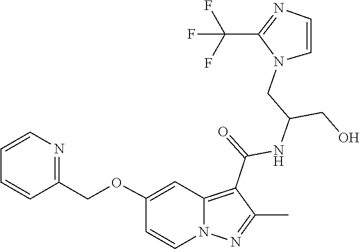 [SMILES: Cc1nn2ccc(OCc3ccccn3)cc2c1C(=O)NC(CO)Cn1ccnc1C(F)(F)F]
Cpd 078 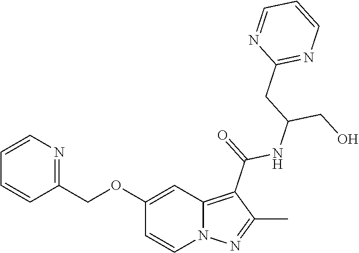 [SMILES: Cc1nn2ccc(OCc3ccccn3)cc2c1C(=O)NC(CO)Cc1ncccn1]
Cpd 079 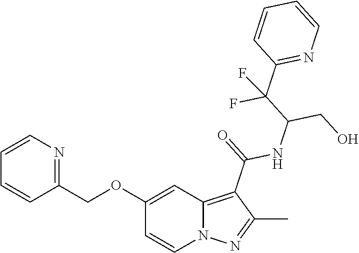 [SMILES: Cc1nn2ccc(OCc3ccccn3)cc2c1C(=O)NC(CO)C(F)(F)c1ccccn1]
Cpd 080 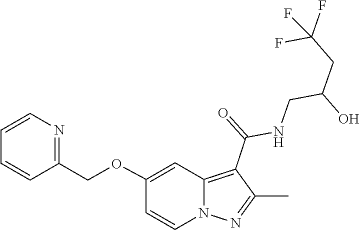 [SMILES: Cc1nn2ccc(OCc3ccccn3)cc2c1C(=O)NCC(O)CC(F)(F)F]
Cpd 081 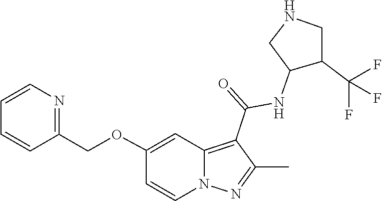 [SMILES: Cc1nn2ccc(OCc3ccccn3)cc2c1C(=O)NC1CNCC1C(F)(F)F]
Cpd 082 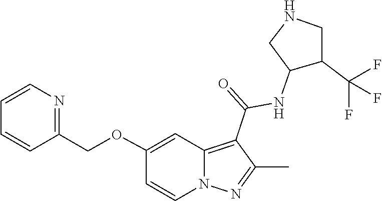 [SMILES: Cc1nn2ccc(OCc3ccccn3)cc2c1C(=O)NC1CNCC1C(F)(F)F]
Cpd 083 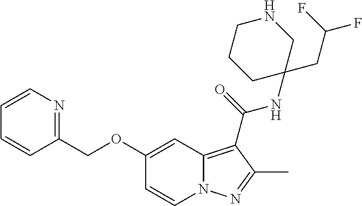 [SMILES: Cc1nn2ccc(OCc3ccccn3)cc2c1C(=O)NC1(CC(F)F)CCCNC1]
Cpd 084 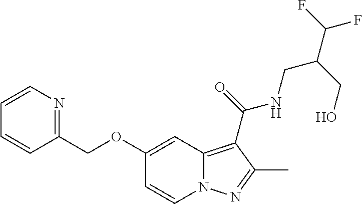 [SMILES: Cc1nn2ccc(OCc3ccccn3)cc2c1C(=O)NCC(CO)C(F)F]
Cpd 085 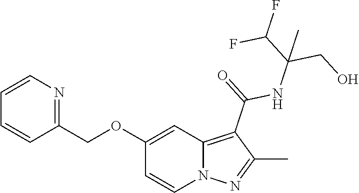 [SMILES: Cc1nn2ccc(OCc3ccccn3)cc2c1C(=O)NC(C)(CO)C(F)F]
Cpd 086 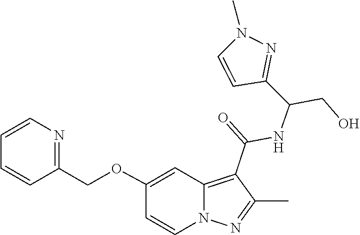 [SMILES: Cc1nn2ccc(OCc3ccccn3)cc2c1C(=O)NC(CO)c1ccn(C)n1]
Cpd 087 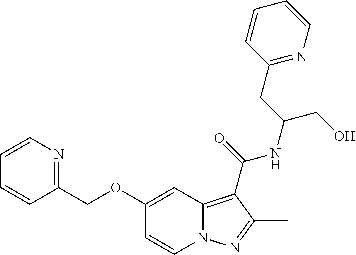 [SMILES: Cc1nn2ccc(OCc3ccccn3)cc2c1C(=O)NC(CO)Cc1ccccn1]
Cpd 088 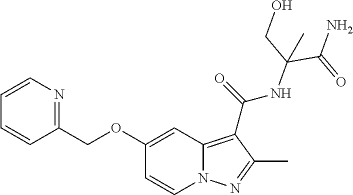 [SMILES: Cc1nn2ccc(OCc3ccccn3)cc2c1C(=O)NC(C)(CO)C(N)=O]
Cpd 089 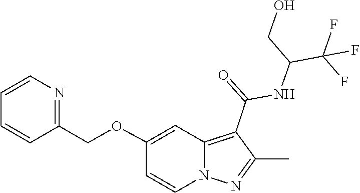 [SMILES: Cc1nn2ccc(OCc3ccccn3)cc2c1C(=O)NC(CO)C(F)(F)F]
Cpd 090 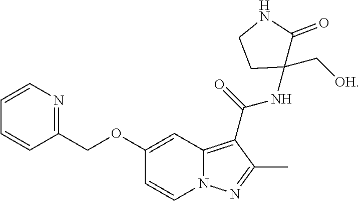 [SMILES: Cc1nn2ccc(OCc3ccccn3)cc2c1C(=O)NC1(CO)CCNC1=O]
Cpd 091

23. A pharmaceutical composition comprising a compound according to any one of claims 1 to 22.

24. The compound according to any one of claims 1 to 22 or the pharmaceutical composition according to claim 23, for use in the treatment of pain.

25. The compound or the pharmaceutical composition for use in the treatment of pain according to claim 24, wherein the pain is selected from nociceptive pain, inflammatory pain, and neuropathic pain; preferably post-operative pain.

26. A method of treating of pain comprising administering a compound according to any one of claims 1-22, or a pharmaceutical composition according to any one of claims 23 to 25, to a subject in need thereof.

27. The method of claim 26 wherein the pain is selected from nociceptive pain, inflammatory pain, and neuropathic pain; preferably post-operative pain.

28. The compound according to any one of claims 1 to 22 or the pharmaceutical composition according to claim 23, for use in the treatment of epilepsy.

29. A method of treating of epilepsy comprising administering a compound according to any one of claims 1 to 22, or a pharmaceutical composition according to claim 23, to a subject in need thereof.
